(19) United States
(12) Patent Application Publication
(10) Pub. No.: US 2016/0242416 A1
(43) Pub. Date: Aug. 25, 2016

(54) FUNGICIDAL ISOXAZOLINE CARBINOLS

(71) Applicant: E I DU PONT DE NEMOURS AND COMPANY, Wilmington, DE (US)

(72) Inventors: ROBERT JAMES PASTERIS, NEWARK, DE (US); JEFFREY KEITH LONG, WILMINGTON, DE (US); SRINIVAS CHITTABOINA, NIZAMABAD DISTRICT (IN)

(21) Appl. No.: 15/050,770

(22) Filed: Feb. 23, 2016

Related U.S. Application Data
(60) Provisional application No. 62/119,292, filed on Feb. 23, 2015.

(51) Int. Cl. A01N 43/80; C07D 413/06; C07D 413/04

(52) U.S. Cl. CPC A01N 43/80; C07D 413/04; C07D 413/06

(57) ABSTRACT

Disclosed are compounds of Formula 1, including all stereoisomers, N-oxides, and salts thereof,

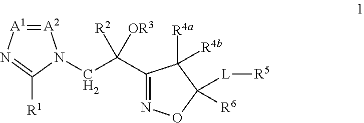

wherein
A¹, A², R¹, R², R³, R^{4a}, R^{4b}, L, R⁵ and R⁶ are as defined in the disclosure.
   Also disclosed are compositions containing the compounds of Formula 1 and methods for controlling plant disease caused by a fungal pathogen comprising applying an effective amount of a compound or a composition of the invention.

### FIELD OF THE INVENTION

This invention relates to certain isoxazoline carbinols, their N-oxides, salts and compositions, and methods of their use as fungicides.

### BACKGROUND OF THE INVENTION

The control of plant diseases caused by fungal plant pathogens is extremely important in achieving high crop efficiency. Plant disease damage to ornamental, vegetable, field, cereal, and fruit crops can cause significant reduction in productivity and thereby result in increased costs to the consumer. Many products are commercially available for these purposes, but the need continues for new compounds which are more effective, less costly, less toxic, environmentally safer or have different sites of action.

### SUMMARY OF THE INVENTION

This invention is directed to compounds of Formula 1 (including all stereoisomers), N-oxides, and salts thereof, agricultural compositions containing them and their use as fungicides:

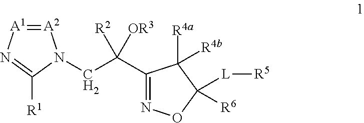

wherein
A¹ is CH or N;
A² is CH or N;
R¹ is hydrogen, halogen, SH, CN, SCN, C₁-C₆ alkylthio, C₁-C₆ haloalkylthio, C₂-C₆ alkenylthio, C₂-C₆ haloalkenylthio, C₂-C₆ alkynylthio or C₂-C₆ haloalkynylthio;
R² is C₁-C₆ alkyl, C₂-C₆ alkenyl or C₂-C₆ alkynyl, each optionally substituted with up to 5 substituents independently selected from R^{2a}; or C₃-C₈ cycloalkyl optionally substituted with up to 5 substituents independently selected from R^{2b}; or ZQ¹;
each R^{2a} is independently hydroxy, halogen, cyano, nitro, C₁-C₆ alkoxy, C₁-C₆ haloalkoxy, C₃-C₆ cycloalkyl or C₃-C₆ halocycloalkyl;
each R^{2b} is independently hydroxy, halogen, cyano, nitro, C₁-C₆ alkyl, C₁-C₆ haloalkyl, C₁-C₆ alkoxy or C₁-C₆ haloalkoxy;
Z is a direct bond, CH₂ or CH₂O wherein the carbon atom is attached to the remainder of Formula 1 and the oxygen atom is attached to Q¹;
Q¹ is a phenyl ring; or a 5- or 6-membered heteroaromatic ring containing ring members selected from carbon atoms and up to 4 heteroatoms independently selected from up to 2 O, up to 2 S and up to 4 N atoms, each ring optionally substituted with up to 3 substituents independently selected from R^{9a} on carbon atom ring members and R^{9b} on nitrogen atom ring members;
R³ is hydrogen, CHO, C₂-C₄ alkylcarbonyl, C₂-C₄ haloalkylcarbonyl, C₂-C₄ alkoxycarbonyl, C₂-C₄ haloalkoxycarbonyl, C₁-C₄ alkyl, C₁-C₄ haloalkyl, C₃-C₄ alkenyl, C₃-C₄ alkynyl or C₂-C₄ alkoxyalkyl;
R^{4a} is hydrogen, halogen, cyano, C₁-C₂ alkyl, C₁-C₂ haloalkyl or C₁-C₂ alkoxy;
R^{4b} is hydrogen, halogen, cyano, C₁-C₂ alkyl, C₁-C₂ haloalkyl or C₁-C₂ alkoxy;
L is a direct bond; or a 1-, 2-, 3- or 4-membered saturated, partially unsaturated or fully unsaturated chain containing chain members selected from carbon atoms and up to 2 heteroatoms independently selected from up to 1 O, up to 1 S, and up to 2 N, wherein the chain is optionally substituted with up to 4 substituents independently selected from R^{7a} on carbon atom chain members and R^{7b} on nitrogen atom chain members;
R⁵ is C₁-C₈ alkyl, C₁-C₈ haloalkyl, C₂-C₈ alkenyl, C₂-C₈ haloalkenyl, C₂-C₈ alkynyl, C₂-C₈ haloalkynyl, C₃-C₈ cycloalkyl, C₃-C₈ halocycloalkyl, C₄-C₁₀ alkylcycloalkyl, C₄-C₁₀ cycloalkylalkyl, C₄-C₁₀ halocycloalkylalkyl, C₅-C₁₀ alkylcycloalkylalkyl, C₂-C₈ alkoxyalkyl, C₂-C₈ haloalkoxyalkyl, C₄-C₁₀ cycloalkoxyalkyl, C₃-C₈ alkoxyalkoxyalkyl, C₂-C₈ alkylthioalkyl, C₂-C₈ haloalkylthioalkyl, C₂-C₈ alkylsulfinylalkyl, C₂-C₈ alkylsulfonylalkyl, C₂-C₈ alkylaminoalkyl, C₂-C₈ haloalkylaminoalkyl, C₃-C₈ dialkylaminoalkyl, C₄-C₁₀ cycloalkylaminoalkyl, C₃-C₈ alkoxycarbonylalkyl, C₃-C₈ haloalkoxycarbonylalkyl, C₁-C₈ alkoxy, C₁-C₈ haloalkoxy, C₂-C₈ alkenyloxy, C₂-C₈ haloalkenyloxy, C₃-C₈ alkynyloxy, C₃-C₈ haloalkynyloxy, C₃-C₈ cycloalkoxy, C₃-C₈ halocycloalkoxy, C₄-C₁₀ cycloalkylalkoxy, C₂-C₈ alkoxyalkoxy, C₂-C₈ alkylcarbonyloxy, C₂-C₈ haloalkylcarbonyloxy, C₁-C₈ alkylthio, C₁-C₈ haloalkylthio, C₃-C₈ cycloalkylthio, C₁-C₈ alkylamino, C₁-C₈ haloalkylamino, C₂-C₈ dialkylamino, C₂-C₈ halodialkylamino, C₃-C₈ cycloalkylamino, C₂-C₈ alkylcarbonylamino, C₂-C₈ haloalkylcarbonylamino, C₁-C₈ alkylsulfonylamino, C₁-C₈ haloalkylsulfonylamino or C₃-C₈ trialkylsilyl; or G;
G is phenyl or naphthalenyl, each optionally substituted with up to 5 substituents independently selected from R^{8a}; or a 5- or 6-membered heteroaromatic ring containing ring members selected from carbon atoms and up to 4 heteroatoms independently selected from up to 2 O, up to 2 S and up to 4 N atoms, each ring optionally substituted with up to 5 substituents independently selected from R^{8a} on carbon atom ring members and R^{8b} on nitrogen atom ring members;
R⁶ is hydrogen, halogen, cyano, C₁-C₂ alkyl or C₁-C₂ haloalkyl; or
R⁶ and R^{4a} are taken together with the linking atoms to which they are attached to form a 3- to 7-membered ring containing ring members, in addition to the linking atoms, selected from carbon atoms and up to 3 heteroatoms independently selected from up to 1 O, up to 1 S and up to 1 N atom, the ring optionally substituted with up to 2 substituents independently selected from halogen, C₁-C₂ alkyl, C₁-C₂ haloalkyl, C₁-C₂ alkoxy and C₁-C₂ haloalkoxy on carbon atom ring members and cyano, C₁-C₂ alkyl and C₁-C₂ alkoxy on nitrogen atom ring members;
each R^{7a} is independently cyano, halogen, hydroxy, C₁-C₃ alkyl, C₁-C₃ haloalkyl, C₁-C₃ alkoxy or C₁-C₃ haloalkoxy;
each R^{7b} is independently cyano, C₁-C₃ alkyl, C₁-C₃ haloalkyl, C₁-C₃ alkoxy or C₂-C₃ alkylcarbonyl;
each R^{8a} is independently halogen, cyano, hydroxy, amino, nitro, C₁-C₄ alkyl, C₂-C₄ alkenyl, C₂-C₄ alkynyl, C₃-C₆ cycloalkyl, C₄-C₈ alkylcycloalkyl, C₄-C₈ cycloalkylalkyl, C₁-C₄ haloalkyl, C₂-C₄ haloalkenyl, C₂-C₄ haloalkynyl, C₃-C₆ halocycloalkyl, C₂-C₄ alkoxyalkyl, C₂-C₄ hydroxyalkyl, C₁-C₄ alkoxy, C₁-C₄ haloalkoxy, C₂-C₄ alkylcarbonyloxy, C₁-C₄ alkylthio, C₁-C₄ haloalkylthio, C₂-C₄ alkylcarbonylthio, C₁-C₄ alkylsulfinyl, C₁-C₄ haloalkylsulfinyl, C₁-C₄ alkylsulfonyl, C₁-C₄ haloalkylsulfonyl, C₁-C₄ alkylamino, C₂-C₆ dialkylamino, C₃-C₆ cycloalkylamino, C₂-C₄ alkylcarbonyl, C₂-C₄ alkoxycarbonyl, C₂-C₄ alkylaminocarbonyl, C₃-C₆ dialkylaminocarbonyl, C₃-C₆ trialkylsilyl or Q²;
each R^{8b} is independently cyano, C₁-C₄ alkyl, C₁-C₄ haloalkyl, C₁-C₄ alkoxy, C₂-C₄ alkylcarbonyl, C₂-C₄ alkoxycarbonyl or C₃-C₆ cycloalkyl;
each Q² is independently a phenyl, benzyloxy, phenoxy, benzylthio, phenylthio or a 5- or 6-membered heteroaromatic ring each optionally substituted with up to 3 substituents independently selected from the group consisting of hydroxy, halogen, cyano, nitro, C₁-C₆ alkyl, C₁-C₆ haloalkyl, C₁-C₆ alkoxy and C₁-C₆ haloalkoxy;
each R^{9a} is independently hydroxy, halogen, cyano, nitro, C₁-C₆ alkyl, C₁-C₆ haloalkyl, C₁-C₆ alkoxy, C₁-C₆ haloalkoxy or phenoxy; and
each R^{9b} is independently cyano, C₁-C₆ alkyl, C₁-C₆ haloalkyl, C₁-C₆ alkoxy or C₂-C₆ alkylcarbonyl;
provided that
   a) when A¹ is N then A² is N;
   b) L is other than —O—, —S— or —NR^{7b}—;
   c) when Z is a direct bond, then Q¹ is bonded to the remainder of Formula 1 via a carbon atom;
   d) when L is a direct bond, then R⁵ is bonded to the remainder of Formula 1 via a carbon atom; and
   e) when Z is CH₂, then Q¹ is other than a 6-membered heteroaromatic ring.

More particularly, this invention pertains to a compound of Formula 1 (including all stereoisomers), an N-oxide or a salt thereof.

This invention also relates to a fungicidal composition comprising (a) a compound of the invention (i.e. in a fungicidally effective amount); and (b) at least one additional component selected from the group consisting of surfactants, solid diluents and liquid diluents.

This invention also relates to a fungicidal composition comprising (a) a compound of the invention; and (b) at least one other fungicide (e.g., at least one other fungicide having a different site of action).

This invention further relates to a method for controlling plant diseases caused by fungal plant pathogens comprising applying to the plant or portion thereof, or to the plant seed, a fungicidally effective amount of a compound of the invention (e.g., as a composition described herein).

### DETAILS OF THE INVENTION

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process, method, article, or apparatus that comprises a list of elements is not necessarily limited to only those elements but may include other elements not expressly listed or inherent to such composition, mixture, process, method, article, or apparatus.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition, method or apparatus that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in the present disclosure and claims, “plant” includes members of Kingdom Plantae, particularly seed plants (Spermatopsida), at all life stages, including young plants (e.g., germinating seeds developing into seedlings) and mature, reproductive stages (e.g., plants producing flowers and seeds). Portions of plants include geotropic members typically growing beneath the surface of the growing medium (e.g., soil), such as roots, tubers, bulbs and corms, and also members growing above the growing medium, such as foliage (including stems and leaves), flowers, fruits and seeds.

As referred to herein, the term “seedling”, used either alone or in a combination of words means a young plant developing from the embryo of a seed.

As referred to in this disclosure, the terms “fungal pathogen” and “fungal plant pathogen” include pathogens in the Ascomycota, Basidiomycota and Zygomycota phyla, and the fungal-like Oomycota class that are the causal agents of a broad spectrum of plant diseases of economic importance, affecting ornamental, turf, vegetable, field, cereal and fruit crops. In the context of this disclosure, “protecting a plant from disease” or “control of a plant disease” includes preventative action (interruption of the fungal cycle of infection, colonization, symptom development and spore production) and/or curative action (inhibition of colonization of plant host tissues).

As referred to in this disclosure, the term mode of action (MOA) is as defined broadly by the Fungicide Resistance Action Committee (FRAC), and is used to distinguish fungicide groups according to their biochemical mode of action in the biosynthetic pathways of plant pathogens. These FRAC-defined MOAs are (A) nucleic acid synthesis, (B) mitosis and cell division, (C) respiration, (D) amino acid and protein synthesis, (E) signal transduction, (F) lipid synthesis and membrane integrity, (G) sterol biosynthesis in membranes, (H) cell wall biosynthesis in membranes, (I) melanin synthesis in cell wall, (P) host plant defense induction, multi-site contact activity and unknown mode of action. Each MOA class consists of one or more groups based either on individual validated target sites of action, or in cases where the precise target site is unknown, based on cross resistance profiles within a group or in relation to other groups. Each of these groupings within a FRAC-defined MOA, whether the target site is known or unknown, is designated by a FRAC code. Additional information on target sites and FRAC codes can be obtained from publicly available databases maintained, for example, by FRAC.

As referred to in this disclosure, the term “cross resistance” refers to a phenomenon wherein a pathogen evolves resistance to one fungicide and in addition acquires resistance to others. These additional fungicides are typically, but not always, in the same chemical class or have the same target site of action, or can be detoxified by the same mechanism.

In the above recitations, the term “alkyl”, used either alone or in compound words such as “alkylthio” or “haloalkyl” includes straight-chain or branched alkyl such as methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” also includes moieties comprised of multiple triple bonds such as 2,5-hexadiynyl. “Alkylene” denotes a straight-chain or branched alkanediyl. Examples of “alkylene” include CH₂, CH₂CH₂, CH(CH₃), CH₂CH₂CH₂, CH₂CH(CH₃), and the different butylene isomers. “Alkenylene” denotes a straight-chain or branched alkenediyl containing one olefinic bond. Examples of “alkenylene” include CH═CH, CH₂CH═CH, CH═C(CH₃) and the different butenylene isomers.

“Alkoxy” includes, for example, methoxy, ethoxy, n-propyloxy, isopropyloxy and the different butoxy, pentoxy and hexyloxy isomers. “Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂. “Alkoxyalkoxy” denotes alkoxy substitution on alkoxy. “Alkenyloxy” includes straight-chain or branched alkenyloxy moieties. Examples of “alkenyloxy” include H₂C═CHCH₂O, (CH₃)₂C═CHCH₂O, (CH₃)CH═CHCH₂O, (CH₃)CH═C(CH₃)CH₂O and CH₂═CHCH₂CH₂O. “Alkynyloxy” includes straight-chain or branched alkynyloxy moieties. Examples of “alkynyloxy” include HC≡CCH₂O, CH₃C≡CCH₂O and CH₃C≡CCH₂CH₂O. “Alkylthio” includes branched or straight-chain alkylthio moieties such as methylthio, ethylthio, and the different propylthio, butylthio, pentylthio and hexylthio isomers. “Alkylsulfinyl” includes both enantiomers of an alkylsulfinyl group. Examples of “alkylsulfinyl” include CH₃S(O)—, CH₃CH₂S(O)—, CH₃CH₂CH₂S(O)—, (CH₃)₂CHS(O)— and the different butylsulfinyl, pentylsulfinyl and hexylsulfinyl isomers. Examples of “alkyl sulfonyl” include CH₃S(O)₂—, CH₃CH₂S(O)₂—, CH₃CH₂CH₂S(O)₂—, (CH₃)₂CHS(O)₂—, and the different butylsulfonyl, pentylsulfonyl and hexylsulfonyl isomers. “Alkylthioalkyl” denotes alkylthio substitution on alkyl. Examples of “alkylthioalkyl” include CH₃SCH₂, CH₃SCH₂CH₂, CH₃CH₂SCH₂, CH₃CH₂CH₂CH₂SCH₂ and CH₃CH₂SCH₂CH₂. “Cyanoalkyl” denotes an alkyl group substituted with one cyano group. Examples of “cyanoalkyl” include NCCH₂, NCCH₂CH₂ and CH₃CH(CN)CH₂. “Alkylamino”, “dialkylamino”, “alkenylthio”, “alkenylsulfinyl”, “alkenylsulfonyl”, “alkynylthio”, “alkynylsulfinyl”, “alkynylsulfonyl”, and the like, are defined analogously to the above examples. “Cycloalkyl” includes, for example, cyclopropyl, cyclobutyl, cyclopentyl and cyclohexyl. The term “alkylcycloalkyl” denotes alkyl substitution on a cycloalkyl moiety and includes, for example, ethylcyclopropyl, i-propylcyclobutyl, 3-methylcyclopentyl and 4-methylcyclohexyl. The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to straight-chain or branched alkyl groups. The term “cycloalkoxy” denotes cycloalkyl linked through an oxygen atom such as cyclopentyloxy and cyclohexyloxy. “Cycloalkylalkoxy” denotes cycloalkylalkyl linked through an oxygen atom attached to the alkyl chain. Examples of “cycloalkylalkoxy” include cyclopropylmethoxy, cyclopentylethoxy, and other cycloalkyl moieties bonded to straight-chain or branched alkoxy groups. “Cycloalkenyl” includes groups such as cyclopentenyl and cyclohexenyl as well as groups with more than one double bond such as 1,3- and 1,4-cyclohexadienyl.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include F₃C—, ClCH₂—, CF₃CH₂— and CF₃CCl₂—. The terms “halocycloalkyl”, “haloalkoxy”, “haloalkylthio”, “haloalkenyl”, “haloalkynyl”, and the like, are defined analogously to the term “haloalkyl”. Examples of “haloalkoxy” include CF₃O—, CCl₃CH₂O—, HCF₂CH₂CH₂O— and CF₃CH₂O—. Examples of “haloalkylthio” include CCl₃S—, CF₃S—, CCl₃CH₂S— and ClCH₂CH₂CH₂S—. Examples of “haloalkylsulfinyl” include CF₃S(O)—, CCl₃S(O)—, CF₃CH₂S(O)— and CF₃CF₂S(O)—. Examples of “haloalkylsulfonyl” include CF₃S(O)₂—, CCl₃S(O)₂—, CF₃CH₂S(O)₂— and CF₃CF₂S(O)₂—. Examples of “haloalkenyl” include (Cl)₂C═CHCH₂— and CF₃CH₂CH═CHCH₂—. Examples of “haloalkynyl” include HC≡CCHCl—, CF₃C≡C—, CCl₃C≡C— and FCH₂C≡CCH₂—. Examples of “haloalkoxyalkoxy” include CF₃OCH₂O—, ClCH₂CH₂OCH₂CH₂O—, Cl₃CCH₂OCH₂O— as well as branched alkyl derivatives.

“Alkylcarbonyl” denotes a straight-chain or branched alkyl moieties bonded to a C(═O) moiety. Examples of “alkylcarbonyl” include CH₃C(═O)—, CH₃CH₂CH₂C(═O)— and (CH₃)₂CHC(═O)—. Examples of “alkoxycarbonyl” include CH₃OC(═O)—, CH₃CH₂OC(═O)—, CH₃CH₂CH₂OC(═O)—, (CH₃)₂CHOC(═O)— and the different butoxy- or pentoxycarbonyl isomers.

The total number of carbon atoms in a substituent group is indicated by the “C₁-Cⱼ” prefix where i and j are numbers from 1 to 10. For example, C₁-C₄ alkylsulfonyl designates methylsulfonyl through butylsulfonyl; C₂ alkoxyalkyl designates CH₃OCH₂—; C₃ alkoxyalkyl designates, for example, CH₃CH(OCH₃)—, CH₃OCH₂CH₂— or CH₃CH₂OCH₂—; and C₄ alkoxyalkyl designates the various isomers of an alkyl group substituted with an alkoxy group containing a total of four carbon atoms, examples including CH₃CH₂CH₂OCH₂— and CH₃CH₂OCH₂CH₂—.

When a compound is substituted with a substituent bearing a subscript that indicates the number of said substituents can exceed 1, said substituents (when they exceed 1) are independently selected from the group of defined substituents, e.g., (R⁸)ₘ, m is 0, 1, 2 or 3. Further, when the subscript indicates a range, e.g. (R)₁₋ⱼ, then the number of substituents may be selected from the integers between i and j inclusive. When a group contains a substituent which can be hydrogen, for example R¹ or R³, then when this substituent is taken as hydrogen, it is recognized that this is equivalent to said group being unsubstituted. When a variable group is shown to be optionally attached to a position, for example (R⁹)ₖ wherein k may be 0, then hydrogen may be at the position even if not recited in the variable group definition. When one or more positions on a group are said to be “not substituted” or “unsubstituted”, then hydrogen atoms are attached to take up any free valency.

A “chain” is an acyclic string of atoms bonded in a single line with single (saturated) or multiple bonds (unsaturated) between atoms (chain members). The term “chain” is used to define group L in Formula 1 and connects to the isoxazoline ring on one end and group R⁵ on the other end. A “chain” as a component of Formula 1 may contain carbon or heteroatom chain members. The chain itself is unbranched, but chain members may also be further substituted with other functional groups as indicated in variables R^{7a} and R^{7b}. The chain length can vary from two to four chain members as described in the Summary of the Invention.

Unless otherwise indicated, a “ring” or “ring system” as a component of Formula 1 (e.g., substituent G) is carbocyclic or heterocyclic. The term “ring system” denotes two or more fused rings. The terms “bicyclic ring system” and “fused bicyclic ring system” denote a ring system consisting of two fused rings which can be “ortho-fused”, “bridged bicyclic” or “spirobicyclic”. An “ortho-fused bicyclic ring system” denotes a ring system wherein the two constituent rings have two adjacent atoms in common. A “bridged bicyclic ring system” is formed by bonding a segment of one or more atoms to nonadjacent ring members of a ring. A “spirobicyclic ring system” is formed by bonding a segment of two or more atoms to the same ring member of a ring. The term “fused heterobicyclic ring system” denotes a fused bicyclic ring system in which at least one ring atom is not carbon. The term “ring member” refers to an atom or other moiety (e.g., C(═O), C(═S), S(O) or S(O)₂) forming the backbone of a ring or ring system.

The terms “carbocyclic ring”, “carbocycle” or “carbocyclic ring system” denote a ring or ring system wherein the atoms forming the ring backbone are selected only from carbon. The terms “heterocyclic ring”, “heterocycle” or “heterocyclic ring system” denote a ring or ring system in which at least one atom forming the ring backbone is not carbon, e.g., nitrogen, oxygen or sulfur. Typically a heterocyclic ring contains no more than 4 nitrogens, no more than 2 oxygens and no more than 2 sulfurs. Unless otherwise indicated, a carbocyclic ring or heterocyclic ring can be a saturatedor unsaturated ring. “Saturated” refers to a ring having a backbone consisting of atoms linked to one another by single bonds; unless otherwise specified, the remaining carbon valences are occupied by hydrogen atoms. Unless otherwise stated, an “unsaturated ring” may be partially unsaturated or fully unsaturated. The expression “fully unsaturated ring” means a ring of atoms in which the bonds between atoms in the ring are single or double bonds according to valence bond theory and furthermore the bonds between atoms in the ring include as many double bonds as possible without double bonds being cumulative (i.e. no C═C═C or C═C═N). The term “partially unsaturated ring” denotes a ring comprising at least one ring member bonded to an adjacent ring member through a double bond and which conceptually potentially accommodates a number of non-cumulated double bonds between adjacent ring members (i.e. in its fully unsaturated counterpart form) greater than the number of double bonds present (i.e. in its partially unsaturated form).

Unless otherwise indicated, heterocyclic rings and ring systems can be attached through any available carbon or nitrogen by replacement of a hydrogen atom on said carbon or nitrogen.

“Aromatic” indicates that each of the ring atoms is essentially in the same plane and has a p-orbital perpendicular to the ring plane, and that (4n+2) π electrons, where n is a positive integer, are associated with the ring to comply with Hückel's rule. The term “aromatic ring system” denotes a carbocyclic or heterocyclic ring system in which at least one ring of the ring system is aromatic. When a fully unsaturated carbocyclic ring satisfies Hückel's rule, then said ring is also called an “aromatic ring” or “aromatic carbocyclic ring”. The term “aromatic carbocyclic ring system” denotes a carbocyclic ring system in which at least one ring of the ring system is aromatic. When a fully unsaturated heterocyclic ring satisfies Hückel's rule, then said ring is also called a “heteroaromatic ring” or “aromatic heterocyclic ring”. The term “aromatic heterocyclic ring system” denotes a heterocyclic ring system in which at least one ring of the ring system is aromatic. The term “nonaromatic ring system” denotes a carbocyclic or heterocyclic ring system that may be fully saturated, as well as partially or fully unsaturated, provided that none of the rings in the ring system are aromatic. The term “nonaromatic carbocyclic ring system” denotes a carbocyclic ring in which no ring in the ring system is aromatic. The term “nonaromatic heterocyclic ring system” denotes a heterocyclic ring system in which no ring in the ring system is aromatic.

The term “optionally substituted” in connection with the heterocyclic rings refers to groups which are unsubstituted or have at least one non-hydrogen substituent that does not extinguish the biological activity possessed by the unsubstituted analog. As used herein, the following definitions shall apply unless otherwise indicated. The term “optionally substituted” is used interchangeably with the phrase “substituted or unsubstituted” or with the term “(un)substituted.” Unless otherwise indicated, an optionally substituted group may have a substituent at each substitutable position of the group, and each substitution is independent of the other.

A wide variety of synthetic methods are known in the art to enable preparation of aromatic and nonaromatic heterocyclic rings and ring systems; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996.

Compounds of this invention can exist as one or more stereoisomers. Stereoisomers are isomers of identical constitution but differing in the arrangement of their atoms in space and include enantiomers, diastereomers, cis-trans isomers (also known as geometric isomers) and atropisomers. Atropisomers result from restricted rotation about single bonds where the rotational barrier is high enough to permit isolation of the isomeric species. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. For a comprehensive discussion of all aspects of stereoisomerism, see Ernest L. Eliel and Samuel H. Wilen, Stereochemistry of Organic Compounds, John Wiley & Sons, 1994.

The compounds of the invention may be present as a mixture of stereoisomers, or individual stereoisomers. In the simplified example below there are three chiral centers in the compound of Formula 1 depending on the identity of the variables. The asymmetric centers are identified with an asterisk (*).

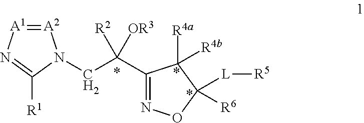

For example, for compounds of Formula 1 wherein variables R^{4a} and R^{4b} are both hydrogen there are four isomers as shown below. Enantiomeric pair 1w and 1x is in a diastereisomeric relationship to enantiomeric pair 1y and 1z.

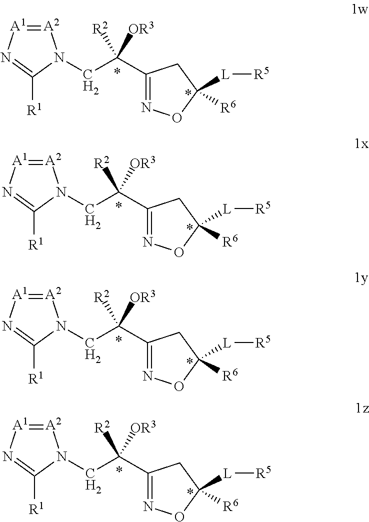

Molecular depictions drawn herein follow standard conventions for depicting stereochemistry. To indicate stereoconfiguration, bonds rising from the plane of the drawing and towards the viewer are denoted by solid wedges wherein the broad end of the wedge is attached to the atom rising from the plane of the drawing towards the viewer. Bonds going below the plane of the drawing and away from the viewer are denoted by dashed wedges wherein the narrow end of the wedge is attached to the atom further away from the viewer. Constant width lines indicate bonds with a direction opposite or neutral relative to bonds shown with solid or dashed wedges; constant width lines also depict bonds in molecules or parts of molecules in which no particular stereoconfiguration is intended to be specified.

This invention comprises racemic mixtures, for example, equal amounts of the enantiomers of Formulae 1w and 1x. In addition, this invention includes compounds that are enriched compared to the racemic mixture in an enantiomer of Formula 1. Also included are the essentially pure enantiomers of compounds of Formula 1, for example, Formula 1w and Formula 1x.

When enantiomerically enriched, one enantiomer is present in greater amounts than the other, and the extent of enrichment can be defined by an expression of enantiomeric excess (“ee”), which is defined as (2x−1)·100%, where x is the mole fraction of the dominant enantiomer in the mixture (e.g., an ee of 20% corresponds to a 60:40 ratio of enantiomers).

Preferably the compositions of this invention have at least a 50% enantiomeric excess; more preferably at least a 75% enantiomeric excess; still more preferably at least a 90% enantiomeric excess; and the most preferably at least a 94% enantiomeric excess of the more active isomer. Of particular note are enantiomerically pure embodiments of the more active isomer.

Compounds of Formula 1 can comprise additional chiral centers. For example, substituents and other molecular constituents such as R², R⁵ and L may themselves contain chiral centers. This invention comprises racemic mixtures as well as enriched and essentially pure stereoconfigurations at these additional chiral centers.

Compounds of this invention can exist as one or more conformational isomers due to restricted rotation about an amide bond (e.g., C(W)—N) in a substituent in Formula 1. This invention comprises mixtures of conformational isomers. In addition, this invention includes compounds that are enriched in one conformer relative to others.

This invention comprises all stereoisomers, conformational isomers and mixtures thereof in all proportions as well as isotopic forms such as deuterated compounds.

One skilled in the art will appreciate that not all nitrogen containing heterocycles can form N-oxides since the nitrogen requires an available lone pair for oxidation to the oxide; one skilled in the art will recognize those nitrogen-containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and m-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus a wide variety of salts of the compounds of Formula 1 are useful for control of plant diseases caused by fungal plant pathogens (i.e. are agriculturally suitable). The salts of the compounds of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. When a compound of Formula 1 contains an acidic moiety such as a carboxylic acid or phenol, salts also include those formed with organic or inorganic bases such as pyridine, triethylamine or ammonia, or amides, hydrides, hydroxides or carbonates of sodium, potassium, lithium, calcium, magnesium or barium. Accordingly, the present invention comprises compounds selected from Formula 1, N-oxides and agriculturally suitable salts thereof.

Compounds selected from Formula 1, stereoisomers, tautomers, N-oxides, and salts thereof, typically exist in more than one form, and Formula 1 thus includes all crystalline and non-crystalline forms of the compounds that Formula 1 represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due to the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula 1 can exhibit beneficial effects (e.g., suitability for preparation of useful formulations, improved biological performance) relative to another polymorph or a mixture of polymorphs of the same compound represented by Formula 1. Preparation and isolation of a particular polymorph of a compound represented by Formula 1 can be achieved by methods known to those skilled in the art including, for example, crystallization using selected solvents and temperatures. For a comprehensive discussion of polymorphism see R. Hilfiker, Ed., Polymorphism in the Pharmaceutical Industry, Wiley-VCH, Weinheim, 2006.

Embodiments of the present invention as described in the Summary of the Invention include (where Formula 1 as used in the following Embodiments includes N-oxides and salts thereof):
Embodiment 1. A compound of Formula 1 wherein A¹ is CH and A² is CH or A¹ is CH and A² is N.
Embodiment 2. A compound of Embodiment 1wherein A¹ is CH and A² is N.
Embodiment 3. A compound of Formula 1 or any one of Embodiments 1 through 2 either alone or in combination, wherein R¹ is hydrogen, SH, SCN, C₁-C₆ alkylthio or C₂-C₆ alkenylthio.
Embodiment 4. A compound of Embodiment 3 wherein R¹ is hydrogen, SH, SCN, SCH₃ or SCH₂CH═CH₂.
Embodiment 5. A compound of Embodiment 4 wherein R¹ is hydrogen or SH.
Embodiment 6. A compound of Embodiment 5 wherein R¹ is hydrogen.
Embodiment 7. A compound of Embodiment 5 wherein R¹ is SH.
Embodiment 8. A compound of Formula 1 or any one of Embodiments 1 through 7 either alone or in combination, wherein R² is C₁-C₆ alkyl optionally substituted with up to 5 substituents independently selected from R^{2a}; or C₃-C₈ cycloalkyl optionally substituted with up to 5 substituents independently selected from R^{2b}; or ZQ¹.
Embodiment 9. A compound of Embodiment 8 wherein R² is C₁-C₆ alkyl optionally substituted with up to 3 substituents independently selected from R^{2a}; or C₃-C₈ cycloalkyl optionally substituted with up to 3 substituents independently selected from R^{2b}; or ZQ¹.
Embodiment 10. A compound of Embodiment 9 wherein R² is C₁-C₆ alkyl optionally substituted with up to 3 substituents independently selected from R^{2a}; or C₃-C₆ cycloalkyl optionally substituted with up to 3 substituents independently selected from R^{2b}; or ZQ¹.
Embodiment 11. A compound of Embodiment 10 wherein R² is C₃-C₆ cycloalkyl.
Embodiment 12. A compound of Embodiment 11 wherein R² is C₄-C₆ alkyl.
Embodiment 13. A compound of Embodiment 10 wherein R² is ZQ¹.
Embodiment 14. A compound of Formula 1 or any one of Embodiments 1 through 11 either alone or in combination, wherein each R^{2a} is independently halogen, cyano, C₁-C₆ alkoxy, C₁-C₆ haloalkoxy or C₃-C₆ cycloalkyl.
Embodiment 15. A compound of Formula 1 or any one of Embodiments 1 through 9 either alone or in combination, wherein each R^{2b} is independently halogen, cyano, C₁-C₆ alkyl, C₁-C₆ haloalkyl, C₁-C₆ alkoxy or C₁-C₆ haloalkoxy.
Embodiment 16. A compound of Formula 1 or any one of Embodiments 1 through 13 either alone or in combination, wherein Z is a direct bond or CH₂.
Embodiment 17. A compound of Embodiment 16 wherein Z is a direct bond
Embodiment 18. A compound of Embodiment 16 wherein Z is CH₂.
Embodiment 19. A compound of Formula 1 or any one of Embodiments 1 through 18 either alone or in combination, wherein Q¹ is selected from Q-1 through Q-65 depicted in Exhibit 1;

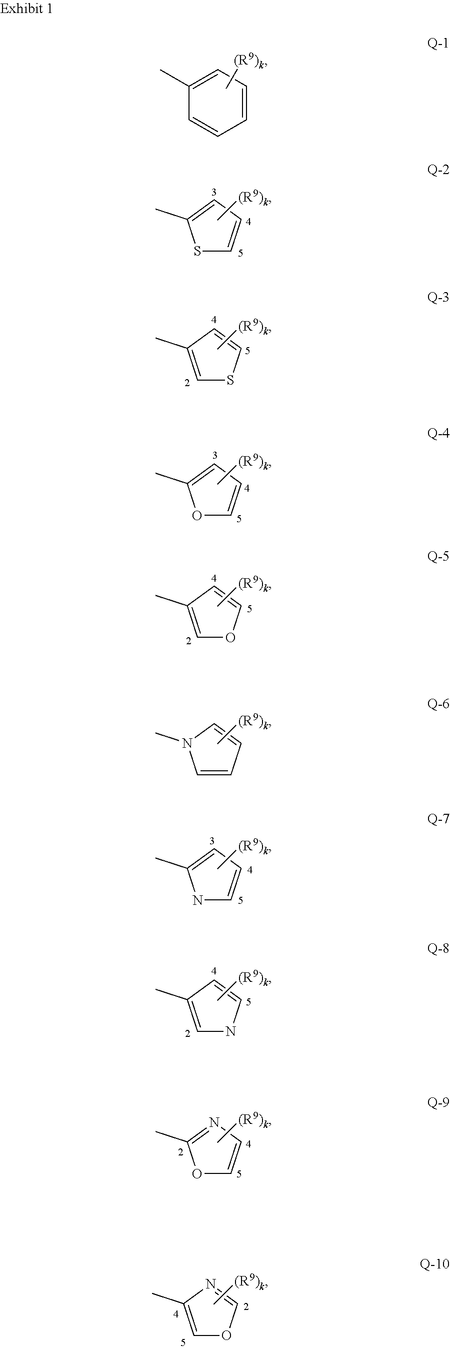

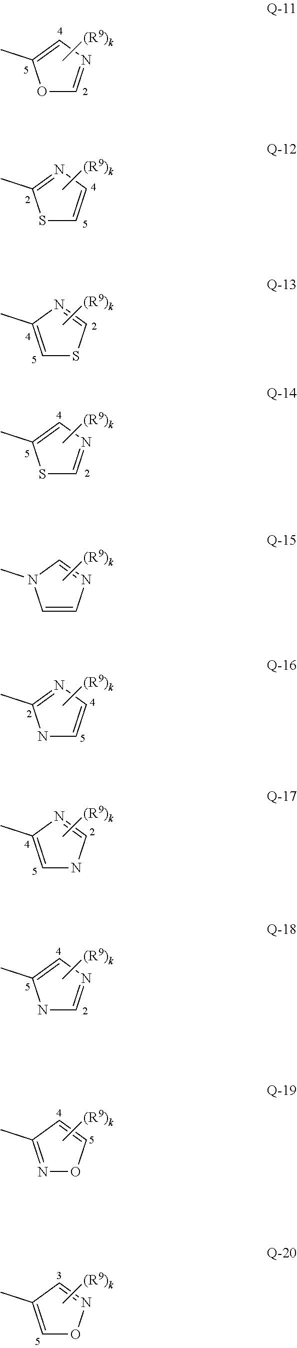

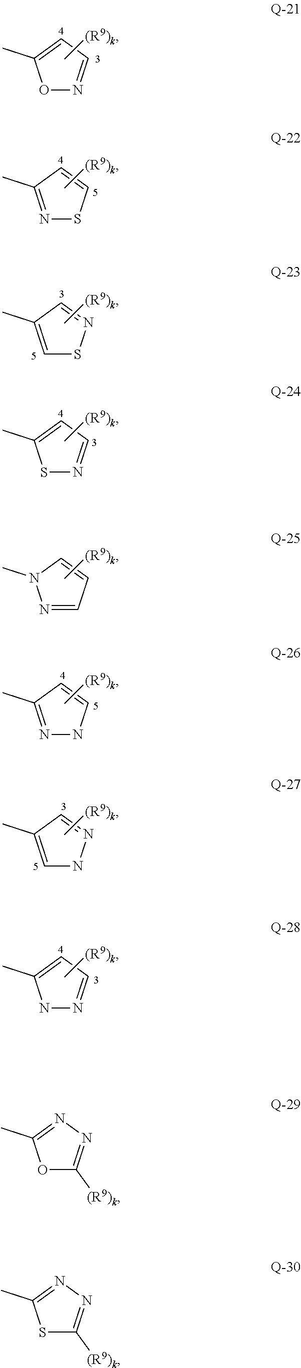

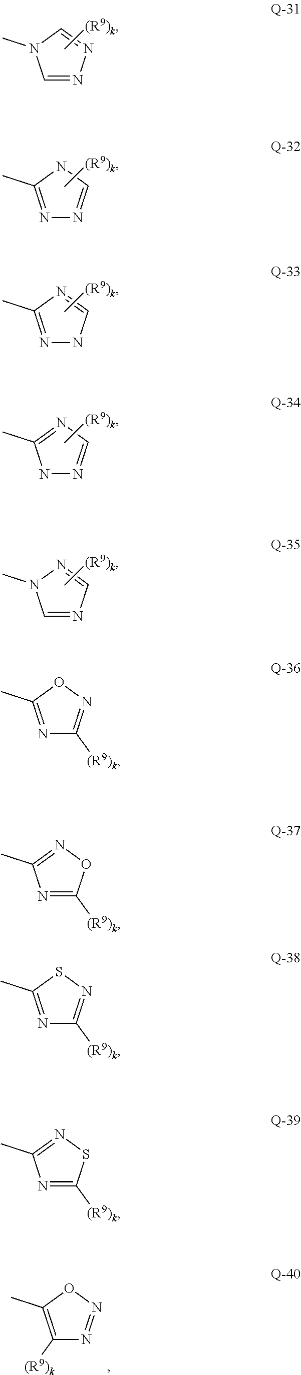

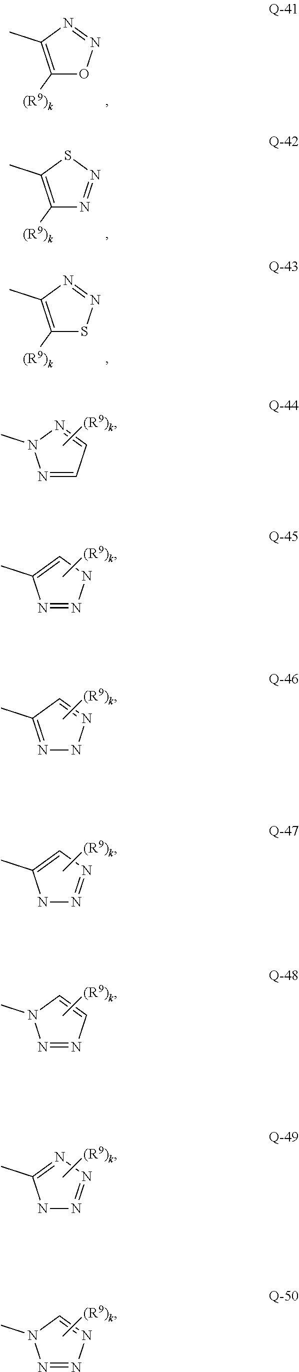

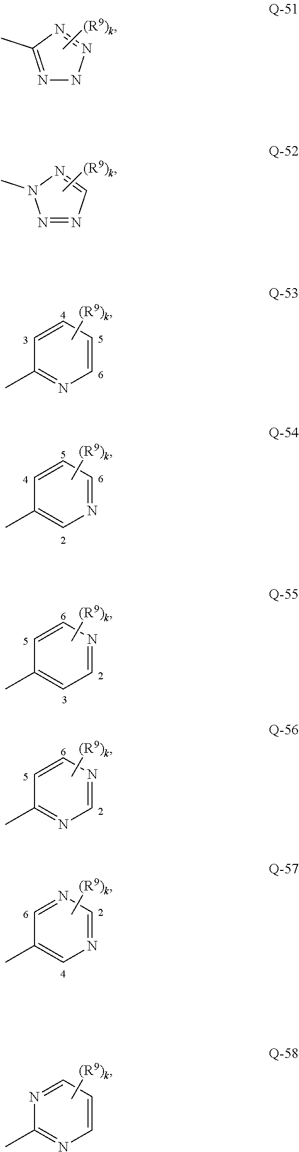

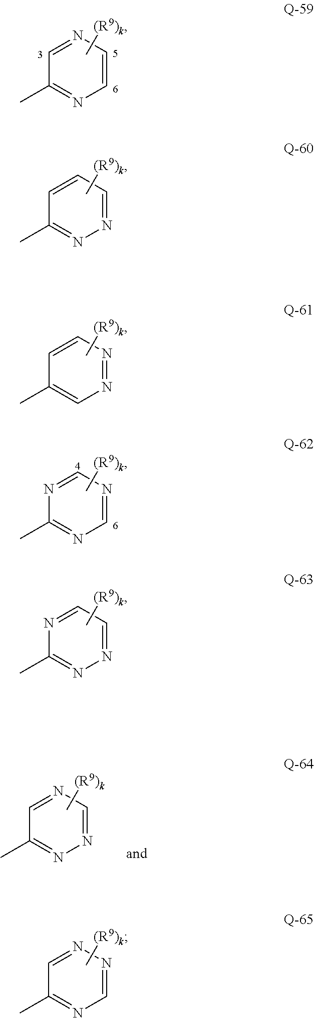

wherein when R⁹ is attached to a carbon ring member, said R⁹ is selected from R^{9a}, and when R⁹ is attached to a nitrogen ring member (e.g., in Q-7, Q-8, Q-16, Q-17, Q-18, Q-26, Q-27, Q-28, Q-32, Q-33, Q-34, Q-45, Q-46, Q-47, Q-49 and Q-51), said R⁹ is selected from R^{9b}; and k is 0, 1, 2 or 3.
Embodiment 20. A compound of Embodiment 19 wherein Q′ is selected from Q-1 through Q-5, Q-12 through Q-15, Q-25, Q-35, Q-44, Q-45, Q-50, Q-52 through Q-55 and Q-58.
Embodiment 21. A compound of Embodiment 20 wherein Q¹ is selected from Q-1 through Q-3, Q-15, Q-25, Q-35, Q-50 and Q-54.
Embodiment 22. A compound of Embodiment 21 wherein Q¹ is selected from Q-1, Q-15, Q-25, Q-35, Q-50 and Q-54.
Embodiment 22a. A compound of Embodiment 22 wherein Q¹ is selected from Q-1, Q-35 and Q-54.
Embodiment 22b. A compound of Formula 1 or any one of Embodiments 1 through 22a either alone or in combination, wherein R² is C₄-C₆ alkyl; C₃-C₆ cycloalkyl; or ZQ¹ wherein Z is a direct bond or CH₂ and Q¹ is selected from Q-1, Q-15, Q-25, Q-35, Q-50 and Q-54.
Embodiment 23. A compound of Formula 1 or any one of Embodiments 1 through 22b either alone or in combination, wherein R³ is hydrogen, CHO, C₂-C₄ alkylcarbonyl, C₂-C₄ alkoxycarbonyl, C₁-C₄ alkyl, or C₂-C₄ alkoxyalkyl.
Embodiment 24. A compound of Embodiment 23 wherein when R³ is hydrogen or C₁-C₄ alkyl.
Embodiment 25. A compound of Embodiment 24 wherein when R³ is hydrogen or CH₃.
Embodiment 26. A compound of Embodiment 25 wherein when R³ is hydrogen.
Embodiment 27. A compound of Formula 1 or any one of Embodiments 1 through 26 either alone or in combination, wherein R^{4a} is hydrogen, halogen or C₁-C₂ alkyl.
Embodiment 28. A compound of Embodiment 27 wherein R^{4a} is hydrogen.
Embodiment 29. A compound of Formula 1 or any one of Embodiments 1 through 28 either alone or in combination, wherein R^{4b} is hydrogen, halogen or C₁-C₂ alkyl.
Embodiment 30. A compound of Embodiment 29 wherein R^{4b} is hydrogen.
Embodiment 31. A compound of Formula 1 or any one of Embodiments 1 through 30 either alone or in combination, wherein L is a direct bond; or a 1-, 2- or 3-membered saturated or partially unsaturated chain containing chain members selected from carbon atoms and up to 2 heteroatoms independently selected from up to 1 O, up to 1 S, and up to 2 N, wherein the chain is optionally substituted with up to 3 substituents independently selected from R^{7a} on carbon atom chain members and R^{7b} on nitrogen atom chain members.
Embodiment 32. A compound of Embodiment 31 wherein L is a direct bond; or a 1- or 2-membered saturated chain containing chain members selected from carbon atoms and up to 1 heteroatom selected from up to 1 O, up to 1 S, and up to 1 N, wherein the chain is optionally substituted with up to 2 substituents independently selected from R^{7a} on carbon atom chain members and R^{7b} on nitrogen atom chain members.
Embodiment 33. A compound of Embodiment 32 wherein L is a direct bond; or —CH₂—, —CH₂O—, —CH₂S—, —CH₂NR^{7b}— or —CH₂CH₂— wherein the left bond is connected to the isoxazoline ring and the right bond is connected to R⁵.
Embodiment 34. A compound of Embodiment 33 wherein L is a direct bond.
Embodiment 35. A compound of Embodiment 33 wherein L is —CH₂—, —CH₂O—, —CH₂S— or —CH₂CH₂— wherein the left bond is connected to the isoxazoline ring and the right bond is connected to R⁵.
Embodiment 36. A compound of Embodiment 35 wherein L is —CH₂—.
Embodiment 37. A compound of Embodiment 35 wherein L is —CH₂O— wherein the left bond is connected to the isoxazoline ring and the right bond is connected to R⁵.
Embodiment 38. A compound of Embodiment 35 wherein L is —CH₂S— wherein the left bond is connected to the isoxazoline ring and the right bond is connected to R⁵.
Embodiment 39. A compound of Embodiment 35 wherein L is —CH₂CH₂—.
Embodiment 40. A compound of Formula 1 or any one of Embodiments 1 through 39 either alone or in combination, wherein each R^{7a} is independently halogen, C₁-C₂ alkyl or C₁-C₂ haloalkyl.
Embodiment 41. A compound of Formula 1 or any one of Embodiments 1 through 40 either alone or in combination, wherein each R^{7b} is independently C₁-C₂ alkyl, C₁-C₂ haloalkyl or C₂-C₃ alkylcarbonyl.
Embodiment 42. A compound of Formula 1 or any one of Embodiments 1 through 41 either alone or in combination, wherein R⁵ is C₁-C₈ alkyl, C₁-C₈ haloalkyl, C₃-C₈ cycloalkyl, C₃-C₈ halocycloalkyl, C₄-C₁₀ alkylcycloalkyl, C₂-C₈ alkoxyalkyl, C₂-C₈ haloalkoxyalkyl, C₁-C₈ alkoxy, C₁-C₈ haloalkoxy, C₃-C₈ cycloalkoxy, C₃-C₈ halocycloalkoxy, C₁-C₈ alkylthio, C₁-C₈ haloalkylthio, C₁-C₈ alkylamino, C₁-C₈ haloalkylamino, C₂-C₈ dialkylamino, C₂-C₈ halodialkylamino, or C₃-C₈ trialkylsilyl; or G;
Embodiment 43. A compound of Embodiment 42 wherein R⁵ is C₁-C₈ alkyl, C₁-C₈ haloalkyl, C₃-C₈ cycloalkyl, C₃-C₈ halocycloalkyl, C₁-C₈ alkoxy or C₁-C₈ haloalkoxy; or G.
Embodiment 44. A compound of Embodiment 43 wherein R⁵ is G.
Embodiment 45. A compound of Formula 1 or any one of Embodiments 1 through 44 either alone or in combination, wherein G is selected from G-1 through G-65 depicted in Exhibit 2.

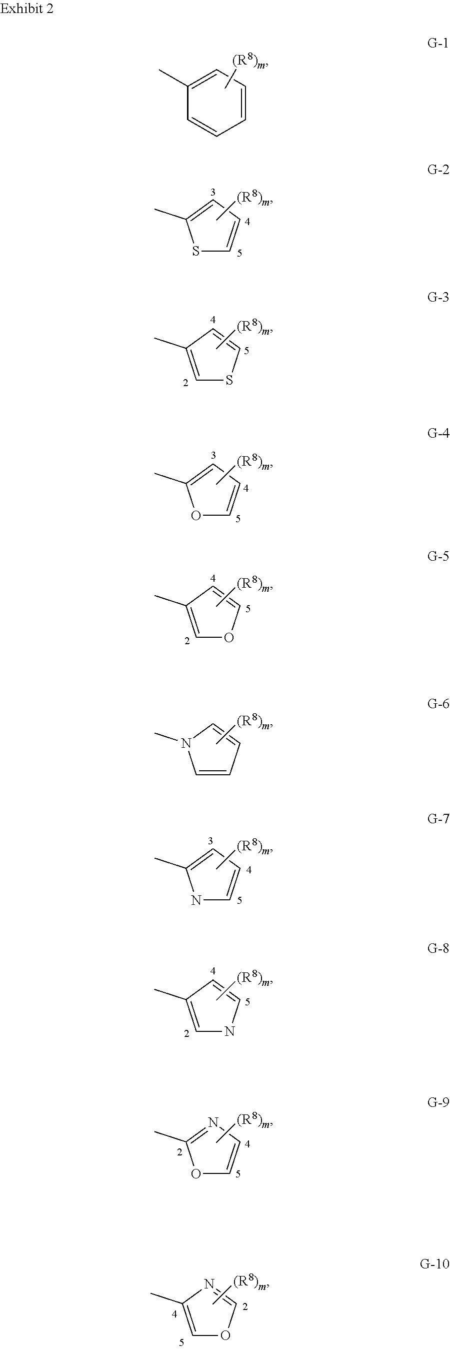

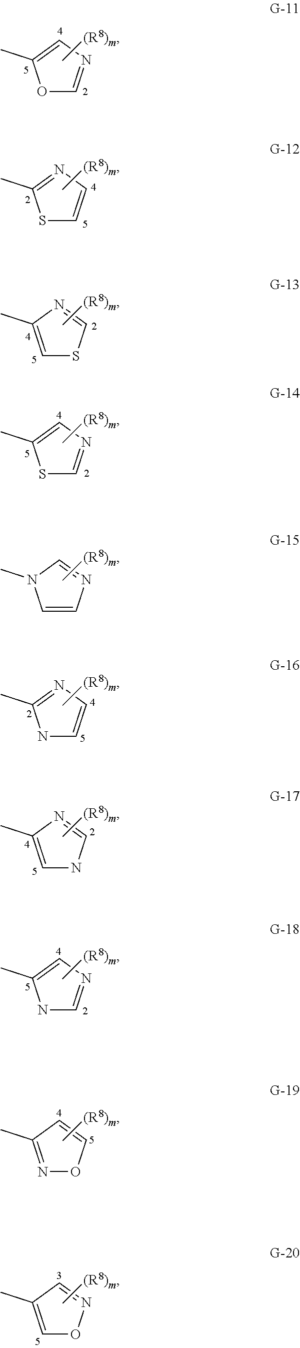

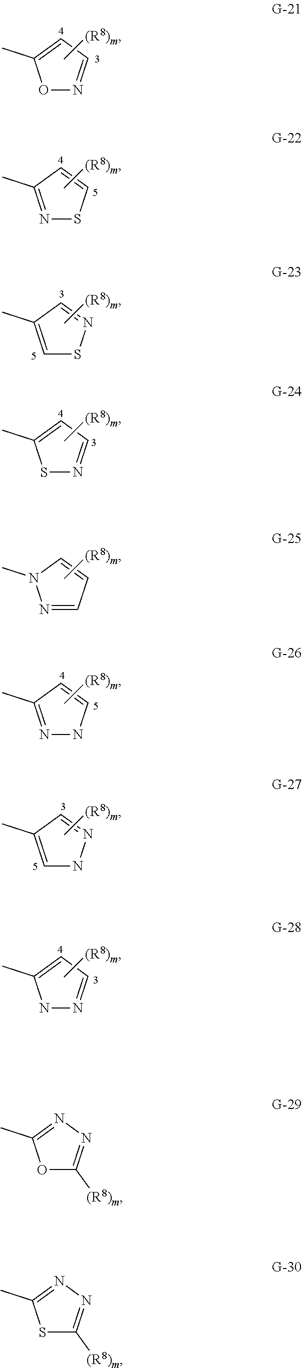

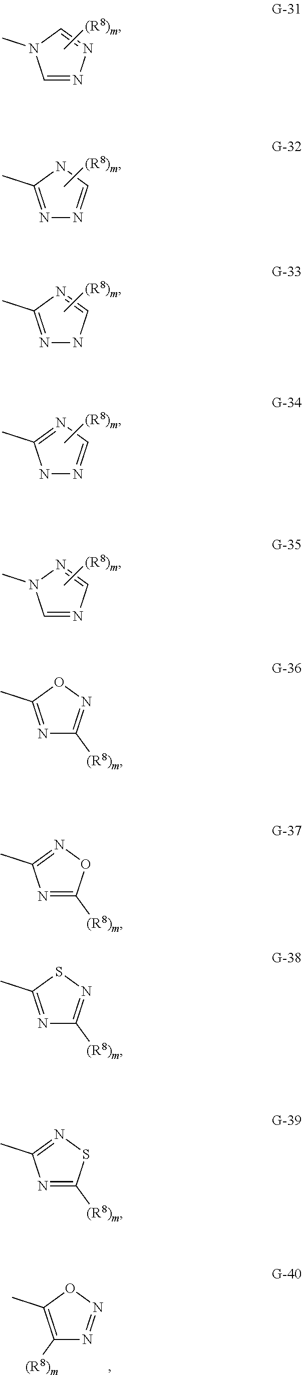

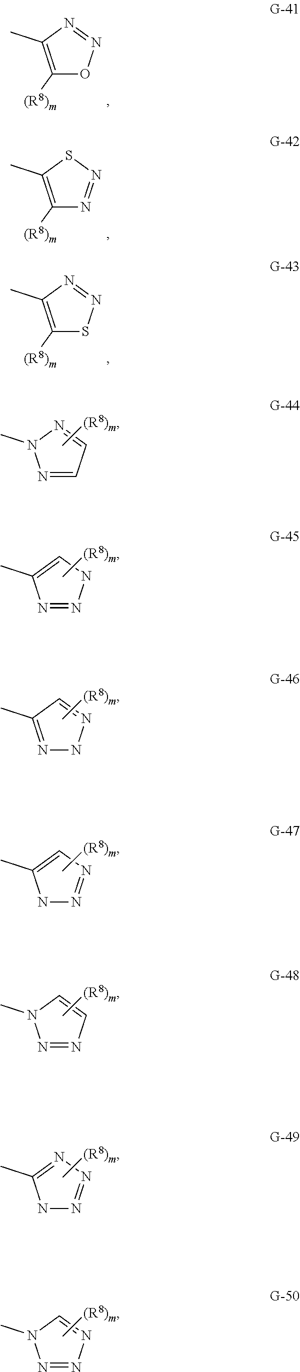

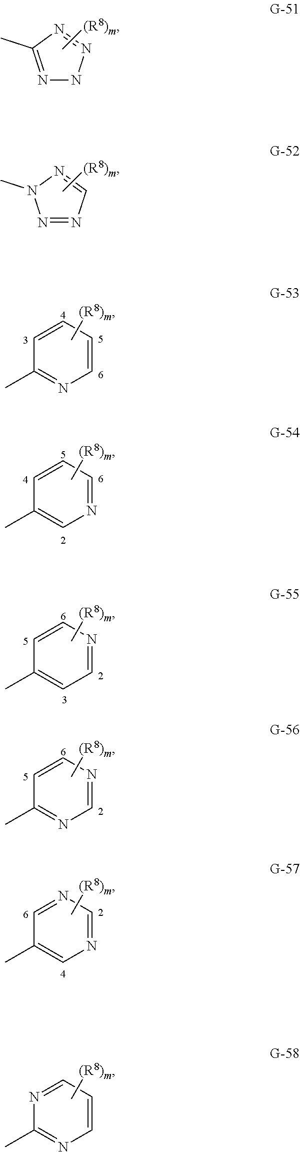

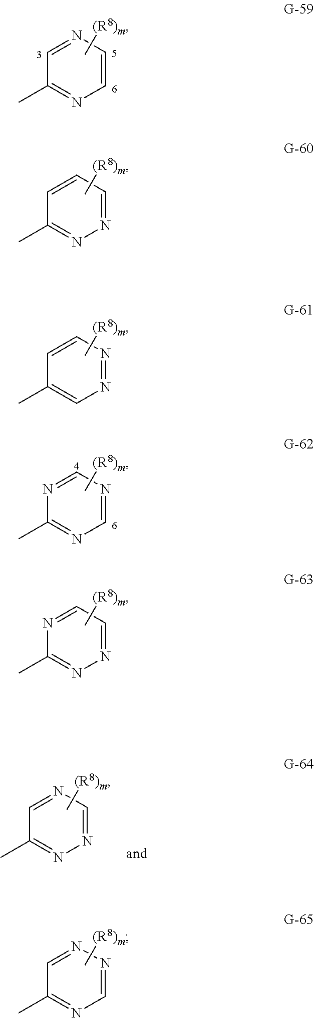

wherein when R⁸ is attached to a carbon ring member, said R⁸ is selected from R^{8a}, and when R⁸ is attached to a nitrogen ring member (e.g., in G-7, G-8, G-16, G-17, G-18, G-26, G-27, G-28, G-32, G-33, G-34, G-45, G-46, G-47, G-49 and G-51), said R⁸ is selected from R^{8b}; and m is 0, 1, 2 or 3.
Embodiment 46. A compound of Embodiment 45 wherein G is selected from G-1 through G-5, G-12, G-13, G-14, G-21, G-25, G-45, and G-53.
Embodiment 47. A compound of Embodiment 46 wherein G is selected from G-1, G-2,

G-3, G-12, G-13, G-14, G-25 and G-53.
Embodiment 47a. A compound of Embodiment 47 wherein G is selected from G-1, G-2, G-3, G-25 and G-53.
Embodiment 47b. A compound of Embodiment 47a wherein G is selected from G-1, G-25 and G-53.
Embodiment 48. A compound of Embodiment 47b wherein R⁵ is G-1.
Embodiment 49. A compound of Formula 1 or any one of Embodiments 1 through 48 either alone or in combination, wherein each R^{8a} is independently halogen, cyano, C₁-C₄ alkyl, C₂-C₄ alkenyl, C₂-C₄ alkynyl, C₁-C₄ haloalkyl, C₂-C₄ haloalkenyl, C₂-C₄ haloalkynyl, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalkylsulfinyl, C₁-C₄ alkylsulfonyl, C₁-C₄ haloalkylsulfonyl, C₁-C₄ alkylamino, C₂-C₆ dialkylamino, C₂-C₄ alkylcarbonyl, C₂-C₄ alkoxycarbonyl, C₃-C₆ trialkylsilyl; or Q².
Embodiment 50. A compound of Embodiment 49 wherein each R^{8a} is independently halogen, cyano, C₁-C₄ alkyl, C₁-C₄ haloalkyl, C₁-C₄ alkoxy, C₁-C₄ haloalkoxy, C₁-C₄ alkylthio, C₁-C₄ haloalkylthio, C₃-C₆ trialkylsilyl or Q².
Embodiment 51. A compound of Embodiment 50 wherein each R^{8a} is independently halogen, cyano, C₁-C₄ alkyl, C₁-C₄ haloalkyl or Q².
Embodiment 52. A compound of Embodiment 51 wherein each R^{8a} is independently halogen or CF₃.
Embodiment 53. A compound of Formula 1 or any one of Embodiments 1 through 52 either alone or in combination, wherein each Q² is independently a phenyl, benzyloxy or phenoxy.
Embodiment 54. A compound of Formula 1 or any one of Embodiments 1 through 53 either alone or in combination, wherein each R^{8b} is independently C₁-C₄ alkyl.
Embodiment 55. A compound of Formula 1 or any one of Embodiments 1 through 54 either alone or in combination, wherein R⁶ is hydrogen, C₁-C₂ alkyl or C₁-C₂ haloalkyl; or R⁶ and R^{4a} are taken together with the linking atoms to which they are attached to form a 3- to 6-membered ring containing ring members, in addition to the linking atoms, selected from carbon atoms and up to 2 heteroatoms independently selected from up to 1 O, up to 1 S and up to 1 N atom, the ring optionally substituted with up to 3 substituents independently selected from halogen, cyano, C₁-C₂ alkyl, C₁-C₂ haloalkyl, on carbon atom ring members and C₁-C₂ alkyl on nitrogen atom ring members.
Embodiment 56. A compound of Embodiment 55 wherein R⁶ is hydrogen, C₁-C₂ alkyl or C₁-C₂ haloalkyl; or R⁶ and R^{4a} are taken together with the linking atoms to which they are attached to form a 3- to 6-membered ring containing ring members, in addition to the linking atoms, selected from carbon atoms, the ring being unsubstituted.
Embodiment 57. A compound of Embodiment 56 wherein R⁶ is hydrogen, C₁-C₂ alkyl or C₁-C₂ haloalkyl.
Embodiment 58. A compound of Embodiment 57 wherein R⁶ is hydrogen or CH₃.
Embodiment 59. A compound of Embodiment 58 wherein R⁶ is hydrogen.
Embodiment 60. A compound of Formula 1 or any one of Embodiments 1 through 59 either alone or in combination, wherein each R^{9a} is independently halogen, C₁-C₂ alkyl or C₁-C₂ haloalkyl.
Embodiment 61. A compound of Formula 1 or any one of Embodiments 1 through 60 either alone or in combination, wherein each R^{9b} is independently C₁-C₂ alkyl, C₁-C₂ haloalkyl or C₂-C₃ alkylcarbonyl.

Embodiments of this invention, including Embodiments 1-61 above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this invention, including Embodiments 1-61 above as well as any other embodiments described herein, and any combination thereof, pertain to the compositions and methods of the present invention.

Combinations of Embodiments 1-61 are illustrated by:
Embodiment A. A compound of Formula 1 wherein
   A¹ is CH and A² is CH or A¹ is CH and A² is N;
   R¹ is hydrogen, SH, SCN, C₁-C₆ alkylthio or C₂-C₆ alkenylthio;
   R² is C₁-C₆ alkyl optionally substituted with up to 5 substituents independently selected from R^{2a}; or C₃-C₈ cycloalkyl optionally substituted with up to 5 substituents independently selected from R^{2b}; or ZQ¹;
   R³ is hydrogen, CHO, C₂-C₄ alkylcarbonyl, C₂-C₄ alkoxycarbonyl, C₁-C₄ alkyl, or C₂-C₄ alkoxyalkyl;
   R^{4a} is hydrogen, halogen or C₁-C₂ alkyl;
   R^{4b} is hydrogen, halogen or C₁-C₂ alkyl;
   L is a direct bond; or a 1-, 2- or 3-membered saturated or partially unsaturated chain containing chain members selected from carbon atoms and up to 2 heteroatoms independently selected from up to 1 O, up to 1 S, and up to 2 N, wherein the chain is optionally substituted with up to 3 substituents independently selected from R^{7a} on carbon atom chain members and R^{7b} on nitrogen atom chain members;
   R⁵ is C₁-C₈ alkyl, C₁-C₈ haloalkyl, C₃-C₈ cycloalkyl, C₃-C₈ halocycloalkyl, C₄-C₁₀ alkylcycloalkyl, C₂-C₈ alkoxyalkyl, C₂-C₈ haloalkoxyalkyl, C₁-C₈ alkoxy, C₁-C₈ haloalkoxy, C₃-C₈ cycloalkoxy, C₃-C₈ halocycloalkoxy, C₁-C₈ alkylthio, C₁-C₈ haloalkylthio, C₁-C₈ alkylamino, C₁-C₈ haloalkylamino, C₂-C₈ dialkylamino, C₂-C₈ halodialkylamino, or C₃-C₈ trialkylsilyl; or G; and
   R⁶ is hydrogen, C₁-C₂ alkyl or C₁-C₂ haloalkyl; or R⁶ and R^{4a} are taken together with the linking atoms to which they are attached to form a 3- to 6-membered ring containing ring members, in addition to the linking atoms, selected from carbon atoms and up to 2 heteroatoms independently selected from up to 1 O, up to 1 S and up to 1 N atom, the ring optionally substituted with up to 3 substituents independently selected from halogen, cyano, C₁-C₂ alkyl, C₁-C₂ haloalkyl, on carbon atom ring members and C₁-C₂ alkyl on nitrogen atom ring members.
Embodiment B. A compound of Embodiment A wherein
   A¹ is CH and A² is N;
   R¹ is hydrogen, SH, SCN, SCH₃ or SCH₂CH═CH₂;
   R² is C₁-C₆ alkyl optionally substituted with up to 3 substituents independently selected from R^{2a}; or C₃-C₆ cycloalkyl optionally substituted with up to 3 substituents independently selected from R^{2b}; or ZQ¹;
   each R^{2a} is independently halogen, cyano, C₁-C₆ alkoxy, C₁-C₆ haloalkoxy, C₃-C₆ cycloalkyl, phenyl or phenoxy;
   each R^{2b} is independently halogen, cyano, C₁-C₆ alkyl, C₁-C₆ haloalkyl, C₁-C₆ alkoxy or C₁-C₆ haloalkoxy;
   Z is a direct bond or CH₂;
   Q¹ is selected from Q-1 through Q-65 depicted in Exhibit 1 wherein when R⁹ is attached to a carbon ring member, said R⁹ is selected from R^{9a}, and when R⁹ is attached to a nitrogen ring member, said R⁹ is selected from R^{9b}; and k is 0, 1, 2 or 3;
   R³ is hydrogen or C₁-C₄ alkyl;
   R^{4a} is hydrogen;
   R^{4b} is hydrogen;
   L is a direct bond; or a 1- or 2-membered saturated chain containing chain members selected from carbon atoms and up to 1 heteroatom selected from up to 1 O, up to 1 S, and up to 1 N, wherein the chain is optionally substituted with up to 2 substituents independently selected from R^{7a} on carbon atom chain members and R^{7b} on nitrogen atom chain members;
   each R^{7a} is independently halogen, C₁-C₂ alkyl or C₁-C₂ haloalkyl;
   each R^{7b} is independently C₁-C₂ alkyl, C₁-C₂ haloalkyl or C₂-C₃ alkylcarbonyl;
   R⁵ is C₁-C₈ alkyl, C₁-C₈ haloalkyl, C₃-C₈ cycloalkyl, C₃-C₈ halocycloalkyl, C₁-C₈ alkoxy or C₁-C₈ haloalkoxy; or G;
   G is selected from G-1 through G-65 depicted in Exhibit 2 wherein when R⁸ is attached to a carbon ring member, said R⁸ is selected from R^{8a}, and when R⁸ is attached to a nitrogen ring member, said R⁸ is selected from R^{8b}; and m is 0, 1, 2 or 3;
   each R^{8a} is independently halogen, cyano, C₁-C₄ alkyl, C₂-C₄ alkenyl, C₂-C₄ alkynyl, C₁-C₄ haloalkyl, C₂-C₄ haloalkenyl, C₂-C₄ haloalkynyl, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalkylsulfinyl, C₁-C₄ alkylsulfonyl, C₁-C₄ haloalkylsulfonyl, C₁-C₄ alkylamino, C₂-C₆ dialkylamino, C₂-C₄ alkylcarbonyl, C₂-C₄ alkoxycarbonyl, C₃-C₆ trialkylsilyl; or Q²; and
   R⁶ is hydrogen, C₁-C₂ alkyl or C₁-C₂ haloalkyl.
Embodiment C. A compound of Embodiment B wherein
   R¹ is hydrogen or SH;
   Q¹ is selected from Q-1 through Q-3, Q-15, Q-25, Q-35, Q-50 and Q-54;
   R³ is hydrogen or CH₃;
   L is a direct bond; or —CH₂—, —CH₂O—, —CH₂S—, —CH₂NR^{7b}— or —CH₂CH₂— wherein the left bond is connected to the isoxazoline ring and the right bond is connected to R⁵;
   R⁵ is G;
   G is selected from G-1, G-2, G-3, G-12, G-13, G-14, G-25 and G-53;
   each R^{8a} is independently halogen, cyano, C₁-C₄ alkyl, C₁-C₄ haloalkyl, C₁-C₄ alkoxy, C₁-C₄ haloalkoxy, C₁-C₄ alkylthio, C₁-C₄ haloalkylthio, C₃-C₆ trialkylsilyl; or Q²;
   each Q² is independently a phenyl, benzyloxy or phenoxy;
   each R^{8b} is independently C₁-C₄ alkyl; and
   R⁶ is hydrogen or CH₃.
Embodiment D. A compound of Embodiment C wherein
   R¹ is hydrogen;
   R² is C₄-C₆ alkyl; C₃-C₆ cycloalkyl; or ZQ¹ wherein Z is a direct bond or CH₂ and Q¹ is selected from Q-1, Q-15, Q-25, Q-35, Q-50 and Q-54;
   R³ is hydrogen;
   L is —CH₂—, —CH₂O—, —CH₂S— or —CH₂CH₂— wherein the left bond is connected to the isoxazoline ring and the right bond is connected to R⁵;
   R⁵ is G-1;
   each R^{8a} is independently halogen or CF₃;
   R⁶ is hydrogen; and
   R^{9a} is independently halogen, C₁-C₂ alkyl or C₁-C₂ haloalkyl.

Specific embodiments include compounds of Formula 1 selected from the group consisting of:
α-[5-[(4-bromophenoxy)methyl]-4,5-dihydro-3-isoxazolyl]-α-(1,1-dimethylpropyl)-1H-1,2,4-triazole-1-ethanol,
α-[4,5-dihydro-5-[2-[4-(trifluoromethyl)phenyl]ethyl]-3-isoxazolyl]-α-(1,1-dimethylethyl)-1H-1,2,4-triazole-1-ethanol,
α-[5-[(4-chlorophenoxy)methyl]-4,5-dihydro-3-isoxazolyl]-α-(4-chlorophenyl)-1H-1,2,4-triazole-1-ethanol,
α-[4,5-dihydro-5-[[4-(trifluoromethyl)phenoxy]methyl]-3-isoxazolyl]-α-(1,1-dimethylethyl)-1H-1,2,4-triazole-1-ethanol,
α-(4-chlorophenyl)-α-[5-(4-chlorophenyl)-4,5-dihydro-3-isoxazolyl]-1H-1,2,4-triazole-1-ethanol,
α-[5-(4-chlorophenyl)-4,5-dihydro-3-isoxazolyl]-α-(1-methylcyclopropyl)-1H-1,2,4-triazole-1-ethanol,
α-(4-chlorophenyl)-α-[4,5-dihydro-5-(4-phenoxyphenyl)-3-isoxazolyl]-1H-1,2,4-triazole-1-ethanol,
α-(4-chlorophenyl)-α-[4,5-dihydro-5-[4-(trifluoromethyl)phenyl]-3-isoxazolyl]-1H-1,2,4-triazole-1-ethanol,
α-(4-chlorophenyl)-α-[5-[[(5-chloro-2-pyridinyl)oxy]methyl]-4,5-dihydro-3-isoxazolyl]-1H-1,2,4-triazole-1-ethanol,
α-[5-[(4-chlorophenoxy)methyl]-4,5-dihydro-3-isoxazolyl]-α-(1,1-dimethylethyl)-1H-1,2,4-triazole-1-ethanol and
α-[5-[(4-bromophenoxy)methyl]-4,5-dihydro-3-isoxazolyl]-α-(1,1-dimethylethyl)-1H-1,2,4-triazole-1-ethanol.

This invention provides a fungicidal composition comprising a compound of Formula 1 (including all stereoisomers, N-oxides, and salts thereof), and at least one other fungicide. Of note as embodiments of such compositions are compositions comprising a compound corresponding to any of the compound embodiments described above.

This invention provides a fungicidal composition comprising a compound of Formula 1 (including all stereoisomers, N-oxides, and salts thereof) (i.e. in a fungicidally effective amount), and at least one additional component selected from the group consisting of surfactants, solid diluents and liquid diluents. Of note as embodiments of such compositions are compositions comprising a compound corresponding to any of the compound embodiments described above.

This invention provides a method for controlling plant diseases caused by fungal plant pathogens comprising applying to the plant or portion thereof, or to the plant seed, a fungicidally effective amount of a compound of Formula 1 (including all stereoisomers, N-oxides, and salts thereof). Of note as embodiment of such methods are methods comprising applying a fungicidally effective amount of a compound corresponding to any of the compound embodiments describe above. Of particular notes are embodiments where the compounds are applied as compositions of this invention.

One or more of the following methods and variations as described in Schemes 1-7 can be used to prepare the compounds of Formula 1. The definitions of A1, A2, R¹, R², R³, R^{4a}, R^{4b}, R⁵, R⁶ and L in the compounds of Formulae 1-11 below are as defined above in the Summary of the Invention unless otherwise noted. Compounds of Formulae 1a-1c are various subsets of the compounds of Formula 1, and all substituents for Formulae 1a-1c are as defined above for Formula 1.

As shown in Scheme 1, compounds of Formula 1a (Formula 1 wherein R¹ is H and R³ is H) can be prepared by opening of an epoxide of Formula 3 with a heterocycle of Formula 2 in the presence of a base. Typical bases include amine bases such as triethylamine, N,N-diisopropylethylamine, DBU, hydroxides such as sodium and potassium hydroxide and carbonates such as sodium carbonate and potassium carbonate. The corresponding heterocyclic sodium or potassium salt may also be used. Typically, a polar, aprotic solvent is used, such as acetonitrile or dimethyl formamide. Compounds of Formula 2 are commercially available.

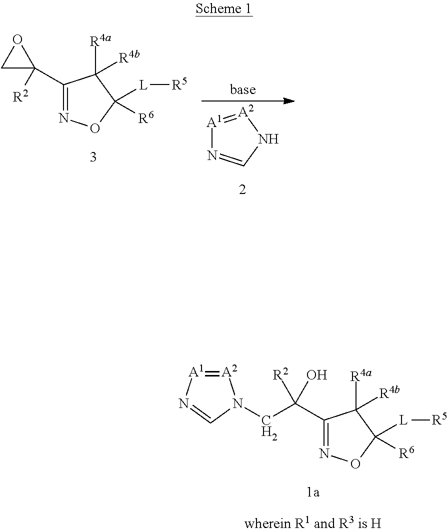

An alternate procedure for the preparation of compounds of Formula 1a is depicted in Scheme 2 and involves reacting a heterocycle of Formula 2 with an alkyl halide of Formula 4 in the presence of an acid scavenger. Typical acid scavengers include amine bases such as triethylamine, N,N-diisopropylethylamine, DBU, hydroxides such as sodium and potassium hydroxide and carbonates such as sodium carbonate and potassium carbonate. Suitable solvents include acetonitrile or dimethyl formamide.

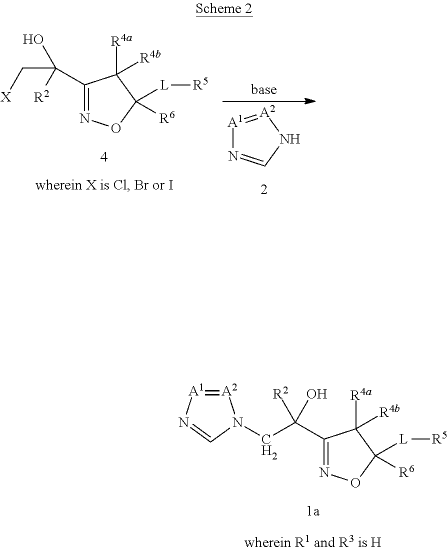

Compounds of Formula 1b (Formula 1 wherein R¹ is H) can be prepared by reaction of the compounds of Formula 1a and a compound of Formula 5 as shown in Scheme 3. The reaction is carried out in the presence of an acid scavenger. Typical acid scavengers include amine bases such as triethylamine, N,N-diisopropylethylamine and pyridine. Other scavengers include hydroxides such as sodium and potassium hydroxide and carbonates such as sodium carbonate and potassium carbonate in a solvent such as tetrahydrofuran, N,N-dimethylformamide or acetonitrile at 0 to 80° C. The compounds of Formula 5 are known in the chemical literature and some are commercially available.

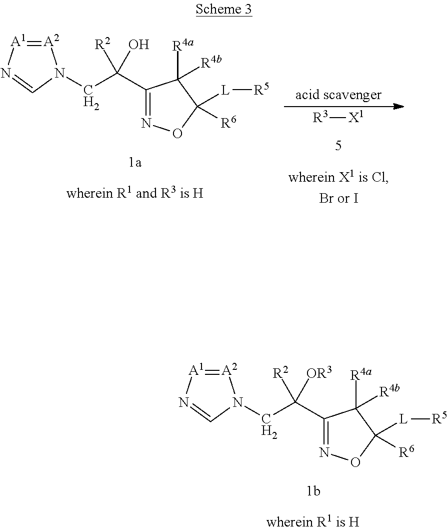

Compounds of Formula 1c (compounds of Formula 1 wherein R¹ is SH), can be prepared by reaction of a compound of Formula 1b with a strong base such as n-butyl lithium or lithium diisopropylethylamide as depicted in Scheme 4. This reaction is typically carried out between 0° C. and -70° C. in a solvent such as tetrahydrofuran as described in U.S. Pat. No. 5,789,430. Compounds of Formula 1c wherein R¹ is SH can be alkylated on sulfur to give additional compounds of Formula 1 as known to one skilled in the art.

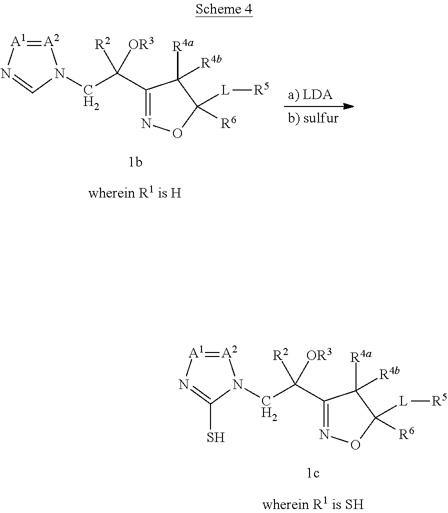

Compounds of Formula 3 can be prepared treating a ketone of Formula 6 with a sulfur ylide precursor of Formula 7 as shown in Scheme 5. Typical conditions involve treating a compound of Formula 7 with a strong base such as sodium hydride or potassium t-butoxide in dimethylsulfoxide or dimethylformamide at 0° C. to ambient temperature for 30 to 60 minutes and reacting the resulting ylide with the ketone. This type of raction is very well known; see, for example, Corey, E. J.; Chaykovsky, M. J. Am. Chem. Soc. 1965, 87, 1353-1364. One skilled in the art will recognize that certain other sulfur ylide precursors other than the specific one shown in Formula 7 can be used.

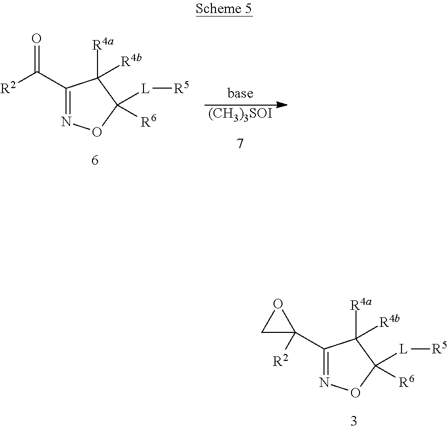

The ketones of Formula 6 can be prepared by the reaction of a compound of Formula 8 with an olefin of Formula 9 in the presence of an acid scavenger as shown in Scheme 6. The reaction is carried out in a solvent such as ethyl acetate or acetone with a base such as solid sodium bicarbonate, or in dichloromethane with a base such as triethylamine at 0° C. to ambient temperature. Compounds of Formula 9 are known in the chemical literature and some are commercially available. Many compounds of Formula 8 are known or can be prepared by nitosating the corresponding haloketone as known to one skilled in the art.

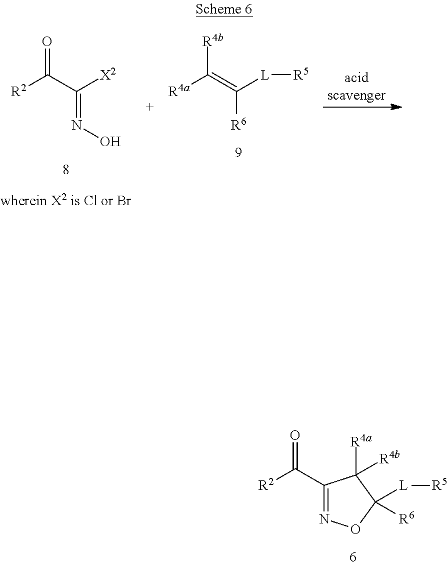

Compounds of Formula 4 can be prepared by reaction of a compound of Formula 10 with an organometallic reagent of Formula 11 as shown in Scheme 7. The reaction is carried out at 0° C. to −70° C. in a solvent such as tetrahydrofuran (see, for example, WO 2014/095548). The organometallic reagent can be an organo lithium, an organo zinc reagent or a Grignard reagent. The choice of reagent will depend on the nature of the R² moiety and the other functionality present in the Formula 11 compounds as will be known to one skilled in the art. Many compounds of Formula 11 are known or can be prepared by known methods. The haloketones of Formula 10 can be prepared by a method similar to that shown in Scheme 6 above, where the compound of Formula 8 is replaced with, for example, the known 3-chloro-N-hydroxy-2-oxopropanimidoyl chloride.

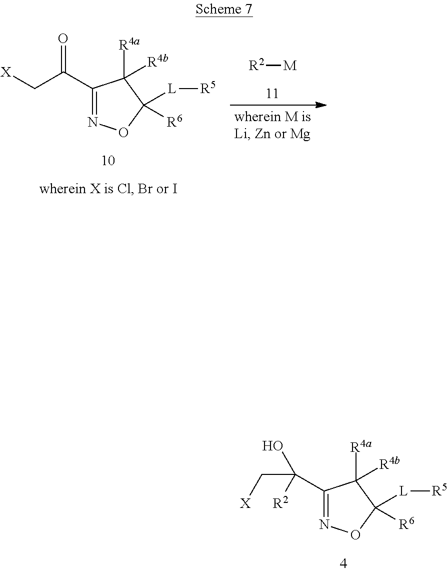

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular sequence presented to prepare the compounds of Formula 1.

One skilled in the art will also recognize that compounds of Formula 1 and the intermediates described herein can be subjected to various electrophilic, nucleophilic, radical, organometallic, oxidation, and reduction reactions to add substituents or modify existing sub stituents.

Without further elaboration, it is believed that one skilled in the art using the preceding description can utilize the present invention to its fullest extent. The following Examples are, therefore, to be construed as merely illustrative, and not limiting of the disclosure in any way whatsoever. Steps in the following Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane; “s” means singlet, “d” means doublet, “t” means triplet, “q” means quartet, “m” means multiplet, “dd” means doublet of doublets, “dt” means doublet of triplets, “br s” means broad singlet.

### SYNTHESIS EXAMPLE 1

### Preparation of α-[5-[(4-chlorophenoxy)methyl]-4,5-dihydro-3-isoxazolyl]-α-(1,1-dimethylethyl)-1H-1,2,4-triazole-1-ethanol (Compounds 174 and 175)

### Step A: Preparation of 1-[5-[(4-chlorophenoxy)methyl]-4,5-dihydro-3-isoxazolyl]-2,2-dimethyl-1-propanone

To a solution of (1Z)-N-hydroxy-3,3-dimethyl-2-oxobutanimidoyl chloride (4.09 g, 25 mmol) and 1-chloro-4-prop-2-enoxybenzene (4.22 g, 25 nmol) in ethyl acetate (100 mL) was added anhydrous sodium bicarbonate (6.30 g, 75 mmol). The reaction mixture was stirred at room temperature for 3 days, filtered and concentrated under reduced pressure to give 7.7 g of a colorless oil. The oil was dissolved in hot petroleum ether (25 mL) and allowed to stand to give 5.23 g of the title compound as white powder.

¹H NMR (CDCl₃): δ 1.34 (s, 9H), 3.16-3.21 (m, 1H), 3.29-3.34 (m, 1H), 4.05-4.06 (m, 2H), 4.97-5.03 (m, 1H),), 6.80-6.83 (m, 2H), 7.21-7.25 (m, 2H).

### Step B: Preparation of 5-[(4-chlorophenoxy)-methyl]-3-[2-(1,1-dimethylethyl)-2-oxiranyl]-4,5-dihydroisoxazole

To a solution of potassium t-butoxide (2.24 g, 20 mmol) in anhydrous dimethylsulfoxide (20 mL) was added trimethylsulfoxonium iodide (4.40 g, 20 mmol) in one portion. The mixture was stirred at room temperature for 1 hour, then 1-[5-[(4-chlorophenoxy)methyl]-4,5-dihydro-3-isoxazolyl]-2,2-dimethyl-1-propanone (i.e. the product of Step A) (5.23 g, 18 mmole) was added in one portion. The mixture was stirred at room temperature for 1 hour, added slowly to rapidly stirred ice-water (200 mL) and the resulting solid filtered, washed with water and air dried to give 5.20 g of the title compound as a yellow powder (mix of diastereomers). The diastereomer mixture was purified by column chromatography on silica gel using 0-30% ethyl acetate in hexanes as eluant to give 1.97 g of the less polar diastereomer (first to elute) as a white solid and 2.51 g of the more polar diastereomer (second to elute) as a white solid.

### Less Polar Diastereomer:

¹H NMR (CDCl₃): δ 1.08 (s, 9H), 2.81-2.82 (m, 1H), 2.97-3.02 (m, 1H), 3.08-3.09 (m, 1H), 3.18-3.23 (m, 1H), 3.92-3.95 (m, 1H), 3.97-4.00 (m, 1H) 4.87-4.93 (m, 1H), 6.79-6.82 (m, 2H), 7.21-7.24 (m, 2H).

### More Polar Diastereomer:

¹H NMR (CDCl₃): δ 1.09 (s, 9H), 2.83-2.84 (m, 1H), 3.08-3.19 (m, 3H), 4.01-4.02 (m, 2H), 4.86-4.91 (m, 1H), 6.81-6.85 (m, 2H), 7.22-7.25 (m, 2H).

### Step C: Preparation of α-[5-[(4-chlorophenoxy)methyl]-4,5-dihydro-3-isoxazolyl]-α-(1,1-dimethylethyl)-1H-1,2,4-triazole-1-ethanol

To a solution of 3-[2-(1,1-dimethylethyl)-2-oxiranyl]-5-[(4-chlorophenoxy)methyl]-4,5-dihydroisoxazole (i.e. less polar diastereomer; the first product of Step B) (1.97 g, 6.35 mmol) and 1,2,4-triazole (0.48 g, 7.0 mmol) in anhydrous acetonitrile (10 mL) was added 4 drops of DBU. The mixture was heated between 70-80° C. for 24 hour, cooled, concentrated under reduced pressure and purified by column chromatography on silica gel using 50-100% ethyl acetate in hexanes as eluant to give 1.57 g of the title compound (a compound of the present invention) as white solid. The solid was recrystallized from methanol to give 1.38 g of colorless crystals, mp 125-127° C. An x-ray crystal structure analysis on a similarly prepared sample showed this to be the (R,S/S,R) diastereomer (racemic mixture).

¹H NMR (CDCl₃): δ 1.13 (s, 9H), 2.81-2.86 (m, 1H), 3.16-3.22 (m, 1H), 3.48-3.51 (m, 1H), 3.76-3.79 (m, 1H), 4.33-4.36 (m, 1H), 4.76-4.84 (m, 2H) 4.92 (s, 1H), 6.75-6.78 (m, 2H), 7.22-7.25 (m, 2H), 7.87 (s, 1H), 8.13 (s, 1H).

### SYNTHESIS EXAMPLE 2

### Preparation of α-[5-(4-chlorophenyl)-4,5-dihydro-3-isoxazolyl]-α-1-propyn-1-yl-1H-1,2,4-triazole-1-ethanol (Compounds 103 and 104)

### Step A: Preparation of 2-chloro-1-[5-(4-chlorophenyl)-4,5-dihydro-3-isoxazolyl]-ethanone

To a solution of (1Z)-3-chloro-N-hydroxy-2-oxopropanimidoyl chloride (3.12 g, 20 mmol) and 4-chlorostyrene (2.77 g, 20 nmol) in ethyl acetate (100 mL) was added anhydrous sodium bicarbonate (5.0 g, 60 mmol). The reaction mixture was stirred at room temperature for 3 days, filtered and concentrated under reduced pressure to give 5.2 g of a yellow oil. The oil was heated in hexane (100 mL) and allowed to stand to give 1.0 g of the title compound as colorless needles over a solid mass. The needles were collected and the remaining solid heated with hexane to give an additional 1.5 g of the title compound as colorless needles.

¹H NMR (CDCl₃): δ 3.14-3.20 (m, 1H), 3.59-3.65 (m, 1H), 4.72 (s, 2H), 5.76-5.80 (m, 1H),), 7.23-7.26 (m, 2H), 7.36-7.39 (m, 2H).

### Step B: Preparation of α-(chloromethyl)-5-(4-chlorophenyl)-4,5-dihydro-α-1-propyn-1-yl-3-isoxazolemethanol

To a −70° C. cooled solution of 1-propynylmagnessium bromide (0.5 M in THF, 12 mL, 6.0 mmol) was added a solution of 2-chloro-1-[5-(4-chlorophenyl)-4,5-dihydro-3-isoxazolyl]ethanone (i.e. the product of Step A) (1.0 g, 4.0 mmole) in anhydrous tetrahydrofuran (20 mL) dropwise over 15 minutes. The mixture was stirred at −70° C. for 1 hour, warmed to −40° C. and added to a mixture of ice and saturated aqueous ammonium chloride solution. The mixture was ether extracted and the extract was washed with saturated aqueous sodium chloride solution, dried over anhydrous magnesium sulfate and concentrated under reduced pressure to give 1.20 g of a yellow oil. The oil was purified by column chromatography on silica gel using 0-50% ethyl acetate in hexanes as eluant to give 1.07 g of the title compound as colorless oil (mix of diastereomers).

¹H NMR (CDCl₃): δ 1.88-1.89 (m, 3H), 3.05-3.16 (m, 1H), 3.25-3.30 (m, 1H), 3.55-3.65 (m, 1H), 3.88-3.96 (m, 2H), 5.64-5.68 (m, 1H), 7.27-7.30 (m, 2H), 7.34-7.36 (m, 2H).

### Step C: Preparation of α-[5-(4-chlorophenyl)-4,5-dihydro-3-isoxazolyl]-α-1-propyn-1-yl-1H-1,2,4-triazole-1-ethanol

To a suspension of sodium hydride (60% oil dispersion; 0.28 g, 7.0 mmol) in anhydrous dimethylformamide (10 mL) was added 1,2,4-triazole (0.48 g, 7.0 mmol). The mixture was stirred at room temperature for 30 minutes, then a solution of α-(chloromethyl)-5-(4-chlorophenyl)-4,5-dihydro-α-1-propyn-1-yl-3-isoxazolemethanol (i.e. the product of Step B) (0.98 g, 3.4 mmol) in anhydrous dimethylformamide (10 mL) was added. The mixture was heated at 90° C. for 3 hour, cooled, diluted with water and ethyl acetate extracted. The extract was washed with water, saturated aqueous sodium chloride solution, dried over anhydrous magnesium sulfate and concentrated under reduced pressure to give a yellow oil. The oil was heated in diethyl ether (20 mL) to give 0.7 g of a tan powder. The solid was recrystallized from acetonitrile two times to give 232 mg of a 83:4:13 mixture of isomers. The mother liquor was purified by column chromatography on silica gel using 100% ethyl acetate as eluant to give 235 mg of the title compound, a compound of the present invention, as a white powder (1:1 mix of diastereomers).

¹H NMR (CDCl₃): δ 1.80-1.83 (m, 3H), 3.12-3.23 (m, 1H), 3.58-3.69 (m, 1H), 4.66-4.73 (m, 2H), 4.78 (br s, 1H), 5.59-5.65 (m, 1H), 7.20-7.28 (m, 2H), 7.32-7.37 (m, 2H), 7.93-7.95 (m, 1H), 8.23-8.24 (m, 1H).

By the procedures described herein together with methods known in the art, the following compounds of Tables 1A to 2Y can be prepared. The following abbreviations are used in the Tables which follow: t means tertiary, s means secondary, n means normal, i means iso, c means cyclo, Me means methyl, Et means ethyl, Pr means propyl, i-Pr means isopropyl, c-Pr means cyclopropyl, Bu means butyl, Ph means phenyl, OMe means methoxy, OEt means ethoxy, SMe means methylthio, SEt means ethylthio, —CN means cyano, Ph means phenyl, Py means pyridinyl, —NO₂ means nitro, TMS means trimethylsilyl, S(O)Me means methylsulfinyl, and S(O)₂Me means methylsulfonyl.

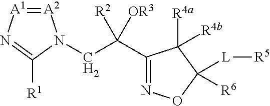

TABLE 1A

| R² | R³ | R^{4a} | R^{4b} | L | R⁵ | R⁶ |
|---|---|---|---|---|---|---|
| Methyl | H | H | H | bond | 4-Cl-phenyl | H |
| Ethyl | H | H | H | bond | 4-Cl-phenyl | H |
| Propyl | H | H | H | bond | 4-Cl-phenyl | H |
| iPropyl | H | H | H | bond | 4-Cl-phenyl | H |
| nButyl | H | H | H | bond | 4-Cl-phenyl | H |
| iButyl | H | H | H | bond | 4-Cl-phenyl | H |
| tButyl | H | H | H | bond | 4-Cl-phenyl | H |
| t-Amyl | H | H | H | bond | 4-Cl-phenyl | CH₃ |
| Neopentyl | H | H | H | bond | 4-Cl-phenyl | CH₃ |
| nHexyl | H | H | H | bond | 4-Cl-phenyl | H |
| iHexyl | H | H | H | bond | 4-Cl-phenyl | H |
| c-Pr | H | H | H | bond | 4-Cl-phenyl | H |
| Cyclobutyl | H | H | H | bond | 4-Cl-phenyl | H |
| Cyclopentyl | H | H | H | bond | 4-Cl-phenyl | H |
| cyclohexyl | H | H | H | bond | 4-Cl-phenyl | H |
| Vinyl | H | H | H | bond | 4-Cl-phenyl | H |
| Allyl | H | H | H | bond | 4-Cl-phenyl | H |
| Propargyl | H | H | H | bond | 4-Cl-phenyl | H |
| 1-hexen-6-yl | H | H | H | bond | 4-Cl-phenyl | H |
| 1-hexyn-6-yl | H | H | H | bond | 4-Cl-phenyl | H |
| Methoxymethyl | H | H | H | bond | 4-Cl-phenyl | H |
| 2,2,2-trifluoro- | H | H | H | bond | 4-Cl-phenyl | H |
| ethoxy methyl | | | | | | |
| c-Pr methyl | H | H | H | bond | 4-Cl-phenyl | CH₃ |
| Phenyl | H | H | H | bond | 4-Cl-phenyl | H |
| Benzyl | H | H | H | bond | 4-Cl-phenyl | H |
| Phenoxymethyl | H | H | H | bond | 4-Cl-phenyl | H |
| CF₃ | H | H | H | bond | 4-Cl-phenyl | H |
| CCl₃ | H | H | H | bond | 4-Cl-phenyl | H |
| t-Butyl | CH₃ | H | H | bond | 4-Cl-phenyl | H |
| t-Butyl | Ethyl | H | H | bond | 4-Cl-phenyl | H |
| t-Butyl | Propyl | H | H | bond | 4-Cl-phenyl | H |
| t-Butyl | Butyl | H | H | bond | 4-Cl-phenyl | H |
| t-Butyl | i-Pr | H | H | bond | 4-Cl-phenyl | H |
| t-Butyl | 2-Cl—Et | H | H | bond | 4-Cl-phenyl | H |
| t-Butyl | Allyl | H | H | bond | 4-Cl-phenyl | H |
| t-Butyl | CH₂C≡CH | H | H | bond | 4-Cl-phenyl | H |
| t-Butyl | Methoxy- | H | H | bond | 4-Cl-phenyl | H |
| | methyl | | | | | |
| t-Butyl | Formyl | H | H | bond | 4-Cl-phenyl | H |
| t-Butyl | Acetyl | H | H | bond | 4-Cl-phenyl | H |
| t-Butyl | Trifluoro- | H | H | bond | 4-Cl-phenyl | H |
| | acetyl | | | | | |
| t-Butyl | Methoxy- | H | H | bond | 4-Cl-phenyl | H |
| | acetyl | | | | | |
| t-Butyl | H | Me | H | bond | 4-Cl-phenyl | H |
| t-Butyl | H | H | Me | bond | 4-Cl-phenyl | H |
| t-Butyl | H | Me | Me | bond | 4-Cl-phenyl | H |
| t-Butyl | H | F | F | bond | 4-Cl-phenyl | H |
| t-Butyl | H | CN | H | bond | 4-Cl-phenyl | H |
| t-Butyl | H | H | CF₃ | bond | 4-Cl-phenyl | H |
| t-Butyl | H | H | H | CH₂ | 4-Cl-phenyl | CH₃ |
| t-Butyl | H | H | H | CH₂CH₂ | 4-Cl-phenyl | CH₃ |
| t-Butyl | H | H | H | CH₂CH₂CH₂ | 4-Cl-phenyl | H |
| t-Butyl | H | H | H | CH₂CH₂CH₂CH₂ | 4-Cl-phenyl | H |
| t-Butyl | H | H | H | OCH₂ | 4-Cl-phenyl | H |
| t-Butyl | H | H | H | CH₂O | 4-Cl-phenyl | CH₃ |
| t-Butyl | H | H | H | CH₂CH₂O | 4-Cl-phenyl | CH₃ |
| t-Butyl | H | H | H | CH₂CH₂CH₂O | 4-Cl-phenyl | H |
| t-Butyl | H | H | H | CH₂OCH₂ | 4-Cl-phenyl | H |
| t-Butyl | H | H | H | CH₂S | 4-Cl-phenyl | H |
| t-Butyl | H | H | H | CH₂CH₂S | 4-Cl-phenyl | H |
| t-Butyl | H | H | H | CH₂SCH₂ | 4-Cl-phenyl | H |
| t-Butyl | H | H | H | CH₂NH | 4-Cl-phenyl | H |
| t-Butyl | H | H | H | CH₂CH₂NH | 4-Cl-phenyl | H |
| t-Butyl | H | H | H | CH₂NHCH₂ | 4-Cl-phenyl | H |
| t-Butyl | H | H | H | CH₂N(CH₃) | 4-Cl-phenyl | H |
| t-Butyl | H | H | H | CH₂N(CH₃)CH₂ | 4-Cl-phenyl | H |
| t-Butyl | H | H | H | CH(CH₃) | 4-Cl-phenyl | H |
| t-Butyl | H | H | H | CH(Cl) | 4-Cl-phenyl | H |
| t-Butyl | H | H | H | CH═CH | 4-Cl-phenyl | H |
| t-Butyl | H | H | H | C≡C | 4-Cl-phenyl | H |
| t-Butyl | H | H | H | CH₂ON═CH | 4-Cl-phenyl | H |
| t-Butyl | H | H | H | CH₂ON═C(CH₃) | 4-Cl-phenyl | H |
| t-Butyl | H | H | H | CH═NO | 4-Cl-phenyl | H |
| t-Butyl | H | H | H | CH═NOCH₂ | 4-Cl-phenyl | H |
| t-Butyl | H | H | H | C(CH₃)═NO | 4-Cl-phenyl | H |
| t-Butyl | H | H | H | C(CH₃)═NOCH₂ | 4-Cl-phenyl | H |
| t-Butyl | H | H | H | C(CH₃)₂ | 4-Cl-phenyl | H |
| t-Butyl | H | H | H | C(F)₂ | 4-Cl-phenyl | H |
| t-Butyl | H | H | H | CH(CN) | 4-Cl-phenyl | H |
| t-Butyl | H | H | H | CH(OH) | 4-Cl-phenyl | H |
| t-Butyl | H | H | H | CH(CF₃) | 4-Cl-phenyl | H |
| t-Butyl | H | H | H | CH(OCH₃) | 4-Cl-phenyl | H |
| t-Butyl | H | H | H | bond | 4-Cl-phenyl | F |
| t-Butyl | H | H | H | bond | 4-Cl-phenyl | Cl |
| t-Butyl | H | H | H | bond | 4-Cl-phenyl | Br |
| t-Butyl | H | H | H | bond | 4-Cl-phenyl | CN |
| t-Butyl | CH₃ | H | H | bond | 4-Cl-phenyl | CH₃ |
| t-Butyl | H | H | H | bond | 4-Cl-phenyl | Et |
| t-Butyl | H | H | H | bond | 4-Cl-phenyl | CF₃ |
| t-Butyl | H | H | H | bond | phenyl | H |
| t-Butyl | H | H | H | bond | 4-F-phenyl | H |
| t-Butyl | H | H | H | bond | 4-Br-phenyl | H |
| t-Butyl | H | H | H | bond | 4-I-phenyl | H |
| t-Butyl | H | H | H | bond | 4-CH₃-phenyl | H |
| t-Butyl | H | H | H | bond | 4-CF₃-phenyl | H |
| t-Butyl | H | H | H | bond | 4-CH₃O-phenyl | H |
| t-Butyl | H | H | H | bond | 4-CH₃S-phenyl | H |
| t-Butyl | H | H | H | bond | 4-CF₃O-phenyl | H |
| t-Butyl | H | H | H | bond | 4-CH₃CH₂-phenyl | H |
| t-Butyl | H | H | H | bond | 4-CN-phenyl | H |
| t-Butyl | H | H | H | bond | 4-OH-phenyl | H |
| t-Butyl | H | H | H | bond | 4-NH₂-phenyl | H |
| t-Butyl | H | H | H | bond | 4-N(CH₃)₂-phenyl | H |
| t-Butyl | H | H | H | bond | 4-NO₂-phenyl | H |
| t-Butyl | H | H | H | bond | 4-tBu-phenyl | H |
| t-Butyl | H | H | H | bond | 4-cPr-phenyl | H |
| t-Butyl | H | H | H | bond | 4-CH₃OCH₂- | H |
| | | | | | phenyl | |
| t-Butyl | H | H | H | bond | 4-CH₃OC(O)- | H |
| | | | | | phenyl | |
| t-Butyl | H | H | H | bond | 4-CH₃C(O)-phenyl | H |
| t-Butyl | H | H | H | bond | 4-CH₃OC(O)- | H |
| | | | | | phenyl | |
| t-Butyl | H | H | H | bond | 4-CH₃C(O)O- | H |
| | | | | | phenyl | |
| t-Butyl | H | H | H | bond | 4-CH₃NHC(O)- | H |
| | | | | | phenyl | |
| t-Butyl | H | H | H | bond | 4-(CH₃)2NC(O)- | H |
| | | | | | phenyl | |
| t-Butyl | H | H | H | bond | 4-CH₃Si-phenyl | H |
| t-Butyl | H | H | H | bond | 4-Ph-phenyl | H |
| t-Butyl | H | H | H | bond | 4-PhO-phenyl | H |
| t-Butyl | H | H | H | bond | 4-PhCH₂O-phenyl | H |
| t-Butyl | H | H | H | bond | 4-PhS-phenyl | H |
| t-Butyl | H | H | H | bond | 4-PhCH₂S-phenyl | H |
| t-Butyl | H | H | H | bond | 4-(4-Br-pyrazol-1- | H |
| | | | | | yl)-phenyl | |
| t-Butyl | H | H | H | bond | 2-Cl-phenyl | H |
| t-Butyl | H | H | H | bond | 3-Cl-phenyl | H |
| t-Butyl | H | H | H | bond | 2,4-diCl-phenyl | H |
| t-Butyl | H | H | H | bond | 2,4-diF-phenyl | H |
| t-Butyl | H | H | H | bond | 3-Br-phenyl | H |
| t-Butyl | H | H | H | bond | 3-CF₃-phenyl | H |
| t-Butyl | H | H | H | bond | 2-F,4-Cl-phenyl | H |
| t-Butyl | H | H | H | bond | 3,4-diCl-phenyl | H |
| t-Butyl | H | H | H | bond | 3,5-diCl-phenyl | H |
| t-Butyl | H | H | H | bond | 3-PhO-phenyl | H |
| t-Butyl | H | H | H | bond | 2,6-diCl-phenyl | H |
| t-Butyl | H | H | H | bond | 2,6-diF-phenyl | H |
| t-Butyl | H | H | H | bond | 2-F,4-CF₃-phenyl | H |
| t-Butyl | H | H | H | bond | Naphthalen-1yl | H |
| t-Butyl | H | H | H | bond | Naphthalen-2yl | H |
| 4-Cl-phenyl | H | H | H | bond | 4-F-phenyl | H |
| 4-Cl-phenyl | H | H | H | bond | 4-Cl-phenyl | H |
| 4-Cl-phenyl | H | H | H | bond | 4-Br-phenyl | H |
| 4-Cl-phenyl | H | H | H | bond | 4-I-phenyl | H |
| 4-Cl-phenyl | H | H | H | bond | 4-CH₃-phenyl | H |
| 4-Cl-phenyl | H | H | H | bond | 4-CF₃-phenyl | H |
| 4-Cl-phenyl | H | H | H | bond | 4-CH₃O-phenyl | H |
| 4-Cl-phenyl | H | H | H | bond | 4-CH₃S-phenyl | H |
| 4-Cl-phenyl | H | H | H | bond | 4-CF₃O-phenyl | H |
| 4-Cl-phenyl | H | H | H | bond | 4-CH₃CH₂-phenyl | H |
| 4-Cl-phenyl | H | H | H | bond | 4-CN-phenyl | H |
| 4-Cl-phenyl | H | H | H | bond | 2-Cl-phenyl | H |
| 4-Cl-phenyl | H | H | H | bond | 3-Cl-phenyl | H |
| 4-Cl-phenyl | H | H | H | bond | 2,4-diCl-phenyl | H |
| 4-Cl-phenyl | H | H | H | bond | 2,4-diF-phenyl | H |
| 4-Cl-phenyl | H | H | H | bond | 3-Br-phenyl | H |
| 4-Cl-phenyl | H | H | H | bond | 3-CF₃-phenyl | H |
| 4-Cl-phenyl | H | H | H | bond | 2-F,4-Cl-phenyl | H |
| 4-Cl-phenyl | H | H | H | bond | 3,4-diCl-phenyl | H |
| 4-Cl-phenyl | H | H | H | bond | 3,5-diCl-phenyl | H |
| 4-Cl-phenyl | H | H | H | bond | 3-PhO-phenyl | H |
| 4-Cl-phenyl | H | H | H | bond | 2,6-diCl-phenyl | H |
| 4-Cl-phenyl | H | H | H | bond | 2,6-diF-phenyl | H |
| 4-Cl-phenyl | H | H | H | bond | 2-F,4-CF₃-phenyl | H |
| 4-F-phenyl | H | H | H | bond | 4-F-phenyl | H |
| 4-F-phenyl | H | H | H | bond | 4-Cl-phenyl | H |
| 4-F-phenyl | H | H | H | bond | 4-Br-phenyl | H |
| 4-F-phenyl | H | H | H | bond | 4-I-phenyl | H |
| 4-F-phenyl | H | H | H | bond | 4-CH₃-phenyl | H |
| 4-F-phenyl | H | H | H | bond | 4-CF₃-phenyl | H |
| 4-F-phenyl | H | H | H | bond | 4-CH₃O-phenyl | H |
| 4-F-phenyl | H | H | H | bond | 4-CH₃S-phenyl | H |
| 4-F-phenyl | H | H | H | bond | 4-CF₃O-phenyl | H |
| 4-F-phenyl | H | H | H | bond | 4-CH₃CH₂-phenyl | H |
| 4-F-phenyl | H | H | H | bond | 4-CN-phenyl | H |
| 4-F-phenyl | H | H | H | bond | 2-Cl-phenyl | H |
| 4-F-phenyl | H | H | H | bond | 3-Cl-phenyl | H |
| 4-F-phenyl | H | H | H | bond | 2,4-diCl-phenyl | H |
| 4-F-phenyl | H | H | H | bond | 2,4-diF-phenyl | H |
| 4-F-phenyl | H | H | H | bond | 3-Br-phenyl | H |
| 4-F-phenyl | H | H | H | bond | 3-CF₃-phenyl | H |
| 4-F-phenyl | H | H | H | bond | 2-F,4-Cl-phenyl | H |
| 4-F-phenyl | H | H | H | bond | 3,4-diCl-phenyl | H |
| 4-F-phenyl | H | H | H | bond | 3,5-diCl-phenyl | H |
| 4-F-phenyl | H | H | H | bond | 3-PhO-phenyl | H |
| 4-F-phenyl | H | H | H | bond | 2,6-diCl-phenyl | H |
| 4-F-phenyl | H | H | H | bond | 2,6-diF-phenyl | H |
| 4-F-phenyl | H | H | H | bond | 2-F,4-CF₃-phenyl | H |
| 4-Br-phenyl | H | H | H | bond | 4-F-phenyl | H |
| 4-Br-phenyl | H | H | H | bond | 4-Cl-phenyl | H |
| 4-Br-phenyl | H | H | H | bond | 4-Br-phenyl | H |
| 4-Br-phenyl | H | H | H | bond | 4-I-phenyl | H |
| 4-Br-phenyl | H | H | H | bond | 4-CH₃-phenyl | H |
| 4-Br-phenyl | H | H | H | bond | 4-CF₃-phenyl | H |
| 4-Br-phenyl | H | H | H | bond | 4-CH₃O-phenyl | H |
| 4-Br-phenyl | H | H | H | bond | 4-CH₃S-phenyl | H |
| 4-Br-phenyl | H | H | H | bond | 4-CF₃O-phenyl | H |
| 4-Br-phenyl | H | H | H | bond | 4-CH₃CH₂-phenyl | H |
| 4-Br-phenyl | H | H | H | bond | 4-CN-phenyl | H |
| 4-Br-phenyl | H | H | H | bond | 2-Cl-phenyl | H |
| 4-Br-phenyl | H | H | H | bond | 3-Cl-phenyl | H |
| 4-Br-phenyl | H | H | H | bond | 2,4-diCl-phenyl | H |
| 4-Br-phenyl | H | H | H | bond | 2,4-diF-phenyl | H |
| 4-Br-phenyl | H | H | H | bond | 3-Br-phenyl | H |
| 4-Br-phenyl | H | H | H | bond | 3-CF₃-phenyl | H |
| 4-Br-phenyl | H | H | H | bond | 2-F,4-Cl-phenyl | H |
| 4-Br-phenyl | H | H | H | bond | 3,4-diCl-phenyl | H |
| 4-Br-phenyl | H | H | H | bond | 3,5-diCl-phenyl | H |
| 4-Br-phenyl | H | H | H | bond | 3-PhO-phenyl | H |
| 4-Br-phenyl | H | H | H | bond | 2,6-diCl-phenyl | H |
| 4-Br-phenyl | H | H | H | bond | 2,6-diF-phenyl | H |
| 4-Br-phenyl | H | H | H | bond | 2-F,4-CF₃-phenyl | H |
| 2,4-diF-phenyl | H | H | H | bond | 4-F-phenyl | H |
| 2,4-diF-phenyl | H | H | H | bond | 4-Cl-phenyl | H |
| 2,4-diF-phenyl | H | H | H | bond | 4-Br-phenyl | H |
| 2,4-diF-phenyl | H | H | H | bond | 4-I-phenyl | H |
| 2,4-diF-phenyl | H | H | H | bond | 4-CH₃-phenyl | H |
| 2,4-diF-phenyl | H | H | H | bond | 4-CF₃-phenyl | H |
| 2,4-diF-phenyl | H | H | H | bond | 4-CH₃O-phenyl | H |
| 2,4-diF-phenyl | H | H | H | bond | 4-CH₃S-phenyl | H |
| 2,4-diF-phenyl | H | H | H | bond | 4-CF₃O-phenyl | H |
| 2,4-diF-phenyl | H | H | H | bond | 4-CH₃CH₂-phenyl | H |
| 2,4-diF-phenyl | H | H | H | bond | 4-CN-phenyl | H |
| 2,4-diF-phenyl | H | H | H | bond | 2-Cl-phenyl | H |
| 2,4-diF-phenyl | H | H | H | bond | 3-Cl-phenyl | H |
| 2,4-diF-phenyl | H | H | H | bond | 2,4-diCl-phenyl | H |
| 2,4-diF-phenyl | H | H | H | bond | 2,4-diF-phenyl | H |
| 2,4-diF-phenyl | H | H | H | bond | 3-Br-phenyl | H |
| 2,4-diF-phenyl | H | H | H | bond | 3-CF₃-phenyl | H |
| 2,4-diF-phenyl | H | H | H | bond | 2-F,4-Cl-phenyl | H |
| 2,4-diF-phenyl | H | H | H | bond | 3,4-diCl-phenyl | H |
| 2,4-diF-phenyl | H | H | H | bond | 3,5-diCl-phenyl | H |
| 2,4-diF-phenyl | H | H | H | bond | 3-PhO-phenyl | H |
| 2,4-diF-phenyl | H | H | H | bond | 2,6-diCl-phenyl | H |
| 2,4-diF-phenyl | H | H | H | bond | 2,6-diF-phenyl | H |
| 2,4-diF-phenyl | H | H | H | bond | 2-F,4-CF₃-phenyl | H |
| 4-CF₃-phenyl | H | H | H | bond | 4-F-phenyl | H |
| 4-CF₃-phenyl | H | H | H | bond | 4-Cl-phenyl | H |
| 4-CF₃-phenyl | H | H | H | bond | 4-Br-phenyl | H |
| 4-CF₃-phenyl | H | H | H | bond | 4-I-phenyl | H |
| 4-CF₃-phenyl | H | H | H | bond | 4-CH₃-phenyl | H |
| 4-CF₃-phenyl | H | H | H | bond | 4-CF₃-phenyl | H |
| 4-CF₃-phenyl | H | H | H | bond | 4-CH₃O-phenyl | H |
| 4-CF₃-phenyl | H | H | H | bond | 4-CH₃S-phenyl | H |
| 4-CF₃-phenyl | H | H | H | bond | 4-CF₃O-phenyl | H |
| 4-CF₃-phenyl | H | H | H | bond | 4-CH₃CH₂-phenyl | H |
| 4-CF₃-phenyl | H | H | H | bond | 4-CN-phenyl | H |
| 4-CF₃-phenyl | H | H | H | bond | 2-Cl-phenyl | H |
| 4-CF₃-phenyl | H | H | H | bond | 3-Cl-phenyl | H |
| 4-CF₃-phenyl | H | H | H | bond | 2,4-diCl-phenyl | H |
| 4-CF₃-phenyl | H | H | H | bond | 2,4-diF-phenyl | H |
| 4-CF₃-phenyl | H | H | H | bond | 3-Br-phenyl | H |
| 4-CF₃-phenyl | H | H | H | bond | 3-CF₃-phenyl | H |
| 4-CF₃-phenyl | H | H | H | bond | 2-F,4-Cl-phenyl | H |
| 4-CF₃-phenyl | H | H | H | bond | 3,4-diCl-phenyl | H |
| 4-CF₃-phenyl | H | H | H | bond | 3,5-diCl-phenyl | H |
| 4-CF₃-phenyl | H | H | H | bond | 3-PhO-phenyl | H |
| 4-CF₃-phenyl | H | H | H | bond | 2,6-diCl-phenyl | H |
| 4-CF₃-phenyl | H | H | H | bond | 2,6-diF-phenyl | H |
| 4-CF₃-phenyl | H | H | H | bond | 2-F,4-CF₃-phenyl | H |
| 4-Cl-phenyl | H | H | H | CH₂ | 4-F-phenyl | H |
| 4-Cl-phenyl | H | H | H | CH₂ | 4-Cl-phenyl | H |
| 4-Cl-phenyl | H | H | H | CH₂ | 4-Br-phenyl | H |
| 4-Cl-phenyl | H | H | H | CH₂ | 4-I-phenyl | H |
| 4-Cl-phenyl | H | H | H | CH₂ | 4-CH₃-phenyl | H |
| 4-Cl-phenyl | H | H | H | CH₂ | 4-CF₃-phenyl | H |
| 4-Cl-phenyl | H | H | H | CH₂ | 4-CH₃O-phenyl | H |
| 4-Cl-phenyl | H | H | H | CH₂ | 4-CH₃S-phenyl | H |
| 4-Cl-phenyl | H | H | H | CH₂ | 4-CF₃O-phenyl | H |
| 4-Cl-phenyl | H | H | H | CH₂ | 4-CH₃CH₂-phenyl | H |
| 4-Cl-phenyl | H | H | H | CH₂ | 4-CN-phenyl | H |
| 4-Cl-phenyl | H | H | H | CH₂ | 2-Cl-phenyl | H |
| 4-Cl-phenyl | H | H | H | CH₂ | 3-Cl-phenyl | H |
| 4-Cl-phenyl | H | H | H | CH₂ | 2,4-diCl-phenyl | H |
| 4-Cl-phenyl | H | H | H | CH₂ | 2,4-diF-phenyl | H |
| 4-Cl-phenyl | H | H | H | CH₂ | 3-Br-phenyl | H |
| 4-Cl-phenyl | H | H | H | CH₂ | 3-CF₃-phenyl | H |
| 4-Cl-phenyl | H | H | H | CH₂ | 2-F,4-Cl-phenyl | H |
| 4-Cl-phenyl | H | H | H | CH₂ | 3,4-diCl-phenyl | H |
| 4-Cl-phenyl | H | H | H | CH₂ | 3,5-diCl-phenyl | H |
| 4-Cl-phenyl | H | H | H | CH₂ | 3-PhO-phenyl | H |
| 4-Cl-phenyl | H | H | H | CH₂ | 2,6-diCl-phenyl | H |
| 4-Cl-phenyl | H | H | H | CH₂ | 2,6-diF-phenyl | H |
| 4-Cl-phenyl | H | H | H | CH₂ | 2-F,4-CF₃-phenyl | H |
| 4-Cl-phenyl | H | H | H | CH₂CH₂ | 4-F-phenyl | H |
| 4-Cl-phenyl | H | H | H | CH₂CH₂ | 4-Cl-phenyl | H |
| 4-Cl-phenyl | H | H | H | CH₂CH₂ | 4-Br-phenyl | H |
| 4-Cl-phenyl | H | H | H | CH₂CH₂ | 4-I-phenyl | H |
| 4-Cl-phenyl | H | H | H | CH₂CH₂ | 4-CH₃-phenyl | H |
| 4-Cl-phenyl | H | H | H | CH₂CH₂ | 4-CF₃-phenyl | H |
| 4-Cl-phenyl | H | H | H | CH₂CH₂ | 4-CH₃O-phenyl | H |
| 4-Cl-phenyl | H | H | H | CH₂CH₂ | 4-CH₃S-phenyl | H |
| 4-Cl-phenyl | H | H | H | CH₂CH₂ | 4-CF₃O-phenyl | H |
| 4-Cl-phenyl | H | H | H | CH₂CH₂ | 4-CH₃CH₂-phenyl | H |
| 4-Cl-phenyl | H | H | H | CH₂CH₂ | 4-CN-phenyl | H |
| 4-Cl-phenyl | H | H | H | CH₂CH₂ | 2-Cl-phenyl | H |
| 4-Cl-phenyl | H | H | H | CH₂CH₂ | 3-Cl-phenyl | H |
| 4-Cl-phenyl | H | H | H | CH₂CH₂ | 2,4-diCl-phenyl | H |
| 4-Cl-phenyl | H | H | H | CH₂CH₂ | 2,4-diF-phenyl | H |
| 4-Cl-phenyl | H | H | H | CH₂CH₂ | 3-Br-phenyl | H |
| 4-Cl-phenyl | H | H | H | CH₂CH₂ | 3-CF₃-phenyl | H |
| 4-Cl-phenyl | H | H | H | CH₂CH₂ | 2-F,4-Cl-phenyl | H |
| 4-Cl-phenyl | H | H | H | CH₂CH₂ | 3,4-diCl-phenyl | H |
| 4-Cl-phenyl | H | H | H | CH₂CH₂ | 3,5-diCl-phenyl | H |
| 4-Cl-phenyl | H | H | H | CH₂CH₂ | 3-PhO-phenyl | H |
| 4-Cl-phenyl | H | H | H | CH₂CH₂ | 2,6-diCl-phenyl | H |
| 4-Cl-phenyl | H | H | H | CH₂CH₂ | 2,6-diF-phenyl | H |
| 4-Cl-phenyl | H | H | H | CH₂CH₂ | 2-F,4-CF₃-phenyl | H |
| 4-Cl-phenyl | H | H | H | CH₂O | 4-F-phenyl | H |
| 4-Cl-phenyl | H | H | H | CH₂O | 4-Cl-phenyl | H |
| 4-Cl-phenyl | H | H | H | CH₂O | 4-Br-phenyl | H |
| 4-Cl-phenyl | H | H | H | CH₂O | 4-I-phenyl | H |
| 4-Cl-phenyl | H | H | H | CH₂O | 4-CH₃-phenyl | H |
| 4-Cl-phenyl | H | H | H | CH₂O | 4-CF₃-phenyl | H |
| 4-Cl-phenyl | H | H | H | CH₂O | 4-CH₃O-phenyl | H |
| 4-Cl-phenyl | H | H | H | CH₂O | 4-CH₃S-phenyl | H |
| 4-Cl-phenyl | H | H | H | CH₂O | 4-CF₃O-phenyl | H |
| 4-Cl-phenyl | H | H | H | CH₂O | 4-CH₃CH₂-phenyl | H |
| 4-Cl-phenyl | H | H | H | CH₂O | 4-CN-phenyl | H |
| 4-Cl-phenyl | H | H | H | CH₂O | 2-Cl-phenyl | H |
| 4-Cl-phenyl | H | H | H | CH₂O | 3-Cl-phenyl | H |
| 4-Cl-phenyl | H | H | H | CH₂O | 2,4-diCl-phenyl | H |
| 4-Cl-phenyl | H | H | H | CH₂O | 2,4-diF-phenyl | H |
| 4-Cl-phenyl | H | H | H | CH₂O | 3-Br-phenyl | H |
| 4-Cl-phenyl | H | H | H | CH₂O | 3-CF₃-phenyl | H |
| 4-Cl-phenyl | H | H | H | CH₂O | 2-F,4-Cl-phenyl | H |
| 4-Cl-phenyl | H | H | H | CH₂O | 3,4-diCl-phenyl | H |
| 4-Cl-phenyl | H | H | H | CH₂O | 3,5-diCl-phenyl | H |
| 4-Cl-phenyl | H | H | H | CH₂O | 3-PhO-phenyl | H |
| 4-Cl-phenyl | H | H | H | CH₂O | 2,6-diCl-phenyl | H |
| 4-Cl-phenyl | H | H | H | CH₂O | 2,6-diF-phenyl | H |
| 4-Cl-phenyl | H | H | H | CH₂O | 2-F,4-CF₃-phenyl | H |
| 4-Cl-phenyl | H | H | H | CH₂CH₂O | 4-F-phenyl | H |
| 4-Cl-phenyl | H | H | H | CH₂CH₂O | 4-Cl-phenyl | H |
| 4-Cl-phenyl | H | H | H | CH₂CH₂O | 4-Br-phenyl | H |
| 4-Cl-phenyl | H | H | H | CH₂CH₂O | 4-I-phenyl | H |
| 4-Cl-phenyl | H | H | H | CH₂CH₂O | 4-CH₃-phenyl | H |
| 4-Cl-phenyl | H | H | H | CH₂CH₂O | 4-CF₃-phenyl | H |
| 4-Cl-phenyl | H | H | H | CH₂CH₂O | 4-CH₃O-phenyl | H |
| 4-Cl-phenyl | H | H | H | CH₂CH₂O | 4-CH₃S-phenyl | H |
| 4-Cl-phenyl | H | H | H | CH₂CH₂O | 4-CF₃O-phenyl | H |
| 4-Cl-phenyl | H | H | H | CH₂CH₂O | 4-CH₃CH₂-phenyl | H |
| 4-Cl-phenyl | H | H | H | CH₂CH₂O | 4-CN-phenyl | H |
| 4-Cl-phenyl | H | H | H | CH₂CH₂O | 2-Cl-phenyl | H |
| 4-Cl-phenyl | H | H | H | CH₂CH₂O | 3-Cl-phenyl | H |
| 4-Cl-phenyl | H | H | H | CH₂CH₂O | 2,4-diCl-phenyl | H |
| 4-Cl-phenyl | H | H | H | CH₂CH₂O | 2,4-diF-phenyl | H |
| 4-Cl-phenyl | H | H | H | CH₂CH₂O | 3-Br-phenyl | H |
| 4-Cl-phenyl | H | H | H | CH₂CH₂O | 3-CF₃-phenyl | H |
| 4-Cl-phenyl | H | H | H | CH₂CH₂O | 2-F,4-Cl-phenyl | H |
| 4-Cl-phenyl | H | H | H | CH₂CH₂O | 3,4-diCl-phenyl | H |
| 4-Cl-phenyl | H | H | H | CH₂CH₂O | 3,5-diCl-phenyl | H |
| 4-Cl-phenyl | H | H | H | CH₂CH₂O | 3-PhO-phenyl | H |
| 4-Cl-phenyl | H | H | H | CH₂CH₂O | 2,6-diCl-phenyl | H |
| 4-Cl-phenyl | H | H | H | CH₂CH₂O | 2,6-diF-phenyl | H |
| 4-Cl-phenyl | H | H | H | CH₂CH₂O | 2-F,4-CF₃-phenyl | H |
| t-Butyl | H | H | H | CH₂ | 4-F-phenyl | H |
| t-Butyl | H | H | H | CH₂ | 4-Cl-phenyl | H |
| t-Butyl | H | H | H | CH₂ | 4-Br-phenyl | H |
| t-Butyl | H | H | H | CH₂ | 4-I-phenyl | H |
| t-Butyl | H | H | H | CH₂ | 4-CH₃-phenyl | H |
| t-Butyl | H | H | H | CH₂ | 4-CF₃-phenyl | H |
| t-Butyl | H | H | H | CH₂ | 4-CH₃O-phenyl | H |
| t-Butyl | H | H | H | CH₂ | 4-CH₃S-phenyl | H |
| t-Butyl | H | H | H | CH₂ | 4-CF₃O-phenyl | H |
| t-Butyl | H | H | H | CH₂ | 4-CH₃CH₂-phenyl | H |
| t-Butyl | H | H | H | CH₂ | 4-CN-phenyl | H |
| t-Butyl | H | H | H | CH₂ | 2-Cl-phenyl | H |
| t-Butyl | H | H | H | CH₂ | 3-Cl-phenyl | H |
| t-Butyl | H | H | H | CH₂ | 2,4-diCl-phenyl | H |
| t-Butyl | H | H | H | CH₂ | 2,4-diF-phenyl | H |
| t-Butyl | H | H | H | CH₂ | 3-Br-phenyl | H |
| t-Butyl | H | H | H | CH₂ | 3-CF₃-phenyl | H |
| t-Butyl | H | H | H | CH₂ | 2-F,4-Cl-phenyl | H |
| t-Butyl | H | H | H | CH₂ | 3,4-diCl-phenyl | H |
| t-Butyl | H | H | H | CH₂ | 3,5-diCl-phenyl | H |
| t-Butyl | H | H | H | CH₂ | 3-PhO-phenyl | H |
| t-Butyl | H | H | H | CH₂ | 2,6-diCl-phenyl | H |
| t-Butyl | H | H | H | CH₂ | 2,6-diF-phenyl | H |
| t-Butyl | H | H | H | CH₂ | 2-F,4-CF₃-phenyl | H |
| t-Butyl | H | H | H | CH₂CH₂ | 4-F-phenyl | H |
| t-Butyl | H | H | H | CH₂CH₂ | 4-Cl-phenyl | H |
| t-Butyl | H | H | H | CH₂CH₂ | 4-Br-phenyl | H |
| t-Butyl | H | H | H | CH₂CH₂ | 4-I-phenyl | H |
| t-Butyl | H | H | H | CH₂CH₂ | 4-CH₃-phenyl | H |
| t-Butyl | H | H | H | CH₂CH₂ | 4-CF₃-phenyl | H |
| t-Butyl | H | H | H | CH₂CH₂ | 4-CH₃O-phenyl | H |
| t-Butyl | H | H | H | CH₂CH₂ | 4-CH₃S-phenyl | H |
| t-Butyl | H | H | H | CH₂CH₂ | 4-CF₃O-phenyl | H |
| t-Butyl | H | H | H | CH₂CH₂ | 4-CH₃CH₂-phenyl | H |
| t-Butyl | H | H | H | CH₂CH₂ | 4-CN-phenyl | H |
| t-Butyl | H | H | H | CH₂CH₂ | 2-Cl-phenyl | H |
| t-Butyl | H | H | H | CH₂CH₂ | 3-Cl-phenyl | H |
| t-Butyl | H | H | H | CH₂CH₂ | 2,4-diCl-phenyl | H |
| t-Butyl | H | H | H | CH₂CH₂ | 2,4-diF-phenyl | H |
| t-Butyl | H | H | H | CH₂CH₂ | 3-Br-phenyl | H |
| t-Butyl | H | H | H | CH₂CH₂ | 3-CF₃-phenyl | H |
| t-Butyl | H | H | H | CH₂CH₂ | 2-F,4-Cl-phenyl | H |
| t-Butyl | H | H | H | CH₂CH₂ | 3,4-diCl-phenyl | H |
| t-Butyl | H | H | H | CH₂CH₂ | 3,5-diCl-phenyl | H |
| t-Butyl | H | H | H | CH₂CH₂ | 3-PhO-phenyl | H |
| t-Butyl | H | H | H | CH₂CH₂ | 2,6-diCl-phenyl | H |
| t-Butyl | H | H | H | CH₂CH₂ | 2,6-diF-phenyl | H |
| t-Butyl | H | H | H | CH₂CH₂ | 2-F,4-CF₃-phenyl | H |
| t-Butyl | H | H | H | CH₂O | 4-F-phenyl | H |
| t-Butyl | H | H | H | CH₂O | 4-Cl-phenyl | H |
| t-Butyl | H | H | H | CH₂O | 4-Br-phenyl | H |
| t-Butyl | H | H | H | CH₂O | 4-I-phenyl | H |
| t-Butyl | H | H | H | CH₂O | 4-CH₃-phenyl | H |
| t-Butyl | H | H | H | CH₂O | 4-CF₃-phenyl | H |
| t-Butyl | H | H | H | CH₂O | 4-CH₃O-phenyl | H |
| t-Butyl | H | H | H | CH₂O | 4-CH₃S-phenyl | H |
| t-Butyl | H | H | H | CH₂O | 4-CF₃O-phenyl | H |
| t-Butyl | H | H | H | CH₂O | 4-CH₃CH₂-phenyl | H |
| t-Butyl | H | H | H | CH₂O | 4-CN-phenyl | H |
| t-Butyl | H | H | H | CH₂O | 2-Cl-phenyl | H |
| t-Butyl | H | H | H | CH₂O | 3-Cl-phenyl | H |
| t-Butyl | H | H | H | CH₂O | 2,4-diCl-phenyl | H |
| t-Butyl | H | H | H | CH₂O | 2,4-diF-phenyl | H |
| t-Butyl | H | H | H | CH₂O | 3-Br-phenyl | H |
| t-Butyl | H | H | H | CH₂O | 3-CF₃-phenyl | H |
| t-Butyl | H | H | H | CH₂O | 2-F,4-Cl-phenyl | H |
| t-Butyl | H | H | H | CH₂O | 3,4-diCl-phenyl | H |
| t-Butyl | H | H | H | CH₂O | 3,5-diCl-phenyl | H |
| t-Butyl | H | H | H | CH₂O | 3-PhO-phenyl | H |
| t-Butyl | H | H | H | CH₂O | 2,6-diCl-phenyl | H |
| t-Butyl | H | H | H | CH₂O | 2,6-diF-phenyl | H |
| t-Butyl | H | H | H | CH₂O | 2-F,4-CF₃-phenyl | H |
| t-Butyl | H | H | H | CH₂CH₂O | 4-F-phenyl | H |
| t-Butyl | H | H | H | CH₂CH₂O | 4-Cl-phenyl | H |
| t-Butyl | H | H | H | CH₂CH₂O | 4-Br-phenyl | H |
| t-Butyl | H | H | H | CH₂CH₂O | 4-I-phenyl | H |
| t-Butyl | H | H | H | CH₂CH₂O | 4-CH₃-phenyl | H |
| t-Butyl | H | H | H | CH₂CH₂O | 4-CF₃-phenyl | H |
| t-Butyl | H | H | H | CH₂CH₂O | 4-CH₃O-phenyl | H |
| t-Butyl | H | H | H | CH₂CH₂O | 4-CH₃S-phenyl | H |
| t-Butyl | H | H | H | CH₂CH₂O | 4-CF₃O-phenyl | H |
| t-Butyl | H | H | H | CH₂CH₂O | 4-CH₃CH₂-phenyl | H |
| t-Butyl | H | H | H | CH₂CH₂O | 4-CN-phenyl | H |
| t-Butyl | H | H | H | CH₂CH₂O | 2-Cl-phenyl | H |
| t-Butyl | H | H | H | CH₂CH₂O | 3-Cl-phenyl | H |
| t-Butyl | H | H | H | CH₂CH₂O | 2,4-diCl-phenyl | H |
| t-Butyl | H | H | H | CH₂CH₂O | 2,4-diF-phenyl | H |
| t-Butyl | H | H | H | CH₂CH₂O | 3-Br-phenyl | H |
| t-Butyl | H | H | H | CH₂CH₂O | 3-CF₃-phenyl | H |
| t-Butyl | H | H | H | CH₂CH₂O | 2-F,4-Cl-phenyl | H |
| t-Butyl | H | H | H | CH₂CH₂O | 3,4-diCl-phenyl | H |
| t-Butyl | H | H | H | CH₂CH₂O | 3,5-diCl-phenyl | H |
| t-Butyl | H | H | H | CH₂CH₂O | 3-PhO-phenyl | H |
| t-Butyl | H | H | H | CH₂CH₂O | 2,6-diCl-phenyl | H |
| t-Butyl | H | H | H | CH₂CH₂O | 2,6-diF-phenyl | H |
| t-Butyl | H | H | H | CH₂CH₂O | 2-F,4-CF₃-phenyl | H |
| t-Amyl | H | H | H | Bond | 4-F-phenyl | H |
| t-Amyl | H | H | H | Bond | 4-Cl-phenyl | H |
| t-Amyl | H | H | H | Bond | 4-Br-phenyl | H |
| t-Amyl | H | H | H | Bond | 4-I-phenyl | H |
| t-Amyl | H | H | H | Bond | 4-CH₃-phenyl | H |
| t-Amyl | H | H | H | Bond | 4-CF₃-phenyl | H |
| t-Amyl | H | H | H | Bond | 4-CH₃O-phenyl | H |
| t-Amyl | H | H | H | Bond | 4-CH₃S-phenyl | H |
| t-Amyl | H | H | H | Bond | 4-CF₃O-phenyl | H |
| t-Amyl | H | H | H | Bond | 4-CH₃CH₂-phenyl | H |
| t-Amyl | H | H | H | Bond | 4-CN-phenyl | H |
| t-Amyl | H | H | H | Bond | 2-Cl-phenyl | H |
| t-Amyl | H | H | H | Bond | 3-Cl-phenyl | H |
| t-Amyl | H | H | H | Bond | 2,4-diCl-phenyl | H |
| t-Amyl | H | H | H | Bond | 2,4-diF-phenyl | H |
| t-Amyl | H | H | H | Bond | 3-Br-phenyl | H |
| t-Amyl | H | H | H | Bond | 3-CF₃-phenyl | H |
| t-Amyl | H | H | H | Bond | 2-F,4-Cl-phenyl | H |
| t-Amyl | H | H | H | Bond | 3,4-diCl-phenyl | H |
| t-Amyl | H | H | H | Bond | 3,5-diCl-phenyl | H |
| t-Amyl | H | H | H | Bond | 3-PhO-phenyl | H |
| t-Amyl | H | H | H | Bond | 2,6-diCl-phenyl | H |
| t-Amyl | H | H | H | Bond | 2,6-diF-phenyl | H |
| t-Amyl | H | H | H | Bond | 2-F,4-CF₃-phenyl | H |
| t-Amyl | H | H | H | CH₂ | 4-F-phenyl | H |
| t-Amyl | H | H | H | CH₂ | 4-Cl-phenyl | H |
| t-Amyl | H | H | H | CH₂ | 4-Br-phenyl | H |
| t-Amyl | H | H | H | CH₂ | 4-I-phenyl | H |
| t-Amyl | H | H | H | CH₂ | 4-CH₃-phenyl | H |
| t-Amyl | H | H | H | CH₂ | 4-CF₃-phenyl | H |
| t-Amyl | H | H | H | CH₂ | 4-CH₃O-phenyl | H |
| t-Amyl | H | H | H | CH₂ | 4-CH₃S-phenyl | H |
| t-Amyl | H | H | H | CH₂ | 4-CF₃O-phenyl | H |
| t-Amyl | H | H | H | CH₂ | 4-CH₃CH₂-phenyl | H |
| t-Amyl | H | H | H | CH₂ | 4-CN-phenyl | H |
| t-Amyl | H | H | H | CH₂ | 2-Cl-phenyl | H |
| t-Amyl | H | H | H | CH₂ | 3-Cl-phenyl | H |
| t-Amyl | H | H | H | CH₂ | 2,4-diCl-phenyl | H |
| t-Amyl | H | H | H | CH₂ | 2,4-diF-phenyl | H |
| t-Amyl | H | H | H | CH₂ | 3-Br-phenyl | H |
| t-Amyl | H | H | H | CH₂ | 3-CF₃-phenyl | H |
| t-Amyl | H | H | H | CH₂ | 2-F,4-Cl-phenyl | H |
| t-Amyl | H | H | H | CH₂ | 3,4-diCl-phenyl | H |
| t-Amyl | H | H | H | CH₂ | 3,5-diCl-phenyl | H |
| t-Amyl | H | H | H | CH₂ | 3-PhO-phenyl | H |
| t-Amyl | H | H | H | CH₂ | 2,6-diCl-phenyl | H |
| t-Amyl | H | H | H | CH₂ | 2,6-diF-phenyl | H |
| t-Amyl | H | H | H | CH₂ | 2-F,4-CF₃-phenyl | H |
| t-Amyl | H | H | H | CH₂CH₂ | 4-F-phenyl | H |
| t-Amyl | H | H | H | CH₂CH₂ | 4-Cl-phenyl | H |
| t-Amyl | H | H | H | CH₂CH₂ | 4-Br-phenyl | H |
| t-Amyl | H | H | H | CH₂CH₂ | 4-I-phenyl | H |
| t-Amyl | H | H | H | CH₂CH₂ | 4-CH₃-phenyl | H |
| t-Amyl | H | H | H | CH₂CH₂ | 4-CF₃-phenyl | H |
| t-Amyl | H | H | H | CH₂CH₂ | 4-CH₃O-phenyl | H |
| t-Amyl | H | H | H | CH₂CH₂ | 4-CH₃S-phenyl | H |
| t-Amyl | H | H | H | CH₂CH₂ | 4-CF₃O-phenyl | H |
| t-Amyl | H | H | H | CH₂CH₂ | 4-CH₃CH₂-phenyl | H |
| t-Amyl | H | H | H | CH₂CH₂ | 4-CN-phenyl | H |
| t-Amyl | H | H | H | CH₂CH₂ | 2-Cl-phenyl | H |
| t-Amyl | H | H | H | CH₂CH₂ | 3-Cl-phenyl | H |
| t-Amyl | H | H | H | CH₂CH₂ | 2,4-diCl-phenyl | H |
| t-Amyl | H | H | H | CH₂CH₂ | 2,4-diF-phenyl | H |
| t-Amyl | H | H | H | CH₂CH₂ | 3-Br-phenyl | H |
| t-Amyl | H | H | H | CH₂CH₂ | 3-CF₃-phenyl | H |
| t-Amyl | H | H | H | CH₂CH₂ | 2-F,4-Cl-phenyl | H |
| t-Amyl | H | H | H | CH₂CH₂ | 3,4-diCl-phenyl | H |
| t-Amyl | H | H | H | CH₂CH₂ | 3,5-diCl-phenyl | H |
| t-Amyl | H | H | H | CH₂CH₂ | 3-PhO-phenyl | H |
| t-Amyl | H | H | H | CH₂CH₂ | 2,6-diCl-phenyl | H |
| t-Amyl | H | H | H | CH₂CH₂ | 2,6-diF-phenyl | H |
| t-Amyl | H | H | H | CH₂CH₂ | 2-F,4-CF₃-phenyl | H |
| t-Amyl | H | H | H | CH₂O | 4-F-phenyl | H |
| t-Amyl | H | H | H | CH₂O | 4-Cl-phenyl | H |
| t-Amyl | H | H | H | CH₂O | 4-Br-phenyl | H |
| t-Amyl | H | H | H | CH₂O | 4-I-phenyl | H |
| t-Amyl | H | H | H | CH₂O | 4-CH₃-phenyl | H |
| t-Amyl | H | H | H | CH₂O | 4-CF₃-phenyl | H |
| t-Amyl | H | H | H | CH₂O | 4-CH₃O-phenyl | H |
| t-Amyl | H | H | H | CH₂O | 4-CH₃S-phenyl | H |
| t-Amyl | H | H | H | CH₂O | 4-CF₃O-phenyl | H |
| t-Amyl | H | H | H | CH₂O | 4-CH₃CH₂-phenyl | H |
| t-Amyl | H | H | H | CH₂O | 4-CN-phenyl | H |
| t-Amyl | H | H | H | CH₂O | 2-Cl-phenyl | H |
| t-Amyl | H | H | H | CH₂O | 3-Cl-phenyl | H |
| t-Amyl | H | H | H | CH₂O | 2,4-diCl-phenyl | H |
| t-Amyl | H | H | H | CH₂O | 2,4-diF-phenyl | H |
| t-Amyl | H | H | H | CH₂O | 3-Br-phenyl | H |
| t-Amyl | H | H | H | CH₂O | 3-CF₃-phenyl | H |
| t-Amyl | H | H | H | CH₂O | 2-F,4-Cl-phenyl | H |
| t-Amyl | H | H | H | CH₂O | 3,4-diCl-phenyl | H |
| t-Amyl | H | H | H | CH₂O | 3,5-diCl-phenyl | H |
| t-Amyl | H | H | H | CH₂O | 3-PhO-phenyl | H |
| t-Amyl | H | H | H | CH₂O | 2,6-diCl-phenyl | H |
| t-Amyl | H | H | H | CH₂O | 2,6-diF-phenyl | H |
| t-Amyl | H | H | H | CH₂O | 2-F,4-CF₃-phenyl | H |
| t-Amyl | H | H | H | CH₂CH₂O | 4-F-phenyl | H |
| t-Amyl | H | H | H | CH₂CH₂O | 4-Cl-phenyl | H |
| t-Amyl | H | H | H | CH₂CH₂O | 4-Br-phenyl | H |
| t-Amyl | H | H | H | CH₂CH₂O | 4-I-phenyl | H |
| t-Amyl | H | H | H | CH₂CH₂O | 4-CH₃-phenyl | H |
| t-Amyl | H | H | H | CH₂CH₂O | 4-CF₃-phenyl | H |
| t-Amyl | H | H | H | CH₂CH₂O | 4-CH₃O-phenyl | H |
| t-Amyl | H | H | H | CH₂CH₂O | 4-CH₃S-phenyl | H |
| t-Amyl | H | H | H | CH₂CH₂O | 4-CF₃O-phenyl | H |
| t-Amyl | H | H | H | CH₂CH₂O | 4-CH₃CH₂-phenyl | H |
| t-Amyl | H | H | H | CH₂CH₂O | 4-CN-phenyl | H |
| t-Amyl | H | H | H | CH₂CH₂O | 2-Cl-phenyl | H |
| t-Amyl | H | H | H | CH₂CH₂O | 3-Cl-phenyl | H |
| t-Amyl | H | H | H | CH₂CH₂O | 2,4-diCl-phenyl | H |
| t-Amyl | H | H | H | CH₂CH₂O | 2,4-diF-phenyl | H |
| t-Amyl | H | H | H | CH₂CH₂O | 3-Br-phenyl | H |
| t-Amyl | H | H | H | CH₂CH₂O | 3-CF₃-phenyl | H |
| t-Amyl | H | H | H | CH₂CH₂O | 2-F,4-Cl-phenyl | H |
| t-Amyl | H | H | H | CH₂CH₂O | 3,4-diCl-phenyl | H |
| t-Amyl | H | H | H | CH₂CH₂O | 3,5-diCl-phenyl | H |
| t-Amyl | H | H | H | CH₂CH₂O | 3-PhO-phenyl | H |
| t-Amyl | H | H | H | CH₂CH₂O | 2,6-diCl-phenyl | H |
| t-Amyl | H | H | H | CH₂CH₂O | 2,6-diF-phenyl | H |
| t-Amyl | H | H | H | CH₂CH₂O | 2-F,4-CF₃-phenyl | H |
| 1-Me-1-c-Pr | H | H | H | Bond | 4-F-phenyl | H |
| 1-Me-1-c-Pr | H | H | H | Bond | 4-Cl-phenyl | H |
| 1-Me-1-c-Pr | H | H | H | Bond | 4-Br-phenyl | H |
| 1-Me-1-c-Pr | H | H | H | Bond | 4-I-phenyl | H |
| 1-Me-1-c-Pr | H | H | H | Bond | 4-CH₃-phenyl | H |
| 1-Me-1-c-Pr | H | H | H | Bond | 4-CF₃-phenyl | H |
| 1-Me-1-c-Pr | H | H | H | Bond | 4-CH₃O-phenyl | H |
| 1-Me-1-c-Pr | H | H | H | Bond | 4-CH₃S-phenyl | H |
| 1-Me-1-c-Pr | H | H | H | Bond | 4-CF₃O-phenyl | H |
| 1-Me-1-c-Pr | H | H | H | Bond | 4-CH₃CH₂-phenyl | H |
| 1-Me-1-c-Pr | H | H | H | Bond | 4-CN-phenyl | H |
| 1-Me-1-c-Pr | H | H | H | Bond | 2-Cl-phenyl | H |
| 1-Me-1-c-Pr | H | H | H | Bond | 3-Cl-phenyl | H |
| 1-Me-1-c-Pr | H | H | H | Bond | 2,4-diCl-phenyl | H |
| 1-Me-1-c-Pr | H | H | H | Bond | 2,4-diF-phenyl | H |
| 1-Me-1-c-Pr | H | H | H | Bond | 3-Br-phenyl | H |
| 1-Me-1-c-Pr | H | H | H | Bond | 3-CF₃-phenyl | H |
| 1-Me-1-c-Pr | H | H | H | Bond | 2-F,4-Cl-phenyl | H |
| 1-Me-1-c-Pr | H | H | H | Bond | 3,4-diCl-phenyl | H |
| 1-Me-1-c-Pr | H | H | H | Bond | 3,5-diCl-phenyl | H |
| 1-Me-1-c-Pr | H | H | H | Bond | 3-PhO-phenyl | H |
| 1-Me-1-c-Pr | H | H | H | Bond | 2,6-diCl-phenyl | H |
| 1-Me-1-c-Pr | H | H | H | Bond | 2,6-diF-phenyl | H |
| 1-Me-1-c-Pr | H | H | H | Bond | 2-F,4-CF₃-phenyl | H |
| 1-Me-1-c-Pr | H | H | H | CH₂ | 4-F-phenyl | H |
| 1-Me-1-c-Pr | H | H | H | CH₂ | 4-Cl-phenyl | H |
| 1-Me-1-c-Pr | H | H | H | CH₂ | 4-Br-phenyl | H |
| 1-Me-1-c-Pr | H | H | H | CH₂ | 4-I-phenyl | H |
| 1-Me-1-c-Pr | H | H | H | CH₂ | 4-CH₃-phenyl | H |
| 1-Me-1-c-Pr | H | H | H | CH₂ | 4-CF₃-phenyl | H |
| 1-Me-1-c-Pr | H | H | H | CH₂ | 4-CH₃O-phenyl | H |
| 1-Me-1-c-Pr | H | H | H | CH₂ | 4-CH₃S-phenyl | H |
| 1-Me-1-c-Pr | H | H | H | CH₂ | 4-CF₃O-phenyl | H |
| 1-Me-1-c-Pr | H | H | H | CH₂ | 4-CH₃CH₂-phenyl | H |
| 1-Me-1-c-Pr | H | H | H | CH₂ | 4-CN-phenyl | H |
| 1-Me-1-c-Pr | H | H | H | CH₂ | 2-Cl-phenyl | H |
| 1-Me-1-c-Pr | H | H | H | CH₂ | 3-Cl-phenyl | H |
| 1-Me-1-c-Pr | H | H | H | CH₂ | 2,4-diCl-phenyl | H |
| 1-Me-1-c-Pr | H | H | H | CH₂ | 2,4-diF-phenyl | H |
| 1-Me-1-c-Pr | H | H | H | CH₂ | 3-Br-phenyl | H |
| 1-Me-1-c-Pr | H | H | H | CH₂ | 3-CF₃-phenyl | H |
| 1-Me-1-c-Pr | H | H | H | CH₂ | 2-F,4-Cl-phenyl | H |
| 1-Me-1-c-Pr | H | H | H | CH₂ | 3,4-diCl-phenyl | H |
| 1-Me-1-c-Pr | H | H | H | CH₂ | 3,5-diCl-phenyl | H |
| 1-Me-1-c-Pr | H | H | H | CH₂ | 3-PhO-phenyl | H |
| 1-Me-1-c-Pr | H | H | H | CH₂ | 2,6-diCl-phenyl | H |
| 1-Me-1-c-Pr | H | H | H | CH₂ | 2,6-diF-phenyl | H |
| 1-Me-1-c-Pr | H | H | H | CH₂ | 2-F,4-CF₃-phenyl | H |
| 1-Me-1-c-Pr | H | H | H | CH₂CH₂ | 4-F-phenyl | H |
| 1-Me-1-c-Pr | H | H | H | CH₂CH₂ | 4-Cl-phenyl | H |
| 1-Me-1-c-Pr | H | H | H | CH₂CH₂ | 4-Br-phenyl | H |
| 1-Me-1-c-Pr | H | H | H | CH₂CH₂ | 4-I-phenyl | H |
| 1-Me-1-c-Pr | H | H | H | CH₂CH₂ | 4-CH₃-phenyl | H |
| 1-Me-1-c-Pr | H | H | H | CH₂CH₂ | 4-CF₃-phenyl | H |
| 1-Me-1-c-Pr | H | H | H | CH₂CH₂ | 4-CH₃O-phenyl | H |
| 1-Me-1-c-Pr | H | H | H | CH₂CH₂ | 4-CH₃S-phenyl | H |
| 1-Me-1-c-Pr | H | H | H | CH₂CH₂ | 4-CF₃O-phenyl | H |
| 1-Me-1-c-Pr | H | H | H | CH₂CH₂ | 4-CH₃CH₂-phenyl | H |
| 1-Me-1-c-Pr | H | H | H | CH₂CH₂ | 4-CN-phenyl | H |
| 1-Me-1-c-Pr | H | H | H | CH₂CH₂ | 2-Cl-phenyl | H |
| 1-Me-1-c-Pr | H | H | H | CH₂CH₂ | 3-Cl-phenyl | H |
| 1-Me-1-c-Pr | H | H | H | CH₂CH₂ | 2,4-diCl-phenyl | H |
| 1-Me-1-c-Pr | H | H | H | CH₂CH₂ | 2,4-diF-phenyl | H |
| 1-Me-1-c-Pr | H | H | H | CH₂CH₂ | 3-Br-phenyl | H |
| 1-Me-1-c-Pr | H | H | H | CH₂CH₂ | 3-CF₃-phenyl | H |
| 1-Me-1-c-Pr | H | H | H | CH₂CH₂ | 2-F,4-Cl-phenyl | H |
| 1-Me-1-c-Pr | H | H | H | CH₂CH₂ | 3,4-diCl-phenyl | H |
| 1-Me-1-c-Pr | H | H | H | CH₂CH₂ | 3,5-diCl-phenyl | H |
| 1-Me-1-c-Pr | H | H | H | CH₂CH₂ | 3-PhO-phenyl | H |
| 1-Me-1-c-Pr | H | H | H | CH₂CH₂ | 2,6-diCl-phenyl | H |
| 1-Me-1-c-Pr | H | H | H | CH₂CH₂ | 2,6-diF-phenyl | H |
| 1-Me-1-c-Pr | H | H | H | CH₂CH₂ | 2-F,4-CF₃-phenyl | H |
| 1-Me-1-c-Pr | H | H | H | CH₂O | 4-F-phenyl | H |
| 1-Me-1-c-Pr | H | H | H | CH₂O | 4-Cl-phenyl | H |
| 1-Me-1-c-Pr | H | H | H | CH₂O | 4-Br-phenyl | H |
| 1-Me-1-c-Pr | H | H | H | CH₂O | 4-I-phenyl | H |
| 1-Me-1-c-Pr | H | H | H | CH₂O | 4-CH₃-phenyl | H |
| 1-Me-1-c-Pr | H | H | H | CH₂O | 4-CF₃-phenyl | H |
| 1-Me-1-c-Pr | H | H | H | CH₂O | 4-CH₃O-phenyl | H |
| 1-Me-1-c-Pr | H | H | H | CH₂O | 4-CH₃S-phenyl | H |
| 1-Me-1-c-Pr | H | H | H | CH₂O | 4-CF₃O-phenyl | H |
| 1-Me-1-c-Pr | H | H | H | CH₂O | 4-CH₃CH₂-phenyl | H |
| 1-Me-1-c-Pr | H | H | H | CH₂O | 4-CN-phenyl | H |
| 1-Me-1-c-Pr | H | H | H | CH₂O | 2-Cl-phenyl | H |
| 1-Me-1-c-Pr | H | H | H | CH₂O | 3-Cl-phenyl | H |
| 1-Me-1-c-Pr | H | H | H | CH₂O | 2,4-diCl-phenyl | H |
| 1-Me-1-c-Pr | H | H | H | CH₂O | 2,4-diF-phenyl | H |
| 1-Me-1-c-Pr | H | H | H | CH₂O | 3-Br-phenyl | H |
| 1-Me-1-c-Pr | H | H | H | CH₂O | 3-CF₃-phenyl | H |
| 1-Me-1-c-Pr | H | H | H | CH₂O | 2-F,4-Cl-phenyl | H |
| 1-Me-1-c-Pr | H | H | H | CH₂O | 3,4-diCl-phenyl | H |
| 1-Me-1-c-Pr | H | H | H | CH₂O | 3,5-diCl-phenyl | H |
| 1-Me-1-c-Pr | H | H | H | CH₂O | 3-PhO-phenyl | H |
| 1-Me-1-c-Pr | H | H | H | CH₂O | 2,6-diCl-phenyl | H |
| 1-Me-1-c-Pr | H | H | H | CH₂O | 2,6-diF-phenyl | H |
| 1-Me-1-c-Pr | H | H | H | CH₂O | 2-F,4-CF₃-phenyl | H |
| 1-Me-1-c-Pr | H | H | H | CH₂CH₂O | 4-F-phenyl | H |
| 1-Me-1-c-Pr | H | H | H | CH₂CH₂O | 4-Cl-phenyl | H |
| 1-Me-1-c-Pr | H | H | H | CH₂CH₂O | 4-Br-phenyl | H |
| 1-Me-1-c-Pr | H | H | H | CH₂CH₂O | 4-I-phenyl | H |
| 1-Me-1-c-Pr | H | H | H | CH₂CH₂O | 4-CH₃-phenyl | H |
| 1-Me-1-c-Pr | H | H | H | CH₂CH₂O | 4-CF₃-phenyl | H |
| 1-Me-1-c-Pr | H | H | H | CH₂CH₂O | 4-CH₃O-phenyl | H |
| 1-Me-1-c-Pr | H | H | H | CH₂CH₂O | 4-CH₃S-phenyl | H |
| 1-Me-1-c-Pr | H | H | H | CH₂CH₂O | 4-CF₃O-phenyl | H |
| 1-Me-1-c-Pr | H | H | H | CH₂CH₂O | 4-CH₃CH₂-phenyl | H |
| 1-Me-1-c-Pr | H | H | H | CH₂CH₂O | 4-CN-phenyl | H |
| 1-Me-1-c-Pr | H | H | H | CH₂CH₂O | 2-Cl-phenyl | H |
| 1-Me-1-c-Pr | H | H | H | CH₂CH₂O | 3-Cl-phenyl | H |
| 1-Me-1-c-Pr | H | H | H | CH₂CH₂O | 2,4-diCl-phenyl | H |
| 1-Me-1-c-Pr | H | H | H | CH₂CH₂O | 2,4-diF-phenyl | H |
| 1-Me-1-c-Pr | H | H | H | CH₂CH₂O | 3-Br-phenyl | H |
| 1-Me-1-c-Pr | H | H | H | CH₂CH₂O | 3-CF₃-phenyl | H |
| 1-Me-1-c-Pr | H | H | H | CH₂CH₂O | 2-F,4-Cl-phenyl | H |
| 1-Me-1-c-Pr | H | H | H | CH₂CH₂O | 3,4-diCl-phenyl | H |
| 1-Me-1-c-Pr | H | H | H | CH₂CH₂O | 3,5-diCl-phenyl | H |
| 1-Me-1-c-Pr | H | H | H | CH₂CH₂O | 3-PhO-phenyl | H |
| 1-Me-1-c-Pr | H | H | H | CH₂CH₂O | 2,6-diCl-phenyl | H |
| 1-Me-1-c-Pr | H | H | H | CH₂CH₂O | 2,6-diF-phenyl | H |
| 1-Me-1-c-Pr | H | H | H | CH₂CH₂O | 2-F,4-CF₃-phenyl | H |
| 1-Cl-1-c-Pr | H | H | H | Bond | 4-F-phenyl | H |
| 1-Cl-1-c-Pr | H | H | H | Bond | 4-Cl-phenyl | H |
| 1-Cl-1-c-Pr | H | H | H | Bond | 4-Br-phenyl | H |
| 1-Cl-1-c-Pr | H | H | H | Bond | 4-I-phenyl | H |
| 1-Cl-1-c-Pr | H | H | H | Bond | 4-CH₃-phenyl | H |
| 1-Cl-1-c-Pr | H | H | H | Bond | 4-CF₃-phenyl | H |
| 1-Cl-1-c-Pr | H | H | H | Bond | 4-CH₃O-phenyl | H |
| 1-Cl-1-c-Pr | H | H | H | Bond | 4-CH₃S-phenyl | H |
| 1-Cl-1-c-Pr | H | H | H | Bond | 4-CF₃O-phenyl | H |
| 1-Cl-1-c-Pr | H | H | H | Bond | 4-CH₃CH₂-phenyl | H |
| 1-Cl-1-c-Pr | H | H | H | Bond | 4-CN-phenyl | H |
| 1-Cl-1-c-Pr | H | H | H | Bond | 2-Cl-phenyl | H |
| 1-Cl-1-c-Pr | H | H | H | Bond | 3-Cl-phenyl | H |
| 1-Cl-1-c-Pr | H | H | H | Bond | 2,4-diCl-phenyl | H |
| 1-Cl-1-c-Pr | H | H | H | Bond | 2,4-diF-phenyl | H |
| 1-Cl-1-c-Pr | H | H | H | Bond | 3-Br-phenyl | H |
| 1-Cl-1-c-Pr | H | H | H | Bond | 3-CF₃-phenyl | H |
| 1-Cl-1-c-Pr | H | H | H | Bond | 2-F,4-Cl-phenyl | H |
| 1-Cl-1-c-Pr | H | H | H | Bond | 3,4-diCl-phenyl | H |
| 1-Cl-1-c-Pr | H | H | H | Bond | 3,5-diCl-phenyl | H |
| 1-Cl-1-c-Pr | H | H | H | Bond | 3-PhO-phenyl | H |
| 1-Cl-1-c-Pr | H | H | H | Bond | 2,6-diCl-phenyl | H |
| 1-Cl-1-c-Pr | H | H | H | Bond | 2,6-diF-phenyl | H |
| 1-Cl-1-c-Pr | H | H | H | Bond | 2-F,4-CF₃-phenyl | H |
| 1-Cl-1-c-Pr | H | H | H | CH₂ | 4-F-phenyl | H |
| 1-Cl-1-c-Pr | H | H | H | CH₂ | 4-Cl-phenyl | H |
| 1-Cl-1-c-Pr | H | H | H | CH₂ | 4-Br-phenyl | H |
| 1-Cl-1-c-Pr | H | H | H | CH₂ | 4-I-phenyl | H |
| 1-Cl-1-c-Pr | H | H | H | CH₂ | 4-CH₃-phenyl | H |
| 1-Cl-1-c-Pr | H | H | H | CH₂ | 4-CF₃-phenyl | H |
| 1-Cl-1-c-Pr | H | H | H | CH₂ | 4-CH₃O-phenyl | H |
| 1-Cl-1-c-Pr | H | H | H | CH₂ | 4-CH₃S-phenyl | H |
| 1-Cl-1-c-Pr | H | H | H | CH₂ | 4-CF₃O-phenyl | H |
| 1-Cl-1-c-Pr | H | H | H | CH₂ | 4-CH₃CH₂-phenyl | H |
| 1-Cl-1-c-Pr | H | H | H | CH₂ | 4-CN-phenyl | H |
| 1-Cl-1-c-Pr | H | H | H | CH₂ | 2-Cl-phenyl | H |
| 1-Cl-1-c-Pr | H | H | H | CH₂ | 3-Cl-phenyl | H |
| 1-Cl-1-c-Pr | H | H | H | CH₂ | 2,4-diCl-phenyl | H |
| 1-Cl-1-c-Pr | H | H | H | CH₂ | 2,4-diF-phenyl | H |
| 1-Cl-1-c-Pr | H | H | H | CH₂ | 3-Br-phenyl | H |
| 1-Cl-1-c-Pr | H | H | H | CH₂ | 3-CF₃-phenyl | H |
| 1-Cl-1-c-Pr | H | H | H | CH₂ | 2-F,4-Cl-phenyl | H |
| 1-Cl-1-c-Pr | H | H | H | CH₂ | 3,4-diCl-phenyl | H |
| 1-Cl-1-c-Pr | H | H | H | CH₂ | 3,5-diCl-phenyl | H |
| 1-Cl-1-c-Pr | H | H | H | CH₂ | 3-PhO-phenyl | H |
| 1-Cl-1-c-Pr | H | H | H | CH₂ | 2,6-diCl-phenyl | H |
| 1-Cl-1-c-Pr | H | H | H | CH₂ | 2,6-diF-phenyl | H |
| 1-Cl-1-c-Pr | H | H | H | CH₂ | 2-F,4-CF₃-phenyl | H |
| 1-Cl-1-c-Pr | H | H | H | CH₂CH₂ | 4-F-phenyl | H |
| 1-Cl-1-c-Pr | H | H | H | CH₂CH₂ | 4-Cl-phenyl | H |
| 1-Cl-1-c-Pr | H | H | H | CH₂CH₂ | 4-Br-phenyl | H |
| 1-Cl-1-c-Pr | H | H | H | CH₂CH₂ | 4-I-phenyl | H |
| 1-Cl-1-c-Pr | H | H | H | CH₂CH₂ | 4-CH₃-phenyl | H |
| 1-Cl-1-c-Pr | H | H | H | CH₂CH₂ | 4-CF₃-phenyl | H |
| 1-Cl-1-c-Pr | H | H | H | CH₂CH₂ | 4-CH₃O-phenyl | H |
| 1-Cl-1-c-Pr | H | H | H | CH₂CH₂ | 4-CH₃S-phenyl | H |
| 1-Cl-1-c-Pr | H | H | H | CH₂CH₂ | 4-CF₃O-phenyl | H |
| 1-Cl-1-c-Pr | H | H | H | CH₂CH₂ | 4-CH₃CH₂-phenyl | H |
| 1-Cl-1-c-Pr | H | H | H | CH₂CH₂ | 4-CN-phenyl | H |
| 1-Cl-1-c-Pr | H | H | H | CH₂CH₂ | 2-Cl-phenyl | H |
| 1-Cl-1-c-Pr | H | H | H | CH₂CH₂ | 3-Cl-phenyl | H |
| 1-Cl-1-c-Pr | H | H | H | CH₂CH₂ | 2,4-diCl-phenyl | H |
| 1-Cl-1-c-Pr | H | H | H | CH₂CH₂ | 2,4-diF-phenyl | H |
| 1-Cl-1-c-Pr | H | H | H | CH₂CH₂ | 3-Br-phenyl | H |
| 1-Cl-1-c-Pr | H | H | H | CH₂CH₂ | 3-CF₃-phenyl | H |
| 1-Cl-1-c-Pr | H | H | H | CH₂CH₂ | 2-F,4-Cl-phenyl | H |
| 1-Cl-1-c-Pr | H | H | H | CH₂CH₂ | 3,4-diCl-phenyl | H |
| 1-Cl-1-c-Pr | H | H | H | CH₂CH₂ | 3,5-diCl-phenyl | H |
| 1-Cl-1-c-Pr | H | H | H | CH₂CH₂ | 3-PhO-phenyl | H |
| 1-Cl-1-c-Pr | H | H | H | CH₂CH₂ | 2,6-diCl-phenyl | H |
| 1-Cl-1-c-Pr | H | H | H | CH₂CH₂ | 2,6-diF-phenyl | H |
| 1-Cl-1-c-Pr | H | H | H | CH₂CH₂ | 2-F,4-CF₃-phenyl | H |
| 1-Cl-1-c-Pr | H | H | H | CH₂O | 4-F-phenyl | H |
| 1-Cl-1-c-Pr | H | H | H | CH₂O | 4-Cl-phenyl | H |
| 1-Cl-1-c-Pr | H | H | H | CH₂O | 4-Br-phenyl | H |
| 1-Cl-1-c-Pr | H | H | H | CH₂O | 4-I-phenyl | H |
| 1-Cl-1-c-Pr | H | H | H | CH₂O | 4-CH₃-phenyl | H |
| 1-Cl-1-c-Pr | H | H | H | CH₂O | 4-CF₃-phenyl | H |
| 1-Cl-1-c-Pr | H | H | H | CH₂O | 4-CH₃O-phenyl | H |
| 1-Cl-1-c-Pr | H | H | H | CH₂O | 4-CH₃S-phenyl | H |
| 1-Cl-1-c-Pr | H | H | H | CH₂O | 4-CF₃O-phenyl | H |
| 1-Cl-1-c-Pr | H | H | H | CH₂O | 4-CH₃CH₂-phenyl | H |
| 1-Cl-1-c-Pr | H | H | H | CH₂O | 4-CN-phenyl | H |
| 1-Cl-1-c-Pr | H | H | H | CH₂O | 2-Cl-phenyl | H |
| 1-Cl-1-c-Pr | H | H | H | CH₂O | 3-Cl-phenyl | H |
| 1-Cl-1-c-Pr | H | H | H | CH₂O | 2,4-diCl-phenyl | H |
| 1-Cl-1-c-Pr | H | H | H | CH₂O | 2,4-diF-phenyl | H |
| 1-Cl-1-c-Pr | H | H | H | CH₂O | 3-Br-phenyl | H |
| 1-Cl-1-c-Pr | H | H | H | CH₂O | 3-CF₃-phenyl | H |
| 1-Cl-1-c-Pr | H | H | H | CH₂O | 2-F,4-Cl-phenyl | H |
| 1-Cl-1-c-Pr | H | H | H | CH₂O | 3,4-diCl-phenyl | H |
| 1-Cl-1-c-Pr | H | H | H | CH₂O | 3,5-diCl-phenyl | H |
| 1-Cl-1-c-Pr | H | H | H | CH₂O | 3-PhO-phenyl | H |
| 1-Cl-1-c-Pr | H | H | H | CH₂O | 2,6-diCl-phenyl | H |
| 1-Cl-1-c-Pr | H | H | H | CH₂O | 2,6-diF-phenyl | H |
| 1-Cl-1-c-Pr | H | H | H | CH₂O | 2-F,4-CF₃-phenyl | H |
| 1-Cl-1-c-Pr | H | H | H | CH₂CH₂O | 4-F-phenyl | H |
| 1-Cl-1-c-Pr | H | H | H | CH₂CH₂O | 4-Cl-phenyl | H |
| 1-Cl-1-c-Pr | H | H | H | CH₂CH₂O | 4-Br-phenyl | H |
| 1-Cl-1-c-Pr | H | H | H | CH₂CH₂O | 4-I-phenyl | H |
| 1-Cl-1-c-Pr | H | H | H | CH₂CH₂O | 4-CH₃-phenyl | H |
| 1-Cl-1-c-Pr | H | H | H | CH₂CH₂O | 4-CF₃-phenyl | H |
| 1-Cl-1-c-Pr | H | H | H | CH₂CH₂O | 4-CH₃O-phenyl | H |
| 1-Cl-1-c-Pr | H | H | H | CH₂CH₂O | 4-CH₃S-phenyl | H |
| 1-Cl-1-c-Pr | H | H | H | CH₂CH₂O | 4-CF₃O-phenyl | H |
| 1-Cl-1-c-Pr | H | H | H | CH₂CH₂O | 4-CH₃CH₂-phenyl | H |
| 1-Cl-1-c-Pr | H | H | H | CH₂CH₂O | 4-CN-phenyl | H |
| 1-Cl-1-c-Pr | H | H | H | CH₂CH₂O | 2-Cl-phenyl | H |
| 1-Cl-1-c-Pr | H | H | H | CH₂CH₂O | 3-Cl-phenyl | H |
| 1-Cl-1-c-Pr | H | H | H | CH₂CH₂O | 2,4-diCl-phenyl | H |
| 1-Cl-1-c-Pr | H | H | H | CH₂CH₂O | 2,4-diF-phenyl | H |
| 1-Cl-1-c-Pr | H | H | H | CH₂CH₂O | 3-Br-phenyl | H |
| 1-Cl-1-c-Pr | H | H | H | CH₂CH₂O | 3-CF₃-phenyl | H |
| 1-Cl-1-c-Pr | H | H | H | CH₂CH₂O | 2-F,4-Cl-phenyl | H |
| 1-Cl-1-c-Pr | H | H | H | CH₂CH₂O | 3,4-diCl-phenyl | H |
| 1-Cl-1-c-Pr | H | H | H | CH₂CH₂O | 3,5-diCl-phenyl | H |
| 1-Cl-1-c-Pr | H | H | H | CH₂CH₂O | 3-PhO-phenyl | H |
| 1-Cl-1-c-Pr | H | H | H | CH₂CH₂O | 2,6-diCl-phenyl | H |
| 1-Cl-1-c-Pr | H | H | H | CH₂CH₂O | 2,6-diF-phenyl | H |
| 1-Cl-1-c-Pr | H | H | H | CH₂CH₂O | 2-F,4-CF₃-phenyl | H |
| 4-Cl—Ph | H | Note 1 | H | Bond | 4-Cl—Ph | Note 1 |
| 4-Cl—Ph | H | Note 2 | H | Bond | 4-Cl—Ph | Note 2 |
| 4-Cl—Ph | H | Note 3 | H | Bond | 4-Cl—Ph | Note 3 |
| 4-Cl—Ph | H | Note 4 | H | Bond | 4-Cl—Ph | Note 4 |
| 4-Cl—Ph | H | Note 5 | H | Bond | 4-Cl—Ph | Note 5 |
| 4-Cl—Ph | H | Note 1 | H | CH₂ | 4-Cl—Ph | Note 1 |
| 4-Cl—Ph | H | Note 2 | H | CH₂ | 4-Cl—Ph | Note 2 |
| 4-Cl—Ph | H | Note 3 | H | CH₂ | 4-Cl—Ph | Note 3 |
| 4-Cl—Ph | H | Note 4 | H | CH₂ | 4-Cl—Ph | Note 4 |
| 4-Cl—Ph | H | Note 5 | H | CH₂ | 4-Cl—Ph | Note 5 |
| 4-Cl—Ph | H | Note 1 | H | CH₂O | 4-Cl—Ph | Note 1 |
| 4-Cl—Ph | H | Note 2 | H | CH₂O | 4-Cl—Ph | Note 2 |
| 4-Cl—Ph | H | Note 3 | H | CH₂O | 4-Cl—Ph | Note 3 |
| 4-Cl—Ph | H | Note 4 | H | CH₂O | 4-Cl—Ph | Note 4 |
| 4-Cl—Ph | H | Note 5 | H | CH₂O | 4-Cl—Ph | Note 5 |
| t-Butyl | H | Note 1 | H | Bond | 4-Cl—Ph | Note 1 |
| t-Butyl | H | Note 2 | H | Bond | 4-Cl—Ph | Note 2 |
| t-Butyl | H | Note 3 | H | Bond | 4-Cl—Ph | Note 3 |
| t-Butyl | H | Note 4 | H | Bond | 4-Cl—Ph | Note 4 |
| t-Butyl | H | Note 5 | H | Bond | 4-Cl—Ph | Note 5 |
| t-Butyl | H | Note 1 | H | CH₂ | 4-Cl—Ph | Note 1 |
| t-Butyl | H | Note 2 | H | CH₂ | 4-Cl—Ph | Note 2 |
| t-Butyl | H | Note 3 | H | CH₂ | 4-Cl—Ph | Note 3 |
| t-Butyl | H | Note 4 | H | CH₂ | 4-Cl—Ph | Note 4 |
| t-Butyl | H | Note 5 | H | CH₂ | 4-Cl—Ph | Note 5 |
| t-Butyl | H | Note 1 | H | CH₂O | 4-Cl—Ph | Note 1 |
| t-Butyl | H | Note 2 | H | CH₂O | 4-Cl—Ph | Note 2 |
| t-Butyl | H | Note 3 | H | CH₂O | 4-Cl—Ph | Note 3 |
| t-Butyl | H | Note 4 | H | CH₂O | 4-Cl—Ph | Note 4 |
| t-Butyl | H | Note 5 | H | CH₂O | 4-Cl—Ph | Note 5 |

Note 1:
R^{4a} and R⁶ are taken together with the linking atoms to form a cyclopropyl ring.
Note 2:
R^{4a} and R⁶ are taken together with the linking atoms to form a cyclobutyl ring.
Note 3:
R^{4a} and R⁶ are taken together with the linking atoms to form a cyclopentyl ring.
Note 4:
R^{4a} and R⁶ are taken together with the linking atoms to form a cyclohexyl ring.
Note 5:
R^{4a} and R⁶ are taken together with the linking atoms to form a cycloheptyl ring.
A¹ is CH,
A² is N and
R¹ is H

The present disclosure also includes Tables 1B through 1N, each of which is constructed the same as Table 1A above except that the row heading in Table 1A (i.e. “A¹ is CH, A² is N and R¹ is H”) below the Markush structure is replaced with the respective row heading shown below. For example, in Table 2A the row heading is “A¹ is CH, A² is CH and R¹ is H” and the variables R², R³, R^{4a}, R^{4b}, L, R⁵ and R⁶ are as defined in Table 1A above.

| Table | Table Heading |
|---|---|
| 1B | A¹ is CH, A² is CH and R¹ is H |
| 1C | A¹ is N, A² is N and R¹ is H |
| 1D | A¹ is CH, A² is N and R¹ is SH |
| 1E | A¹ is CH, A² is CH and R¹ is SH |
| 1F | A¹ is N, A² is N and R¹ is SH |
| 1G | A¹ is CH, A² is N and R¹ is Cl |
| 1H | A¹ is CH, A² is N and R¹ is CN |
| 1I | A¹ is CH, A² is N and R¹ is SCN |
| 1J | A¹ is CH, A² is N and R¹ is CH₃S |
| 1K | A¹ is CH, A² is N and R¹ is CH₃CH₂S |
| 1L | A¹ is CH, A² is N and R¹ is CF₃S |
| 1M | A¹ is CH, A² is N and R¹ is CH₂═CHCH₂S |
| 1N | A¹ is CH, A² is N and R¹ is CH≡CCH₂S |

Table 2 refers to Q structures in Embodiment 19 and G structures in Embodiment 45. A dash “-” in the table column means there is no appropriate entry.

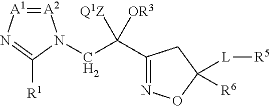

TABLE 2A

| Z | Q1 | R^{9a} | R^{9b} | R⁵ | R^{8a} | R^{8b} |
|---|---|---|---|---|---|---|
| Bond | Q-1 | 4-Cl | — | Methyl | — | — |
| Bond | Q-1 | 4-Cl | — | Ethyl | — | — |
| Bond | Q-1 | 4-Cl | — | Propyl | — | — |
| Bond | Q-1 | 4-Cl | — | i-Butyl | — | — |
| Bond | Q-1 | 4-Cl | — | t-Butyl | — | — |
| Bond | Q-1 | 4-Cl | — | n-Hexyl | — | — |
| Bond | Q-1 | 4-Cl | — | t-Octyl | — | — |
| Bond | Q-1 | 4-Cl | — | c-Propyl | — | — |
| Bond | Q-1 | 4-Cl | — | c-Pentyl | — | — |
| Bond | Q-1 | 4-Cl | — | c-hexyl | — | — |
| Bond | Q-1 | 4-Cl | — | CF₃ | — | — |
| Bond | Q-1 | 4-Cl | — | CF₃CH₂ | — | — |
| Bond | Q-1 | 4-Cl | — | c-Propylmethyl | — | — |
| Bond | Q-1 | 4-Cl | — | di-Cl-c- | — | — |
| | | | | propylmethyl | | |
| Bond | Q-1 | 4-Cl | — | t-Butyloxymethyl | — | — |
| Bond | Q-1 | 4-Cl | — | t-Octyloxyethyl | — | — |
| Bond | Q-1 | 4-Cl | — | Tri- | — | — |
| | | | | fluororethyoxyethyl | | |
| Bond | Q-1 | 4-Cl | — | t-Butyloxyethoxy- | — | — |
| | | | | methyl | | |
| Bond | Q-1 | 4-Cl | — | t-Butylthiomethyl | — | — |
| Bond | Q-1 | 4-Cl | — | CF₃Shexyl | — | — |
| Bond | Q-1 | 4-Cl | — | Dimethyl- | — | — |
| | | | | aminomethyl | | |
| Bond | Q-1 | 4-Cl | — | Methoxycarbonyl- | — | — |
| | | | | methyl | | |
| Bond | Q-1 | 4-Cl | — | t-Butyloxy | — | — |
| Bond | Q-1 | 4-Cl | — | Trifluoroethoxy | — | — |
| Bond | Q-1 | 4-Cl | — | Allyloxymethyl | — | — |
| Bond | Q-1 | 4-Cl | — | Propargyloxymethyl | — | — |
| Bond | Q-1 | 4-Cl | — | Trifluoromethyl- | — | — |
| | | | | propargyloxymethyl | | |
| Bond | Q-1 | 4-Cl | — | t-Butylcalbonyl- | — | — |
| | | | | oxymethyl | | |
| Bond | Q-1 | 4-Cl | — | Trifluoromethyl- | — | — |
| | | | | carbonyloxymethyl | | |
| Bond | Q-1 | 4-Cl | — | Dimetylamino | — | — |
| Bond | Q-1 | 4-Cl | — | Trimethylsilyl | — | — |
| Bond | Q-1 | 4-Cl | — | G-1 | H | — |
| Bond | Q-1 | 4-Cl | — | G-2 | 5-Cl | — |
| Bond | Q-1 | 4-Cl | — | G-2 | 5-Br | — |
| Bond | Q-1 | 4-Cl | — | G-3 | 5-Cl | — |
| Bond | Q-1 | 4-Cl | — | G-3 | 5-Br | — |
| Bond | Q-1 | 4-Cl | — | G-4 | 5-Cl | — |
| Bond | Q-1 | 4-Cl | — | G-5 | 5-Cl | — |
| Bond | Q-1 | 4-Cl | — | G-6 | 3-CF₃ | — |
| Bond | Q-1 | 4-Cl | — | G-7 | 5-Cl | 1-Me |
| Bond | Q-1 | 4-Cl | — | G-8 | — | 1-Me |
| Bond | Q-1 | 4-Cl | — | G-9 | 5-Cl | — |
| Bond | Q-1 | 4-Cl | — | G-10 | 2-CF₃ | — |
| Bond | Q-1 | 4-Cl | — | G-11 | 2-CF₃ | — |
| Bond | Q-1 | 4-Cl | — | G-12 | 5-Br | — |
| Bond | Q-1 | 4-Cl | — | G-13 | 2-Cl | — |
| Bond | Q-1 | 4-Cl | — | G-14 | 2-Cl | — |
| Bond | Q-1 | 4-Cl | — | G-15 | 4-Br | — |
| Bond | Q-1 | 4-Cl | — | G-16 | 4,5- | — |
| | | | | | diCl | |
| Bond | Q-1 | 4-Cl | — | G-17 | 2-CF₃ | — |
| Bond | Q-1 | 4-Cl | — | G-18 | 2-Me | 1-Me |
| Bond | Q-1 | 4-Cl | — | G-19 | 5-Cl | — |
| Bond | Q-1 | 4-Cl | — | G-20 | — | — |
| Bond | Q-1 | 4-Cl | — | G-21 | 3-CF₃ | — |
| Bond | Q-1 | 4-Cl | — | G-22 | 5-Cl | — |
| Bond | Q-1 | 4-Cl | — | G-23 | — | — |
| Bond | Q-1 | 4-Cl | — | G-24 | 3-CF₃ | — |
| Bond | Q-1 | 4-Cl | — | G-25 | 4-Br | — |
| Bond | Q-1 | 4-Cl | — | G-26 | — | 1-Me |
| Bond | Q-1 | 4-Cl | — | G-27 | — | 1-Me |
| Bond | Q-1 | 4-Cl | — | G-28 | 3-CF₃ | 1-H |
| Bond | Q-1 | 4-Cl | — | G-29 | 5-CF₃ | — |
| Bond | Q-1 | 4-Cl | — | G-30 | 5-CF₃ | — |
| Bond | Q-1 | 4-Cl | — | G-31 | — | — |
| Bond | Q-1 | 4-Cl | — | G-32 | 5-CF₃ | 4-H |
| Bond | Q-1 | 4-Cl | — | G-33 | — | 1- |
| | | | | | | CF₃CH₂ |
| Bond | Q-1 | 4-Cl | — | G-34 | 5-CF₃ | 2-H |
| Bond | Q-1 | 4-Cl | — | G-35 | 3,5- | — |
| | | | | | diCl | |
| Bond | Q-1 | 4-Cl | — | G-36 | 3-CF₃ | — |
| Bond | Q-1 | 4-Cl | — | G-37 | 5-CF₃ | — |
| Bond | Q-1 | 4-Cl | — | G-38 | 3-CF₃ | — |
| Bond | Q-1 | 4-Cl | — | G-39 | 5-CF₃ | — |
| Bond | Q-1 | 4-Cl | — | G-40 | 4-F | — |
| Bond | Q-1 | 4-Cl | — | G-41 | 5-F | — |
| Bond | Q-1 | 4-Cl | — | G-42 | 4-F | — |
| Bond | Q-1 | 4-Cl | — | G-43 | 5-F | — |
| Bond | Q-1 | 4-Cl | — | G-44 | 4,5- | — |
| | | | | | diCl | |
| Bond | Q-1 | 4-Cl | — | G-45 | — | 3-Me |
| Bond | Q-1 | 4-Cl | — | G-46 | — | 2-Me |
| Bond | Q-1 | 4-Cl | — | G-47 | — | 3-H |
| Bond | Q-1 | 4-Cl | — | G-48 | 4-CF₃ | — |
| Bond | Q-1 | 4-Cl | — | G-49 | — | 1-H |
| Bond | Q-1 | 4-Cl | — | G-50 | 5-H | — |
| Bond | Q-1 | 4-Cl | — | G-51 | — | 2-Me |
| Bond | Q-1 | 4-Cl | — | G-52 | 5-CF₃ | — |
| Bond | Q-1 | 4-Cl | — | G-53 | 5-Cl | — |
| Bond | Q-1 | 4-Cl | — | G-54 | 6-Cl | — |
| Bond | Q-1 | 4-Cl | — | G-55 | — | — |
| Bond | Q-1 | 4-Cl | — | G-56 | 2-Cl | — |
| Bond | Q-1 | 4-Cl | — | G-57 | 2-CF₃ | — |
| Bond | Q-1 | 4-Cl | — | G-58 | 5-CF₃ | — |
| Bond | Q-1 | 4-Cl | — | G-59 | 5-Cl | — |
| Bond | Q-1 | 4-Cl | — | G-60 | 6-Cl | — |
| Bond | Q-1 | 4-Cl | — | G-61 | 6-Cl | — |
| Bond | Q-1 | 4-Cl | — | G-62 | — | — |
| Bond | Q-1 | 4-Cl | — | G-63 | 5-Cl | — |
| Bond | Q-1 | 4-Cl | — | G-64 | 6-Cl | — |
| Bond | Q-1 | 4-Cl | — | G-65 | 3-Cl | — |
| Bond | Q-2 | 5-Cl | — | G-1 | 4-Cl | — |
| Bond | Q-2 | 5-Br | — | G-1 | 4-Cl | — |
| Bond | Q-3 | 5-Cl | — | G-1 | 4-Cl | — |
| Bond | Q-3 | 5-Br | — | G-1 | 4-Cl | — |
| Bond | Q-4 | 5-Cl | — | G-1 | 4-Cl | — |
| Bond | Q-5 | 5-Cl | — | G-1 | 4-Cl | — |
| CH₂ | Q-6 | 3-CF₃ | — | G-1 | 4-Cl | — |
| Bond | Q-7 | 5-Cl | 1-Me | G-1 | 4-Cl | — |
| Bond | Q-8 | — | 1-Me | G-1 | 4-Cl | — |
| Bond | Q-9 | 5-Cl | — | G-1 | 4-Cl | — |
| Bond | Q-10 | 2-CF₃ | — | G-1 | 4-Cl | — |
| Bond | Q-11 | 2-CF₃ | — | G-1 | 4-Cl | — |
| Bond | Q-12 | 5-Br | — | G-1 | 4-Cl | — |
| Bond | Q-13 | 2-Cl | — | G-1 | 4-Cl | — |
| Bond | Q-14 | 2-Cl | — | G-1 | 4-Cl | — |
| CH₂ | Q-15 | 4-Br | — | G-1 | 4-Cl | — |
| Bond | Q-16 | 4,5-diCl | — | G-1 | 4-Cl | — |
| Bond | Q-17 | 2-CF₃ | — | G-1 | 4-Cl | — |
| Bond | Q-18 | 2-Me | 1-Me | G-1 | 4-Cl | — |
| Bond | Q-19 | 5-Cl | — | G-1 | 4-Cl | — |
| Bond | Q-20 | — | — | G-1 | 4-Cl | — |
| Bond | Q-21 | 3-CF₃ | — | G-1 | 4-Cl | — |
| Bond | Q-22 | 5-Cl | — | G-1 | 4-Cl | — |
| Bond | Q-23 | — | — | G-1 | 4-Cl | — |
| Bond | Q-24 | 3-CF₃ | — | G-1 | 4-Cl | — |
| CH₂ | Q-25 | 4-Br | — | G-1 | 4-Cl | — |
| Bond | Q-26 | — | 1-Me | G-1 | 4-Cl | — |
| Bond | Q-27 | — | 1-Me | G-1 | 4-Cl | — |
| Bond | Q-28 | 3-CF₃ | 1-H | G-1 | 4-Cl | — |
| Bond | Q-29 | 5-CF₃ | — | G-1 | 4-Cl | — |
| Bond | Q-30 | 5-CF₃ | — | G-1 | 4-Cl | — |
| CH₂ | Q-31 | — | — | G-1 | 4-Cl | — |
| Bond | Q-32 | 5-CF₃ | 4-H | G-1 | 4-Cl | — |
| Bond | Q-33 | — | 1- | G-1 | 4-Cl | — |
| | | | CF₃CH₂ | | | |
| Bond | Q-34 | 5-CF₃ | 2-H | G-1 | 4-Cl | — |
| CH₂ | Q-35 | 3,5-diCl | — | G-1 | 4-Cl | — |
| Bond | Q-36 | 3-CF₃ | — | G-1 | 4-Cl | — |
| Bond | Q-37 | 5-CF₃ | — | G-1 | 4-Cl | — |
| Bond | Q-38 | 3-CF₃ | — | G-1 | 4-Cl | — |
| Bond | Q-39 | 5-CF₃ | — | G-1 | 4-Cl | — |
| Bond | Q-40 | 4-F | — | G-1 | 4-Cl | — |
| Bond | Q-41 | 5-F | — | G-1 | 4-Cl | — |
| Bond | Q-42 | 4-F | — | G-1 | 4-Cl | — |
| Bond | Q-43 | 5-F | — | G-1 | 4-Cl | — |
| CH₂ | Q-44 | 4,5-diCl | — | G-1 | 4-Cl | — |
| Bond | Q-45 | — | 3-Me | G-1 | 4-Cl | — |
| Bond | Q-46 | — | 2-Me | G-1 | 4-Cl | — |
| Bond | Q-47 | — | 3-H | G-1 | 4-Cl | — |
| CH₂ | Q-48 | 4-CF₃ | — | G-1 | 4-Cl | — |
| Bond | Q-49 | — | 1-H | G-1 | 4-Cl | — |
| CH₂ | Q-50 | 5-H | — | G-1 | 4-Cl | — |
| Bond | Q-51 | — | 2-Me | G-1 | 4-Cl | — |
| CH₂ | Q-52 | 5-CF₃ | — | G-1 | 4-Cl | — |
| Bond | Q-53 | 5-Cl | — | G-1 | 4-Cl | — |
| Bond | Q-54 | 6-Cl | — | G-1 | 4-Cl | — |
| Bond | Q-55 | — | — | G-1 | 4-Cl | — |
| Bond | Q-56 | 2-Cl | — | G-1 | 4-Cl | — |
| Bond | Q-57 | 2-CF₃ | — | G-1 | 4-Cl | — |
| Bond | Q-58 | 5-CF₃ | — | G-1 | 4-Cl | — |
| Bond | Q-59 | 5-Cl | — | G-1 | 4-Cl | — |
| Bond | Q-60 | 6-Cl | — | G-1 | 4-Cl | — |
| Bond | Q-61 | 6-Cl | — | G-1 | 4-Cl | — |
| Bond | Q-62 | — | — | G-1 | 4-Cl | — |
| Bond | Q-63 | 5-Cl | — | G-1 | 4-Cl | — |
| Bond | Q-64 | 6-Cl | — | G-1 | 4-Cl | — |
| Bond | Q-65 | 3-Cl | — | G-1 | 4-Cl | — |
| CH₂ | Q-1 | — | — | G-1 | 4-Cl | — |
| CH₂ | Q-1 | 4-Cl | — | G-1 | 4-Cl | — |
| CH₂ | Q-1 | 2-Cl | — | G-1 | 4-Cl | — |
| CH₂ | Q-1 | 4-F | — | G-1 | 4-Cl | — |
| CH₂ | Q-1 | 2-F | — | G-1 | 4-Cl | — |
| CH₂ | Q-1 | 2,4-diCl | — | G-1 | 4-Cl | — |
| CH₂ | Q-1 | 2,4-diF | — | G-1 | 4-Cl | — |
| CH₂ | Q-1 | 4-CF₃ | — | G-1 | 4-Cl | — |
| CH₂ | Q-1 | 2-F,4-Cl | — | G-1 | 4-Cl | — |
| CH₂ | Q-1 | 2-F,4- | — | G-1 | 4-Cl | — |
| | | CF₃ | | | | |
| CH₂ | Q-1 | 4-Br | — | G-1 | 4-Cl | — |
| CH₂ | Q-1 | 4-I | — | G-1 | 4-Cl | — |
| CH₂ | Q-1 | — | — | G-1 | 4-Br | — |
| CH₂ | Q-1 | 4-Cl | — | G-1 | 4-Br | — |
| CH₂ | Q-1 | 2-Cl | — | G-1 | 4-Br | — |
| CH₂ | Q-1 | 4-F | — | G-1 | 4-Br | — |
| CH₂ | Q-1 | 2-F | — | G-1 | 4-Br | — |
| CH₂ | Q-1 | 2,4-diCl | — | G-1 | 4-Br | — |
| CH₂ | Q-1 | 2,4-diF | — | G-1 | 4-Br | — |
| CH₂ | Q-1 | 4-CF₃ | — | G-1 | 4-Br | — |
| CH₂ | Q-1 | 2-F,4-Cl | — | G-1 | 4-Br | — |
| CH₂ | Q-1 | 2-F,4- | — | G-1 | 4-Br | — |
| | | CF₃ | | | | |
| CH₂ | Q-1 | 4-Br | — | G-1 | 4-Br | — |
| CH₂ | Q-1 | 4-I | — | G-1 | 4-Br | — |
| CH₂ | Q-1 | — | — | G-1 | 4-I | — |
| CH₂ | Q-1 | 4-Cl | — | G-1 | 4-I | — |
| CH₂ | Q-1 | 2-Cl | — | G-1 | 4-I | — |
| CH₂ | Q-1 | 4-F | — | G-1 | 4-I | — |
| CH₂ | Q-1 | 2-F | — | G-1 | 4-I | — |
| CH₂ | Q-1 | 2,4-diCl | — | G-1 | 4-I | — |
| CH₂ | Q-1 | 2,4-diF | — | G-1 | 4-I | — |
| CH₂ | Q-1 | 4-CF₃ | — | G-1 | 4-I | — |
| CH₂ | Q-1 | 2-F,4-Cl | — | G-1 | 4-I | — |
| CH₂ | Q-1 | 2-F,4- | — | G-1 | 4-I | — |
| | | CF₃ | | | | |
| CH₂ | Q-1 | 4-Br | — | G-1 | 4-I | — |
| CH₂ | Q-1 | 4-I | — | G-1 | 4-I | — |
| CH₂ | Q-1 | — | — | G-1 | 4-CF₃ | — |
| CH₂ | Q-1 | 4-Cl | — | G-1 | 4-CF₃ | — |
| CH₂ | Q-1 | 2-Cl | — | G-1 | 4-CF₃ | — |
| CH₂ | Q-1 | 4-F | — | G-1 | 4-CF₃ | — |
| CH₂ | Q-1 | 2-F | — | G-1 | 4-CF₃ | — |
| CH₂ | Q-1 | 2,4-diCl | — | G-1 | 4-CF₃ | — |
| CH₂ | Q-1 | 2,4-diF | — | G-1 | 4-CF₃ | — |
| CH₂ | Q-1 | 4-CF₃ | — | G-1 | 4-CF₃ | — |
| CH₂ | Q-1 | 2-F,4-Cl | — | G-1 | 4-CF₃ | — |
| CH₂ | Q-1 | 2-F,4- | — | G-1 | 4-CF₃ | — |
| | | CF₃ | | | | |
| CH₂ | Q-1 | 4-Br | — | G-1 | 4-CF₃ | — |
| CH₂ | Q-1 | 4-I | — | G-1 | 4-CF₃ | — |
| CH₂O | Q-1 | — | — | G-1 | 4-Cl | — |
| CH₂O | Q-1 | 4-Cl | — | G-1 | 4-Cl | — |
| CH₂O | Q-1 | 2-Cl | — | G-1 | 4-Cl | — |
| CH₂O | Q-1 | 4-F | — | G-1 | 4-Cl | — |
| CH₂O | Q-1 | 2-F | — | G-1 | 4-Cl | — |
| CH₂O | Q-1 | 2,4-diCl | — | G-1 | 4-Cl | — |
| CH₂O | Q-1 | 2,4-diF | — | G-1 | 4-Cl | — |
| CH₂O | Q-1 | 4-CF₃ | — | G-1 | 4-Cl | — |
| CH₂O | Q-1 | 2-F,4-Cl | — | G-1 | 4-Cl | — |
| CH₂O | Q-1 | 2-F,4- | — | G-1 | 4-Cl | — |
| | | CF₃ | | | | |
| CH₂O | Q-1 | 4-Br | — | G-1 | 4-Cl | — |
| CH₂O | Q-1 | 4-I | — | G-1 | 4-Cl | — |
| CH₂O | Q-1 | — | — | G-1 | 4-Br | — |
| CH₂O | Q-1 | 4-Cl | — | G-1 | 4-Br | — |
| CH₂O | Q-1 | 2-Cl | — | G-1 | 4-Br | — |
| CH₂O | Q-1 | 4-F | — | G-1 | 4-Br | — |
| CH₂O | Q-1 | 2-F | — | G-1 | 4-Br | — |
| CH₂O | Q-1 | 2,4-diCl | — | G-1 | 4-Br | — |
| CH₂O | Q-1 | 2,4-diF | — | G-1 | 4-Br | — |
| CH₂O | Q-1 | 4-CF₃ | — | G-1 | 4-Br | — |
| CH₂O | Q-1 | 2-F,4-Cl | — | G-1 | 4-Br | — |
| CH₂O | Q-1 | 2-F,4- | — | G-1 | 4-Br | — |
| | | CF₃ | | | | |
| CH₂O | Q-1 | 4-Br | — | G-1 | 4-Br | — |
| CH₂O | Q-1 | 4-I | — | G-1 | 4-Br | — |
| CH₂O | Q-1 | — | — | G-1 | 4-I | — |
| CH₂O | Q-1 | 4-Cl | — | G-1 | 4-I | — |
| CH₂O | Q-1 | 2-Cl | — | G-1 | 4-I | — |
| CH₂O | Q-1 | 4-F | — | G-1 | 4-I | — |
| CH₂O | Q-1 | 2-F | — | G-1 | 4-I | — |
| CH₂O | Q-1 | 2,4-diCl | — | G-1 | 4-I | — |
| CH₂O | Q-1 | 2,4-diF | — | G-1 | 4-I | — |
| CH₂O | Q-1 | 4-CF₃ | — | G-1 | 4-I | — |
| CH₂O | Q-1 | 2-F,4-Cl | — | G-1 | 4-I | — |
| CH₂O | Q-1 | 2-F,4- | — | G-1 | 4-I | — |
| | | CF₃ | | | | |
| CH₂O | Q-1 | 4-Br | — | G-1 | 4-I | — |
| CH₂O | Q-1 | 4-I | — | G-1 | 4-I | — |
| CH₂O | Q-1 | — | — | G-1 | 4-CF₃ | — |
| CH₂O | Q-1 | 4-Cl | — | G-1 | 4-CF₃ | — |
| CH₂O | Q-1 | 2-Cl | — | G-1 | 4-CF₃ | — |
| CH₂O | Q-1 | 4-F | — | G-1 | 4-CF₃ | — |
| CH₂O | Q-1 | 2-F | — | G-1 | 4-CF₃ | — |
| CH₂O | Q-1 | 2,4-diCl | — | G-1 | 4-CF₃ | — |
| CH₂O | Q-1 | 2,4-diF | — | G-1 | 4-CF₃ | — |
| CH₂O | Q-1 | 4-CF₃ | — | G-1 | 4-CF₃ | — |
| CH₂O | Q-1 | 2-F,4-Cl | — | G-1 | 4-CF₃ | — |
| CH₂O | Q-1 | 2-F,4- | — | G-1 | 4-CF₃ | — |
| | | CF₃ | | | | |
| CH₂O | Q-1 | 4-Br | — | G-1 | 4-CF₃ | — |
| CH₂O | Q-1 | 4-I | — | G-1 | 4-CF₃ | — |

A¹ is CH,
A² is N,
R¹ is H,
R³ is H and
L is a bond

The present disclosure also includes Tables 2B through 2Y, each of which is constructed the same as Table 2A above except that the row heading in Table 2A (i.e. “A¹ is CH, A² is N, R¹ is H, R³ is H and L is a bond”) below the Markush structure is replaced with the respective row heading shown below. For example, in Table 2A the row heading is “A¹ is CH, A² is CH, R¹ is H, R³ is H and L is a bond” and the variables Z, Q¹, R^{9a}, R^{9b}, R⁵, R^{8a} and R^{8b} are as defined in Table 2A above.

| Table | Table Heading |
|---|---|
| 2B | A¹ is CH, A² is CH, R¹ is H, R³ is H and L is a bond |
| 2C | A¹ is N, A² is N, R¹ is H, R³ is H and L is a bond |
| 2D | A¹ is CH, A² is N, R¹ is SH, R³ is H and L is a bond |
| 2E | A¹ is CH, A² is N, R¹ is SH, R³ is Me and L is a bond |
| 2F | A¹ is CH, A² is N, R¹ is H, R³ is H and L is a CH₂ |
| 2G | A¹ is CH, A² is CH, R¹ is H, R³ is H and L is a CH₂ |
| 2H | A¹ is N, A² is N, R¹ is H, R³ is H and L is a CH₂ |
| 2I | A¹ is CH, A² is N, R¹ is SH, R³ is H and L is a CH₂ |
| 2J | A¹ is CH, A² is N, R¹ is SH, R³ is Me and L is a CH₂ |
| 2K | A¹ is CH, A² is N, R¹ is H, R³ is H and L is a CH₂CH₂ |
| 2L | A¹ is CH, A² is CH, R¹ is H, R³ is H and L is a CH₂CH₂ |
| 2M | A¹ is N, A² is N, R¹ is H, R³ is H and L is a CH₂CH₂ |
| 2N | A¹ is CH, A² is N, R¹ is SH, R³ is H and L is a CH₂CH₂ |
| 2O | A¹ is CH, A² is N, R¹ is SH, R³ is Me and L is a CH₂CH₂ |
| 2P | A¹ is CH, A² is N, R¹ is H, R³ is H and L is a CH₂O |
| 2Q | A¹ is CH, A² is CH, R¹ is H, R³ is H and L is a CH₂O |
| 2R | A¹ is N, A² is N, R¹ is H, R³ is H and L is a CH₂O |
| 2S | A¹ is CH, A² is N, R¹ is SH, R³ is H and L is a CH₂O |
| 2T | A¹ is CH, A² is N, R¹ is SH, R³ is Me and L is a CH₂O |
| 2U | A¹ is CH, A² is N, R¹ is H, R³ is H and L is a CH₂CH₂O |
| 2V | A¹ is CH, A² is CH, R¹ is H, R³ is H and L is a CH₂CH₂O |
| 2W | A¹ is N, A² is N, R¹ is H, R³ is H and L is a CH₂CH₂O |
| 2X | A¹ is CH, A² is N, R¹ is SH, R³ is H and L is a CH₂CH₂O |
| 2Y | A¹ is CH, A² is N, R¹ is SH, R³ is Me and L is a CH₂CH₂O |

### Formulation/Utility

A compound of Formula 1 of this invention (including N-oxides and salts thereof) will generally be used as a fungicidal active ingredient in a composition, i.e. formulation, with at least one additional component selected from the group consisting of surfactants, solid diluents and liquid diluents, which serve as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions, oil-in-water emulsions, flowable concentrates and/or suspoemulsions) and the like, which optionally can be thickened into gels. The general types of aqueous liquid compositions are soluble concentrate, suspension concentrate, capsule suspension, concentrated emulsion, microemulsion, oil-in-water emulsion, flowable concentrate and suspo-emulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water, but occasionally another suitable medium like an aromatic or paraffinic hydrocarbon or vegetable oil. Spray volumes can range from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto seeds of crops and other desirable vegetation as seed treatments before planting to protect developing roots and other subterranean plant parts and/or foliage through systemic uptake.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and Water- | 0.001-90 | 0-99.999 | 0-15 |
| soluble Granules, Tablets and | | | |
| Powders | | | |
| Oil Dispersions, Suspensions, | 1-50 | 40-99 | 0-50 |
| Emulsions, Solutions | | | |
| (including Emulsifiable | | | |
| Concentrates) | | | |
| Dusts | 1-25 | 70-99 | 0-5 |
| Granules and Pellets | 0.001-95 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, N.J.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), alkyl phosphates (e.g., triethyl phosphate), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters, alkyl and aryl benzoates and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol, cresol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. No. 4,144,050, U.S. Pat. No. 3,920,442 and DE 3,246,493. Tablets can be prepared as taught in U.S. Pat. No. 5,180,587, U.S. Pat. No. 5,232,701 and U.S. Pat. No. 5,208,030. Films can be prepared as taught in GB 2,095,558 and U.S. Pat. No. 3,299,566.

One embodiment of the present invention relates to a method for controlling fungal pathogens, comprising diluting the fungicidal composition of the present invention (a compound of Formula 1 formulated with surfactants, solid diluents and liquid diluents or a formulated mixture of a compound of Formula 1 and at least one other fungicide) with water, and optionally adding an adjuvant to form a diluted composition, and contacting the fungal pathogen or its environment with an effective amount of said diluted composition.

Although a spray composition formed by diluting with water a sufficient concentration of the present fungicidal composition can provide sufficient efficacy for controlling fungal pathogens, separately formulated adjuvant products can also be added to spray tank mixtures. These additional adjuvants are commonly known as “spray adjuvants” or “tank-mix adjuvants”, and include any substance mixed in a spray tank to improve the performance of a pesticide or alter the physical properties of the spray mixture. Adjuvants can be anionic or nonionic surfactants, emulsifying agents, petroleum-based crop oils, crop-derived seed oils, acidifiers, buffers, thickeners or defoaming agents. Adjuvants are used to enhancing efficacy (e.g., biological availability, adhesion, penetration, uniformity of coverage and durability of protection), or minimizing or eliminating spray application problems associated with incompatibility, foaming, drift, evaporation, volatilization and degradation. To obtain optimal performance, adjuvants are selected with regard to the properties of the active ingredient, formulation and target (e.g., crops, insect pests).

The amount of adjuvants added to spray mixtures is generally in the range of about 2.5% to 0.1% by volume. The application rates of adjuvants added to spray mixtures are typically between about 1 to 5 L per hectare. Representative examples of spray adjuvants include: Adigor® (Syngenta) 47% methylated rapeseed oil in liquid hydrocarbons, Silwet® (Helena Chemical Company) polyalkyleneoxide modified heptamethyltrisiloxane and Assist® (BASF) 17% surfactant blend in 83% paraffin based mineral oil.

One method of seed treatment is by spraying or dusting the seed with a compound of the invention (i.e. as a formulated composition) before sowing the seeds. Compositions formulated for seed treatment generally comprise a film former or adhesive agent. Therefore typically a seed coating composition of the present invention comprises a biologically effective amount of a compound of Formula 1 and a film former or adhesive agent. Seed can be coated by spraying a flowable suspension concentrate directly into a tumbling bed of seeds and then drying the seeds. Alternatively, other formulation types such as wetted powders, solutions, suspoemulsions, emulsifiable concentrates and emulsions in water can be sprayed on the seed. This process is particularly useful for applying film coatings on seeds. Various coating machines and processes are available to one skilled in the art. Suitable processes include those listed in P. Kosters et al., Seed Treatment: Progress and Prospects, 1994 BCPC Mongraph No. 57, and references listed therein.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, UK, 2000.

In the following Examples, all percentages are by weight and all formulations are prepared in conventional ways. Compound numbers refer to compounds in Index Tables A-C. Without further elaboration, it is believed that one skilled in the art using the preceding description can utilize the present invention to its fullest extent. The following Examples are, therefore, to be constructed as merely illustrative, and not limiting of the disclosure in any way whatsoever. Percentages are by weight except where otherwise indicated.

### EXAMPLE A

High Strength Concentrate

| | |
|---|---|
| Compound 11 | 98.5% |
| silica aerogel | 0.5% |
| synthetic amorphous fine silica | 1.0% |

### EXAMPLE B

Wettable Powder

| | |
|---|---|
| Compound 13 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### EXAMPLE C

Granule

| | |
|---|---|
| Compound 61 | 10.0% |
| attapulgite granules (low volatile matter, 0.71/0.30 mm; | 90.0% |
| U.S.S. No. 25-50 sieves) | |

### EXAMPLE D

Extruded Pellet

| | |
|---|---|
| Compound 81 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### EXAMPLE E

Emulsifiable Concentrate

| | |
|---|---|
| Compound 123 | 10.0% |
| polyoxyethylene sorbitol hexoleate | 20.0% |
| C₆-C₁₀ fatty acid methyl ester | 70.0% |

### EXAMPLE F

Microemulsion

| | |
|---|---|
| Compound 126 | 5.0% |
| polyvinylpyrrolidone-vinyl acetate copolymer | 30.0% |
| alkylpolyglycoside | 30.0% |
| glyceryl monooleate | 15.0% |
| water | 20.0% |

### EXAMPLE G

Seed Treatment

| | |
|---|---|
| Compound 142 | 20.00% |
| polyvinylpyrrolidone-vinyl acetate copolymer | 5.00% |
| montan acid wax | 5.00% |
| calcium ligninsulfonate | 1.00% |
| polyoxyethylene/polyoxypropylene block copolymers | 1.00% |
| stearyl alcohol (POE 20) | 2.00% |
| polyorganosilane | 0.20% |
| colorant red dye | 0.05% |
| water | 65.75% |

### EXAMPLE H

Fertilizer Stick

| | |
|---|---|
| compound 146 | 2.50% |
| pyrrolidone-styrene copolymer | 4.80% |
| tristyrylphenyl 16-ethoxylate | 2.30% |
| talc | 0.80% |
| corn starch | 5.00% |
| slow-release fertilizer | 36.00% |
| kaolin | 38.00% |
| water | 10.60% |

### EXAMPLE I

Suspension Concentrate

| | |
|---|---|
| compound 164 | 35% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| water | 53.7% |

### EXAMPLE J

Emulsion in Water

| | |
|---|---|
| compound 174 | 10.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0 |
| water | 58.7% |

### EXAMPLE K

Oil Dispersion

| | |
|---|---|
| compound 182 | 25% |
| polyoxyethylene sorbitol hexaoleate | 15% |
| organically modified bentonite clay | 2.5% |
| fatty acid methyl ester | 57.5% |

### EXAMPLE L

Suspoemulsion

| | |
|---|---|
| compound 11 | 10.0% |
| imidacloprid | 5.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0% |
| water | 53.7% |

Water-soluble and water-dispersible formulations are typically diluted with water to form aqueous compositions before application. Aqueous compositions for direct applications to the plant or portion thereof (e.g., spray tank compositions) typically contain at least about 1 ppm or more (e.g., from 1 ppm to 100 ppm) of the compound(s) of this invention.

Seed is normally treated at a rate of from about 0.001 g (more typically about 0.1 g) to about 10 g per kilogram of seed (i.e. from about 0.0001 to 1% by weight of the seed before treatment). A flowable suspension formulated for seed treatment typically comprises from about 0.5 to about 70% of the active ingredient, from about 0.5 to about 30% of a film-forming adhesive, from about 0.5 to about 20% of a dispersing agent, from 0 to about 5% of a thickener, from 0 to about 5% of a pigment and/or dye, from 0 to about 2% of an antifoaming agent, from 0 to about 1% of a preservative, and from 0 to about 75% of a volatile liquid diluent.

The compounds of this invention are useful as plant disease control agents. The present invention therefore further comprises a method for controlling plant diseases caused by fungal plant pathogens comprising applying to the plant or portion thereof to be protected, or to the plant seed to be protected, an effective amount of a compound of the invention or a fungicidal composition containing said compound. The compounds and/or compositions of this invention provide control of diseases caused by a broad spectrum of fungal plant pathogens in the Ascomycota, Basidiomycota, Zygomycota phyla, and the fungal-like Oomycata class. They are effective in controlling a broad spectrum of plant diseases, particularly foliar pathogens of ornamental, turf, vegetable, field, cereal, and fruit crops. These pathogens include but are not limited to those listed in Table 1. For Ascomycetes and Basidiomycetes, names for both the sexual/teleomorph/perfect stage as well as names for the asexual/anamorph/imperfect stage (in parentheses) are listed where known. Synonymous names for pathogens are indicated by an equal sign. For example, the sexual/teleomorph/perfect stage name Phaeosphaeria nodorum is followed by the corresponding asexual/anamorph/imperfect stage name Stagnospora nodorum and the synonymous older name Septoria nodorum.

TABLE 1
Ascomycetes in the order Pleosporales including Alternaria solani, A. alternata and A. brassicae,
Guignardia bidwellii, Venturia inaequalis, Pyrenophora tritici-repentis
(Dreschlera tritici-repentis = Helminthosporium tritici-repentis) and Pyrenophora teres
(Dreschlera teres = Helminthosporium teres), Corynespora cassiicola, Phaeosphaeria
nodorum (Stagonospora nodorum = Septoria nodorum), Cochliobolus carbonum and C. heterostrophus,
Leptosphaeria biglobosa and L. maculans;
Ascomycetes in the order Mycosphaerellales including Mycosphaerella graminicola
(Zymoseptoria tritici = Septoria tritici), M. berkeleyi (Cercosporidium personatum), M. arachidis
(Cercospora arachidicola), Passalora sojina (Cercospora sojina), Cercospora
zeae-maydis and C. beticola;
Ascomycetes in the order Erysiphales (the powdery mildews) such as Blumeria graminis
f.sp. tritici and Blumeria graminis f.sp. hordei, Erysiphe polygoni, E. necator (=Uncinula
necator), Podosphaera fuliginea (=Sphaerotheca fuliginea), and Podosphaera leucotricha
(=Sphaerotheca fuliginea);
Ascomycetes in the order Helotiales such as Botryotinia fuckeliana (Botrytis cinerea),
Oculimacula yallundae (=Tapesia yallundae; anamorph Helgardia herpotrichoides =
Pseudocercosporella herpetrichoides), Monilinia fructicola, Sclerotinia sclerotiorum,
Sclerotinia minor, and Sclerotinia homoeocarpa;
Ascomycetes in the order Hypocreales such as Giberella zeae (Fusarium graminearum), G. monoliformis
(Fusarium moniliforme), Fusarium solani and Verticillium dahliae;
Ascomycetes in the order Eurotiales such as Aspergillus flavus and A. parasiticus;
Ascomycetes in the order Diaporthales such as Cryptosphorella viticola (=Phomopsis
viticola), Phomopsis longicolla, and Diaporthe phaseolorum;
Other Ascomycete pathogens including Magnaporthe grisea, Gaeumannomyces graminis,
Rhynchosporium secalis, and anthracnose pathogens such as Glomerella acutata
(Colletotrichum acutatum), G. graminicola (C. graminicola) and G. lagenaria (C. orbiculare);
Basidiomycetes in the order Urediniales (the rusts) including Puccinia recondita, P. striiformis,
Puccinia hordei, P. graminis and P. arachidis), Hemileia vastatrix and
Phakopsora pachyrhizi;
Basidiomycetes in the order Ceratobasidiales such as Thanatophorum cucumeris
(Rhizoctonia solani) and Ceratobasidium oryzae-sativae (Rhizoctonia oryzae);
Basidiomycetes in the order Polyporales such as Athelia rolfsii (Sclerotium rolfsii);
Basidiomycetes in the order Ustilaginales such as Ustilago maydis;
Zygomycetes in the order Mucorales such as Rhizopus stolonifer;
Oomycetes in the order Pythiales, including Phytophthora infestans, P. megasperma, P. parasitica,
P. sojae, P. cinnamomi and P. capsici, and Pythium pathogens such as Pythium
aphanidermatum, P. graminicola, P. irregulare, P. ultimum and P. dissoticum;
Oomycetes in the order Peronosporales such as Plasmopara viticola, P. halstedii,
Peronospora hyoscyami (=Peronospora tabacina), P. manshurica, Hyaloperonospora
parasitica (=Peronospora parasitica), Pseudoperonospora cubensis and Bremia lactucae;
and other genera and species closely related to all of the above pathogens.

In addition to their fungicidal activity, the compositions or combinations also have activity against bacteria such as Erwinia amylovora, Xanthomonas campestris, Pseudomonas syringae, and other related species. By controlling harmful microorganisms, the compounds of the invention are useful for improving (i.e. increasing) the ratio of beneficial to harmful microorganisms in contact with crop plants or their propagules (e.g., seeds, corms, bulbs, tubers, cuttings) or in the agronomic environment of the crop plants or their propagules.

Compounds of the invention are useful in treating all plants, plant parts and seeds. Plant and seed varieties and cultivars can be obtained by conventional propagation and breeding methods or by genetic engineering methods. Genetically modified plants or seeds (transgenic plants or seeds) are those in which a heterologous gene (transgene) has been stably integrated into the plant's or seed's genome. A transgene that is defined by its particular location in the plant genome is called a transformation or transgenic event.

Genetically modified plant cultivars which can be treated according to the invention include those that are resistant against one or more biotic stresses (pests such as nematodes, insects, mites, fungi, etc.) or abiotic stresses (drought, cold temperature, soil salinity, etc.), or that contain other desirable characteristics. Plants can be genetically modified to exhibit traits of, for example, herbicide tolerance, insect-resistance, modified oil profiles or drought tolerance. Useful genetically modified plants containing single gene transformation events or combinations of transformation events are listed in Table 2. Additional information for the genetic modifications listed in Table 2 can be obtained from publicly available databases maintained, for example, by the U.S. Department of Agriculture.

The following abbreviations, T1 through T37, are used in Table 2 for traits. A “-” means the entry is not available.

| Trait | Description |
|---|---|
| T1 | Glyphosate tolerance |
| T2 | High lauric acid oil |
| T3 | Glufosinate tolerance |
| T4 | Phytate breakdown |
| T5 | Oxynil tolerance |
| T6 | Disease resistance |
| T7 | Insect resistance |
| T9 | Modified flower color |
| T11 | ALS herbicide tol. |
| T12 | Dicamba tolerance |
| T13 | Anti-allergy |
| T14 | Salt tolerance |
| T15 | Cold tolerance |
| T16 | Imidazolinone herbicide tol. |
| T17 | Modified alpha-amylase |
| T18 | Pollination control |
| T19 | 2,4-D tolerance |
| T20 | Increased lysine |
| T21 | Drought tolerance |
| T22 | Delayed ripening/senescence |
| T23 | Modified product quality |
| T24 | High cellulose |
| T25 | Modified starch/carbohydrate |
| T26 | Insect & disease resist. |
| T27 | High tryptophan |
| T28 | Erect leaves semidwarf |
| T29 | Semidwarf |
| T30 | Low iron tolerance |
| T31 | Modified oil/fatty acid |
| T32 | HPPD tolerance |
| T33 | High oil |
| T34 | Aryloxyalkanoate tol. |
| T35 | Mesotrione tolerance |
| T36 | Reduced nicotine |
| T37 | Modified product |

TABLE 2

| Crop | Event Name | Event Code | Trait(s) | Gene(s) |
|---|---|---|---|---|
| Alfalfa | J101 | MON-00101-8 | T1 | cp4 epsps (aroA:CP4) |
| Alfalfa | J163 | MON-ØØ163-7 | T1 | cp4 epsps (aroA:CP4) |
| Canola* | 23-18-17 (Event 18) | CGN-89465-2 | T2 | te |
| Canola* | 23-198 (Event 23) | CGN-89465-2 | T2 | te |
| Canola* | 61061 | DP-Ø61Ø61-7 | T1 | gat4621 |
| Canola* | 73496 | DP-Ø73496-4 | T1 | gat4621 |
| Canola* | GT200 (RT200) | MON-89249-2 | T1 | cp4 epsps (aroA:CP4); goxv247 |
| Canola* | GT73 (RT73) | MON-ØØØ73-7 | T1 | cp4 epsps (aroA:CP4); goxv247 |
| Canola* | HCN10 (Topas | — | T3 | bar |
| | 19/2) | | | |
| Canola* | HCN28 (T45) | ACS-BNØØ8-2 | T3 | pat (syn) |
| Canola* | HCN92 (Topas | ACS-BNØØ7-1 | T3 | bar |
| | 19/2) | | | |
| Canola* | MON88302 | MON-883Ø2-9 | T1 | cp4 epsps (aroA:CP4) |
| Canola* | MPS961 | — | T4 | phyA |
| Canola* | MPS962 | — | T4 | phyA |
| Canola* | MPS963 | — | T4 | phyA |
| Canola* | MPS964 | — | T4 | phyA |
| Canola* | MPS965 | — | T4 | phyA |
| Canola* | MS1 (B91-4) | ACS-BNØØ4-7 | T3 | bar |
| Canola* | MS8 | ACS-BNØØ5-8 | T3 | bar |
| Canola* | OXY-235 | ACS-BNØ11-5 | T5 | bxn |
| Canola* | PHY14 | — | T3 | bar |
| Canola* | PHY23 | — | T3 | bar |
| Canola* | PHY35 | — | T3 | bar |
| Canola* | PHY36 | — | T3 | bar |
| Canola* | RF1 (B93-101) | ACS-BNØØ1-4 | T3 | bar |
| Canola* | RF2 (B94-2) | ACS-BNØØ2-5 | T3 | bar |
| Canola* | RF3 | ACS-BNØØ3-6 | T3 | bar |
| Bean | EMBRAPA 5.1 | EMB-PV051-1 | T6 | ac1 (sense and antisense) |
| Brinjal# | EE-1 | — | T7 | cry1Ac |
| Carnation | 11 (7442) | FLO-07442-4 | T8; T9 | surB; dfr; hfl (f3′5′h) |
| Carnation | 11363 (1363A) | FLO-11363-1 | T8; T9 | surB; dfr; bp40 (f3′5′h) |
| Carnation | 1226A (11226) | FLO-11226-8 | T8; T9 | surB; dfr; bp40 (f3′5′h) |
| Carnation | 123.2.2 (40619) | FLO-4Ø619-7 | T8; T9 | surB; dfr; hfl (f3′5′h) |
| Carnation | 123.2.38 (40644) | FLO-4Ø644-4 | T8; T9 | surB; dfr; hfl (f3′5′h) |
| Carnation | 123.8.12 | FLO-4Ø689-6 | T8; T9 | surB; dfr; bp40 (f3′5′h) |
| Carnation | 123.8.8 (40685) | FLO-4Ø685-1 | T8; T9 | surB; dfr; bp40 (f3′5′h) |
| Carnation | 1351A (11351) | FLO-11351-7 | T8; T9 | surB; dfr; bp40 (f3′5′h) |
| Carnation | 1400A (11400) | FLO-114ØØ-2 | T8; T9 | surB; dfr; bp40 (f3′5′h) |
| Carnation | 15 | FLO-ØØØ15-2 | T8; T9 | surB; dfr; hfl (f3′5′h) |
| Carnation | 16 | FLO-ØØØ16-3 | T8; T9 | surB; dfr; hfl (f3′5′h) |
| Carnation | 4 | FLO-ØØØØ4-9 | T8; T9 | surB; dfr; hfl (f3′5′h) |
| Carnation | 66 | FLO-ØØØ66-8 | T8; T10 | surB; acc |
| Carnation | 959A (11959) | FLO-11959-3 | T8; T9 | surB; dfr; bp40 (f3′5′h) |
| Carnation | 988A (11988) | FLO-11988-7 | T8; T9 | surB; dfr; bp40 (f3′5′h) |
| Carnation | 26407 | IFD-26497-2 | ST8; T9 | surB; dfr; bp40 (f3′5′h) |
| Carnation | 25958 | IFD-25958-3 | T8; T9 | surB; dfr; bp40 (f3′5′h) |
| Chicory | RM3-3 | — | T3 | bar |
| Chicory | RM3-4 | — | T3 | bar |
| Chicory | RM3-6 | — | T3 | bar |
| Cotton | 19-51a | DD-Ø1951A-7 | T11 | S4-HrA |
| Cotton | 281-24-236 | DAS-24236-5 | T3; T7 | pat (syn); cry1F |
| Cotton | 3006-210-23 | DAS-21Ø23-5 | T3; T7 | pat (syn); cry1Ac |
| Cotton | 31707 | — | T5; T7 | bxn; cry1Ac |
| Cotton | 31803 | — | T5; T7 | bxn; cry1Ac |
| Cotton | 31807 | — | T5; T7 | bxn; cry1Ac |
| Cotton | 31808 | — | T5; T7 | bxn; cry1Ac |
| Cotton | 42317 | — | T5; T7 | bxn; cry1Ac |
| Cotton | BNLA-601 | — | T7 | cry1Ac |
| Cotton | BXN10211 | BXN10211-9 | T5 | bxn; cry1Ac |
| Cotton | BXN10215 | BXN10215-4 | T5 | bxn; cry1Ac |
| Cotton | BXN10222 | BXN10222-2 | T5 | bxn; cry1Ac |
| Cotton | BXN10224 | BXN10224-4 | T5 | bxn; cry1Ac |
| Cotton | COT102 | SYN-IR102-7 | T7 | vip3A(a) |
| Cotton | COT67B | SYN-IR67B-1 | T7 | cry1Ab |
| Cotton | COT202 | — | T7 | vip3A |
| Cotton | Event 1 | — | T7 | cry1Ac |
| Cotton | GMF Cry1A | GTL-GMF311-7 | T7 | cry1Ab-Ac |
| Cotton | GHB119 | BCS-GH005-8 | T7 | cry2Ac |
| Cotton | GHB614 | BCS-GH002-5 | T1 | 2mepsps |
| Cotton | GK12 | — | T7 | cry1Ab-Ac |
| Cotton | LLCotton25 | ACS-GH001-3 | T3 | bar |
| Cotton | MLS 9124 | — | T7 | cry1C |
| Cotton | MON1076 | MON-89924-2 | T7 | cry1Ac |
| Cotton | MON1445 | MON-01445-2 | T1 | cp4 epsps (aroA:CP4) |
| Cotton | MON15985 | MON-15985-7 | T7 | cry1Ac; cry2Ab2 |
| Cotton | MON1698 | MON-89383-1 | T7 | cp4 epsps (aroA:CP4) |
| Cotton | MON531 | MON-00531-6 | T7 | cry1Ac |
| Cotton | MON757 | MON-00757-7 | T7 | cry1Ac |
| Cotton | MON88913 | MON-88913-8 | T1 | cp4 epsps (aroA:CP4) |
| Cotton | Nqwe Chi 6 Bt | — | T7 | — |
| Cotton | SKG321 | — | T7 | cry1A; CpTI |
| Cotton | T303-3 | BCS-GH003-6 | T7; T3 | cry1Ab; bar |
| Cotton | T304-40 | BCS-GH004-7 | T7; T3 | cry1Ab; bar |
| Cotton | CE43-67B | — | T7 | cry1Ab |
| Cotton | CE46-02A | — | T7 | cry1Ab |
| Cotton | CE44-69D | — | T7 | cry1Ab |
| Cotton | 1143-14A | — | T7 | cry1Ab |
| Cotton | 1143-51B | — | T7 | cry1Ab |
| Cotton | T342-142 | — | T7 | cry1Ab |
| Cotton | PV-GHGT07 | — | T1 | cp4 epsps (aroA:CP4) |
| | (1445) | | | |
| Cotton | EE-GH3 | — | T1 | mepsps |
| Cotton | EE-GH5 | — | T7 | cry1Ab |
| Cotton | MON88701 | MON-88701-3 | T12; T3 | Modified dmo; bar |
| Cotton | OsCr11 | — | T13 | Modified Cry j |
| Creeping | ASR368 | SMG-368ØØ-2 | T1 | cp4 epsps (aroA:CP4) |
| Bentgrass | | | | |
| Eucalyptus | 20-C | — | T14 | codA |
| Eucalyptus | 12-5C | — | T14 | codA |
| Eucalyptus | 12-5B | — | T14 | codA |
| Eucalyptus | 107-1 | — | T14 | codA |
| Eucalyptus | Jan. 9, 2001 | — | T14 | codA |
| Eucalyptus | Feb. 1, 2001 | — | T14 | codA |
| Eucalyptus | | — | T15 | des9 |
| Flax | FP967 | CDC-FL001-2 | T11 | als |
| Lentil | RH44 | — | T16 | als |
| Maize | 3272 | SYN-E3272-5 | T17 | amy797E |
| Maize | 5307 | SYN-05307-1 | T7 | ecry3.1Ab |
| Maize | 59122 | DAS-59122-7 | T7; T3 | cry34Ab1; cry35Ab1; pat |
| Maize | 676 | PH-000676-7 | T3; T18 | pat; dam |
| Maize | 678 | PH-000678-9 | T3; T18 | pat; dam |
| Maize | 680 | PH-000680-2 | T3; T18 | pat; dam |
| Maize | 98140 | DP-098140-6 | T1; T11 | gat4621; zm-hra |
| Maize | Bt10 | — | T7; T3 | cry1Ab; pat |
| Maize | Bt176 (176) | SYN-EV176-9 | T7; T3 | cry1Ab; bar |
| Maize | BVLA430101 | — | T4 | phyA2 |
| Maize | CBH-351 | ACS-ZM004-3 | T7; T3 | cry9C; bar |
| Maize | DAS40278-9 | DAS40278-9 | T19 | aad-1 |
| Maize | DBT418 | DKB-89614-9 | T7; T3 | cry1Ac; pinII; bar |
| Maize | DLL25 (B16) | DKB-89790-5 | T3 | bar |
| Maize | GA21 | MON-00021-9 | T1 | mepsps |
| Maize | GG25 | — | T1 | mepsps |
| Maize | GJ11 | — | T1 | mepsps |
| Maize | Fl117 | — | T1 | mepsps |
| Maize | GAT-ZM1 | — | T3 | pat |
| Maize | LY038 | REN-00038-3 | T20 | cordapA |
| Maize | MIR162 | SYN-IR162-4 | T7 | vip3Aa20 |
| Maize | MIR604 | SYN-IR604-5 | T7 | mcry3A |
| Maize | MON801 | MON801 | T7; T1 | cry1Ab; cp4 epsps (aroA:CP4); |
| | (MON80100) | | | goxv247 |
| Maize | MON802 | MON-80200-7 | T7; T1 | cry1Ab; cp4 epsps (aroA:CP4); |
| | | | | goxv247 |
| Maize | MON809 | PH-MON-809-2 | T7; T1 | cry1Ab; cp4 epsps (aroA:CP4); |
| | | | | goxv247 |
| Maize | MON810 | MON-00810-6 | T7; T1 | cry1Ab; cp4 epsps (aroA:CP4); |
| | | | | goxv247 |
| Maize | MON832 | — | T1 | cp4 epsps (aroA:CP4); goxv247 |
| Maize | MON863 | MON-00863-5 | T7 | cry3Bb1 |
| Maize | MON87427 | MON-87427-7 | T1 | cp4 epsps (aroA:CP4) |
| Maize | MON87460 | MON-87460-4 | T21 | cspB |
| Maize | MON88017 | MON-88017-3 | T7; T1 | cry3Bb1; cp4 epsps (aroA:CP4) |
| Maize | MON89034 | MON-89034-3 | T7 | cry2Ab2; cry1A.105 |
| Maize | MS3 | ACS-ZM001-9 | T3; T18 | bar; bar-se |
| Maize | MS6 | ACS-ZM005-4 | T3; T18 | bar; bar-se |
| Maize | NK603 | MON-00603-6 | T1 | cp4 epsps (aroA:CP4) |
| Maize | T14 | ACS-ZM002-1 | T3 | pat (syn) |
| Maize | T25 | ACS-ZM003-2 | T3 | pat (syn) |
| Maize | TC1507 | DAS-01507-1 | T7; T3 | cry1Fa2; pat |
| Maize | TC6275 | DAS-06275-8 | T7; T3 | mocry1F; bar |
| Maize | VIP1034 | | T7; T3 | vip3A; pat |
| Maize | 43A47 | DP-043A47-3 | T7; T3 | cry1F; cry34Ab1; cry35Ab1; pat |
| Maize | 40416 | DP-040416-8 | T7; T3 | cry1F; cry34Ab1; cry35Ab1; pat |
| Maize | 32316 | DP-032316-8 | T7; T3 | cry1F; cry34Ab1; cry35Ab1; pat |
| Maize | 4114 | DP-004114-3 | T7; T3 | cry1F; cry34Ab1; cry35Ab1; pat |
| Melon | Melon A | — | T22 | sam-k |
| Melon | Melon B | — | T22 | sam-k |
| Papaya | 55-1 | CUH-CP551-8 | T6 | prsv cp |
| Papaya | 63-1 | CUH-CP631-7 | T6 | prsv cp |
| Papaya | Huanong No. 1 | — | T6 | prsv rep |
| Papaya | X17-2 | UFL-X17CP-6 | T6 | prsv cp |
| Petunia | Petunia-CHS | — | T25 | CHS suppres.sion |
| Plum | C-5 | ARS-PLMC5-6 | T6 | ppv cp |
| Canola** | ZSR500 | — | T1 | cp4 epsps (aroA:CP4); goxv247 |
| Canola** | ZSR502 | — | T1 | cp4 epsps (aroA:CP4); goxv247 |
| Canola** | ZSR503 | — | T1 | cp4 epsps (aroA:CP4); goxv247 |
| Poplar | Bt poplar | — | T7 | cry1Ac; API |
| Poplar | Hybrid poplar clone | — | T7 | cry1Ac; API |
| | 741 | | | |
| Poplar | trg300-1 | — | T24 | AaXEG2 |
| Poplar | trg300-2 | — | T24 | AaXEG2 |
| Potato | 1210 amk | — | T7 | cry3A |
| Potato | 2904/1 kgs | — | T7 | cry3A |
| Canola** | ZSR500 | — | T1 | cp4 epsps (aroA:CP4); goxv247 |
| Canola** | ZSR502 | — | T1 | cp4 epsps (aroA:CP4); goxv247 |
| Potato | ATBT04-27 | NMK-89367-8 | T7 | cry3A |
| Potato | ATBT04-30 | NMK-89613-2 | T7 | cry3A |
| Potato | ATBT04-31 | NMK-89170-9 | T7 | cry3A |
| Potato | ATBT04-36 | NMK-89279-1 | T7 | cry3A |
| Potato | ATBT04-6 | NMK-89761-6 | T7 | cry3A |
| Potato | BT06 | NMK-89812-3 | T7 | cry3A |
| Potato | BT10 | NMK-89175-5 | T7 | cry3A |
| Potato | BT12 | NMK-89601-8 | T7 | cry3A |
| Potato | BT16 | NMK-89167-6 | T7 | cry3A |
| Potato | BT17 | NMK-89593-9 | T7 | cry3A |
| Potato | BT18 | NMK-89906-7 | T7 | cry3A |
| Potato | BT23 | NMK-89675-1 | T7 | cry3A |
| Potato | EH92-527-1 | BPS-25271-9 | T25 | gbss (antisense) |
| Potato | HLMT15-15 | — | T7; T6 | cry3A; pvy cp |
| Potato | HLMT15-3 | — | T7; T6 | cry3A; pvy cp |
| Potato | HLMT15-46 | — | T7; T6 | cry3A; pvy cp |
| Potato | RBMT15-101 | NMK-89653-6 | T7; T6 | cry3A; pvy cp |
| Potato | RBMT21-129 | NMK-89684-1 | T7; T6 | cry3A; plrv orf1; plrv orf2 |
| Potato | RBMT21-152 | — | T7; T6 | cry3A; plrv orf1; plrv orf2 |
| Potato | RBMT21-350 | NMK-89185-6 | T7; T6 | cry3A; plrv orf1; plrv orf2 |
| Potato | RBMT22-082 | NMK-89896-6 | T7; T6.; T1 | cry3A; plrv orf1; plrv orf2; cp4 |
| | | | | epsps (aroA:CP4) |
| Potato | RBMT22-186 | — | T7; T6.; T1 | cry3A; plrv orf1; plrv orf2; cp4 |
| | | | | epsps (aroA:CP4) |
| Potato | RBMT22-238 | — | T7; T6.; T1 | cry3A; plrv orf1; plrv orf2; cp4 |
| | | | | epsps (aroA:CP4) |
| Potato | RBMT22-262 | — | T7; T6.; T1 | cry3A; plrv orf1; plrv orf2; cp4 |
| | | | | epsps (aroA:CP4) |
| Potato | SEMT15-02 | NMK-89935-9 | T7; T6 | cry3A; pvy cp |
| Potato | SEMT15-07 | — | T7; T6 | cry3A; pvy cp |
| Potato | SEMT15-15 | NMK-89930-4 | T7; T6 | cry3A; pvy cp |
| Potato | SPBT02-5 | NMK-89576-1 | T7 | cry3A |
| Potato | SPBT02-7 | NMK-89724-5 | T7 | cry3A |
| Rice | 7Crp#242-95-7 | — | T13 | 7crp |
| Rice | 7Crp#10 | — | T13 | 7crp |
| Rice | GM Shanyou 63 | — | T7 | cry1Ab; cry1Ac |
| Rice | Huahui-1/TT51-1 | — | T7 | cry1Ab; cry1Ac |
| Rice | LLRICE06 | ACS-OS001-4 | T3 | bar |
| Rice | LLRICE601 | BCS-OS003-7 | T3 | bar |
| Rice | LLRICE62 | ACS-OS002-5 | T3 | bar |
| Rice | Tarom molaii + | — | T7 | cry1Ab (truncated) |
| | cry1Ab | | | |
| Rice | GAT-OS2 | — | T3 | bar |
| Rice | GAT-OS3 | — | T3 | bar |
| Rice | PE-7 | — | T7 | cry1Ac |
| Rice | 7Crp#10 | — | T13 | 7crp |
| Rice | KPD627-8 | — | T27 | OASA1D |
| Rice | KPD722-4 | — | T27 | OASA1D |
| Rice | KA317 | — | T27 | OASA1D |
| Rice | HW5 | — | T27 | OASA1D |
| Rice | HW1 | — | T27 | OASA1D |
| Rice | B-4-1-18 | — | T28 | Δ OsBRI1 |
| Rice | G-3-3-22 | — | T29 | OSGA2ox1 |
| Rice | AD77 | — | T6 | DEF |
| Rice | AD51 | — | T6 | DEF |
| Rice | AD48 | — | T6 | DEF |
| Rice | AD41 | — | T6 | DEF |
| Rice | 13p-s-atAprt1 | — | T30 | Hv-S1; Hv-AT-A; APRT |
| Rice | 13pAprt1 | — | T30 | APRT |
| Rice | gHv-S1-gHv-AT-1 | — | T30 | Hv-S1; Hv-AT-A; Hv-AT-B |
| Rice | gHvIDS3-1 | — | T30 | HvIDS3 |
| Rice | gHv-AT1 | — | T30 | Hv-AT-A; Hv-AT-B |
| Rice | gHv-S1-1 | — | T30 | Hv-S1 |
| Rice | NIA-OS006-4 | — | T6 | WRKY45 |
| Rice | NIA-OS005-3 | — | T6 | WRKY45 |
| Rice | NIA-OS004-2 | — | T6 | WRKY45 |
| Rice | NIA-OS003-1 | — | T6 | WRKY45 |
| Rice | NIA-OS002-9 | — | T6 | WRKY45 |
| Rice | NIA-OS001-8 | — | T6 | WRKY45 |
| Rice | OsCr11 | — | T13 | Modified Cry j |
| Rice | 17053 | — | T1 | cp4 epsps (aroA:CP4) |
| Rice | 17314 | — | T1 | cp4 epsps (aroA:CP4) |
| Rose | WKS82/130-4-1 | IFD-52401-4 | T9 | 5AT; bp40 (f3′5′h) |
| Rose | WKS92/130-9-1 | IFD-52901-9 | T9 | 5AT; bp40 (f3′5′h) |
| Soybean | 260-05 (G94-1, | — | T9 | gm-fad2-1 (silencing locus) |
| | G94-19, G168) | | | |
| Soybean | A2704-12 | ACS-GM005-3 | T3 | pat |
| Soybean | A2704-21 | ACS-GM004-2 | T3 | pat |
| Soybean | A5547-127 | ACS-GM006-4 | T3 | pat |
| Soybean | A5547-35 | ACS-GM008-6 | T3 | pat |
| Soybean | CV127 | BPS-CV127-9 | T16 | csr1-2 |
| Soybean | DAS68416-4 | DAS68416-4 | T3 | pat |
| Soybean | DP305423 | DP-305423-1 | T31; T11 | gm-fad2-1 (silencing locus); gm-hra |
| Soybean | DP356043 | DP-356043-5 | T31; T1 | gm-fad2-1 (silencing locus); gat4601 |
| Soybean | FG72 | MST-FG072-3 | T1; T32 | 2mepsps; hppdPF W336 |
| Soybean | GTS 40-3-2 (40-3- | MON-04032-6 | T1 | cp4 epsps (aroA:CP4) |
| | 2) | | | |
| Soybean | GU262 | ACS-GM003-1 | T3 | pat |
| Soybean | MON87701 | MON-87701-2 | T7 | cry1Ac |
| Soybean | MON87705 | MON-87705-6 | T31; T1 | fatb1-A (sense & antisense); fad2- |
| | | | | 1A (sense & antisense); cp4 epsps |
| | | | | (aroA:CP4) |
| Soybean | MON87708 | MON-87708-9 | T12; T1 | dmo; cp4 epsps (aroA:CP4) |
| Soybean | MON87769 | MON-87769-7 | T31; T1 | Pj.D6D; Nc.Fad3; cp4 epsps |
| | | | | (aroA:CP4) |
| Soybean | MON89788 | MON-89788-1 | T1 | cp4 epsps (aroA:CP4) |
| Soybean | W62 | ACS-GM002-9 | T3 | bar |
| Soybean | W98 | ACS-GM001-8 | T3 | bar |
| Soybean | MON87754 | MON-87754-1 | T33 | dgat2A |
| Soybean | DAS21606 | DAS-21606 | T34; T3 | Modified aad-12; pat |
| Soybean | DAS44406 | DAS-44406-6 | T34; T1; T3 | Modified aad-12; 2mepsps; pat |
| Soybean | SYHT04R | SYN-0004R-8 | T35 | Modified avhppd |
| Soybean | 9582.814.19.1 | | T7; T3 | cry1Ac, cry1F, PAT |
| Squash | CZW3 | SEM-ØCZW3-2 | T6 | cmv cp, zymv cp, wmv cp |
| Squash | ZW20 | SEM-0ZW20-7 | T6 | zymv cp, wmv cp |
| Sugar Beet | GTSB77 | SY-GTSB77-8 | T1 | cp4 epsps (aroA:CP4); goxv247 |
| | (T9100152) | | | |
| Sugar Beet | H7-1 | KM-000H71-4 | T1 | cp4 epsps (aroA:CP4) |
| Sugar Beet | T120-7 | ACS-BV001-3 | T3 | pat |
| Sugar Beet | T227-1 | — | T1 | cp4 epsps (aroA:CP4) |
| Sugarcane | NXI-1T | — | T21 | EcbetA |
| Sunflower | X81359 | — | T16 | als |
| Sweet Pepper | PK-SP01 | — | T6 | cmv cp |
| Tobacco | C/F/93/08-02 | — | T5 | bxn |
| Tobacco | Vector 21-41 | — | T36 | NtQPT1 (antisense) |
| Tomato | 1345-4 | — | T22 | acc (truncated) |
| Tomato | 35-1-N | — | T22 | sam-k |
| Tomato | 5345 | — | T7 | cry1Ac |
| Tomato | 8338 | CGN-89322-3 | T22 | accd |
| Tomato | B | SYN-0000B-6 | T22 | pg (sense or antisense) |
| Tomato | Da | SYN-0000DA-9 | T22 | pg (sense or antisense) |
| Sunflower | X81359 | — | T16 | als |
| Tomato | Da Dong No 9 | — | T37 | — |
| Tomato | F (1401F, h38F, | SYN-0000F-1 | T22 | pg (sense or antisense) |
| | 11013F, 7913F) | | | |
| Tomato | FLAVR SAVR ™ | CGN-89564-2 | T22 | pg (sense or antisense) |
| Tomato | Huafan No 1 | — | T22 | anti-efe |
| Tomato | PK-TM8805R | — | T6 | cmv cp |
| | (8805R) | | | |
| Wheat | MON71800 | MON-718ØØ-3 | T1 | cp4 epsps (aroA:CP4) |

*Argentine,
**Polish,
#Eggplant

Treatment of genetically modified plants and seeds with compounds of the invention may result in super-additive or synergistic effects. For example, reduction in application rates, broadening of the activity spectrum, increased tolerance to biotic/abiotic stresses or enhanced storage stability may be greater than expected from just simple additive effects of the application of compounds of the invention on genetically modified plants and seeds.

Compounds of this invention are useful in seed treatments for protecting seeds from plant diseases. In the context of the present disclosure and claims, treating a seed means contacting the seed with a biologically effective amount of a compound of this invention, which is typically formulated as a composition of the invention. This seed treatment protects the seed from soil-borne disease pathogens and generally can also protect roots and other plant parts in contact with the soil of the seedling developing from the germinating seed. The seed treatment may also provide protection of foliage by translocation of the compound of this invention or a second active ingredient within the developing plant. Seed treatments can be applied to all types of seeds, including those from which plants genetically transformed to express specialized traits will germinate. Representative examples include those expressing proteins toxic to invertebrate pests, such as Bacillus thuringiensis toxin or those expressing herbicide resistance such as glyphosate acetyltransferase, which provides resistance to glyphosate. Seed treatments with compounds of this invention can also increase vigor of plants growing from the seed.

Compounds of this invention and their compositions, both alone and in combination with other fungicides, nematicides and insecticides, are particularly useful in seed treatment for crops including, but not limited to, maize or corn, soybeans, cotton, cereal (e.g., wheat, oats, barley, rye and rice), potatoes, vegetables and oilseed rape.

Furthermore, the compounds of this invention are useful in treating postharvest diseases of fruits and vegetables caused by fungi and bacteria. These infections can occur before, during and after harvest. For example, infections can occur before harvest and then remain dormant until some point during ripening (e.g., host begins tissue changes in such a way that infection can progress); also infections can arise from surface wounds created by mechanical or insect injury. In this respect, the compounds of this invention can reduce losses (i.e. losses resulting from quantity and quality) due to postharvest diseases which may occur at any time from harvest to consumption. Treatment of postharvest diseases with compounds of the invention can increase the period of time during which perishable edible plant parts (e.g, fruits, seeds, foliage, stems, bulbs, tubers) can be stored refrigerated or un-refrigerated after harvest, and remain edible and free from noticeable or harmful degradation or contamination by fungi or other microorganisms. Treatment of edible plant parts before or after harvest with compounds of the invention can also decrease the formation of toxic metabolites of fungi or other microorganisms, for example, mycotoxins such as aflatoxins.

Plant disease control is ordinarily accomplished by applying an effective amount of a compound of this invention either pre- or post-infection, to the portion of the plant to be protected such as the roots, stems, foliage, fruits, seeds, tubers or bulbs, or to the media (soil or sand) in which the plants to be protected are growing. The compounds can also be applied to seeds to protect the seeds and seedlings developing from the seeds. The compounds can also be applied through irrigation water to treat plants. Control of postharvest pathogens which infect the produce before harvest is typically accomplished by field application of a compound of this invention, and in cases where infection occurs after harvest the compounds can be applied to the harvested crop as dips, sprays, fumigants, treated wraps and box liners.

Rates of application for these compounds (i.e. a fungicidally effective amount) can be influenced by factors such as the plant diseases to be controlled, the plant species to be protected, ambient moisture and temperature and should be determined under actual use conditions. One skilled in the art can easily determine through simple experimentation the fungicidally effective amount necessary for the desired level of plant disease control. Foliage can normally be protected when treated at a rate of from less than about 1 g/ha to about 5,000 g/ha of active ingredient. Seed and seedlings can normally be protected when seed is treated at a rate of from about 0.001 g (more typically about 0.1 g) to about 10 g per kilogram of seed.

Compounds of this invention can also be mixed with one or more other biologically active compounds or agents including fungicides, insecticides, nematocides, bactericides, acaricides, herbicides, herbicide safeners, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Thus the present invention also pertains to a composition comprising a compound of Formula 1 (in a fungicidally effective amount) and at least one additional biologically active compound or agent (in a biologically effective amount) and can further comprise at least one of a surfactant, a solid diluent or a liquid diluent. The other biologically active compounds or agents can be formulated in compositions comprising at least one of a surfactant, solid or liquid diluent. For mixtures of the present invention, one or more other biologically active compounds or agents can be formulated together with a compound of Formula 1, to form a premix, or one or more other biologically active compounds or agents can be formulated separately from the compound of Formula 1, and the formulations combined together before application (e.g., in a spray tank) or, alternatively, applied in succession.

As mentioned in the Summary of the Invention, one aspect of the present invention is a fungicidal composition comprising (i.e. a mixture or combination of) a compound of Formula 1, an N-oxide, or a salt thereof (i.e. component a), and at least one other fungicide (i.e. component b). Of note is such a combination where the other fungicidal active ingredient has different site of action from the compound of Formula 1. In certain instances, a combination with at least one other fungicidal active ingredient having a similar spectrum of control but a different site of action will be particularly advantageous for resistance management. Thus, a composition of the present invention can further comprise a fungicidally effective amount of at least one additional fungicidal active ingredient having a similar spectrum of control but a different site of action.

Of note is a composition which in addition to the Formula 1 compound of component (a), includes as component (b) at least one fungicidal compound selected from the group consisting of the FRAC-defined mode of action (MOA) classes (A) nucleic acid synthesis, (B) mitosis and cell division, (C) respiration, (D) amino acid and protein synthesis, (E) signal transduction, (F) lipid synthesis and membrane integrity, (G) sterol biosynthesis in membranes, (H) cell wall biosynthesis in membranes, (I) melanin synthesis in cell wall, (P) host plant defense induction, multi-site contact activity and unknown mode of action.

FRAC-recognized or proposed target sites of action along with their FRAC target site codes belonging to the above MOA classes are (Al) RNA polymerase I, (A2) adenosine deaminase, (A3) DNA/RNA synthesis (proposed), (A4) DNA topoisomerase, (B1-B3) β-tubulin assembly in mitosis, (B4) cell division (proposed), (B5) delocalization of spectrin-like proteins, (C1) complex I NADH odxido-reductase, (C2) complex II: succinate dehydrogenase, (C3) complex III: cytochrome bc1 (ubiquinol oxidase) at Qo site, (C4) complex III: cytochrome bc1 (ubiquinone reductase) at Qi site, (C5) uncouplers of oxidative phosphorylation, (C6) inhibitors of oxidative phosphorylation, ATP synthase, (C7) ATP production (proposed), (C8) complex III: cytochrome bc1 (ubiquinone reductase) at Qx (unknown) site, (D1) methionine biosynthesis (proposed), (D2-D5) protein synthesis, (E1) signal transduction (mechanism unknown), (E2-E3) MAP/histidine kinase in osmotic signal transduction, (F2) phospholipid biosynthesis, methyl transferase, (F3) lipid peroxidation (proposed), (F4) cell membrane permeability, fatty acids (proposed), (F6) microbial disrupters of pathogen cell membranes, (F7) cell membrane disruption (proposed), (G1) C14-demethylase in sterol biosynthesis, (G2) Δ14-reductase and 48→Δ7-isomerase in sterol biosynthesis, (G3) 3-keto reductase, C4-demethylation, (G4) squalene epoxidase in sterol biosynthesis, (H3) trehalase and inositol biosynthesis, (H4) chitin synthase, (H5) cellulose synthase, (I1) reductase in melanin biosynthesis and (I2) dehydratase in melanin biosynthesis.

Of particular note is a composition which in addition to the Formula 1 compound of component (a), includes as component (b) at least one fungicidal compound selected from the group consisting of the classes (b1) methyl benzimidazole carbamate (MBC) fungicides; (b2) dicarboximide fungicides; (b3) demethylation inhibitor (DMI) fungicides; (b4) phenylamide fungicides; (b5) amine/morpholine fungicides; (b6) phospholipid biosynthesis inhibitor fungicides; (b7) succinate dehydrogenase inhibitor fungicides; (b8) hydroxy(2-amino-)pyrimidine fungicides; (b9) anilinopyrimidine fungicides; (b10) N-phenyl carbamate fungicides; (b11) quinone outside inhibitor (QoI) fungicides; (b12) phenylpyrrole fungicides; (b13) azanaphthalene fungicides; (b14) lipid peroxidation inhibitor fungicides; (b15) melanin biosynthesis inhibitor-reductase (MBI-R) fungicides; (b16) melanin biosynthesis inhibitor-dehydratase (MBI-D) fungicides; (b17) sterol biosynthesis inhibitor (SBI): Class III fungicides; (b18) squalene-epoxidase inhibitor fungicides; (b19) polyoxin fungicides; (b20) phenylurea fungicides; (b21) quinone inside inhibitor (QiI) fungicides; (b22) benzamide and thiazole carboxamide fungicides; (b23) enopyranuronic acid antibiotic fungicides; (b24) hexopyranosyl antibiotic fungicides; (b25) glucopyranosyl antibiotic: protein synthesis fungicides; (b26) glucopyranosyl antibiotic: trehalase and inositol biosynthesis fungicides; (b27) cyanoacetamideoxime fungicides; (b28) carbamate fungicides; (b29) oxidative phosphorylation uncoupling fungicides; (b30) organo tin fungicides; (b31) carboxylic acid fungicides; (b32) heteroaromatic fungicides; (b33) phosphonate fungicides; (b34) phthalamic acid fungicides; (b35) benzotriazine fungicides; (b36) benzene-sulfonamide fungicides; (b37) pyridazinone fungicides; (b38) thiophene-carboxamide fungicides; (b39) complex I NADH oxidoreductase inhibitor fungicides; (b40) carboxylic acid amide (CAA) fungicides; (b41) tetracycline antibiotic fungicides; (b42) thiocarbamate fungicides; (b43) benzamide fungicides; (b44) microbial fungicides; (b45) Q_{X}I fungicides; (b46) plant extract fungicides; (b47) host plant defense induction fungicides; (b48) multi-site contact activity fungicides; (b49) fungicides other than fungicides of classes (b1) through (b48); and salts of compounds of classes (b1) through (b48).

Further descriptions of these classes of fungicidal compounds are provided below.

(b1) “Methyl benzimidazole carbamate (MBC) fungicides” (FRAC code 1) inhibit mitosis by binding to β-tubulin during microtubule assembly. Inhibition of microtubule assembly can disrupt cell division, transport within the cell and cell structure. Methyl benzimidazole carbamate fungicides include benzimidazole and thiophanate fungicides. The benzimidazoles include benomyl, carbendazim, fuberidazole and thiabendazole. The thiophanates include thiophanate and thiophanate-methyl.

(b2) “Dicarboximide fungicides” (FRAC code 2) inhibit a MAP/histidine kinase in osmotic signal transduction. Examples include chlozolinate, iprodione, procymidone and vinclozolin.

(b3) “Demethylation inhibitor (DMI) fungicides” (FRAC code 3) (Sterol Biosynthesis Inhibitors (SBI): Class I) inhibit C14-demethylase, which plays a role in sterol production. Sterols, such as ergosterol, are needed for membrane structure and function, making them essential for the development of functional cell walls. Therefore, exposure to these fungicides results in abnormal growth and eventually death of sensitive fungi. DMI fungicides are divided between several chemical classes: azoles (including triazoles and imidazoles), pyrimidines, piperazines, pyridines and triazolinthiones. The triazoles include azaconazole, bitertanol, bromuconazole, cyproconazole, difenoconazole, diniconazole (including diniconazole-M), epoxiconazole, etaconazole, fenbuconazole, fluquinconazole, flusilazole, flutriafol, hexaconazole, imibenconazole, ipconazole, metconazole, myclobutanil, penconazole, propiconazole, quinconazole, simeconazole, tebuconazole, tetraconazole, triadimefon, triadimenol, triticonazole, uniconazole, uniconazole-P, α-(1-chlorocyclopropyl)-α-[2-(2,2-dichlorocyclopropyl)ethyl]-1H-1,2,4-triazole-1-ethanol, rel-1-[[(2R,3S)-3-(2-chlorophenyl)-2-(2,4-difluorophenyl)-2-oxiranyl]methyl]-1H-1,2,4-triazole, rel-2-[[(2R,3S)-3-(2-chlorophenyl)-2-(2,4-difluorophenyl)-2-oxiranyl]methyl]-1,2-dihydro-3H-1,2,4-triazole-3-thione, and rel-1-[[(2R,3S)-3-(2-chlorophenyl)-2-(2,4-difluorophenyl)-2-oxiranyl]methyl]-5-(2-propen-1-ylthio)-1H-1,2,4-triazole. The imidazoles include econazole, imazalil, oxpoconazole, prochloraz, pefurazoate and triflumizole. The pyrimidines include fenarimol, nuarimol and triarimol. The piperazines include triforine. The pyridines include buthiobate, pyrifenox, pyrisoxazole (3-[(3R)-5-(4-chlorophenyl)-2,3-dimethyl-3-isoxazolidinyl]pyridine, mixture of 3R,5R- and 3R,5S-isomers) and (αS)43-(4-chloro-2-fluorophenyl)-5-(2,4-difluorophenyl)-4-isoxazolyl]-3-pyridinemethanol. The triazolinthiones include prothioconazole and 2-[2-(1-chlorocyclopropyl)-4-(2,2-dichlorocyclopropyl)-2-hydroxybutyl]-1,2-dihydro-3H-1,2,4-triazole-3-thione. Biochemical investigations have shown that all of the above mentioned fungicides are DMI fungicides as described by K. H. Kuck et al. in Modern Selective Fungicides—Properties, Applications and Mechanisms of Action, H. Lyr (Ed.), Gustav Fischer Verlag: New York, 1995, 205-258.

(b4) “Phenylamide fungicides” (FRAC code 4) are specific inhibitors of RNA polymerase in Oomycete fungi. Sensitive fungi exposed to these fungicides show a reduced capacity to incorporate uridine into rRNA. Growth and development in sensitive fungi is prevented by exposure to this class of fungicide. Phenylamide fungicides include acylalanine, oxazolidinone and butyrolactone fungicides. The acylalanines include benalaxyl, benalaxyl-M (also known as kiralaxyl), furalaxyl, metalaxyl and metalaxyl-M (also known as mefenoxam). The oxazolidinones include oxadixyl. The butyrolactones include ofurace.

(b5) “Amine/morpholine fungicides” (FRAC code 5) (SBI: Class II) inhibit two target sites within the sterol biosynthetic pathway, Δ⁸→Δ⁷ isomerase and Δ¹⁴ reductase. Sterols, such as ergosterol, are needed for membrane structure and function, making them essential for the development of functional cell walls. Therefore, exposure to these fungicides results in abnormal growth and eventually death of sensitive fungi. Amine/morpholine fungicides (also known as non-DMI sterol biosynthesis inhibitors) include morpholine, piperidine and spiroketal-amine fungicides. The morpholines include aldimorph, dodemorph, fenpropimorph, tridemorph and trimorphamide. The piperidines include fenpropidin and piperalin. The spiroketal-amines include spiroxamine.

(b6) “Phospholipid biosynthesis inhibitor fungicides” (FRAC code 6) inhibit growth of fungi by affecting phospholipid biosynthesis. Phospholipid biosynthesis fungicides include phophorothiolate and dithiolane fungicides. The phosphorothiolates include edifenphos, iprobenfos and pyrazophos. The dithiolanes include isoprothiolane.

(b7) “Succinate dehydrogenase inhibitor (SDHI) fungicides” “ (FRAC code 7) inhibit Complex II fungal respiration by disrupting a key enzyme in the Krebs Cycle (TCA cycle) named succinate dehydrogenase. Inhibiting respiration prevents the fungus from making ATP, and thus inhibits growth and reproduction. SDHI fungicides include phenylbenzamide, furan carboxamide, oxathiin carboxamide, thiazole carboxamide, pyrazole-4-carboxamide, pyridine carboxamide-phenyl oxoethyl thiophene amides and pyridinylethyl benzamides The benzamides include benodanil, flutolanil and mepronil. The furan carboxamides include fenfuram. The oxathiin carboxamides include carboxin and oxycarboxin. The thiazole carboxamides include thifluzamide. The pyrazole-4-carboxamides include benzovindiflupyr (N-[9-(dichloromethylene)-1,2,3,4-tetrahydro-1,4-methanonaphthalen-5-yl]-3-(difluoromethyl)-1-methyl-1H-pyrazole-4-carboxamide), bixafen, fluxapyroxad (3-(difluoromethyl)-1-methyl-N-(3′,4′,5′-trifluoro[1,1′-biphenyl]-2-yl)-1H-pyrazole-4-carboxamide), furametpyr, isopyrazam (3-(difluoromethyl)-1-methyl-N-[1,2,3,4-tetrahydro-9-(1-methylethyl)-1,4-methanonaphthalen-5-yl]-1H-pyrazole-4-carboxamide), penflufen (N-[2-(1,3-dimethylbutyl)phenyl]-5-fluoro-1,3-dimethyl-1H-pyrazole-4-carboxamide), penthiopyrad, sedaxane (N-[2-[1,1′-bicyclopropyl]-2-ylphenyl]-3-(difluoromethyl)-1-methyl-1H-pyrazole-4-carboxamide), N-[2-(1S,2R)-[1,1′-bicyclopropyl]-2-ylphenyl]-3-(difluoromethyl)-1-methyl-1H-pyrazole-4-carboxamide, 3-(difluoromethyl)-N-(2,3-dihydro-1,1,3-trimethyl-1H-inden-4-yl)-1-methyl-1H-pyrazol-4-carboxamide, N-[2-(2,4-dichlorophenyl)-2-methoxy-1-methylethyl]-3-(difluoromethyl)-1-methyl-1H-pyrazole-4-carboxamide and N-cyclopropyl-3-(difluoromethyl)-5-fluoro-1-methyl-N-[[2-(1-methyl-ethyl)phenyl]methyl]-1H-pyrazole-4-carboxamide. The pyridine carboxamides include boscalid. The phenyl oxoethyl thiophene amides include isofetamid (N-[1,1-dimethyl-2-[2-methyl-4-(1-methylethoxy)phenyl]-2-oxoethyl]-3-methyl-2-thiophenecarboxamide). The pyridinylethyl benzamides include fluopyram.

(b8) “Hydroxy-(2-amino-)pyrimidine fungicides” (FRAC code 8) inhibit nucleic acid synthesis by interfering with adenosine deaminase. Examples include bupirimate, dimethirimol and ethirimol.

(b9) “Anilinopyrimidine fungicides” (FRAC code 9) are proposed to inhibit biosynthesis of the amino acid methionine and to disrupt the secretion of hydrolytic enzymes that lyse plant cells during infection. Examples include cyprodinil, mepanipyrim and pyrimethanil.

(b10) “N-Phenyl carbamate fungicides” (FRAC code 10) inhibit mitosis by binding to β-tubulin and disrupting microtubule assembly. Inhibition of microtubule assembly can disrupt cell division, transport within the cell and cell structure. Examples include diethofencarb.

(b11) “Quinone outside inhibitor (QoI) fungicides” (FRAC code 11) inhibit Complex III mitochondrial respiration in fungi by affecting ubiquinol oxidase. Oxidation of ubiquinol is blocked at the “quinone outside” (Qₒ) site of the cytochrome bc₁ complex, which is located in the inner mitochondrial membrane of fungi. Inhibiting mitochondrial respiration prevents normal fungal growth and development. Quinone outside inhibitor fungicides include methoxyacrylate, methoxycarbamate, oximinoacetate, oximinoacetamide and dihydrodioxazine fungicides (collectively also known as strobilurin fungicides), and oxazolidinedione, imidazolinone and benzylcarbamate fungicides. The methoxyacrylates include azoxystrobin, coumoxystrobin (methyl(αE)-2-[[(3-butyl-4-methyl-2-oxo-2H-1-benzopyran-7-yl)oxy]methyl]-α-(methoxymethylene)benzeneacetate), enoxastrobin (methyl(αE)-2-[[[(E)-[(2E)-3-(4-chlorophenyl)-1-methyl-2-propen-1-ylidene]amino]oxy]methyl]-α-(methoxymethylene)benzeneaceate) (also known as enestroburin), flufenoxystrobin (methyl(αE)-2-[[2-chloro-4-(trifluoromethyl)phenoxy]methyl]-α-(methoxymethylene)benzeneacetate), picoxystrobin, and pyraoxystrobin (methyl (αE)-2-[[[3-(4-chlorophenyl)-1-methyl-1H-pyrazol-5-yl]oxy]methyl]-α-(methoxymethylene)benzeneacetate). The methoxycarbamates include pyraclostrobin, pyrametostrobin (methyl N-[2-[[(1,4-dimethyl-3-phenyl-1H-pyrazol-5-yl)oxy]methyl]phenyl]-N-methoxycarbamate) and triclopyricarb (methyl N-methoxy-N-[2-[[(3,5,6-trichloro-2-pyridinyl)oxy]methyl]phenyl]carbamate). The oximinoacetates include kresoxim-methyl, and trifloxystrobin. The oximinoacetamides include dimoxystrobin, fenaminstrobin ((αE)-2-[[[(E)-[(2E)-3-(2,6-dichlorophenyl)-1-methyl-2-propen-1-ylidene]amino]oxy]methyl]-α-(methoxyimino)-N-methylbenzeneacetamide), metominostrobin, orysastrobin and α-[methoxyimino]-N-methyl-2-[[[1-[3-(trifluoromethyl)phenyl]ethoxy]imino]methyl]benzeneacetamide. The dihydrodioxazines include fluoxastrobin. The oxazolidinediones include famoxadone. The imidazolinones include fenamidone. The benzylcarbamates include pyribencarb. Class (b11) also includes mandestrobin (2-[(2,5-dimethylphenoxy)methyl]-α-methoxy-N-benzeneacetamide).

(b12) “Phenylpyrrole fungicides” (FRAC code 12) inhibit a MAP/histidine kinase associated with osmotic signal transduction in fungi. Fenpiclonil and fludioxonil are examples of this fungicide class.

(b13) “Azanaphthalene fungicides” (FRAC code 13) are proposed to inhibit signal transduction by a mechanism which is as yet unknown. They have been shown to interfere with germination and/or appressorium formation in fungi that cause powdery mildew diseases. Azanaphthalene fungicides include aryloxyquinolines and quinazolinones. The aryloxyquinolines include quinoxyfen. The quinazolinones include proquinazid.

(b14) “Lipid peroxidation inhibitor fungicides” (FRAC code 14) are proposed to inhibit lipid peroxidation which affects membrane synthesis in fungi. Members of this class, such as etridiazole, may also affect other biological processes such as respiration and melanin biosynthesis. Lipid peroxidation fungicides include aromatic hydrocarbon and 1,2,4-thiadiazole fungicides. The aromatic hydrocarboncarbon fungicides include biphenyl, chloroneb, dicloran, quintozene, tecnazene and tolclofos-methyl. The 1,2,4-thiadiazoles include etridiazole.

(b15) “Melanin biosynthesis inhibitors-reductase (MBI-R) fungicides” (FRAC code 16.1) inhibit the naphthal reduction step in melanin biosynthesis. Melanin is required for host plant infection by some fungi. Melanin biosynthesis inhibitors-reductase fungicides include isobenzofuranone, pyrroloquinolinone and triazolobenzothiazole fungicides. The isobenzofuranones include fthalide. The pyrroloquinolinones include pyroquilon. The triazolobenzothiazoles include tricyclazole.

(b16) “Melanin biosynthesis inhibitors-dehydratase (MBI-D) fungicides” (FRAC code 16.2) inhibit scytalone dehydratase in melanin biosynthesis. Melanin in required for host plant infection by some fungi. Melanin biosynthesis inhibitors-dehydratase fungicides include cyclopropanecarboxamide, carboxamide and propionamide fungicides. The cyclopropanecarboxamides include carpropamid. The carboxamides include diclocymet. The propionamides include fenoxanil.

(b17) “Sterol Biosynthesis Inhibitor (SBI): Class III fungicides (FRAC code 17) inhibit 3-ketoreductase during C4-demethylation in sterol production. SBI: Class III inhibitors include hydroxyanilide fungicides and amino-pyrazolinone fungicides. Hydroxyanilides include fenhexamid. Amino-pyrazolinones include fenpyrazamine (S-2-propen-1-yl 5-amino-2,3 -dihydro-2-(1-methylethyl)-4-(2-methylphenyl)-3-oxo-1H-pyrazole-1-carbothioate).

(b18) “Squalene-epoxidase inhibitor fungicides” (FRAC code 18) (SBI: Class IV) inhibit squalene-epoxidase in the sterol biosynthesis pathway. Sterols such as ergosterol are needed for membrane structure and function, making them essential for the development of functional cell walls. Therefore exposure to these fungicides results in abnormal growth and eventually death of sensitive fungi. Squalene-epoxidase inhibitor fungicides include thiocarbamate and allylamine fungicides. The thiocarbamates include pyributicarb. The allylamines include naftifine and terbinafine.

(b19) “Polyoxin fungicides” (FRAC code 19) inhibit chitin synthase. Examples include polyoxin.

(b20) “Phenylurea fungicides” (FRAC code 20) are proposed to affect cell division. Examples include pencycuron.

(b21) “Quinone inside inhibitor (QiI) fungicides” (FRAC code 21) inhibit Complex III mitochondrial respiration in fungi by affecting ubiquinone reductase. Reduction of ubiquinone is blocked at the “quinone inside” (Qᵢ) site of the cytochrome bc₁ complex, which is located in the inner mitochondrial membrane of fungi. Inhibiting mitochondrial respiration prevents normal fungal growth and development. Quinone inside inhibitor fungicides include cyanoimidazole and sulfamoyltriazole fungicides. The cyanoimidazoles include cyazofamid. The sulfamoyltriazoles include amisulbrom.

(b22) “Benzamide and thiazole carboxamide fungicides” (FRAC code 22) inhibit mitosis by binding to β-tubulin and disrupting microtubule assembly. Inhibition of microtubule assembly can disrupt cell division, transport within the cell and cell structure. The benzamides include zoxamide. The thiazole carboxamides include ethaboxam.

(b23) “Enopyranuronic acid antibiotic fungicides” (FRAC code 23) inhibit growth of fungi by affecting protein biosynthesis. Examples include blasticidin-S.

(b24) “Hexopyranosyl antibiotic fungicides” (FRAC code 24) inhibit growth of fungi by affecting protein biosynthesis. Examples include kasugamycin.

(b25) “Glucopyranosyl antibiotic: protein synthesis fungicides” (FRAC code 25) inhibit growth of fungi by affecting protein biosynthesis. Examples include streptomycin.

(b26) “Glucopyranosyl antibiotic: trehalase and inositol biosynthesis fungicides” (FRAC code 26) inhibit trehalase and inositol biosynthesis. Examples include validamycin.

(b27) “Cyanoacetamideoxime fungicides (FRAC code 27) include cymoxanil.

(b28) “Carbamate fungicides” (FRAC code 28) are considered multi-site inhibitors of fungal growth. They are proposed to interfere with the synthesis of fatty acids in cell membranes, which then disrupts cell membrane permeability. Propamacarb, iodocarb, and prothiocarb are examples of this fungicide class.

(b29) “Oxidative phosphorylation uncoupling fungicides” (FRAC code 29) inhibit fungal respiration by uncoupling oxidative phosphorylation. Inhibiting respiration prevents normal fungal growth and development. This class includes 2,6-dinitroanilines such as fluazinam, and dinitrophenyl crotonates such as dinocap, meptyldinocap and binapacryl.

(b30) “Organo tin fungicides” (FRAC code 30) inhibit adenosine triphosphate (ATP) synthase in oxidative phosphorylation pathway. Examples include fentin acetate, fentin chloride and fentin hydroxide.

(b31) “Carboxylic acid fungicides” (FRAC code 31) inhibit growth of fungi by affecting deoxyribonucleic acid (DNA) topoisomerase type II (gyrase). Examples include oxolinic acid.

(b32) “Heteroaromatic fungicides” (Fungicide Resistance Action Committee (FRAC) code 32) are proposed to affect DNA/ribonucleic acid (RNA) synthesis. Heteroaromatic fungicides include isoxazoles and isothiazolones. The isoxazoles include hymexazole and the isothiazolones include octhilinone.

(b33) “Phosphonate fungicides” (FRAC code 33) include phosphorous acid and its various salts, including fosetyl-aluminum.

(b34) “Phthalamic acid fungicides” (FRAC code 34) include teclofthalam.

(b35) “Benzotriazine fungicides” (FRAC code 35) include triazoxide.

(b36) “Benzene-sulfonamide fungicides” (FRAC code 36) include flusulfamide.

(b37) “Pyridazinone fungicides” (FRAC code 37) include diclomezine.

(b38) “Thiophene-carboxamide fungicides” (FRAC code 38) are proposed to affect ATP production. Examples include silthiofam.

(b39) “Complex I NADH oxidoreductase inhibitor fungicides” (FRAC code 39) inhibit electron transport in mitochondria and include pyrimidinamines such as diflumetorim, and pyrazole-5-carboxamides such as tolfenpyrad.

(b40) “Carboxylic acid amide (CAA) fungicides” (FRAC code 40) inhibit cellulose synthase which prevents growth and leads to death of the target fungus. Carboxylic acid amide fungicides include cinnamic acid amide, valinamide and other carbamate, and mandelic acid amide fungicides. The cinnamic acid amides include dimethomorph, flumorph and pyrimorph (3-(2-chloro-4-pyridinyl)-3-[4-(1,1-dimethylethyl)phenyl]-1-(4-morpholinyl)-2-propene-1-one). The valinamide and other carbamates include benthiavalicarb, benthiavalicarb-isopropyl, iprovalicarb, tolprocarb (2,2,2-trifluoroethyl N-[(1S)-2-methyl-1-[[(4-methylbenzoyl)amino]methyl]propyl]carbamate) and valifenalate (methyl N-[(1-methylethoxy)carbonyl]-L-valyl-3-(4-chlorophenyl)-β-alaninate) (also known as valiphenal). The mandelic acid amides include mandipropamid, N-[2-[4-[[3-(4-chlorophenyl)-2-propyn-1-yl]oxy]-3-methoxyphenyl]ethyl]-3-methyl-2-[(methylsulfonyl)-amino]butanamide and N-[2-[4-[[3-(4-chlorophenyl)-2-propyn-1-yl]oxy]-3-methoxyphenyl]-ethyl]-3-methyl-2-[(ethylsulfonyl)amino]butanamide.

(b41) “Tetracycline antibiotic fungicides” (FRAC code 41) inhibit growth of fungi by affecting protein synthesis. Examples include oxytetracycline.

(b42) “Thiocarbamate fungicides” (FRAC code 42) include methasulfocarb.

(b43) “Benzamide fungicides” (FRAC code 43) inhibit growth of fungi by delocalization of spectrin-like proteins. Examples include pyridinylmethyl benzamide fungicides such as fluopicolide (now FRAC code 7, pyridinylethyl benzamides).

(b44) “Microbial fungicides” (FRAC code 44) disrupt fungal pathogen cell membranes. Microbial fungicides include Bacillus species such as Bacillus amyloliquefaciens strains QST 713, FZB24, MB 1600, D747 and the fungicidal lipopeptides which they produce.

(b45) “Q_{X}I fungicides” (FRAC code 45) inhibit Complex III mitochondrial respiration in fungi by affecting ubiquinone reductase at an unknown (Q_{X}) site of the cytochrome bc₁ complex. Inhibiting mitochondrial respiration prevents normal fungal growth and development. Q_{X}I fungicides include triazolopyrimidylamines such as ametoctradin (5-ethyl-6-octyl [1,2,4]triazolo[1,5-a]pyrimidin-7-amine).

(b46) “Plant extract fungicides” are proposed to act by cell membrane disruption. Plant extract fungicides include terpene hydrocarbons and terpene alcohols such as the extract from Melaleuca alternifolia (tea tree).

(b47) “Host plant defense induction fungicides” (FRAC code P) induce host plant defense mechanisms. Host plant defense induction fungicides include benzothiadiazoles, benzisothiazole and thiadiazole-carboxamide fungicides. The benzothiadiazoles include acibenzolar-S-methyl. The benzisothiazoles include probenazole. The thiadiazole-carboxamides include tiadinil and isotianil.

(b48) “Multi-site contact fungicides” inhibit fungal growth through multiple sites of action and have contact/preventive activity. This class of fungicides includes: (b48.1) “copper fungicides” (FRAC code M1)“, (b48.2) “sulfur fungicides” (FRAC code M2), (b48.3) “dithiocarbamate fungicides” (FRAC code M3), (b48.4) “phthalimide fungicides” (FRAC code M4), (b48.5) “chloronitrile fungicides” (FRAC code M5), (b48.6) “sulfamide fungicides” (FRAC code M6), (b48.7) multi-site contact “guanidine fungicides” (FRAC code M7), (b48.8) “triazine fungicides” (FRAC code M8), (b48.9) “quinone fungicides” (FRAC code M9), (b48.10) “quinoxaline fungicides” (FRAC code M10) and (b48.11) “maleimide fungicides” (FRAC code M11). “Copper fungicides” are inorganic compounds containing copper, typically in the copper(II) oxidation state; examples include copper oxychloride, copper sulfate and copper hydroxide, including compositions such as Bordeaux mixture (tribasic copper sulfate). “Sulfur fungicides” are inorganic chemicals containing rings or chains of sulfur atoms; examples include elemental sulfur. “Dithiocarbamate fungicides” contain a dithiocarbamate molecular moiety; examples include mancozeb, metiram, propineb, ferbam, maneb, thiram, zineb and ziram. “Phthalimide fungicides” contain a phthalimide molecular moiety; examples include folpet, captan and captafol. “Chloronitrile fungicides” contain an aromatic ring substituted with chloro and cyano; examples include chlorothalonil. “Sulfamide fungicides” include dichlofluanid and tolyfluanid. Multi-site contact “guanidine fungicides” include, guazatine, iminoctadine albesilate and iminoctadine triacetate. “Triazine fungicides” include anilazine. “Quinone fungicides” include dithianon. “Quinoxaline fungicides” include quinomethionate (also known as chinomethionate). “Maleimide fungicides” include fluoroimide.

(b49) “Fungicides other than fungicides of classes (b1) through (b48)” include certain fungicides whose mode of action may be unknown. These include: (b49.1), “phenyl-acetamide fungicides” (FRAC code U6), (b49.2) “ aryl-phenyl-ketone fungicides” (FRAC code U8), (b49.3) “guanidine fungicides” (FRAC code U12), (b49.4) “thiazolidine fungicides” (FRAC code U13), (b49.5) “pyrimidinone-hydrazone fungicides” (FRAC code U14) and (b49.6) compounds that bind to oxysterol-binding protein as described in PCT Patent Publication WO 2013/009971. The phenyl-acetamides include cyflufenamid and N-[[(cyclopropylmethoxy)amino][6-(difluoromethoxy)-2,3-difluorophenyl]-methylene]-benzeneacetamide. The aryl-phenyl ketones include benzophenones such as metrafenone, and benzoylpyridines such as pyriofenone (5-chloro-2-methoxy-4-methyl-3-pyridinyl)(2,3,4-trimethoxy-6-methylphenyl)methanone). The quanidines include dodine. The thiazolidines include flutianil ((2Z)-2-[[2-fluoro-5-(trifluoromethyl)phenyl]thio]-2-[3-(2-methoxyphenyl)-2-thiazolidinylidene]acetonitrile). The pyrimidinonehydrazones include ferimzone. The (b49.6) class includes oxathiapiprolin (1-[4-[4-[5-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone) and its R-enantiomer which is 1-[4-[4-[5R-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]-ethanone (Registry Number 1003319-79-6).

The (b49) class also includes bethoxazin, flometoquin (2-ethyl-3,7-dimethyl-6-[4-(trifluoromethoxy)phenoxy]-4-quinolinyl methyl carbonate), fluoroimide, neo-asozin (ferric methanearsonate), picarbutrazox (1,1-dimethylethyl N-[6-[[[[((Z)-1-methyl-1H-tetrazol-5-yl)phenylmethylene]amino]oxy]methyl]-2-pyridinyl]carbamate), pyrrolnitrin, quinomethionate, tebufloquin (6-(1,1-dimethylethyl)-8-fluoro-2,3-dimethyl-4-quinolinyl acetate), tolnifanide (N-(4-chloro-2-nitrophenyl)-N-ethyl-4-methylbenzenesulfonamide), 2-butoxy-6-iodo-3-propyl-4H-1-benzopyran-4-one, 3-butyn-1-yl N-[6-[[[[(1-methyl-1H-tetrazol-5-yl)phenylmethylene]amino]oxy]methyl]-2-pyridinyl]carbamate, (N-(4-chloro-2-nitrophenyl)-N-ethyl-4-methylbenzenesulfonamide), N-[4-[4-chloro-3-(trifluoromethyl)-phenoxy]-2,5-dimethylphenyl]-N-ethyl-N-methylmethanimidamide, N-[[(cyclopropylmethoxy)amino][6-(difluoromethoxy)-2,3-difluorophenyl]methylene]benzeneacetamide, 2,6-dimethyl-1H,5H-[1,4]dithiino[2,3-c:5,6-c′]dipyrrole-1,3,5,7(2H,6H)-tetrone, 5-fluoro-2-[(4-methylphenyl)methoxy]-4-pyrimidinamine, 5-fluoro-2-[(4-fluorophenyl)methoxy]-4-pyrimidinamine and 4-fluorophenyl N-[1-[[[1-(4-cyanophenyl)ethyl]sulfonyl]methyl]-propyl]carbamate, pentyl N-[6-[[[[(1-methyl-1H-tetrazol-5-yl)phenyl-methylene]amino]oxy]methyl]-2-pyridinyl]carbamate, pentyl N-[4-[[[[(1-methyl-1H-tetrazol-5-yl)phenylmethylene]amino]oxy]methyl]-2-thiazolyl]carbamate and pentyl N-[6-[[[[(Z)-(1-methyl-1H-tetrazol-5-yl)phenylmethylene]amino]oxy]methyl]-2-pyridinyl]-carbamate. The (b46) class further includes mitosis- and cell division-inhibiting fungicides besides those of the particular classes described above (e.g., (b1), (b10) and (b22)).

Additional “Fungicides other than fungicides of classes (1) through (46)” whose mode of action may be unknown, or may not yet be classified include a fungicidal compound selected from components (b49.7) through (b49.12), as shown below.

Component (b49.7) relates to a compound of Formula b49.7

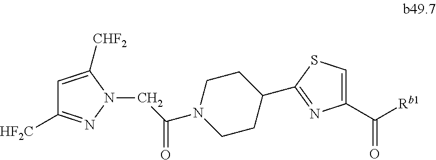

wherein R^{b1} is

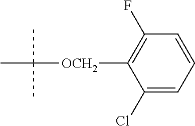

or

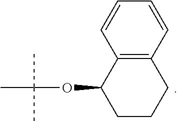

Examples of a compound of Formula b49.7 include (b49.7a) (2-chloro-6-fluorophenyl)-methyl 2-[1-[2-[3,5-bis(difluoromethyl)-1H-pyrazol-1-yl]acetyl]-4-piperidinyl]-4-thiazole-carboxylate (Registry Number 1299409-40-7) and (b49.7b) (1R)-1,2,3,4-tetrahydro-1-naphthalenyl 2-[1-[2-[3,5-bis(difluoromethyl)-1H-pyrazol-1-yl]acetyl]-4-piperidinyl]-4-thiazolecarboxylate (Registry Number 1299409-42-9). Methods for preparing compounds of Formula b46.2 are described in PCT Patent Publications WO 2009/132785 and WO 2011/051243.

Component (b49.8) relates to a compound of Formula b49.8

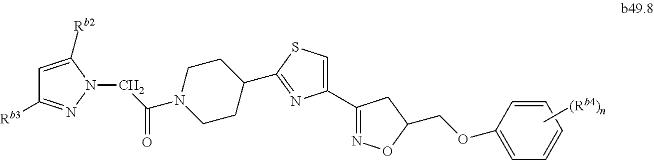

wherein R^{b2} is CH₃, CF₃ or CHF₂; R^{b3} is CH₃, CF₃ or CHF₂; R^{b4} is halogen or cyano; and n is 0, 1, 2 or 3.
   Examples of a compound of Formula b49.8 include (b49.8a) 1-[4-[4-[5-[(2,6-difluorophenoxy)methyl]-4,5-dihydro-3-isoxazolyl]-2-thiazolyl]-1-piperdinyl]-2-[5-methyl-3-(trifluoromethyl)-1H-pyrazol-1-yl]ethanone. Methods for preparing compounds of Formula b49.8 are described in PCT Patent Application PCT/US11/64324.

Component (b4799) relates to a compound of Formula b49.9

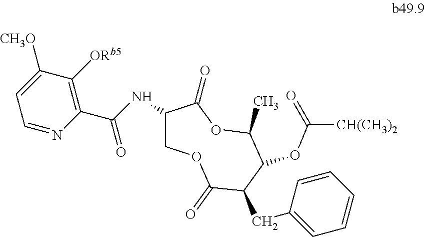

wherein R^{b5} is —CH₂OC(O)CH(CH₃)₂, —C(O)CH₃, —CH₂OC(O)CH₃,

—C(O)OCH₂CH(CH₃)₂ or

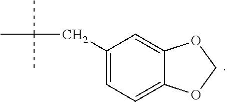

Examples of a compound of Formula b49.9 include (b49.9a) [[4-methoxy-2-[[[(3S,7R,8R,9S)-9-methyl-8-(2-methyl-1-oxopropoxy)-2,6-dioxo-7-(phenylmethyl)-1,5-dioxonan-3-yl]amino]carbonyl]-3-pyridinyl]oxy]methyl 2-methylpropanoate (Registry Number 517875-34-2), (b49.9b) (3S,6S,7R,8R)-3-[[[3-(acetyloxy)-4-methoxy-2-pyridinyl]-carbonyl]amino]-6-methyl-4,9-dioxo-8-(phenylmethyl)-1,5-dioxonan-7-yl 2-methyl-propanoate (Registry Number 234112-93-7), (b49.9c) (3S,6S,7R,8R)-3-[[[3-[(acetyloxy)methoxy]-4-methoxy-2-pyridinyl]carbonyl]amino]-6-methyl-4,9-dioxo-8-(phenylmethyl)-1,5-dioxonan-7-yl 2-methylpropanoate (Registry Number 517875-31-9), (b49. 9d) (3S,6S,7R,8R)-3-[[[4-methoxy-3-[[(2-methylpropoxy)carbonyl]oxy]-2-pyridinyl]-carbonyl]amino]-6-methyl-4,9-dioxo-8-(phenylmethyl)-1,5-dioxonan-7-yl 2-methylpropanoate (Registry Number 328256-72-0), and (b49.9e) N-[[3-(1,3-benzodioxol-5-ylmethoxy)-4-methoxy-2-pyridinyl]carbonyl]-O-[2,5-dideoxy-3-O-(2-methyl-1-oxopropyl)-2-(phenylmethyl)-L-arabinonoyl]-L-serine, (1→4)-lactone (Registry Number 1285706-70-8). Methods for preparing compounds of Formula b49.9 are described in PCT Patent Publications WO 99/40081, WO 2001/014339, WO 2003/035617 and WO 2011044213.

Component (b49.10) relates to a compound of Formula b49.10

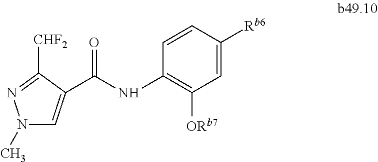

wherein R^{b6} is H or F, and R^{b7} is —CF₂CHFCF₃ or —CF₂CF₂H. Examples of a compound of Formula b49.10 are (b49.10a) 3-(difluoromethyl)-N-[4-fluoro-2-(1,1,2,3,3,3-hexafluoro-propoxy)phenyl]-1-methyl-1H-pyrazole-4-carboxamide (Registry Number 1172611-40-3) and (b49.10b) 3-(difluoromethyl)-1-methyl-N-[2-(1,1,2,2-tetrafluoroethoxy)phenyl]-1H-pyrazole-4-carboxamide (Registry Number 923953-98-4). Compounds of Formula 49.10 can be prepared by methods described in PCT Patent Publication WO 2007/017450.

Component b49.11 relates a compound of Formula b49.11

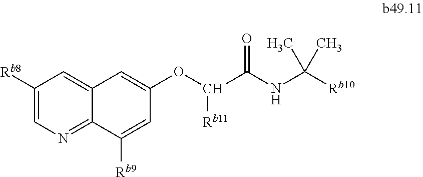

wherein
R^{b8} is halogen, C₁-C₄ alkoxy or C₂-C₄ alkynyl;
R^{b9} is H, halogen or C₁-C₄ alkyl;
R^{b10} is C₁-C₁₂ alkyl, C₁-C₁₂ haloalkyl, C₁-C₁₂ alkoxy, C₂-C₁₂ alkoxyalkyl, C₂-C₁₂ alkenyl, C₂-C₁₂ alkynyl, C₄-C₁₂ alkoxyalkenyl, C₄-C₁₂ alkoxyalkynyl, C₁-C₁₂ alkylthio or C₂-C₁₂ alkylthioalkyl;
R^{b11} is methyl or —Y^{b13}—R^{b12};
R^{b12} is C₁-C₂ alkyl; and
Y^{b13} is CH₂, O or S.
   Examples of compounds of Formula b49.11 include (b49.11a) 2-[(3-bromo-6-quinolinyl)oxy]-N-(1,1-dimethyl-2-butyn-1-yl)-2-(methylthio)acetamide, (b49.11b) 2-[(3-ethynyl-6-quinolinyl)oxy]-N-[1-(hydroxymethyl)-1-methyl-2-propyn-1-yl]-2-(methylthio)-acetamide, (b49.11c) N-(1,1-dimethyl-2-butyn-1-yl)-2-[(3-ethynyl-6-quinolinyl)oxy]-2-(methylthio)acetamide, (b49.11d) 2-[(3-bromo-8-methyl-6-quinolinyl)oxy]-N-(1,1-dimethyl-2-propyn-1-yl)-2-(methylthio)acetamide and (b49.11e) 2-[(3-bromo-6-quinolinyl)oxy]-N-(1,1-dimethylethyl)butanamide. Compounds of Formula b49.11, their use as fungicides and methods of preparation are generally known; see, for example, PCT Patent Publications WO 2004/047538, WO 2004/108663, WO 2006/058699, WO 2006/058700, WO 2008/110355, WO 2009/030469, WO 2009/049716 and WO 2009/087098.

Component 49.12 relates to N′-[4-[[3-[(4-chlorophenyl)methyl]-1,2,4-thiadiazol-5-yl]oxy]-2,5-dimethylphenyl]-N-ethyl-N-methylmethanimidamide, which is believed to inhibit C24-methyl transferase involved in the biosynthesis of sterols.

Therefore of note is a mixture (i.e. composition) comprising a compound of Formula 1 and at least one fungicidal compound selected from the group consisting of the aforedescribed classes (1) through (49). Also of note is a composition comprising said mixture (in fungicidally effective amount) and further comprising at least one additional component selected from the group consisting of surfactants, solid diluents and liquid diluents. Of particular note is a mixture (i.e. composition) comprising a compound of Formula 1 and at least one fungicidal compound selected from the group of specific compounds listed above in connection with classes (1) through (49). Also of particular note is a composition comprising said mixture (in fungicidally effective amount) and further comprising at least one additional surfactant selected from the group consisting of surfactants, solid diluents and liquid diluents.

Examples of component (b) fungicides include acibenzolar-S-methyl, aldimorph, ametoctradin, amisulbrom, anilazine, azaconazole, azoxystrobin, benalaxyl (including benalaxyl-M), benodanil, benomyl, benthiavalicarb (including benthiavalicarb-isopropyl), benzovindiflupyr, bethoxazin, binapacryl, biphenyl, bitertanol, bixafen, blasticidin-S, boscalid, bromuconazole, bupirimate, buthiobate, captafol, captan, carbendazim, carboxin, carpropamid, chloroneb, chlorothalonil, chlozolinate, clotrimazole, copper hydroxide, copper oxychloride, copper sulfate, coumoxystrobin, cyazofamid, cyflufenamid, cymoxanil, cyproconazole, cyprodinil, dichlofluanid, diclocymet, diclomezine, dicloran, diethofencarb, difenoconazole, diflumetorim, dimethirimol, dimethomorph, dimoxystrobin, diniconazole (including diniconazole-M), dinocap, dithianon, dithiolanes, dodemorph, dodine, econazole, edifenphos, enoxastrobin (also known as enestroburin), epoxiconazole, etaconazole, ethaboxam, ethirimol, etridiazole, famoxadone, fenamidone, fenarimol, fenaminstrobin, fenbuconazole, fenfuram, fenhexamid, fenoxanil, fenpiclonil, fenpropidin, fenpropimorph, fenpyrazamine, fentin acetate, fentin chloride, fentin hydroxide, ferbam, ferimzone, flometoquin, fluazinam, fludioxonil, flufenoxystrobin, flumorph, fluopicolide, fluopyram, flouroimide, fluoxastrobin, fluquinconazole, flusilazole, flusulfamide, flutianil, flutolanil, flutriafol, fluxapyroxad, folpet, fthalide, fuberidazole, furalaxyl, furametpyr, guazatine, hexaconazole, hymexazole, imazalil, imibenconazole, iminoctadine albesilate, iminoctadine triacetate, iodocarb, ipconazole, iprobenfos, iprodione, iprovalicarb, isoconazole, isofetamid, isoprothiolane, isopyrazam, isotianil, kasugamycin, kresoxim-methyl, mancozeb, mandepropamid, mandestrobin, maneb, mepanipyrim, mepronil, meptyldinocap, metalaxyl (including metalaxyl-M/mefenoxam), metconazole, methasulfocarb, metiram, metominostrobin, metrafenone, miconazole, myclobutanil, naftifine, neo-asozin, nuarimol, octhilinone, ofurace, orysastrobin, oxadixyl, oxathiapiprolin, oxolinic acid, oxpoconazole, oxycarboxin, oxytetracycline, pefurazoate, penconazole, pencycuron, penflufen, penthiopyrad, phosphorous acid (including salts thereof, e.g., fosetyl-aluminum), picarbutrazox, picoxystrobin, piperalin, polyoxin, probenazole, prochloraz, procymidone, propamacarb, propiconazole, propineb, proquinazid, prothiocarb, prothioconazole, pyraclostrobin, pyrametostrobin, pyraoxystrobin, pyrazophos, pyribencarb, pyributicarb, pyrifenox, pyrimethanil, pyriofenone, pyrisoxazole, pyroquilon, pyrrolnitrin, quinconazole, quinomethionate, quinoxyfen, quintozene, sedaxane, silthiofam, simeconazole, spiroxamine, streptomycin, sulfur, tebuconazole, tebufloquin, teclofthalam, tecnazene, terbinafine, tetraconazole, thiabendazole, thifluzamide, thiophanate, thiophanate-methyl, thiram, tiadinil, tolclofos-methyl, tolnifanide, tolprocarb, tolyfluanid, triadimefon, triadimenol, triarimol, triticonazole, triazoxide, tribasic copper sulfate, tricyclazole, triclopyricarb, tridemorph, trifloxystrobin, triflumizole, triforine, trimorphamide, uniconazole, uniconazole-P, validamycin, valifenalate (also known as valiphenal), vinclozolin, zineb, ziram, zoxamide, (3S,6S,7R,8R)-3-[[[3-[(acetyloxy)methoxy]-4-methoxy-2-pyridinyl]carbonyl]amino]-6-methyl-4,9-dioxo-8-(phenylmethyl)-1,5-dioxonan-7-yl 2-methylpropanoate, (3S,6S,7R,8R)-3-[[[3-(acetyloxy)-4-methoxy-2-pyridinyl]carbonyl]amino]-6-methyl-4,9-dioxo-8-(phenylmethyl)-1,5-dioxonan-7-yl 2-methylpropanoate, N-[[3-(1,3-benzodioxol-5-ylmethoxy)-4-methoxy-2-pyridinyl]carbonyl]-O-[2,5-dideoxy-3-O-(2-methyl-1-oxopropyl)-2-(phenylmethyl)-L-arabinonoyl]-L-serine, (1→4)-lactone, N-[2-(1S,2R)-[1,1′-bicyclopropyl]-2-ylphenyl]-3-(difluoromethyl)-1-methyl-1H-pyrazole-4-carboxamide, 2-[(3-bromo-6-quinolinyl)oxy]-N-(1,1-dimethyl-2-butyn-1-yl)-2-(methylthio)acetamide, 2-[(3-bromo-6-quinolinyl)oxy]-N-(1,1-dimethylethyl)butanamide, 2-[(3-bromo-8-methyl-6-quinolinyl)oxy]-N-(1,1-dimethyl-2-propyn-1-yl)-2-(methylthi o)acetami de, 2-butoxy-6-iodo-3-propyl-4H-1-benzopyran-4-one, 3-butyn-1-yl N-[6-[[[[(1-methyl-1H-tetrazol-5-yl)-phenylmethylene]amino]oxy]methyl]-2-pyridinyl]carbamate, α-(1-chlorocyclopropyl)-α-[2-(2,2-dichlorocyclopropyl)ethyl]-1H-1,2,4-triazole-1-ethanol, 2-[2-(1-chlorocyclopropyl)-4-(2,2-dichlorocyclopropyl)-2-hydroxybutyl]-1,2-dihydro-3H-1,2,4-triazole-3-thione, (αS)-[3-(4-chloro-2-fluorophenyl)-5-(2,4-difluorophenyl)-4-isoxazolyl]-3-pyridinemethanol, rel-1-[[(2R,3S)-3-(2-chlorophenyl)-2-(2,4-difluorophenyl)-2-oxiranyl]methyl]-1H-1,2,4-triazole, rel-2-[[(2R,3S)-3-(2-chlorophenyl)-2-(2,4-difluorophenyl)-2-oxiranyl]methyl]-1,2-dihydro-3H-1,2,4-triazole-3-thione, rel-1-[[(2R,3S)-3-(2-chlorophenyl)-2-(2,4-difluorophenyl)-2-oxiranyl]methyl]-5-(2-propen-1-ylthio)-1H-1,2,4-triazole, 3-[5-(4-chlorophenyl)-2,3-dimethyl-3-isoxazolidinyl]pyridine, (2-chloro-6-fluorophenyl)methyl 2-[1-[2-[3,5-bis(difluoromethyl)-1H-pyrazol-1-yl]acetyl]-4-piperidinyl]-4-thiazolecarboxylate, N′-[4-[[3-[(4-chlorophenyl)methyl]-1,2,4-thiadiazol-5-yl]oxy]-2,5-dimethylphenyl]-N-ethyl-N-methyl-methanimidamide, N-[2-[4-[[3-(4-chlorophenyl)-2-propyn-1-yl]oxy]-3-methoxyphenyl]-ethyl]-3-methyl-2-[(methylsulfonyl)amino]butanamide, N-[2-[4-[[3-(4-chlorophenyl)-2-propyn-1-yl]oxy]-3-methoxyphenyl]ethyl]-3-methyl-2-[(ethylsulfonyl)amino]butanamide, N′-[4-[4-chloro-3-(trifluoromethyl)phenoxy]-2,5-dimethylphenyl]-N-ethyl-N-methyl-methanimidamide, N-cyclopropyl-3-(difluoromethyl)-5-fluoro-1-methyl-N-[[2-(1-methylethyl)phenyl]methyl]-1H-pyrazole-4-carboxamide, N-[[(cyclopropylmethoxy)amino][6-(difluoromethoxy)-2,3-difluorophenyl]methylene]benzeneacetamide, N-[2-(2,4-dichlorophenyl)-2-methoxy-1-methylethyl]-3-(difluoromethyl)-1-methyl-1H-pyrazole-4-carboxamide, N-(3′,4′-difluoro[1,1′-biphenyl]-2-yl)-3-(trifluoromethyl)-2-pyrazinecarboxamide, 3-(difluoromethyl)-N-(2,3-dihydro-1,1,3-trimethyl-1H-inden-4-yl)-1-methyl-1H-pyrazole-4-carboxamide, 3-(difluoromethyl)-N-[4-fluoro-2-(1,1,2,3,3,3 -hexafluoropropoxy)phenyl]-1-methyl-1H-pyrazole-4-carboxamide, 5,8-difluoro-N-[2-[3-methoxy-4-[[4-(trifluoromethyl)-2-pyridinyl]oxy]phenyl]ethyl]-4-quinazolinamine, 3-(difluoromethyl)-1-methyl-N-[2-(1,1,2,2-tetrafluoroethoxy)phenyl]-1H-pyrazole-4-carboxamide, 1-[4-[4-[5R-[(2,6-difluorophenoxy)methyl]-4,5-dihydro-3-isoxazolyl]-2-thiazolyl]-1-piperdinyl]-2-[5-methyl-3-(trifluoromethyl)-1H-pyrazol-1-yl]ethanone, N-(1,1-dimethyl-2-butyn-1-yl)-2-[(3-ethynyl-6-quinolinyl)oxy]-2-(methylthio)acetamide, 2,6-dimethyl-1H,5H-[1,4]dithiino[2,3-c:5,6-c′]dipyrrole-1,3,5,7(2H,6H)-tetrone, 2-[(3-ethynyl-6-quinolinyl)oxy]-N-[1-(hydroxymethyl)-1-methyl-2-propyn-1-yl]-2-(methylthio)acetamide, 4-fluorophenyl N-[1-[[[1-(4-cyanophenyl)ethyl]sulfonyl]methyl]propyl]carbamate, 5-fluoro-2-[(4-fluorophenyl)methoxy]-4-pyrimidinamine, 5-fluoro-2-[(4-methylphenyl)methoxy]-4-pyrimidinamine, (3S,6S,7R,8R)-3-[[[4-methoxy-3-[[(2-methylpropoxy)carbonyl]oxy]-2-pyridinyl]carbonyl]amino]-6-methyl-4,9-dioxo-8-(phenylmethyl)-1,5 -dioxonan-7-yl 2-methylpropanoate, α-(methoxyimino)-N-methyl-2-[[[1-[3-(trifluoromethyl)phenyl]ethoxy]imino]methyl]benzeneacetamide, [[4-methoxy-2-[[[(3S,7R,8R,9S)-9-methyl-8-(2-methyl-1-oxopropoxy)-2,6-dioxo-7-(phenylmethyl)-1,5-dioxonan-3-yl]-amino]carbonyl]-3-pyridinyl]oxy]methyl 2-methylpropanoate, pentyl N-[6-[[[[(1-methyl-1H-tetrazol-5-yl)phenylmethylene]amino]oxy]methyl]-2-pyridinyl]carbamate, pentyl N-[4-[[[[(1-methyl-1H-tetrazol -5-yl)phenylmethylene]amino]oxy]methyl]-2-thiazolyl]carbamate, and pentyl N-[6-[[[[(Z)-(1-methyl-1H-tetrazol-5-yl)phenylmethylene]amino]oxy]methyl]-2-pyridinyl]carbamate and (1R)-1,2,3,4-tetrahydro-1-naphthalenyl 2-[1-[2-[3,5-bis(difluoromethyl)-1H-pyrazol-1-yl]acetyl]-4-piperidinyl]-4-thiazolecarboxylate. Therefore of note is a fungicidal composition comprising as component (a) a compound of Formula 1 (or an N-oxide or salt thereof) and as component (b) at least one fungicide selected from the preceding list.

Of particular note are combinations of compounds of Formula 1 (or an N-oxide or salt thereof) (i.e. Component (a) in compositions) with azoxystrobin, benzovindiflupyr, bixafen, captan, carpropamid, chlorothalonil, copper hydroxide, copper oxychloride, copper sulfate, cymoxanil, cyproconazole, cyprodinil, diethofencarb, difenoconazole, dimethomorph, epoxiconazole, ethaboxam, fenarimol, fenhexamid, fluazinam, fludioxonil, fluopyram, flusilazole, flutianil, flutriafol, fluxapyroxad, folpet, iprodione, isofetamid, isopyrazam, kresoxim-methyl, mancozeb, mandestrobin, meptyldinocap, metalaxyl (including metalaxyl-M/mefenoxam), metconazole, metrafenone, myclobutanil, oxathiapiprolin, penflufen, penthiopyrad, phosphorous acid (including salts thereof, e.g., fosetyl-aluminum), picoxystrobin, propiconazole, proquinazid, prothioconazole, pyraclostrobin, pyrimethanil, sedaxane spiroxamine, sulfur, tebuconazole, thiophanate-methyl, trifloxystrobin, zoxamide, α-(1-chlorocyclopropyl)-α-[2-(2,2-dichlorocyclopropyl)ethyl]-1H-1,2,4-triazole-1-ethanol, 2-[2-(1chlorocyclopropyl)-4-(2,2-dichlorocyclopropyl)-2-hydroxybutyl]-1,2-dihydro-3H-1,2,4-triazole-3-thione, N-[2-(2,4-dichlorophenyl)-2-methoxy-1-methylethyl]-3-(difluoromethyl)-1-methyl-1H-pyrazole-4-carboxami de, 3-(difluoromethyl)-N-(2,3-dihydro-1,1,3-trimethyl-1H-inden-4-yl)-1-methyl-1H-pyrazole-4-carboxamide, 1-[4-[4-[5R-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone, 1,1-dimethylethyl N-[6-[[[[(1-methyl -1H-tetrazol-5-yl)phenylmethylene]amino]oxy]methyl]-2-pyridinyl]carbamate, 2,6-dimethyl-1H,5H-[1,4]dithiino[2,3-c:5,6-c′]dipyrrole-1,3,5,7(2H,6H)-tetrone, 5-fluoro-2-[(4-fluoro-phenyl)methoxy]-4-pyrimidinamine, 5-fluoro-2-[(4-methylphenyl)methoxy]-4-pyrimidinamine, (αS)-[3-(4-chloro-2-fluorophenyl)-5-(2,4-difluorophenyl)-4-isoxazolyl]-3-pyridinemethanol, rel-1-[[(2R,3S)-3-(2-chlorophenyl)-2-(2,4-difluorophenyl)-2-oxiranyl]-methyl]-1H-1,2,4-triazole, rel-2-[[(2R,3S)-3-(2-chlorophenyl)-2-(2,4-difluorophenyl)-2-oxiranyl]methyl]-1,2-dihydro-3H-1,2,4-triazole-3-thione, and rel-1-[[(2R,3S)-3-(2-chlorophenyl)-2-(2,4-difluorophenyl)-2-oxiranyl]methyl]-5-(2-propen-1-ylthio)-1H-1,2,4-triazole (i.e. as Component (b) in compositons).
Table A1 also lists specific combinations of a Component (b) compound with Compound 11 as Component (a) illustrative of the mixtures, compositions and methods of the present invention. (Compound numbers refer to compounds in Index Tables A-C). The second column of Table A1 lists the specific Component (b) compound (e.g., “acibenzolar-S-methyl” in the first line). The third, fourth and fifth columns of Table A1 lists ranges of weight ratios for rates at which the Component (a) compound is typically applied to a field-grown crop relative to Component (b). Thus, for example, the first line of Table A1 specifically discloses the combination of Compound 11 with acibenzolar-S-methyl is typically applied in a weight ratio of between 2:1 and 1:180. The remaining lines of Table A1 are to be construed similarly.

TABLE A1

| | | | More | Most |
|---|---|---|---|---|
| | | Typical | Typical | Typical |
| | | Weight | Weight | Weight |
| Component (a) | Component (b) | Ratio | Ratio | Ratio |
| Compound 11 | acibenzolar-S-methyl | 2:1 to 1:180 | 1:1 to 1:60 | 1:1 to 1:18 |
| Compound 11 | aldimorph | 30:1 to 1:3 | 10:1 to 1:1 | 7:1 to 1:1 |
| Compound 11 | ametoctradin | 9:1 to 1:18 | 3:1 to 1:6 | 3:1 to 1:3 |
| Compound 11 | amisulbrom | 6:1 to 1:18 | 2:1 to 1:6 | 1:1 to 1:6 |
| Compound 11 | anilazine | 90:1 to 2:1 | 30:1 to 4:1 | 22:1 to 4:1 |
| Compound 11 | azaconazole | 7:1 to 1:18 | 2:1 to 1:6 | 2:1 to 1:4 |
| Compound 11 | azoxystrobin | 9:1 to 1:12 | 3:1 to 1:4 | 3:1 to 1:3 |
| Compound 11 | benalaxyl | 4:1 to 1:18 | 1:1 to 1:6 | 1:1 to 1:6 |
| Compound 11 | benalaxyl-M | 4:1 to 1:36 | 1:1 to 1:12 | 1:1 to 1:8 |
| Compound 11 | benodanil | 18:1 to 1:6 | 6:1 to 1:2 | 4:1 to 1:2 |
| Compound 11 | benomyl | 45:1 to 1:4 | 15:1 to 1:1 | 11:1 to 1:1 |
| Compound 11 | benthiavalicarb or benthiavalicarb-isopropyl | 2:1 to 1:36 | 1:1 to 1:12 | 1:1 to 1:12 |
| Compound 11 | bethoxazin | 150:1 to 1:36 | 50:1 to 1:12 | 15:1 to 2:1 |
| Compound 11 | binapacryl | 150:1 to 1:36 | 50:1 to 1:12 | 15:1 to 2:1 |
| Compound 11 | biphenyl | 150:1 to 1:36 | 50:1 to 1:12 | 15:1 to 2:1 |
| Compound 11 | bitertanol | 15:1 to 1:5 | 5:1 to 1:2 | 3:1 to 1:2 |
| Compound 11 | bixafen | 12:1 to 1:9 | 4:1 to 1:3 | 2:1 to 1:3 |
| Compound 11 | blasticidin-S | 3:1 to 1:90 | 1:1 to 1:30 | 1:4 to 1:30 |
| Compound 11 | boscalid | 18:1 to 1:6 | 6:1 to 1:2 | 4:1 to 1:2 |
| Compound 11 | bromuconazole | 15:1 to 1:9 | 5:1 to 1:3 | 3:1 to 1:3 |
| Compound 11 | bupirimate | 3:1 to 1:90 | 1:1 to 1:30 | 1:3 to 1:30 |
| Compound 11 | captafol | 90:1 to 1:4 | 30:1 to 1:2 | 15:1 to 2:1 |
| Compound 11 | captan | 90:1 to 1:4 | 30:1 to 1:2 | 15:1 to 2:1 |
| Compound 11 | carbendazim | 45:1 to 1:4 | 15:1 to 1:2 | 11:1 to 2:1 |
| Compound 11 | carboxin | 18:1 to 1:6 | 6:1 to 1:2 | 4:1 to 1:2 |
| Compound 11 | carpropamid | 15:1 to 1:9 | 5:1 to 1:3 | 3:1 to 1:3 |
| Compound 11 | chloroneb | 300:1 to 2:1 | 100:1 to 4:1 | 100:1 to 14:1 |
| Compound 11 | chlorothalonil | 90:1 to 1:4 | 30:1 to 1:2 | 15:1 to 2:1 |
| Compound 11 | chlozolinate | 45:1 to 1:2 | 15:1 to 2:1 | 11:1 to 2:1 |
| Compound 11 | clotrimazole | 15:1 to 1:9 | 5:1 to 1:3 | 3:1 to 1:3 |
| Compound 11 | copper salts such as Bordeaux mixture | 450:1 to 1:1 | 150:1 to 4:1 | 45:1 to 5:1 |
| | (tribasic copper sulfate), copper | | | |
| | oxychloride, copper sulfate and copper | | | |
| | hydroxide | | | |
| Compound 11 | cyazofamid | 4:1 to 1:18 | 1:1 to 1:6 | 1:1 to 1:6 |
| Compound 11 | cyflufenamid | 1:1 to 1:90 | 1:2 to 1:30 | 1:2 to 1:24 |
| Compound 11 | cymoxanil | 6:1 to 1:18 | 2:1 to 1:6 | 1:1 to 1:5 |
| Compound 11 | cyproconazole | 4:1 to 1:18 | 1:1 to 1:6 | 1:1 to 1:6 |
| Compound 11 | cyprodinil | 22:1 to 1:9 | 7:1 to 1:3 | 4:1 to 1:2 |
| Compound 11 | dichlofluanid | 150:1 to 1:36 | 50:1 to 1:12 | 15:1 to 2:1 |
| Compound 11 | diclocymet | 150:1 to 1:36 | 50:1 to 1:12 | 15:1 to 2:1 |
| Compound 11 | diclomezine | 15:1 to 1:9 | 5:1 to 1:3 | 3:1 to 1:3 |
| Compound 11 | dicloran | 150:1 to 1:36 | 50:1 to 1:12 | 15:1 to 2:1 |
| Compound 11 | diethofencarb | 22:1 to 1:9 | 7:1 to 1:3 | 7:1 to 1:2 |
| Compound 11 | difenoconazole | 4:1 to 1:36 | 1:1 to 1:12 | 1:1 to 1:12 |
| Compound 11 | diflumetorim | 150:1 to 1:36 | 50:1 to 1:12 | 15:1 to 2:1 |
| Compound 11 | dimethirimol | 3:1 to 1:90 | 1:1 to 1:30 | 1:3 to 1:30 |
| Compound 11 | dimethomorph | 9:1 to 1:6 | 3:1 to 1:2 | 3:1 to 1:2 |
| Compound 11 | dimoxystrobin | 9:1 to 1:18 | 3:1 to 1:6 | 2:1 to 1:4 |
| Compound 11 | diniconazole | 3:1 to 1:36 | 1:1 to 1:12 | 1:1 to 1:8 |
| Compound 11 | diniconazole M | 3:1 to 1:90 | 1:1 to 1:30 | 1:1 to 1:12 |
| Compound 11 | dinocap | 7:1 to 1:9 | 2:1 to 1:3 | 2:1 to 1:3 |
| Compound 11 | dithianon | 15:1 to 1:4 | 5:1 to 1:2 | 5:1 to 1:2 |
| Compound 11 | dodemorph | 30:1 to 1:3 | 10:1 to 1:1 | 7:1 to 1:1 |
| Compound 11 | dodine | 30:1 to 1:2 | 10:1 to 2:1 | 10:1 to 2:1 |
| Compound 11 | edifenphos | 30:1 to 1:9 | 10:1 to 1:3 | 3:1 to 1:3 |
| Compound 11 | enoxastrobin | 9:1 to 1:18 | 3:1 to 1:6 | 2:1 to 1:4 |
| Compound 11 | epoxiconazole | 3:1 to 1:36 | 1:1 to 1:12 | 1:1 to 1:7 |
| Compound 11 | etaconazole | 3:1 to 1:36 | 1:1 to 1:12 | 1:1 to 1:7 |
| Compound 11 | ethaboxam | 7:1 to 1:9 | 2:1 to 1:3 | 2:1 to 1:3 |
| Compound 11 | ethirimol | 30:1 to 1:3 | 10:1 to 1:1 | 7:1 to 1:1 |
| Compound 11 | etridiazole | 30:1 to 1:9 | 10:1 to 1:3 | 7:1 to 1:2 |
| Compound 11 | famoxadone | 9:1 to 1:18 | 3:1 to 1:6 | 2:1 to 1:4 |
| Compound 11 | fenamidone | 6:1 to 1:18 | 2:1 to 1:6 | 2:1 to 1:4 |
| Compound 11 | fenarimol | 3:1 to 1:90 | 1:1 to 1:30 | 1:2 to 1:24 |
| Compound 11 | fenbuconazole | 3:1 to 1:30 | 1:1 to 1:10 | 1:1 to 1:10 |
| Compound 11 | fenfuram | 18:1 to 1:6 | 6:1 to 1:2 | 4:1 to 1:2 |
| Compound 11 | fenhexamid | 30:1 to 1:2 | 10:1 to 2:1 | 10:1 to 2:1 |
| Compound 11 | fenoxanil | 150:1 to 1:36 | 50:1 to 1:12 | 15:1 to 1:1 |
| Compound 11 | fenpiclonil | 75:1 to 1:9 | 25:1 to 1:3 | 15:1 to 2:1 |
| Compound 11 | fenpropidin | 30:1 to 1:3 | 10:1 to 1:1 | 7:1 to 1:1 |
| Compound 11 | fenpropimorph | 30:1 to 1:3 | 10:1 to 1:1 | 7:1 to 1:1 |
| Compound 11 | fenpyrazamine | 100:1 to 1:100 | 10:1 to 1:10 | 3:1 to 1:3 |
| Compound 11 | fentin salt such as the acetate, chloride or | 15:1 to 1:9 | 5:1 to 1:3 | 3:1 to 1:3 |
| | hydroxide | | | |
| Compound 11 | ferbam | 300:1 to 1:2 | 100:1 to 2:1 | 30:1 to 4:1 |
| Compound 11 | ferimzone | 30:1 to 1:5 | 10:1 to 1:2 | 7:1 to 1:2 |
| Compound 11 | fluazinam | 22:1 to 1:5 | 7:1 to 1:2 | 3:1 to 1:2 |
| Compound 11 | fludioxonil | 7:1 to 1:12 | 2:1 to 1:4 | 2:1 to 1:4 |
| Compound 11 | flumetover | 9:1 to 1:6 | 3:1 to 1:2 | 3:1 to 1:2 |
| Compound 11 | flumorph | 9:1 to 1:18 | 3:1 to 1:6 | 3:1 to 1:3 |
| Compound 11 | fluopicolide | 3:1 to 1:18 | 1:1 to 1:6 | 1:1 to 1:6 |
| Compound 11 | fluopyram | 15:1 to 1:9 | 5:1 to 1:3 | 3:1 to 1:3 |
| Compound 11 | fluoromide | 150:1 to 2:1 | 50:1 to 4:1 | 37:1 to 5:1 |
| Compound 11 | fluoxastrobin | 4:1 to 1:18 | 1:1 to 1:6 | 1:1 to 1:6 |
| Compound 11 | fluquinconazole | 4:1 to 1:12 | 1:1 to 1:4 | 1:1 to 1:4 |
| Compound 11 | flusilazole | 15:1 to 1:9 | 5:1 to 1:3 | 3:1 to 1:3 |
| Compound 11 | flusulfamide | 90:1 to 1:2 | 30:1 to 2:1 | 15:1 to 2:1 |
| Compound 11 | flutianil | 7:1 to 1:36 | 2:1 to 1:12 | 1:1 to 1:6 |
| Compound 11 | flutolanil | 18:1 to 1:6 | 6:1 to 1:2 | 4:1 to 1:2 |
| Compound 11 | flutriafol | 4:1 to 1:12 | 1:1 to 1:4 | 1:1 to 1:4 |
| Compound 11 | fluxapyroxad | 12:1 to 1:9 | 4:1 to 1:3 | 2:1 to 1:3 |
| Compound 11 | folpet | 90:1 to 1:4 | 30:1 to 1:2 | 15:1 to 2:1 |
| Compound 11 | fosetyl-aluminum | 225:1 to 2:1 | 75:1 to 5:1 | 30:1 to 5:1 |
| Compound 11 | fuberidazole | 45:1 to 1:4 | 15:1 to 1:2 | 11:1 to 2:1 |
| Compound 11 | furalaxyl | 15:1 to 1:45 | 5:1 to 1:15 | 1:1 to 1:6 |
| Compound 11 | furametpyr | 150:1 to 1:36 | 50:1 to 1:12 | 15:1 to 2:1 |
| Compound 11 | guazatine or iminoctadine | 150:1 to 1:36 | 50:1 to 1:12 | 15:1 to 2:1 |
| Compound 11 | hexaconazole | 15:1 to 1:36 | 5:1 to 1:12 | 1:1 to 1:5 |
| Compound 11 | hymexazol | 225:1 to 2:1 | 75:1 to 4:1 | 75:1 to 9:1 |
| Compound 11 | imazalil | 7:1 to 1:18 | 2:1 to 1:6 | 1:1 to 1:5 |
| Compound 11 | imibenconazole | 15:1 to 1:36 | 5:1 to 1:12 | 1:1 to 1:5 |
| Compound 11 | iodocarb | 150:1 to 1:36 | 50:1 to 1:12 | 15:1 to 2:1 |
| Compound 11 | ipconazole | 15:1 to 1:36 | 5:1 to 1:12 | 1:1 to 1:5 |
| Compound 11 | iprobenfos | 150:1 to 1:36 | 50:1 to 1:12 | 15:1 to 2:1 |
| Compound 11 | iprodione | 120:1 to 1:2 | 40:1 to 2:1 | 15:1 to 2:1 |
| Compound 11 | iprovalicarb | 9:1 to 1:9 | 3:1 to 1:3 | 2:1 to 1:3 |
| Compound 11 | isoprothiolane | 150:1 to 2:1 | 50:1 to 4:1 | 45:1 to 5:1 |
| Compound 11 | isopyrazam | 12:1 to 1:9 | 4:1 to 1:3 | 2:1 to 1:3 |
| Compound 11 | isotianil | 12:1 to 1:9 | 4:1 to 1:3 | 2:1 to 1:3 |
| Compound 11 | kasugamycin | 7:1 to 1:90 | 2:1 to 1:30 | 1:2 to 1:24 |
| Compound 11 | kresoxim-methyl | 7:1 to 1:18 | 2:1 to 1:6 | 2:1 to 1:4 |
| Compound 11 | mancozeb | 180:1 to 1:3 | 60:1 to 2:1 | 22:1 to 3:1 |
| Compound 11 | mandipropamid | 6:1 to 1:18 | 2:1 to 1:6 | 2:1 to 1:4 |
| Compound 11 | maneb | 180:1 to 1:3 | 60:1 to 2:1 | 22:1 to 3:1 |
| Compound 11 | mepanipyrim | 18:1 to 1:3 | 6:1 to 1:1 | 6:1 to 1:1 |
| Compound 11 | mepronil | 7:1 to 1:36 | 2:1 to 1:12 | 1:1 to 1:6 |
| Compound 11 | meptyldinocap | 7:1 to 1:9 | 2:1 to 1:3 | 2:1 to 1:3 |
| Compound 11 | metalaxyl | 15:1 to 1:45 | 5:1 to 1:15 | 1:1 to 1:6 |
| Compound 11 | metalaxyl-M | 7:1 to 1:90 | 2:1 to 1:30 | 1:1 to 1:12 |
| Compound 11 | metconazole | 3:1 to 1:18 | 1:1 to 1:6 | 1:1 to 1:6 |
| Compound 11 | methasulfocarb | 150:1 to 1:36 | 50:1 to 1:12 | 15:1 to 1:1 |
| Compound 11 | metiram | 150:1 to 1:36 | 50:1 to 1:12 | 15:1 to 1:1 |
| Compound 11 | metominostrobin | 9:1 to 1:12 | 3:1 to 1:4 | 3:1 to 1:3 |
| Compound 11 | metrafenone | 6:1 to 1:12 | 2:1 to 1:4 | 2:1 to 1:4 |
| Compound 11 | myclobutanil | 5:1 to 1:26 | 1:1 to 1:9 | 1:1 to 1:8 |
| Compound 11 | naftifine | 150:1 to 1:36 | 50:1 to 1:12 | 15:1 to 2:1 |
| Compound 11 | neo-asozin (ferric methanearsonate) | 150:1 to 1:36 | 50:1 to 1:12 | 15:1 to 2:1 |
| Compound 11 | nuarimol | 15:1 to 1:9 | 5:1 to 1:3 | 3:1 to 1:3 |
| Compound 11 | octhilinone | 150:1 to 1:36 | 50:1 to 1:12 | 15:1 to 1:1 |
| Compound 11 | ofurace | 15:1 to 1:45 | 5:1 to 1:15 | 1:1 to 1:6 |
| Compound 11 | orysastrobin | 9:1 to 1:12 | 3:1 to 1:4 | 3:1 to 1:3 |
| Compound 11 | oxadixyl | 15:1 to 1:45 | 5:1 to 1:15 | 1:1 to 1:6 |
| Compound 11 | oxolinic acid | 30:1 to 1:9 | 10:1 to 1:3 | 7:1 to 1:2 |
| Compound 11 | oxpoconazole | 15:1 to 1:36 | 5:1 to 1:12 | 1:1 to 1:5 |
| Compound 11 | oxycarboxin | 18:1 to 1:6 | 6:1 to 1:2 | 4:1 to 1:2 |
| Compound 11 | oxytetracycline | 15:1 to 1:9 | 5:1 to 1:3 | 3:1 to 1:3 |
| Compound 11 | pefurazoate | 150:1 to 1:36 | 50:1 to 1:12 | 15:1 to 2:1 |
| Compound 11 | penconazole | 1:1 to 1:45 | 1:2 to 1:15 | 1:2 to 1:15 |
| Compound 11 | pencycuron | 150:1 to 1:2 | 50:1 to 2:1 | 11:1 to 2:1 |
| Compound 11 | penflufen | 12:1 to 1:9 | 4:1 to 1:3 | 2:1 to 1:3 |
| Compound 11 | penthiopyrad | 12:1 to 1:9 | 4:1 to 1:3 | 2:1 to 1:3 |
| Compound 11 | phosphorous acid and salts thereof | 150:1 to 1:36 | 50:1 to 1:12 | 15:1 to 2:1 |
| Compound 11 | phthalide | 150:1 to 1:36 | 50:1 to 1:12 | 15:1 to 2:1 |
| Compound 11 | picoxystrobin | 7:1 to 1:18 | 2:1 to 1:6 | 1:1 to 1:5 |
| Compound 11 | piperalin | 15:1 to 1:9 | 5:1 to 1:3 | 3:1 to 1:3 |
| Compound 11 | polyoxin | 15:1 to 1:9 | 5:1 to 1:3 | 3:1 to 1:3 |
| Compound 11 | probenazole | 15:1 to 1:9 | 5:1 to 1:3 | 3:1 to 1:3 |
| Compound 11 | prochloraz | 22:1 to 1:4 | 7:1 to 1:1 | 7:1 to 1:2 |
| Compound 11 | procymidone | 45:1 to 1:3 | 15:1 to 1:1 | 11:1 to 2:1 |
| Compound 11 | propamocarb or propamocarb- | 30:1 to 1:2 | 10:1 to 2:1 | 10:1 to 2:1 |
| | hydrochloride | | | |
| Compound 11 | propiconazole | 4:1 to 1:18 | 1:1 to 1:6 | 1:1 to 1:5 |
| Compound 11 | propineb | 45:1 to 1:2 | 15:1 to 2:1 | 11:1 to 2:1 |
| Compound 11 | proquinazid | 3:1 to 1:36 | 1:1 to 1:12 | 1:1 to 1:12 |
| Compound 11 | prothiocarb | 9:1 to 1:18 | 3:1 to 1:6 | 3:1 to 1:3 |
| Compound 11 | prothioconazole | 6:1 to 1:18 | 2:1 to 1:6 | 1:1 to 1:5 |
| Compound 11 | pyraclostrobin | 9:1 to 1:18 | 3:1 to 1:6 | 2:1 to 1:4 |
| Compound 11 | pyrametostrobin | 9:1 to 1:18 | 3:1 to 1:6 | 2:1 to 1:4 |
| Compound 11 | pyraoxystrobin | 9:1 to 1:18 | 3:1 to 1:6 | 2:1 to 1:4 |
| Compound 11 | pyrazophos | 150:1 to 1:36 | 50:1 to 1:12 | 15:1 to 1:1 |
| Compound 11 | pyribencarb | 15:1 to 1:6 | 5:1 to 1:2 | 4:1 to 1:2 |
| Compound 11 | pyrifenox | 15:1 to 1:9 | 5:1 to 1:3 | 3:1 to 1:3 |
| Compound 11 | pyrimethanil | 30:1 to 1:6 | 10:1 to 1:2 | 3:1 to 1:2 |
| Compound 11 | pyriofenone | 6:1 to 1:12 | 2:1 to 1:4 | 2:1 to 1:4 |
| Compound 11 | pyroquilon | 15:1 to 1:9 | 5:1 to 1:3 | 3:1 to 1:3 |
| Compound 11 | pyrrolnitrin | 150:1 to 1:36 | 50:1 to 1:12 | 15:1 to 2:1 |
| Compound 11 | quinconazole | 4:1 to 1:12 | 1:1 to 1:4 | 1:1 to 1:4 |
| Compound 11 | quinomethionate | 150:1 to 1:36 | 50:1 to 1:12 | 15:1 to 2:1 |
| Compound 11 | quinoxyfen | 4:1 to 1:18 | 1:1 to 1:6 | 1:1 to 1:6 |
| Compound 11 | quintozene | 150:1 to 1:36 | 50:1 to 1:12 | 15:1 to 2:1 |
| Compound 11 | silthiofam | 7:1 to 1:18 | 2:1 to 1:6 | 2:1 to 1:4 |
| Compound 11 | simeconazole | 15:1 to 1:36 | 5:1 to 1:12 | 1:1 to 1:5 |
| Compound 11 | spiroxamine | 22:1 to 1:4 | 7:1 to 1:2 | 5:1 to 1:2 |
| Compound 11 | streptomycin | 15:1 to 1:9 | 5:1 to 1:3 | 3:1 to 1:3 |
| Compound 11 | sulfur | 300:1 to 3:1 | 100:1 to 9:1 | 75:1 to 9:1 |
| Compound 11 | tebuconazole | 7:1 to 1:18 | 2:1 to 1:6 | 1:1 to 1:5 |
| Compound 11 | tebufloquin | 100:1 to 1:100 | 10:1 to 1:10 | 3:1 to 1:3 |
| Compound 11 | tecloftalam | 150:1 to 1:36 | 50:1 to 1:12 | 15:1 to 2:1 |
| Compound 11 | tecnazene | 150:1 to 1:36 | 50:1 to 1:12 | 15:1 to 2:1 |
| Compound 11 | terbinafine | 150:1 to 1:36 | 50:1 to 1:12 | 15:1 to 2:1 |
| Compound 11 | tetraconazole | 15:1 to 1:36 | 5:1 to 1:12 | 1:1 to 1:5 |
| Compound 11 | thiabendazole | 45:1 to 1:4 | 15:1 to 1:2 | 11:1 to 2:1 |
| Compound 11 | thifluzamide | 15:1 to 1:9 | 5:1 to 1:3 | 3:1 to 1:3 |
| Compound 11 | thiophanate | 45:1 to 1:3 | 15:1 to 2:1 | 11:1 to 2:1 |
| Compound 11 | thiophanate-methyl | 45:1 to 1:3 | 15:1 to 2:1 | 11:1 to 2:1 |
| Compound 11 | thiram | 150:1 to 1:2 | 50:1 to 2:1 | 37:1 to 5:1 |
| Compound 11 | tiadinil | 12:1 to 1:9 | 4:1 to 1:3 | 2:1 to 1:3 |
| Compound 11 | tolclofos-methyl | 150:1 to 1:2 | 50:1 to 2:1 | 37:1 to 5:1 |
| Compound 11 | tolylfluanid | 150:1 to 1:36 | 50:1 to 1:12 | 15:1 to 2:1 |
| Compound 11 | triadimefon | 15:1 to 1:36 | 5:1 to 1:12 | 1:1 to 1:5 |
| Compound 11 | triadimenol | 15:1 to 1:36 | 5:1 to 1:12 | 1:1 to 1:5 |
| Compound 11 | triarimol | 3:1 to 1:90 | 1:1 to 1:30 | 1:2 to 1:24 |
| Compound 11 | triazoxide | 150:1 to 1:36 | 50:1 to 1:12 | 15:1 to 2:1 |
| Compound 11 | tricyclazole | 15:1 to 1:9 | 5:1 to 1:3 | 3:1 to 1:3 |
| Compound 11 | tridemorph | 30:1 to 1:3 | 10:1 to 1:1 | 7:1 to 1:1 |
| Compound 11 | trifloxystrobin | 6:1 to 1:18 | 2:1 to 1:6 | 2:1 to 1:4 |
| Compound 11 | triflumizole | 15:1 to 1:9 | 5:1 to 1:3 | 3:1 to 1:3 |
| Compound 11 | triforine | 15:1 to 1:9 | 5:1 to 1:3 | 3:1 to 1:3 |
| Compound 11 | trimorphamide | 45:1 to 1:9 | 15:1 to 1:3 | 7:1 to 1:2 |
| Compound 11 | triticonazole | 15:1 to 1:36 | 5:1 to 1:12 | 1:1 to 1:5 |
| Compound 11 | uniconazole | 15:1 to 1:36 | 5:1 to 1:12 | 1:1 to 1:5 |
| Compound 11 | validamycin | 150:1 to 1:36 | 50:1 to 1:12 | 3:1 to 1:3 |
| Compound 11 | valifenalate | 6:1 to 1:18 | 2:1 to 1:6 | 2:1 to 1:4 |
| Compound 11 | vinclozolin | 120:1 to 1:2 | 40:1 to 2:1 | 15:1 to 2:1 |
| Compound 11 | zineb | 150:1 to 1:2 | 50:1 to 2:1 | 37:1 to 5:1 |
| Compound 11 | ziram | 150:1 to 1:2 | 50:1 to 2:1 | 37:1 to 5:1 |
| Compound 11 | zoxamide | 6:1 to 1:18 | 2:1 to 1:6 | 2:1 to 1:4 |
| Compound 11 | N-[2-[4-[[3-(4-chlorophenyl)-2-propyn- | 6:1 to 1:18 | 2:1 to 1:6 | 2:1 to 1:4 |
| | 1-yl]oxy]-3-methoxyphenyl]ethyl]- | | | |
| | 3-methyl-2- | | | |
| | [(methylsulfonyl)amino]butanamide | | | |
| Compound 11 | N-[2-[4-[[3-(4-chlorophenyl)-2-propyn-1- | 6:1 to 1:18 | 2:1 to 1:6 | 2:1 to 1:4 |
| | yl]oxy]-3-methoxyphenyl]ethyl]-3-methyl- | | | |
| | 2-[(ethylsulfonyl)amino]butanamide | | | |
| Compound 11 | 2-butoxy-6-iodo-3-propyl-4H-1- | 3:1 to 1:36 | 1:1 to 1:12 | 1:1 to 1:12 |
| | benzopyran-4-one | | | |
| Compound 11 | 3-[5-(4-chlorophenyl)-2,3-dimethyl-3- | 15:1 to 1:9 | 5:1 to 1:3 | 3:1 to 1:3 |
| | isoxazolidinyl]pyridine | | | |
| Compound 11 | N′-[4-[[3-[(4-chlorophenyl)methyl]-1,2,4- | 20:1 to 1:20 | 8:1 to 1:8 | 3:1 to 1:3 |
| | thiadiazol-5-yl]oxy]-2,5-dimethylphenyl]- | | | |
| | N-ethyl-N-methylmethanimidamide | | | |
| Compound 11 | 4-fluorophenyl N-[1-[[[1-(4-cyanophenyl)- | 6:1 to 1:18 | 2:1 to 1:6 | 2:1 to 1:4 |
| | ethyl]sulfonyl]methyl]propyl]carbamate | | | |
| Compound 11 | N-[[(cyclopropylmethoxy)amino][6- | 1:1 to 1:90 | 1:2 to 1:30 | 1:2 to 1:24 |
| | (difluoromethoxy)-2,3-difluorophenyl]- | | | |
| | methylene]benzeneacetamide | | | |
| Compound 11 | α-[methoxyimino]-N-methyl-2-[[[1-[3- | 9:1 to 1:18 | 3:1 to 1:6 | 3:1 to 1:3 |
| | (trifluoromethyl)phenyl]ethoxy]imino]- | | | |
| | methyl]benzeneacetamide | | | |
| Compound 11 | N′-[4-[4-chloro-3-(trifluoromethyl)- | 15:1 to 1:18 | 5:1 to 1:6 | 3:1 to 1:3 |
| | phenoxy]-2,5-dimethylphenyl]-N-ethyl- | | | |
| | N-methylmethanimidamide | | | |
| Compound 11 | N-(4-chloro-2-nitrophenyl)-N-ethyl- | 15:1 to 1:18 | 5:1 to 1:6 | 3:1 to 1:3 |
| | 4-methylbenzenesulfonamide | | | |
| Compound 11 | fenaminstrobin (2-[[[3-(2,6- | 9:1 to 1:18 | 3:1 to 1:6 | 3:1 to 1:3 |
| | dichlorophenyl)-1-methyl-2-propen-1- | | | |
| | ylidene]amino]oxy]methyl]- | | | |
| | α-(methoxyimino)- | | | |
| | N-methylbenzeneacetamide) | | | |
| Compound 11 | pentyl N-[4-[[[[(1-methyl-1H-tetrazol-5-yl)- | 9:1 to 1:18 | 3:1 to 1:6 | 3:1 to 1:3 |
| | phenylmethylene]amino]oxy]methyl]-2- | | | |
| | thiazolyl]carbamate | | | |
| Compound 11 | 2-[(3-bromo-6-quinolinyl)oxy]- | 5:1 to 1:22 | 2:1 to 1:8 | 2:1 to 1:4 |
| | N-(1,1-dimethyl-2-butyn-1-yl)- | | | |
| | 2-(methylthio)acetamide | | | |
| Compound 11 | 2-[(3-ethynyl-6-quinolinyl)oxy]- | 5:1 to 1:22 | 2:1 to 1:8 | 2:1 to 1:4 |
| | N-[1-(hydroxymethyl)-1-methyl-2-propyn- | | | |
| | 1-yl]-2-(methylthio)acetamide | | | |
| Compound 11 | N-(1,1-dimethyl-2-butyn-1-yl)-2-[(3- | 5:1 to 1:22 | 2:1 to 1:8 | 2:1 to 1:4 |
| | ethynyl-6-quinolinyl)oxy]-2- | | | |
| | (methylthio)acetamide | | | |
| Compound 11 | oxathiapiprolin (1-[4-[4-[5-(2,6- | 1:1 to 1:90 | 1:2 to 1:30 | 1:2 to 1:18 |
| | difluorophenyl)-4,5-dihydro-3-isoxazolyl]- | | | |
| | 2-thiazolyl]-1-piperidinyl]-2-[5-methyl-3- | | | |
| | (trifluoromethyl)-1H-pyrazol-1- | | | |
| | yl]ethanone) | | | |
| Compound 11 | 1-[4-[4-[5R-(2,6-difluorophenyl)-4,5- | 1:1 to 1:90 | 1:2 to 1:30 | 1:2 to 1:18 |
| | dihydro-3-isoxazolyl]-2-thiazolyl]- | | | |
| | 1-piperidinyl]-2-[5-methyl-3- | | | |
| | (trifluoromethyl)-1H-pyrazol-1-yl]ethanone | | | |
| Compound 11 | 1-[4-[4-[5-[(2,6-difluorophenoxy)methyl]- | 1:1 to 1:90 | 1:2 to 1:30 | 1:2 to 1:18 |
| | 4,5-dihydro-3-isoxazolyl]-2-thiazolyl]-1- | | | |
| | piperdinyl-2-[5-methyl-3-(trifluoromethyl)- | | | |
| | 1H-pyrazol-1-yl]ethanone | | | |
| Compound 11 | (2-chloro-6-fluorophenyl)methyl 2-[1-[2- | 1:1 to 1:90 | 1:2 to 1:30 | 1:2 to 1:18 |
| | [3,5-bis(difluoromethyl)-1H-pyrazol-1- | | | |
| | yl]acetyl]-4-piperidinyl]-4-thiazole- | | | |
| | carboxylate | | | |
| Compound 11 | (1R)-1,2,3,4-tetrahydro-1-naphthalenyl 2- | 1:1 to 1:90 | 1:2 to 1:30 | 1:2 to 1:18 |
| | [1-[2-[3,5-bis(difluoromethyl)-1H-pyrazol- | | | |
| | 1-yl]acetyl]-4-piperidinyl]- | | | |
| | 4-thiazolecarboxylate | | | |
| Compound 11 | [[4-methoxy-2-[[[(3S,7R,8R,9S)-9-methyl- | 90:1 to 1:4 | 30:1 to 1:2 | 15:1 to 3:1 |
| | 8-(2-methyl-1-oxopropoxy)-2,6-dioxo-7- | | | |
| | (phenylmethyl)-1,5-dioxonan-3-yl]amino]- | | | |
| | carbonyl]-3-pyridinyl]oxy]methyl | | | |
| | 2-methylpropanoate ( | | | |
| Compound 11 | (3S,6S,7R,8R)-3-[[[3-(acetyloxy)-4- | 90:1 to 1:4 | 30:1 to 1:2 | 15:1 to 3:1 |
| | methoxy-2-pyridinyl]carbonyl]amino]- | | | |
| | 6-methyl-4,9-dioxo-8-(phenylmethyl)-1,5- | | | |
| | dioxonan-7-yl 2-methylpropanoate | | | |
| Compound 11 | (3S,6S,7R,8R)-3-[[[3-[(acetyloxy)methoxy]- | 90:1 to 1:4 | 30:1 to 1:2 | 15:1 to 3:1 |
| | 4-methoxy-2-pyridinyl]carbonyl]amino]-6- | | | |
| | methyl-4,9-dioxo-8-(phenylmethyl)-1,5- | | | |
| | dioxonan-7-yl 2-methylpropanoate | | | |
| Compound 11 | (3S,6S,7R,8R)-3-[[[4-methoxy-3-[[(2- | 90:1 to 1:4 | 30:1 to 1:2 | 15:1 to 3:1 |
| | methylpropoxy)carbonyl]oxy]-2-pyridinyl]- | | | |
| | carbonyl]amino]-6-methyl-4,9-dioxo- | | | |
| | 8-(phenylmethyl)-1,5-dioxonan-7-yl | | | |
| | 2-methylpropanoate | | | |
| Compound 11 | N-[[3-(1,3-benzodioxol-5-ylmethoxy)-4- | 90:1 to 1:4 | 30:1 to 1:2 | 15:1 to 3:1 |
| | methoxy-2-pyridinyl]carbonyl]-O-[2,5- | | | |
| | dideoxy-3-O-(2-methyl-1-oxopropyl)-2- | | | |
| | (phenylmethyl)-L-arabinonoyl]-L-serine, | | | |
| | (1→4′)-lactone | | | |
| Compound 11 | 5-fluoro-2-[(4-methylphenyl)methoxy]-4- | 20:1 to 1:20 | 5:1 to 1:5 | 3:1 to 1:3 |
| | pyrimidinamine | | | |
| Compound 11 | 5-fluoro-2-[(4-fluorophenyl)methoxy]-4- | 20:1 to 1:20 | 5:1 to 1:5 | 3:1 to 1:3 |
| | pyrimidinamine | | | |
| Compound 11 | 5,8-difluoro-N-[2-[3-methoxy-4-[[4- | 40:1 to 1:10 | 10:1 to 1:3 | 5:1 to 1:2 |
| | (trifluoromethyl)-2-pyridinyl]oxy]phenyl]- | | | |
| | ethyl]-4-quinazolinamine | | | |
| Compound 11 | pentyl [6-[[[(Z)-[(1-methyl-1H-tetrazol-5- | 40:1 to 1:10 | 10:1 to 1:3 | 5:1 to 1:2 |
| | yl)phenylmethylene]amino]oxy]methyl]-2- | | | |
| | pyridinyl]carbamate | | | |
| Compound 11 | 1,1-dimethylethyl N-[6-[[[(Z)-[(1-methyl- | 40:1 to 1:10 | 10:1 to 1:3 | 5:1 to 1:2 |
| | 1H-tetrazol-5- | | | |
| | yl)phenylmethylene]amino]oxy]methyl]-2- | | | |
| | pyridinyl]carbamate | | | |
| Compound 11 | 3-butyn-1-yl N-[6-[[[(Z)-[(1-methyl-1H- | 40:1 to 1:10 | 10:1 to 1:3 | 5:1 to 1:2 |
| | tetrazol-5-yl)phenylmethylene]amino]oxy]- | | | |
| | methyl]-2-pyridinyl]carbamate | | | |
| Compound 11 | N-(3′,4′-difluoro[1,1′-biphenyl]-2-yl)-3- | 20:1 to 1:20 | 5:1 to 1:5 | 3:1 to 1:3 |
| | (trifluoromethyl)-2-pyrazinecarboxamide | | | |
| Compound 11 | N-[2-(2,4-dichlorophenyl)-2-methoxy-1- | 20:1 to 1:20 | 5:1 to 1:5 | 3:1 to 1:3 |
| | methylethyl]-3-(difluoromethyl)-1-methyl- | | | |
| | 1H-pyrazole-4-carboxamide | | | |
| Compound 11 | 3-(difluoromethyl)-N-[4-fluoro-2- | 20:1 to 1:20 | 5:1 to 1:5 | 3:1 to 1:3 |
| | (1,1,2,3,3,3-hexafluoropropoxy)phenyl]-1- | | | |
| | methyl-1H-pyrazole-4-carboxamide | | | |
| Compound 11 | 3-(difluoromethyl)-1-methyl-N-[2-(1,1,2,2- | 20:1 to 1:20 | 5:1 to 1:5 | 3:1 to 1:3 |
| | tetrafluoroethoxy)phenyl]-1H-pyrazole-4- | | | |
| | carboxamide | | | |
| Compound 11 | isofetamid | 20:1 to 1:20 | 5:1 to 1:5 | 3:1 to 1:3 |
| Compound 11 | tolprocarb | 20:1 to 1:20 | 5:1 to 1:5 | 3:1 to 1:3 |
| Compound 11 | (αR)-2-[(2,5-dimethylphenoxy)methyl]-α- | 20:1 to 1:20 | 5:1 to 1:5 | 3:1 to 1:3 |
| | methoxy-N-methylbenzeneacetamide | | | |
| Compound 11 | 2,6-dimethyl-1H,5H-[1,4]dithiino[2,3-c:5,6- | 1:1 to 1:400 | 1:4 to 1:100 | 1:8 to 1:50 |
| | c′]dipyrrole-1,3,5,7(2H,6H)-tetrone | | | |
| Compound 11 | 1-[[(2S,3R)-3-(2-chlorophenyl)-2-(2,4- | 36:1 to 1:30 | 12:1 to 1:10 | 6:1 to 1:4 |
| | difluorophenyl)-2-oxiranyl]methyl]-1H- | | | |
| | 1,2,4-triazole | | | |
| Compound 11 | 2-[[(2S,3R)-3-(2-chlorophenyl)-2-(2,4- | 36:1 to 1:30 | 12:1 to 1:10 | 6:1 to 1:4 |
| | difluorophenyl)-2-oxiranyl]methyl]-1,2- | | | |
| | dihydro-3H-1,2,4-triazole-3-thione | | | |
| Compound 11 | 1-[[(2S,3R)-3-(2-chlorophenyl)-2-(2,4- | 36:1 to 1:30 | 12:1 to 1:10 | 6:1 to 1:4 |
| | difluorophenyl)-2-oxiranyl]methyl]-5-(2- | | | |
| | propen-1-ylthio)-1H-1,2,4-triazole | | | |
| Compound 11 | α-[3-(4-chloro-2-fluorophenyl)-5-(2,4- | 36:1 to 1:30 | 12:1 to 1:10 | 6:1 to 1:4 |
| | difluorophenyl)-4-isoxazolyl]-3- | | | |
| | pyridinemethanol | | | |
| Compound 11 | (αS)-[3-(4-chloro-2-fluorophenyl)-5-(2,4- | 36:1 to 1:30 | 12:1 to 1:10 | 6:1 to 1:4 |
| | difluorophenyl)-4-isoxazolyl]-3- | | | |
| | pyridinemethanol | | | |
| Compound 11 | (αR)-[3-(4-chloro-2-fluorophenyl)-5-(2,4- | 36:1 to 1:30 | 12:1 to 1:10 | 6:1 to 1:4 |
| | difluorophenyl)-4-isoxazolyl]-3- | | | |
| | pyridinemethanol | | | |
| Compound 11 | 3-[2-[3-(4-chloro-2-fluorophenyl)-5-(2,4- | 36:1 to 1:30 | 12:1 to 1:10 | 6:1 to 1:4 |
| | difluorophenyl)-4-isoxazolyl]-2- | | | |
| | oxiranyl]pyridine | | | |
| Compound 11 | 2-ethyl-3,7-dimethyl-6-[4- | 36:1 to 1:30 | 12:1 to 1:10 | 6:1 to 1:4 |
| | (trifluoromethoxy)phenoxy]-4-quinolinyl | | | |
| | methyl carbonate | | | |

Tables A2 through A29 are each constructed the same as Table A1 above except that entries below the “Component (a)” column heading are replaced with the respective Component (a) Column Entry shown below. Thus, for example, in Table A2 the entries below the “Component (a)” column heading all recite “Compound 13”, and the first line below the column headings in Table A2 specifically discloses combination of Compound 13 with acibenzolar-S-methyl. Tables A3 through A29 are constructed similarly.

| Table Number | Component (a) Column Entry |
|---|---|
| A2 | Compound 13 |
| A3 | Compound 61 |
| A4 | Compound 81 |
| A5 | Compound 123 |
| A6 | Compound 126 |
| A7 | Compound 142 |
| A8 | Compound 146 |
| A9 | Compound 164 |
| A10 | Compound 174 |
| A11 | Compound 182 |

Examples of other biologically active compounds or agents with which compounds of this invention can be formulated are: invertebrate pest control compounds or agents such as abamectin, acephate, acetamiprid, acrinathrin, afi dopyrop en ([(3S,4R,4aR,6S,6aS,12R,12aS,12bS)-3-[(cyclopropylcarbonyl)oxy]-1,3,4,4a,5,6,6a,12,12a,12b-decahydro-6,12-dihydroxy-4,6a,12b-trimethyl-11-oxo-9-(3-pyridinyl)-2H,11H-naphtho[2,1-b]pyrano[3,4-e]pyran-4-yl]methyl cyclopropanecarboxylate), amidoflumet (S-1955), avermectin, azadirachtin, azinphos-methyl, bifenthrin, bifenazate, buprofezin, carbofuran, cartap, chlorantraniliprole, chlorfenapyr, chlorfluazuron, chlorpyrifos, chlorpyrifos-methyl, chromafenozide, clothianidin, cyantraniliprole (3-bromo-1-(3-chloro-2-pyridinyl)-N-[4-cyano-2-methyl-6-[(methylamino)carbonyl]phenyl]-1H-pyrazole-5-carboxamide), cyclaniliprole (3-bromo-N-[2-bromo-4-chloro-6-[[(1-cyclopropylethyl)amino]carbonyl]phenyl]-1-(3-chloro-2-pyridinyl)-1H-pyrazole-5-carboxamide), cycloxaprid ((5S,8R-1-[(6-chloro-3-pyridinyl)methyl]-2,3,5,6,7,8-hexahydro-9-nitro-5,8-epoxy-1H-imidazo[1,2-a]azepine), cyflumetofen, cyfluthrin, beta-cyfluthrin, cyhalothrin, lambda-cyhalothrin, cypermethrin, cyromazine, deltamethrin, diafenthiuron, diazinon, dieldrin, diflubenzuron, dimefluthrin, dimethoate, dinotefuran, diofenolan, emamectin, endosulfan, esfenvalerate, ethiprole, fenothiocarb, fenoxycarb, fenpropathrin, fenvalerate, fipronil, flonicamid, flubendiamide, flucythrinate, flufenoxystrobin (methyl(αE)-2-[[2-chloro-4-(trifluoromethyl)phenoxy]methyl]-α-(methoxymethylene)benzeneacetate), flufensulfone (5-chloro-2-[(3,4,4-trifluoro-3-buten-1-yl)sulfonyl]thiazole), flupiprole (1-[2,6-dichloro-4-(trifluoromethyl)phenyl]-5-[(2-methyl-2-propen-1-yl)amino]-4-[(trifluoromethyl)sulfinyl]-1H-pyrazole-3-carbonitrile), flupyradifurone (4-[[(6-chloro-3-pyridinyl)methyl](2,2-difluoroethyl)amino]-2(5H)-furanone), tau-fluvalinate, flufenerim (UR-50701), flufenoxuron, fonophos, halofenozide, heptafluthrin ([2,3,5,6-tetrafluoro-4-(methoxymethyl)phenyl]methyl 2,2-dimethyl-3-[(1Z)-3,3,3-trifluoro-1-propen-1-yl]cyclopropanecarboxylate), hexaflumuron, hydramethylnon, imidacloprid, indoxacarb, isofenphos, lufenuron, malathion, meperfluthrin ([2,3,5,6-tetrafluoro-4-(methoxymethyl)phenyl]methyl (1R,3S)-3-(2,2-dichloroethenyl)-2,2-dimethylcyclopropanecarboxylate), metaflumizone, metaldehyde, methamidophos, methidathion, methomyl, methoprene, methoxychlor, methoxyfenozide, metofluthrin, milbemycin oxime, momfluorothrin ([2,3,5,6-tetrafluoro-4-(methoxymethyl)phenyl]methyl 3-(2-cyano-1-propen-1-yl)-2,2-dimethylcyclopropanecarboxylate), monocrotophos, nicotine, nitenpyram, nithiazine, novaluron, noviflumuron (XDE-007), oxamyl, pyflubumide (1,3,5-trimethyl-N-(2-methyl-1-oxopropyl)-N-[3-(2-methylpropyl)-4-[2,2,2-trifluoro-1-methoxy-1-(trifluoromethyl)ethyl]phenyl]-1H-pyrazole-4-carboxamide), parathion, parathion-methyl, permethrin, phorate, phosalone, phosmet, phosphamidon, pirimicarb, profenofos, profluthrin, pymetrozine, pyrafluprole, pyrethrin, pyridalyl, pyrifluquinazon, pyriminostrobin (methyl(αE)-2-[[[2-[(2,4-dichlorophenyl)amino]-6-(trifluoromethyl)-4-pyrimidinyl]oxy]methyl]-α-(methoxymethylene)benzeneacetate), pyriprole, pyriproxyfen, rotenone, ryanodine, spinetoram, spinosad, spirodiclofen, spiromesifen (BSN 2060), spirotetramat, sulfoxaflor, sulprofos, tebufenozide, teflubenzuron, tefluthrin, terbufos, tetrachlorvinphos, tetramethylfluthrin, thiacloprid, thiamethoxam, thiodicarb, thiosultap-sodium, tolfenpyrad, tralomethrin, triazamate, trichlorfon and triflumuron; and biological agents including entomopathogenic bacteria, such as Bacillus thuringiensis subsp. aizawai, Bacillus thuringiensis subsp. kurstaki, and the encapsulated delta-endotoxins of Bacillus thuringiensis (e.g., Cellcap, MPV, MPVII); entomopathogenic fungi, such as green muscardine fungus; and entomopathogenic virus including baculovirus, nucleopolyhedro virus (NPV) such as HzNPV, AfNPV; and granulosis virus (GV) such as CpGV.

Compounds of this invention and compositions thereof can be applied to plants genetically transformed to express proteins toxic to invertebrate pests (such as Bacillus thuringiensis delta-endotoxins). The effect of the exogenously applied fungicidal compounds of this invention may be synergistic with the expressed toxin proteins.

General references for agricultural protectants (i.e. insecticides, fungicides, nematocides, acaricides, herbicides and biological agents) include The Pesticide Manual, 13th Edition, C. D. S. Tomlin, Ed., British Crop Protection Council, Farnham, Surrey, U.K., 2003 and The BioPesticide Manual, 2nd Edition, L. G. Copping, Ed., British Crop Protection

Council, Farnham, Surrey, U.K., 2001.

For embodiments where one or more of these various mixing partners are used, the weight ratio of these various mixing partners (in total) to the compound of Formula 1 is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components may expand the spectrum of diseases controlled beyond the spectrum controlled by the compound of Formula 1 alone.

In certain instances, combinations of a compound of this invention with other biologically active (particularly fungicidal) compounds or agents (i.e. active ingredients) can result in a greater-than-additive (i.e. synergistic) effect. Reducing the quantity of active ingredients released in the environment while ensuring effective pest control is always desirable. When synergism of fungicidal active ingredients occurs at application rates giving agronomically satisfactory levels of fungal control, such combinations can be advantageous for reducing crop production cost and decreasing environmental load.

Also in certain instances, combinations of a compound of the invention with other biologically active compounds or agents can result in a less-than-additive (i.e. safening) effect on organisms beneficial to the agronomic environment. For example, a compound of the invention may safen a herbicide on crop plants or protect a beneficial insect species (e.g., insect predators, pollinators such as bees) from an insecticide.

Fungicides of note for formulation with compounds of Formula 1 to provide mixtures useful in seed treatment include but are not limited to amisulbrom, azoxystrobin, boscalid, carbendazim, carboxin, cymoxanil, cyproconazole, difenoconazole, dimethomorph, fluazinam, fludioxonil, flufenoxystrobin, fluquinconazole, fluopicolide, fluoxastrobin, flutriafol, fluxapyroxad, ipconazole, iprodione, metalaxyl, mefenoxam, metconazole, my cl obutanil, paclobutrazole, penflufen, pi coxy strob in, prothioconazole, pyracl o strob in, sedaxane, silthiofam, tebuconazole, thiabendazole, thiophanate-methyl, thiram, trifloxystrobin and triticonazole.

Invertebrate pest control compounds or agents with which compounds of Formula 1 can be formulated to provide mixtures useful in seed treatment include but are not limited to abamectin, acetamiprid, acrinathrin, afidopyropen, amitraz, avermectin, azadirachtin, bensultap, bifenthrin, buprofezin, cadusafos, carbaryl, carbofuran, cartap, chlorantraniliprole, chlorfenapyr, chlorpyrifos, clothianidin, cyantraniliprole, cyclaniliprole, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, dieldrin, dinotefuran, diofenolan, emamectin, endosulfan, esfenvalerate, ethiprole, etofenprox, etoxazole, fenothiocarb, fenoxycarb, fenvalerate, fipronil, flonicamid, flubendiamide, fluensulfone, flufenoxuron, flufiprole, flupyradifurone, fluvalinate, formetanate, fosthiazate, heptafluthrin, hexaflumuron, hydramethylnon, imidacloprid, indoxacarb, lufenuron, meperfluthrin, metaflumizone, methiocarb, methomyl, methoprene, methoxyfenozide, momfluorothrin, nitenpyram, nithiazine, novaluron, oxamyl, pyflubumide, pymetrozine, pyrethrin, pyridaben, pyriminostrobin, pyridalyl, pyriproxyfen, ryanodine, spinetoram, spinosad, spirodiclofen, spiromesifen, spirotetramat, sulfoxaflor, tebufenozide, tetramethrin, tetramethylfluthrin, thiacloprid, thiamethoxam, thiodicarb, thiosultap-sodium, tralomethrin, triazamate, triflumuron, Bacillus thuringiensis delta-endotoxins, strains of Bacillus thuringiensis and strains of Nucleo polyhydrosis viruses.

Compositions comprising compounds of Formula 1 useful for seed treatment can further comprise bacteria and fungi that have the ability to provide protection from the harmful effects of plant pathogenic fungi or bacteria and/or soil born animals such as nematodes. Bacteria exhibiting nematicidal properties may include but are not limited to Bacillus firmus, Bacillus cereus, Bacillius subtiliis and Pasteuria penetrans. A suitable Bacillus firmus strain is strain CNCM 1-1582 (GB-126) which is commercially available as BioNem™. A suitable Bacillus cereus strain is strain NCMM I-1592. Both Bacillus strains are disclosed in U.S. Pat. No. 6,406,690. Other suitable bacteria exhibiting nematicidal activity are B. amyloliquefaciens IN937a and B. subtilis strain GB03. Bacteria exhibiting fungicidal properties may include but are not limited to B. pumilus strain GB34. Fungal species exhibiting nematicidal properties may include but are not limited to Myrothecium verrucaria, Paecilomyces lilacinus and Purpureocillium lilacinum.

Seed treatments can also include one or more nematicidal agents of natural origin such as the elicitor protein called harpin which is isolated from certain bacterial plant pathogens such as Erwinia amylovora. An example is the Harpin-N-Tek seed treatment technology available as N-Hibit™ Gold CST.

Seed treatments can also include one or more species of legume-root nodulating bacteria such as the microsymbiotic nitrogen-fixing bacteria Bradyrhizobium japonicum. These inocculants can optionally include one or more lipo-chitooligosaccharides (LCDs), which are nodulation (Nod) factors produced by rhizobia bacteria during the initiation of nodule formation on the roots of legumes. For example, the Optimize® brand seed treatment technology incorporates LCO Promoter Technology™ in combination with an inocculant.

Seed treatments can also include one or more isoflavones which can increase the level of root colonization by mycorrhizal fungi. Mycorrhizal fungi improve plant growth by enhancing the root uptake of nutrients such as water, sulfates, nitrates, phosphates and metals. Examples of isoflavones include, but are not limited to, genistein, biochanin A, formononetin, daidzein, glycitein, hesperetin, naringenin and pratensein. Formononetin is available as an active ingredient in mycorrhizal inocculant products such as PHC Colonize® AG.

Seed treatments can also include one or more plant activators that induce systemic acquired resistance in plants following contact by a pathogen. An example of a plant activator which induces such protective mechanisms is acibenzolar-S-methyl.

The following Tests demonstrate the control efficacy of compounds of this invention on specific pathogens. The pathogen control protection afforded by the compounds is not limited, however, to these species. See Index Tables A-C for compound descriptions. The following abbreviations are used in the Index Tables which follow: t is tertiary, s is secondary, n is normal, i is iso, c is cyclo, Me is methyl, Et is ethyl, Pr is propyl, i-Pr is isopropyl, Bu is butyl, c-Pr is cyclopropyl, t-Bu is tent-butyl, Ph is phenyl, OMe is methoxy, OEt is ethoxy, SMe is methylthio, SEt is ethylthio, —CN is cyano, —NO₂ is nitro, TMS is trimethylsilyl, t-Amyl is —C(CH₃)₂CH₂CH₃, neopentyl is —CH₂C(CH₃)₃ and allyl is —CH₂CH═CH₂. Additional abbreviations are:

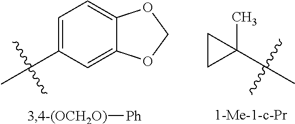

(R) or (S) denotes the absolute chirality of the asymmetric carbon center. The abbreviation “(d)” indicates that the compound appeared to decompose on melting. The bonding of the L group is such that the left bond is connected to the isoxazoline ring and the right bond is connected to R⁵.

INDEX TABLE A

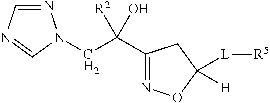

| | | | | | AP+ |
|---|---|---|---|---|---|
| Cmpd | R² | L | R⁵ | Isomer | (M + 1) |
| 1 | Ph | direct bond | (2,6-diF)—Ph | mix | 371 |
| 2 | 4-Cl—Ph | direct bond | 4-(1,2,4-triazol-1-yl)-Ph | mix | 436 |
| 3 | i-Pr | direct bond | 4-Cl—Ph | less polar | 335 |
| 4 | i-Pr | direct bond | 4-Cl—Ph | more polar | 335 |
| 5 | 4-Cl—Ph | direct bond | 4-(4-bromopyrazol-1-yl)-Ph | less polar | 515 |
| 6 | 4-Cl—Ph | direct bond | 4-(4-bromopyrazol-1-yl)-Ph | more polar | 515 |
| 7 | t-Bu | direct bond | 4-(4-bromopyrazol-1-yl)-Ph | less polar | 461 |
| 8 | t-Bu | direct bond | 4-(4-bromopyrazol-1-yl)-Ph | more polar | 461 |
| 9 | t-Bu | —CH₂O— | (t-Bu)(Me)₂Si | less polar | 383 |
| 10 | t-Bu | —CH₂O— | (t-Bu)(Me)₂Si | more polar | 383 |
| 11 | t-Amyl | —CH₂O— | 4-Br—Ph | less polar | 437 |
| 12 | t-Amyl | —CH₂O— | 4-Br—Ph | more polar | 437 |
| 13 | t-Bu | —CH₂CH₂— | 4-(CF₃)—Ph | less polar | 411 |
| 14 | t-Bu | —CH₂CH₂— | 4-(CF₃)—Ph | more polar | 411 |
| 15 | t-Bu | —CH₂CH₂— | 4-Cl—Ph | less polar | 377 |
| 16 | t-Bu | —CH₂CH₂— | 4-Cl—Ph | more polar | 377 |
| 17 | t-Amyl | —CH₂— | 4-Cl—Ph | less polar | 377 |
| 18 | t-Amyl | —CH₂— | 4-Cl—Ph | more polar | 377 |
| 19 | t-Amyl | —CH₂— | 4-(CF₃)—Ph | less polar | 411 |
| 20 | t-Amyl | —CH₂— | 4-(CF₃)—Ph | more polar | 411 |
| 21 | t-Bu | —CH₂O— | (2,4-diF)—Ph | less polar | 381 |
| 22 | t-Bu | —CH₂— | (3,4-diCl)—Ph | less polar | 397 |
| 23 | t-Bu | —CH₂— | (3,4-diCl)—Ph | more polar | 397 |
| 24 | t-Bu | —CH₂O— | 4-(CF₃O)—Ph | less polar | 429 |
| 25 | t-Bu | —CH₂O— | 4-(CF₃O)—Ph | more polar | 429 |
| 26 | t-Bu | —CH₂S— | 4-Cl—Ph | less polar | 395 |
| 27 | t-Bu | —CH₂S— | 4-Cl—Ph | more polar | 395 |
| 28 | t-Bu | —CH₂O— | 4-(pyrazol-1-yl)-Ph | less polar | 411 |
| 29 | t-Bu | —CH₂O— | 4-(pyrazol-1-yl)-Ph | more polar | 411 |
| 30 | t-Bu | —CH₂O— | 4-(4-bromopyrazol-1-yl)-Ph | | 489 |
| 31 | t-Bu | —CH₂O— | 4-(4-chloropyrazol-1-yl)-Ph | | 445 |
| 32 | t-Bu | —CH₂O— | 4-Ph—Ph | less polar | 405 |
| 33 | t-Bu | —CH₂O— | 4-Ph—Ph | more polar | 405 |
| 34 | Et | direct bond | 4-Cl—Ph | mix | 321 |
| 35 | Me | direct bond | 4-Cl—Ph | less polar | 307 |
| 36 | Me | direct bond | 4-Cl—Ph | more polar | 307 |
| 37 | 4-Cl—Ph | —CH₂— | 4-bromopyrazol-1-yl | less polar | 453 |
| 38 | 4-Cl—Ph | —CH₂— | 4-bromopyrazol-1-yl | more polar | 453 |
| 39 | adamantyl | direct bond | 4-Cl—Ph | less polar | 427 |
| 40 | adamantyl | direct bond | 4-Cl—Ph | more polar | 427 |
| 41 | t-Bu | —CH₂— | 4-bromopyrazol-1-yl | less polar | 399 |
| 42 | t-Bu | —CH₂— | 4-bromopyrazol-1-yl | more polar | 399 |
| 43 | 4-Cl—Ph | —CH₂O— | 3,4-(OCH₂O)—Ph | less polar | 443 |
| 44 | 4-Cl—Ph | —CH₂O— | 3,4-(OCH₂O)—Ph | more polar | 443 |
| 45 | t-Bu | —CH₂O— | 3,4-(OCH₂O)—Ph | less polar | 389 |
| 46 | t-Bu | —CH₂O— | 3,4-(OCH₂O)—Ph | more polar | 389 |
| 47 | t-Bu | —CH₂O— | 2-Me-5-(CF₃)-pyrazol-3-yl | less polar | 417 |
| 48 | t-Bu | —CH₂— | 3-(CF₃)-pyrazol-1-yl | less polar | 387 |
| 49 | t-Bu | —CH₂— | 3-(CF₃)-pyrazol-1-yl | more polar | 387 |
| 50 | 4-Cl—Ph | —CH₂S— | Ph | less polar | 415 |
| 51 | 4-Cl—Ph | —CH₂S— | Ph | more polar | 415 |
| 52 | t-Bu | —CH₂— | 4-Br—Ph | less polar | 409 |
| 53 | t-Bu | —CH₂— | 4-Br—Ph | more polar | 409 |
| 54 | 4-Cl—Ph | —CH₂— | 4-Br—Ph | less polar | 463 |
| 55 | 4-Cl—Ph | —CH₂— | 4-Br—Ph | more polar | 463 |
| 56 | t-Bu | —CH₂O— | 2-Me-5-(CF₃)-pyrazol-3-yl | more polar | 417 |
| 57 | 4-Cl—Ph | —CH₂OCH₂— | Ph | less polar | 413 |
| 58 | 4-Cl—Ph | —CH₂OCH₂— | Ph | more polar | 413 |
| 59 | t-Bu | —CH₂OCH₂— | Ph | less polar | 359 |
| 60 | t-Bu | —CH₂OCH₂— | Ph | more polar | 359 |
| 61 | 4-Cl—Ph | —CH₂O— | 4-Cl—Ph | less polar | 433 |
| 62 | 4-Cl—Ph | —CH₂O— | 4-Cl—Ph | more polar | 433 |
| 63 | 4-Cl—Ph | —CH₂O— | 4-(CF₃)—Ph | less polar | 467 |
| 64 | 4-Cl—Ph | —CH₂O— | 4-(CF₃)—Ph | more polar | 467 |
| 65 | 4-Cl—Ph | —CH₂— | 3-(4-chlorophenyl)pyrazol-1-yl | less polar | 483 |
| 66 | 4-Cl—Ph | —CH₂— | 3-(4-chlorophenyl)pyrazol-1-yl | more polar | 483 |
| 67 | 4-Cl—Ph | —CH₂— | 3-[4-(CF₃)phenyl]pyrazol-1-yl | less polar | 517 |
| 68 | 4-Cl—Ph | —CH₂— | 3-[4-(CF₃)phenyl]pyrazol-1-yl | more polar | 517 |
| 69 | 4-Cl—Ph | —CH₂O— | 4-(PhCH₂O)—Ph | less polar | 505 |
| 70 | 4-Cl—Ph | —CH₂O— | 4-(PhCH₂O)—Ph | more polar | 505 |
| 71 | t-Bu | —CH₂O— | 4-(PhCH₂O)—Ph | less polar | 451 |
| 72 | t-Bu | —CH₂O— | 4-(PhCH₂O)—Ph | more polar | 451 |
| 73 | t-Bu | —CH₂S— | Ph | less polar | 361 |
| 74 | t-Bu | —CH₂S— | Ph | more polar | 361 |
| 75 | 4-Cl—Ph | direct bond | neopentyl | less polar | 363 |
| 76 | 4-Cl—Ph | direct bond | neopentyl | more polar | 363 |
| 77 | t-Bu | direct bond | neopentyl | less polar | 309 |
| 78 | t-Bu | direct bond | neopentyl | more polar | 309 |
| 79 | t-Bu | —CH₂O— | 4-F—Ph | less polar | 363 |
| 80 | t-Bu | —CH₂O— | 4-F—Ph | more polar | 363 |
| 81 | t-Bu | —CH₂O— | 4-(CF₃)—Ph | less polar | 413 |
| 82 | n-Pr | direct bond | 4-Cl—Ph | less polar | 335 |
| 83 | n-Pr | direct bond | 4-Cl—Ph | more polar | 335 |
| 84 | allyl | direct bond | 4-Cl—Ph | less polar | 333 |
| 85 | allyl | direct bond | 4-Cl—Ph | more polar | 333 |
| 86 | t-Bu | —CH₂— | Ph | less polar | 329 |
| 87 | t-Bu | —CH₂— | Ph | more polar | 329 |
| 88 | t-Bu | —CH₂O— | 4-(CF₃)—Ph | more polar | 413 |
| 89 | 4-Cl—Ph | —CH₂O— | cyclohexyl | less polar | 405 |
| 90 | 4-Cl—Ph | —CH₂O— | cyclohexyl | more polar | 405 |
| 91 | t-Bu | —CH₂O— | cyclohexyl | less polar | 351 |
| 92 | t-Bu | —CH₂O— | cyclohexyl | more polar | 351 |
| 93 | 4-Cl—Ph—CH₂ | direct bond | 4-Cl—Ph | less polar | 417 |
| 94 | 4-Cl—Ph—CH₂ | direct bond | 4-Cl—Ph | more polar | 417 |
| 95 | t-Bu | —CH₂O— | 5-chloropyridin-2-yl | less polar | 380 |
| 96 | t-Bu | —CH₂O— | 5-chloropyridin-2-yl | more polar | 380 |
| 97 | t-Bu | —CH₂O— | 6-chloropyridin-3-yl | less polar | 380 |
| 98 | t-Bu | —CH₂O— | 6-chloropyridin-3-yl | more polar | 380 |
| 99 | t-Bu | —CH₂O— | 5-chloropyrimidin-2-yl | less polar | 381 |
| 100 | t-Bu | —CH₂O— | 5-chloropyrimidin-2-yl | more polar | 381 |
| 101 | 4-Cl—Ph | —CH₂O— | 6-chloropyridin-3-yl | less polar | 435 |
| 102 | 4-Cl—Ph | —CH₂O— | 6-chloropyridin-3-yl | more polar | 435 |
| 103 | 1-propyn-1-yl | direct bond | 4-Cl—Ph | less polar | 331 |
| 104 | 1-propyn-1-yl | direct bond | 4-Cl—Ph | more polar | 331 |
| 105 | 4-(CF₃)—Ph | direct bond | 4-Cl—Ph | less polar | 437 |
| 106 | 4-(CF₃)—Ph | direct bond | 4-Cl—Ph | more polar | 437 |
| 107 | 4-Cl—Ph | direct bond | 4-(CH₃S)—Ph | less polar | 415 |
| 108 | 4-Cl—Ph | direct bond | 4-(CH₃S)—Ph | more polar | 415 |
| 109 | 3-Ph—O—Ph | direct bond | 4-Cl—Ph | less polar | 461 |
| 110 | 3-Ph—O—Ph | direct bond | 4-Cl—Ph | more polar | 461 |
| 111 | 4-Cl—Ph | direct bond | 4-(CH₃SO)—Ph | less polar | 431 |
| 112 | 4-Cl—Ph | direct bond | 4-(CH₃SO)—Ph | more polar | 431 |
| 113 | 4-Cl—Ph | direct bond | 4-(CH₃SO₂)—Ph | less polar | 447 |
| 114 | 4-Cl—Ph | direct bond | 4-(CH₃SO₂)—Ph | more polar | 447 |
| 115 | Ph | direct bond | Ph | mix | 335 |
| 116 | Ph | direct bond | 4-F—Ph | mix | 353 |
| 117 | Ph | direct bond | 4-Cl—Ph | mix | 369 |
| 118 | Ph | direct bond | 2-F—Ph | mix | 353 |
| 119 | 4-F—Ph | direct bond | 4-F—Ph | more polar | 371 |
| 120 | 4-Cl—Ph | direct bond | 4-Cl—Ph | more polar | 403 |
| 121 | Ph—CH₂ | direct bond | 4-Cl—Ph | less polar | 383 |
| 122 | 4-F—Ph | direct bond | 4-F—Ph | less polar | 371 |
| 123 | 4-Cl—Ph | direct bond | 4-Cl—Ph | less polar | 403 |
| 124 | Ph | direct bond | t-Bu | less polar | 315 |
| 125 | Ph | direct bond | t-Bu | more polar | 315 |
| 126 | 1-Me-1-c-Pr | direct bond | 4-Cl—Ph | less polar | 347 |
| 127 | 1-Me-1-c-Pr | direct bond | 4-Cl—Ph | more polar | 347 |
| 128 | 4-Ph—O—Ph | direct bond | 4-Cl—Ph | less polar | 461 |
| 130 | (2,4-diF)—Ph | direct bond | 4-Cl—Ph | less polar | 405 |
| 131 | (2,4-diF)—Ph | direct bond | 4-Cl—Ph | more polar | 405 |
| 132 | t-Bu | direct bond | 4-Cl—Ph | less polar | 349 |
| 133 | t-Bu | direct bond | 4-Cl—Ph | more polar | 349 |
| 134 | 2-F—Ph | direct bond | 4-Cl—Ph | less polar | 387 |
| 135 | 2-F—Ph | direct bond | 4-Cl—Ph | more polar | 387 |
| 136 | 4-Cl—Ph | —CH₂— | Ph | less polar | 383 |
| 137 | 4-Cl—Ph | —CH₂— | Ph | more polar | 383 |
| 138 | neopentyl | direct bond | 4-Cl—Ph | less polar | 363 |
| 139 | neopentyl | direct bond | 4-Cl—Ph | more polar | 363 |
| 140 | (2,4-diCl)—Ph | direct bond | 4-Cl—Ph | less polar | 437 |
| 141 | (2,4-diCl)—Ph | direct bond | 4-Cl—Ph | more polar | 437 |
| 142 | 4-Cl—Ph | direct bond | 4-Ph—O—Ph | less polar | 461 |
| 143 | 4-Cl—Ph | direct bond | 4-Ph—O—Ph | more polar | 461 |
| 144 | 4-Cl—Ph | —CH₂O— | Ph | less polar | 399 |
| 145 | 4-Cl—Ph | —CH₂O— | Ph | more polar | 399 |
| 146 | 4-Cl—Ph | direct bond | 4-(CF₃)—Ph | less polar | 437 |
| 147 | 4-Cl—Ph | direct bond | 4-(CF₃)—Ph | more polar | 437 |
| 148 | 4-Cl—Ph | direct bond | 4[3-(CF₃)pyrazol-1-yl]phenyl | less polar | 503 |
| 149 | 4-Cl—Ph | direct bond | 4[3-(CF₃)pyrazol-1-yl]phenyl | more polar | 503 |
| 150 | 2-Cl—Ph | direct bond | 4-Cl—Ph | less polar | 403 |
| 151 | 2-Cl—Ph | direct bond | 4-Cl—Ph | more polar | 403 |
| 152 | 4-Ph—O—Ph | direct bond | 4-Cl—Ph | | 461 |
| 153 | 4-(CF₃)—Ph | direct bond | 4-(CF₃)—Ph | less polar | 471 |
| 154 | 4-(CF₃)—Ph | direct bond | 4-(CF₃)—Ph | more polar | 471 |
| 155 | n-Bu | direct bond | 4-Cl—Ph | less polar | 349 |
| 156 | n-Bu | direct bond | 4-Cl—Ph | more polar | 349 |
| 157 | 4-Cl—Ph | direct bond | pyrazin-2-yl | less polar | 371 |
| 158 | 4-Cl—Ph | direct bond | 4-F—Ph | less polar | 387 |
| 159 | 4-Cl—Ph | direct bond | 4-F—Ph | more polar | 387 |
| 160 | 4-F—Ph | direct bond | 4-Cl—Ph | less polar | 387 |
| 161 | 4-F—Ph | direct bond | 4-Cl—Ph | more polar | 387 |
| 162 | 4-Cl—Ph | —CH₂O— | 4-F—Ph | less polar | 417 |
| 163 | 4-Cl—Ph | —CH₂O— | 4-F—Ph | more polar | 417 |
| 164 | 4-Cl—Ph | —CH₂O— | 5-chloropyridin-2-yl | less polar | 434 |
| 165 | 4-Cl—Ph | —CH₂O— | 5-chloropyridin-2-yl | more polar | 434 |
| 166 | 4-Cl—Ph | —CH₂O— | 5-chloropyrimidin-2-yl | less polar | 435 |
| 167 | 4-Cl—Ph | —CH₂O— | 5-chloropyrimidin-2-yl | more polar | 435 |
| 168 | t-Bu | direct bond | pyrazin-2-yl | less polar | 317 |
| 169 | t-Bu | direct bond | pyrazin-2-yl | more polar | 317 |
| 170 | neopentyl | —CH₂O— | 4-Br—Ph | less polar | 437 |
| 171 | neopentyl | —CH₂O— | 4-Br—Ph | more polar | 437 |
| 172 | neopentyl | —CH₂O— | 4-Cl—Ph | less polar | 393 |
| 173 | neopentyl | —CH₂O— | 4-Cl—Ph | more polar | 393 |
| 174 | t-Bu | —CH₂O— | 4-Cl—Ph | less polar | 379 |
| 175 | t-Bu | —CH₂O— | 4-Cl—Ph | more polar | 379 |
| 176 | 4-Cl—Ph | direct bond | 3-Ph—O—Ph | less polar | 461 |
| 177 | 4-Cl—Ph | direct bond | 3-Ph—O—Ph | more polar | 461 |
| 178 | 4-Cl—Ph | direct bond | 4-Br—Ph | less polar | 447 |
| 179 | 4-Cl—Ph | direct bond | 4-Br—Ph | more polar | 447 |
| 180 | 4-Br—Ph | direct bond | 4-Cl—Ph | less polar | 447 |
| 181 | 4-Br—Ph | direct bond | 4-Cl—Ph | more polar | 447 |
| 182 | t-Bu | —CH₂O— | 4-Br—Ph | less polar | 423 |
| 183 | t-Bu | —CH₂O— | 4-Br—Ph | more polar | 423 |
| 184 | t-Bu | —CH₂O— | 4-I—Ph | less polar | 471 |
| 185 | t-Bu | —CH₂O— | 4-I—Ph | more polar | 471 |
| 186 | t-Amyl | —CH₂O— | 4-Cl—Ph | less polar | 393 |
| 187 | t-Bu | —CH₂O— | 4-F—Ph | less polar | 347 |
| 188 | t-Bu | —CH₂O— | 4-Cl—Ph | less polar | 363 |
| 189 | t-Bu | —CH₂O— | 4-(CF₃)—Ph | more polar | 397 |
| 190 | t-Bu | —CH₂O— | 4-F—Ph | more polar | 347 |
| 191 | t-Bu | —CH₂O— | 4-Cl—Ph | more polar | 363 |
| 192 | t-Bu | —CH₂O— | 4-(CF₃)—Ph | less polar | 397 |
| 193 | t-Amyl | —CH₂O— | 4-Cl—Ph | more polar | 393 |
| 194 | 4-Cl—Ph | —CH₂— | 3-(CF₃)-pyrazol-1-yl | less polar | 441 |
| 195 | 4-Cl—Ph | —CH₂— | 3-(CF₃)-pyrazol-1-yl | more polar | 441 |
| 196 | cyclohexyl | direct bond | 4-Cl—Ph | less polar | 375 |
| 197 | cyclohexyl | direct bond | 4-Cl—Ph | more polar | 375 |
| 198 | 4-Cl—Ph | —CH₂— | 4-F—Ph | less polar | 402 |
| 199 | 4-Cl—Ph | —CH₂— | 4-Cl—Ph | less polar | 418 |
| 200 | 4-Cl—Ph | —CH₂— | 4-F—Ph | more polar | 401 |
| 201 | 4-Cl—Ph | —CH₂— | 4-Cl—Ph | more polar | 417 |
| 202 | 4-Cl—Ph | —CH₂— | 4-(CF₃)—Ph | less polar | 452 |
| 203 | 4-Cl—Ph | —CH₂— | 4-(CF₃)—Ph | more polar | 452 |
| 204 | 4-Cl—Ph | —CH₂— | 4-Ph—Ph | less polar | 459 |
| 205 | 4-Cl—Ph | —CH₂— | 4-Ph—Ph | more polar | 459 |
| 206 | neopentyl | —CH₂— | 4-Cl—Ph | less polar | 377 |
| 207 | neopentyl | —CH₂— | 4-Cl—Ph | more polar | 377 |
| 208 | t-Bu | —CH₂CH₂O— | 4-Cl—Ph | less polar | 393 |
| 209 | t-Bu | —CH₂CH₂O— | 4-Cl—Ph | more polar | 393 |
| 224 | 1-Me-1-c-Pr | —CH₂O— | 4-Cl—Ph | less polar | 377 |
| 225 | 1-Me-1-c-Pr | —CH₂O— | 4-Cl—Ph | more polar | 377 |
| 226 | neopentyl | —CH₂— | 4-(CF₃)—Ph | less polar | 411 |
| 227 | neopentyl | —CH₂— | 4-(CF₃)—Ph | more polar | 411 |
| 228 | t-Bu | —CH₂O— | 4-Ph—Ph | less polar | 421 |
| 229 | t-Bu | —CH₂O— | 4-Ph—Ph | more polar | 421 |
| 230 | t-Bu | —CH₂O— | 3-Cl—Ph | less polar | 379 |
| 231 | t-Bu | —CH₂O— | 3-Cl—Ph | more polar | 379 |
| 232 | t-Bu | —CH₂O— | 3-Br—Ph | less polar | 423 |
| 233 | t-Bu | —CH₂O— | 3-Br—Ph | more polar | 423 |
| 234 | t-Bu | —CH₂O— | 4-Me—Ph | less polar | 359 |
| 235 | t-Bu | —CH₂O— | 4-Me—Ph | more polar | 359 |
| 236 | 1-Me-1-c-Pr | —CH₂O— | 4-(CF₃)—Ph | less polar | 411 |
| 237 | 1-Me-1-c-Pr | —CH₂O— | 4-(CF₃)—Ph | more polar | 411 |
| 238 | 1-Me-1-c-Pr | —CH₂O— | 4-Br—Ph | less polar | 421 |
| 239 | 1-Me-1-c-Pr | —CH₂O— | 4-Br—Ph | more polar | 421 |
| 240 | neopentyl | —CH₂O— | 4-(CF₃)—Ph | less polar | 427 |
| 241 | neopentyl | —CH₂O— | 4-(CF₃)—Ph | more polar | 427 |
| 242 | t-Bu | —CH₂O— | 4-Cl—Ph | less polar | 378 |
| 243 | t-Bu | —CH₂O— | 4-Cl—Ph | more polar | 378 |
| 244 | neopentyl | —CH₂O— | 4-F—Ph | less polar | 377 |
| 245 | neopentyl | —CH₂O— | 4-F—Ph | more polar | 377 |
| 249 | t-Amyl | —CH₂O— | 4-F—Ph | less polar | 377 |
| 250 | t-Amyl | —CH₂O— | 4-F—Ph | more polar | 377 |
| 251 | 1-Cl-1-c-Pr | —CH₂O— | 4-Cl—Ph | less polar | 397 |
| 252 | 1-Cl-1-c-Pr | —CH₂O— | 4-Cl—Ph | more polar | 397 |
| 253 | t-Bu | —CH₂O— | 3-(CF₃)—Ph | less polar | 413 |
| 254 | t-Bu | —CH₂O— | 3-(CF3)—Ph | more polar | 413 |
| 255 | t-Bu | —CH₂O— | (2,4-diCl)—Ph | less polar | 413 |
| 256 | t-Bu | —CH₂O— | (2,4-diCl)—Ph | more polar | 413 |
| 257 | t-Bu | —CH₂O— | (3,4-diCl)—Ph | less polar | 413 |
| 258 | t-Bu | —CH₂O— | (3,4-diCl)—Ph | more polar | 413 |
| 259 | t-Bu | —CH₂O— | 4-(OMe)—Ph | less polar | 375 |
| 260 | t-Bu | —CH₂O— | 4-(OMe)—Ph | more polar | 375 |
| 261 | t-Amyl | —CH₂O— | 4-(CF₃)—Ph | less polar | 427 |
| 262 | t-Amyl | —CH₂O— | 4-(CF₃)—Ph | more polar | 427 |
| 263 | t-Bu | —CH₂OCH₂— | 4-Cl—Ph | less polar | 393 |
| 264 | t-Bu | —CH₂OCH₂— | 4-Cl—Ph | more polar | 393 |
| 265 | cyclohexyl | —CH₂O— | 4-Cl—Ph | less polar | 405 |
| 266 | cyclohexyl | —CH₂O— | 4-Cl—Ph | more polar | 405 |
| 267 | i-Bu | —CH₂O— | 4-Cl—Ph | less polar | 379 |
| 268 | i-Bu | —CH₂O— | 4-Cl—Ph | more polar | 379 |
| 269 | t-Bu | —CH₂CH₂O— | 4-F—Ph | less polar | 377 |
| 270 | t-Bu | —CH₂CH₂O— | 4-F—Ph | more polar | 377 |

INDEX TABLE B

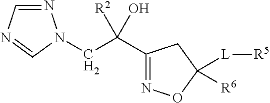

| | | | | | | AP+ |
|---|---|---|---|---|---|---|
| Cmpd | R² | L | R⁵ | R⁶ | Isomer | (M + 1) |
| 210 | Ph | direct bond | 4-Cl—Ph | Me | less polar | 383 |
| 211 | Ph | direct bond | 4-Cl—Ph | Me | more polar | 383 |
| 212 | 4-Cl—Ph | direct bond | 4-Cl—Ph | Me | less polar | 417 |
| 213 | 4-Cl—Ph | direct bond | 4-Cl—Ph | Me | more polar | 417 |
| 214 | 4-Cl—Ph | direct bond | Ph | CF₃ | less polar | 437 |
| 215 | 4-Cl—Ph | direct bond | Ph | CF₃ | more polar | 437 |
| 216 | t-Bu | —CH₂O— | 4-Cl—Ph | Me | less polar | 393 |
| 217 | t-Bu | —CH₂O— | 4-Cl—Ph | Me | more polar | 393 |
| 218 | 4-Cl—Ph | —CH₂O— | 4-Cl—Ph | Me | less polar | 447 |
| 219 | 4-Cl—Ph | —CH₂O— | 4-Cl—Ph | Me | more polar | 447 |

INDEX TABLE C

| | | | AP+ |
|---|---|---|---|
| Cmpd | Structure | Isomer | (M + 1) |
| 220 | 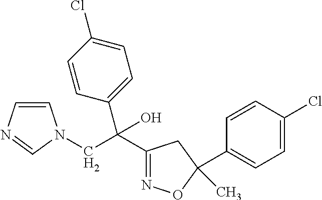 [SMILES: CC1(c2ccc(Cl)cc2)CC(C(O)(Cn2ccnc2)c2ccc(Cl)cc2)=NO1] | less polar | 417 |
| 221 | 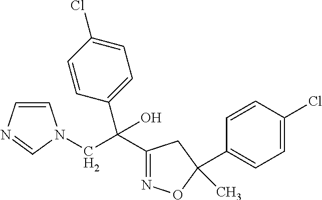 [SMILES: CC1(c2ccc(Cl)cc2)CC(C(O)(Cn2ccnc2)c2ccc(Cl)cc2)=NO1] | more polar | 417 |
| 222 | 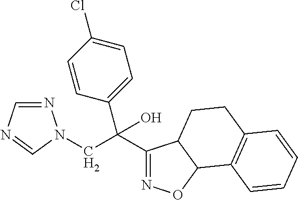 [SMILES: OC(Cn1cncn1)(C1=NOC2c3ccccc3CCC12)c1ccc(Cl)cc1] | less polar | 395 |
| 223 | 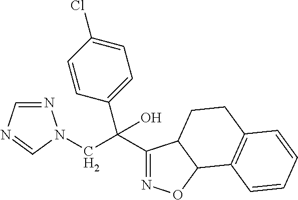 [SMILES: OC(Cn1cncn1)(C1=NOC2c3ccccc3CCC12)c1ccc(Cl)cc1] | more polar | 395 |
| 246 | 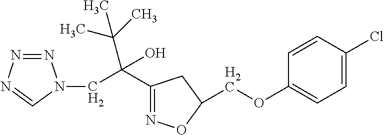 [SMILES: CC(C)(C)C(O)(Cn1cnnn1)C1=NOC(COc2ccc(Cl)cc2)C1] | less polar | 380 |
| 247 | 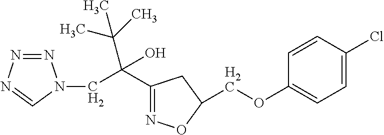 [SMILES: CC(C)(C)C(O)(Cn1cnnn1)C1=NOC(COc2ccc(Cl)cc2)C1] | more polar | 380 |
| 248 | 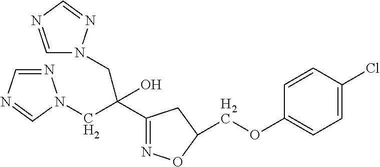 [SMILES: OC(Cn1cncn1)(Cn1cncn1)C1=NOC(COc2ccc(Cl)cc2)C1] | mixture | 404 |
| 271 | 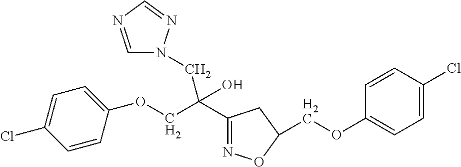 [SMILES: OC(COc1ccc(Cl)cc1)(Cn1cncn1)C1=NOC(COc2ccc(Cl)cc2)C1] | mixture | 463 |
| 272 | 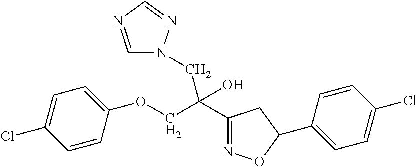 [SMILES: OC(COc1ccc(Cl)cc1)(Cn1cncn1)C1=NOC(c2ccc(Cl)cc2)C1] | less polar | 433 |
| 273 | 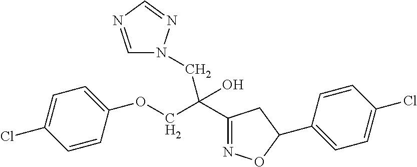 [SMILES: OC(COc1ccc(Cl)cc1)(Cn1cncn1)C1=NOC(c2ccc(Cl)cc2)C1] | more polar | 433 |

### BIOLOGICAL EXAMPLES OF THE INVENTION

General protocol for preparing test solutions for Tests A-G: the test compounds were first dissolved in acetone in an amount equal to 3% of the final volume and then suspended at the desired concentration (in ppm) in acetone and purified water (50/50 mix by volume) containing 250 ppm of the surfactant PEG400 (polyhydric alcohol esters). The resulting test suspensions were then used in Tests A-G. Compounds were sprayed at a concentration of 50 ppm (*) or 10 ppm to the point of run-off on the test plants, the equivalent of a rate of 200 g/ha or 40 g/ha, respectively.

### Test A

The test solution was sprayed to the point of run-off on grape seedlings. The following day the seedlings were inoculated with a spore suspension of Plasmopara viticola (the causal agent of grape downy mildew) and incubated in a saturated atmosphere at 20° C. for 24 h, moved to a growth chamber at 20° C. for 6 days, and then incubated in a saturated atmosphere at 20° C. for 24 h, after which time disease ratings were made.

### Test B

The test solution was sprayed to the point of run-off on tomato seedlings. The following day the seedlings were inoculated with a spore suspension of Botrytis cinerea (the causal agent of gray mold on many crops) and incubated in a saturated atmosphere at 20° C. for 48 h, and moved to a growth chamber at 27° C. for 2 days, after which time visual disease ratings were made.

### Test C

The test solution was sprayed to the point of run-off on tomato seedlings. The following day the seedlings were inoculated with a spore suspension of Phytophthora infestans (the causal agent of tomato late blight) and incubated in a saturated atmosphere at 20° C. for 24 h, and then moved to a growth chamber at 20° C. for 5 days, after which time disease ratings were visually made.

### Test D

The test solution was sprayed to the point of run-off on wheat seedlings. The following day the seedlings were inoculated with a spore suspension of Stagonospora nodorum (also known as Septoria nodorum; the causal agent of wheat glume blotch) and incubated in a saturated atmosphere at 20° C. for 48 h, and then moved to a growth chamber at 20° C. for 6 days, after which time visual disease ratings were made.

### Test E

The test solution was sprayed to the point of run-off on wheat seedlings. The following day the seedlings were inoculated with a spore suspension of Zymoseptoria tritici (the causal agent of wheat leaf blotch) and incubated in a saturated atmosphere at 24° C. for 48 h, and then moved to a growth chamber at 20° C. for 17 days, after which time visual disease ratings were made.

### Test F

The test solution was sprayed to the point of run-off on wheat seedlings. The following day the seedlings were inoculated with a spore suspension of Puccinia recondita f sp. tritici; (the causal agent of wheat leaf rust) and incubated in a saturated atmosphere at 20° C. for 24 h, and then moved to a growth chamber at 20° C. for 6 days, after which time visual disease ratings were made.

### Test G

The test solution was sprayed to the point of run-off on wheat seedlings. The following day the seedlings were inoculated with a spore dust of Erysiphe graminis f sp. tritici, (the causal agent of wheat powdery mildew) and incubated in a growth chamber at 20° C. for 8 days, after which time visual disease ratings were made.

Results for Tests A-G are given in Table A. In the Table, a rating of 100 indicates 100% disease control and a rating of 0 indicates no disease control (relative to the controls). A dash (-) indicates no test results. Compounds with an asterisks (*) were applied at a concentration of 50 ppm, otherwise test solutions were applied at 10 ppm.

TABLE A

| Compound | Test | Test | | | | | |
|---|---|---|---|---|---|---|---|
| No. | A | B | Test C | Test D | Test E | Test F | Test G |
| 1* | — | 0 | 0 | 99 | 18 | 99 | 100 |
| 2 | 33 | 0 | 0 | 0 | 0 | 100 | 99 |
| 3 | 9 | 46 | 0 | 100 | 0 | 97 | 100 |
| 4 | 0 | 9 | 0 | 100 | 0 | 98 | 99 |
| 5 | — | 0 | — | 40 | 48 | 100 | 100 |
| 6 | — | 0 | — | 0 | 0 | 0 | 91 |
| 7 | — | 99 | — | 0 | 14 | 100 | 100 |
| 8 | — | 0 | — | 0 | 0 | 0 | 100 |
| 9 | — | 0 | — | 0 | 0 | 0 | 64 |
| 10 | — | 0 | — | 0 | 0 | 19 | 13 |
| 11 | — | 81 | — | 100 | 88 | 100 | 99 |
| 12 | — | 0 | — | 68 | 57 | 99 | 96 |
| 13 | — | 0 | — | 100 | 98 | 100 | 100 |
| 14 | — | 0 | — | 88 | 4 | 100 | 99 |
| 15 | — | 100 | — | 100 | 19 | 100 | 100 |
| 16 | — | 32 | — | 100 | 8 | 100 | 99 |
| 17 | — | 51 | — | 98 | 7 | 100 | 100 |
| 18 | — | 0 | — | 87 | 6 | 100 | 99 |
| 19 | — | 0 | — | 89 | 10 | 100 | 100 |
| 20 | — | 0 | — | 65 | 98 | 100 | 99 |
| 21 | — | 9 | — | 100 | 0 | 100 | 99 |
| 22 | — | 0 | — | 96 | 29 | 100 | 99 |
| 23 | — | 0 | — | 97 | 89 | 100 | 100 |
| 24 | — | 37 | — | 99 | 0 | 100 | 100 |
| 25 | — | 0 | — | 59 | 0 | 99 | 100 |
| 26 | — | 92 | — | 70 | 0 | 74 | 89 |
| 27 | — | 51 | — | 59 | 0 | 68 | 89 |
| 28 | — | 24 | — | 39 | 0 | 0 | 99 |
| 29 | — | 0 | — | 49 | 0 | 0 | 99 |
| 30 | — | 99 | — | 0 | 0 | 79 | 100 |
| 31 | — | 0 | — | 0 | 0 | 0 | 100 |
| 32 | — | 0 | — | 0 | 5 | 98 | 99 |
| 33 | — | 0 | — | 0 | 1 | 0 | 96 |
| 34* | 34 | 0 | 0 | 99 | 0 | 53 | 100 |
| 35* | 21 | 0 | 0 | 63 | 0 | 40 | 100 |
| 36* | 11 | 0 | 0 | 53 | 0 | 17 | 99 |
| 37 | — | 0 | — | 0 | 0 | 94 | 99 |
| 38 | — | 0 | — | 0 | 1 | 55 | 72 |
| 39 | — | 0 | — | 87 | 18 | 0 | 73 |
| 40 | — | 0 | — | 51 | 0 | 55 | 48 |
| 41 | — | 0 | — | 0 | 0 | 41 | 90 |
| 42 | — | 0 | — | 42 | 0 | 9 | 0 |
| 43 | — | 31 | — | 38 | 0 | 100 | 98 |
| 44 | — | 0 | — | 28 | 2 | 0 | 64 |
| 45 | — | 70 | — | 99 | 0 | 67 | 87 |
| 46 | — | 19 | — | 76 | 0 | 85 | 73 |
| 47 | — | 19 | — | 0 | 3 | 76 | 84 |
| 48 | — | 0 | — | 94 | 0 | 99 | 99 |
| 49 | — | 11 | — | 82 | 0 | 99 | 99 |
| 50 | — | 19 | — | 87 | 3 | 99 | 94 |
| 51 | — | 16 | — | 89 | 1 | 0 | 81 |
| 52 | — | 98 | — | 100 | 4 | 100 | 100 |
| 53 | — | 70 | — | 99 | 20 | 100 | 99 |
| 54 | — | 0 | — | 87 | 0 | 100 | 99 |
| 55 | — | 0 | — | 73 | 2 | 99 | 99 |
| 56 | — | 0 | — | 20 | 0 | 97 | 94 |
| 57 | — | 0 | — | 0 | 0 | 97 | 97 |
| 58 | — | 0 | — | 0 | 0 | 0 | 68 |
| 59 | — | 0 | — | 92 | 2 | 80 | 89 |
| 60 | — | 0 | — | 73 | 3 | 28 | 90 |
| 61 | 4 | 0 | 0 | 94 | 99 | 100 | 100 |
| 62 | 3 | 0 | 0 | 0 | 0 | 92 | 96 |
| 63 | 3 | 0 | 0 | 41 | 97 | 100 | 100 |
| 64 | 12 | 0 | 0 | 50 | 2 | 80 | 94 |
| 65 | 3 | 0 | 0 | 0 | 0 | 100 | 98 |
| 66 | 3 | 0 | 0 | 0 | 1 | 98 | 97 |
| 67 | 13 | 0 | 0 | 0 | 0 | 100 | 99 |
| 68 | 3 | 0 | 0 | 0 | 2 | 99 | 95 |
| 69 | — | 27 | — | 38 | 25 | 100 | 94 |
| 70 | — | 28 | — | 47 | 0 | 9 | 76 |
| 71 | — | 0 | — | 0 | 1 | 26 | 94 |
| 72 | — | 19 | — | 0 | 0 | 0 | 56 |
| 73 | — | 25 | — | 62 | 0 | 9 | 56 |
| 74 | — | 44 | — | 0 | 13 | 0 | 56 |
| 75 | — | 0 | — | 58 | 0 | 90 | 97 |
| 76 | — | 0 | — | 0 | 0 | 0 | 89 |
| 77 | — | 0 | — | 0 | 0 | 0 | 13 |
| 78 | — | 0 | — | 0 | 0 | 0 | 0 |
| 79 | — | 97 | — | 100 | 21 | 100 | 99 |
| 80 | — | 82 | — | 100 | 1 | 100 | 99 |
| 81 | — | 57 | — | 100 | 95 | 100 | 100 |
| 82 | 1 | 33 | 0 | 99 | 0 | 98 | 100 |
| 83 | 1 | 0 | 0 | 87 | 0 | 99 | 99 |
| 84 | 11 | 94 | 0 | 73 | 0 | 9 | 95 |
| 85 | 19 | 0 | 0 | 68 | 0 | 0 | 87 |
| 86 | — | 72 | — | 99 | 1 | 74 | 98 |
| 87 | — | 83 | — | 99 | 1 | 99 | 98 |
| 88 | — | 0 | — | 99 | 76 | 100 | 99 |
| 89 | — | 0 | — | 0 | 0 | 0 | 76 |
| 90 | — | 0 | — | 59 | 0 | 0 | 64 |
| 91 | — | 0 | — | 20 | 2 | 0 | 56 |
| 92 | — | 0 | — | 10 | 3 | 0 | 13 |
| 93 | 0 | 0 | 0 | 99 | 4 | 100 | 100 |
| 94 | 0 | 0 | 0 | 98 | 0 | 100 | 99 |
| 95 | — | 100 | — | 100 | 0 | 100 | 100 |
| 96 | — | 85 | — | 100 | 5 | 100 | 99 |
| 97 | — | 0 | — | 99 | 0 | 98 | 94 |
| 98 | — | 0 | — | 0 | 0 | 88 | 55 |
| 99 | — | 0 | — | 63 | 0 | 95 | 96 |
| 100 | — | 0 | — | 77 | 0 | 79 | 92 |
| 101 | — | 0 | — | 0 | 0 | 100 | 99 |
| 102 | — | 0 | — | 0 | 0 | 18 | 69 |
| 103 | — | 0 | — | — | 0 | 94 | 92 |
| 104 | — | 0 | — | 100 | 0 | 97 | 93 |
| 105 | — | 0 | — | 96 | 8 | 100 | 99 |
| 106 | — | 0 | — | 0 | 0 | 72 | 89 |
| 107 | — | 0 | — | 32 | 58 | 100 | 99 |
| 108 | — | 0 | — | 0 | 25 | 78 | 64 |
| 109 | — | 0 | — | 59 | 66 | 100 | 93 |
| 110 | — | 0 | — | 0 | 72 | 21 | 48 |
| 111 | — | 0 | — | 0 | 9 | 100 | 98 |
| 112 | — | 0 | — | 0 | 80 | 65 | 69 |
| 113 | — | 0 | — | 0 | 9 | 100 | 98 |
| 114 | — | 0 | — | 0 | 39 | 36 | 56 |
| 115* | — | 51 | 0 | 100 | 0 | 68 | 99 |
| 116* | — | 0 | 0 | 100 | 0 | 80 | 99 |
| 117* | — | 61 | 0 | 100 | 11 | 99 | 98 |
| 118* | — | 68 | 0 | 100 | 0 | 89 | 99 |
| 119* | — | 0 | 0 | — | 0 | 41 | 100 |
| 120* | — | 0 | 0 | 95 | 8 | 100 | 98 |
| 121* | — | 100 | — | 100 | 62 | 100 | 100 |
| 122* | — | 0 | 0 | 100 | 77 | 100 | 100 |
| 123* | — | 56 | 0 | 100 | 100 | 100 | 100 |
| 124* | — | 0 | 0 | 0 | 6 | 28 | 96 |
| 125* | — | 0 | 0 | 0 | 0 | 28 | 89 |
| 126 | — | 97 | — | 100 | 89 | 100 | 99 |
| 127 | — | 0 | — | 98 | 9 | 98 | 98 |
| 128* | — | 0 | — | 100 | 95 | 100 | 99 |
| 130* | — | 14 | 0 | 100 | 11 | 100 | 100 |
| 131* | — | 0 | 0 | 100 | 5 | 100 | 100 |
| 132* | — | 99 | 0 | 100 | 81 | 100 | 100 |
| 133* | — | 95 | 0 | 100 | 88 | 100 | 99 |
| 134 | — | 0 | — | 100 | 31 | 99 | 98 |
| 135 | — | 0 | — | 69 | 1 | 98 | 81 |
| 136* | — | 92 | 0 | 100 | 83 | 100 | 100 |
| 137 | — | 0 | — | 77 | 26 | 99 | 99 |
| 138 | — | 17 | — | 100 | 0 | 100 | 100 |
| 139 | — | 0 | — | 99 | 6 | 100 | 99 |
| 140 | — | 0 | — | 82 | 67 | 100 | 99 |
| 141 | — | 0 | — | 0 | 1 | 99 | 84 |
| 142 | — | 0 | — | 86 | 91 | 100 | 99 |
| 143 | — | 0 | — | 0 | 1 | 67 | 76 |
| 144 | — | 0 | — | 99 | 17 | 100 | 99 |
| 145 | — | 0 | — | — | 28 | — | 88 |
| 146 | — | 0 | — | 100 | 99 | 100 | 99 |
| 147 | — | 0 | — | 73 | 17 | 100 | 90 |
| 148 | — | 0 | — | 59 | 92 | 100 | 100 |
| 149 | — | 0 | — | 0 | 38 | 36 | 76 |
| 150 | — | 0 | — | 100 | 42 | 100 | 99 |
| 151 | — | 0 | — | 0 | 76 | 84 | 90 |
| 152 | — | 0 | — | 0 | 34 | 50 | 87 |
| 153 | — | 0 | — | 0 | 93 | 100 | 100 |
| 154 | — | 0 | — | 0 | 1 | 52 | 83 |
| 155 | — | 0 | — | 100 | 2 | 100 | 100 |
| 156 | — | 0 | — | 98 | 19 | 100 | 100 |
| 157 | — | 0 | — | 0 | 0 | 0 | 26 |
| 158 | — | 0 | — | 96 | 3 | 100 | 100 |
| 159 | — | 0 | — | 0 | 0 | 0 | 97 |
| 160 | — | 75 | — | 99 | 11 | 100 | 100 |
| 161 | — | 0 | — | 0 | 0 | 28 | 94 |
| 162 | — | 0 | — | 96 | 76 | 100 | 100 |
| 163 | — | 0 | — | 0 | 0 | 99 | 86 |
| 164 | — | 0 | — | 95 | 96 | 100 | 100 |
| 165 | — | 0 | — | 72 | 5 | 99 | 100 |
| 166 | — | 0 | — | 0 | 0 | 41 | — |
| 167 | — | 0 | — | 0 | 0 | 98 | — |
| 168 | — | 11 | — | 0 | 3 | 0 | 35 |
| 169 | — | 0 | — | 9 | 4 | 0 | 0 |
| 170 | — | 0 | — | 99 | 68 | 100 | 99 |
| 171 | — | 0 | — | 63 | 0 | 100 | 94 |
| 172 | — | 0 | — | 100 | 4 | 100 | 99 |
| 173 | — | 0 | — | 63 | 6 | 100 | 97 |
| 174 | — | 58 | — | 100 | 12 | 100 | 100 |
| 175 | — | 0 | — | 100 | 1 | 100 | 100 |
| 176 | — | 0 | — | 82 | 2 | 100 | 100 |
| 177 | — | 0 | — | 51 | 0 | 0 | 89 |
| 178 | — | 0 | — | 99 | 91 | 100 | 100 |
| 179 | — | 0 | — | 0 | 0 | 100 | 91 |
| 180 | — | 0 | — | 99 | 1 | 100 | 100 |
| 181 | — | 0 | — | 0 | 0 | 45 | 69 |
| 182 | — | 99 | — | 99 | 96 | 100 | 100 |
| 183 | — | 24 | — | 99 | 35 | 100 | 100 |
| 184 | — | 44 | — | 100 | 94 | 100 | 100 |
| 185 | — | 0 | — | 100 | 0 | 100 | 99 |
| 186 | — | 99 | — | 100 | 66 | 100 | 100 |
| 187 | — | 50 | — | 47 | 0 | 93 | 99 |
| 188 | — | 83 | — | 100 | 3 | 100 | 100 |
| 189 | — | 0 | — | 98 | 48 | 100 | 100 |
| 190 | — | 0 | — | 100 | 2 | 100 | 99 |
| 191 | — | 0 | — | 100 | 2 | 100 | 100 |
| 192 | — | 0 | — | 100 | 23 | 100 | 100 |
| 193 | — | 9 | — | 93 | 22 | 99 | 97 |
| 194 | — | 0 | — | 11 | 0 | 100 | 99 |
| 195 | — | 0 | — | 0 | 0 | 99 | 100 |
| 196 | — | 0 | — | 99 | 12 | 100 | 100 |
| 197 | — | 11 | — | 92 | 0 | 100 | 98 |
| 198 | — | 0 | — | 81 | 0 | 100 | 100 |
| 199 | — | 0 | — | 86 | 0 | 100 | 100 |
| 200 | — | 0 | — | 11 | 16 | 99 | 99 |
| 201 | — | 0 | — | 50 | 1 | 100 | 96 |
| 202 | — | 0 | — | 59 | 21 | 100 | 99 |
| 203 | — | 0 | — | 55 | 16 | 100 | 99 |
| 204 | — | — | — | 77 | 0 | 100 | 99 |
| 205 | — | — | — | 10 | 6 | 68 | 98 |
| 206 | — | 9 | — | 86 | 13 | 100 | 99 |
| 207 | — | 0 | — | 99 | 44 | 100 | 100 |
| 208 | — | — | — | 100 | 74 | 100 | 100 |
| 209 | — | — | — | 99 | 11 | 100 | 100 |
| 210* | — | 0 | 0 | 100 | 41 | 100 | 100 |
| 211* | — | 0 | 0 | 0 | 0 | 28 | 76 |
| 212* | — | 28 | 0 | 96 | 0 | 100 | 100 |
| 213* | — | 7 | 0 | 52 | 0 | 99 | 96 |
| 214* | — | 0 | 0 | 0 | 1 | 25 | 99 |
| 215* | — | 0 | 0 | 0 | 0 | 100 | 100 |
| 216 | — | 0 | — | 86 | 0 | 100 | — |
| 217 | — | 0 | — | 92 | 0 | 100 | — |
| 218 | — | 0 | — | 21 | 0 | 100 | — |
| 219 | — | 0 | — | 0 | 0 | 95 | — |
| 220* | — | 21 | 0 | 0 | 1 | 98 | 13 |
| 221* | — | 22 | 0 | 0 | 1 | 27 | 27 |
| 222 | — | 9 | — | 50 | 23 | 100 | 99 |
| 223 | — | 0 | — | 0 | 63 | 100 | 99 |
| 224 | — | — | — | — | 30 | 100 | 100 |
| 225 | — | — | — | — | 0 | 100 | 99 |
| 226 | — | 0 | — | 51 | 0 | 100 | 99 |
| 227 | — | 0 | — | 73 | 29 | 100 | 100 |
| 228 | — | — | — | 0 | 12 | 86 | 99 |
| 229 | — | — | — | 0 | 6 | 0 | 98 |
| 230 | — | 99 | — | 100 | 8 | 100 | 99 |
| 231 | — | 80 | — | 100 | 6 | 100 | 99 |
| 232 | — | 98 | — | 99 | 41 | 100 | 100 |
| 233 | — | 40 | — | 100 | 0 | 100 | 99 |
| 234 | — | — | — | — | 0 | 0 | 99 |
| 235 | — | — | — | — | 8 | 87 | 99 |
| 236 | — | — | — | 99 | 16 | 100 | 100 |
| 237 | — | — | — | 89 | 0 | 100 | 98 |
| 238 | — | — | — | 99 | 0 | 100 | 99 |
| 239 | — | — | — | 98 | 0 | 100 | 99 |
| 240 | — | — | — | — | 0 | 100 | 99 |
| 241 | — | — | — | — | 0 | 99 | 90 |
| 242 | — | — | — | 0 | 0 | 99 | 68 |
| 243 | — | — | — | 0 | 0 | 99 | 0 |
| 244 | — | — | — | 99 | 0 | 100 | 99 |
| 245 | — | — | — | 51 | 2 | 98 | 98 |
| 246 | — | — | — | 98 | 0 | 100 | 95 |
| 247 | — | — | — | 0 | 24 | 97 | 0 |
| 248 | — | — | — | 0 | 0 | 100 | 98 |
| 249 | — | — | — | 100 | 0 | 100 | 100 |
| 250 | — | — | — | 64 | 0 | 99 | 99 |
| 251 | — | — | — | 100 | 3 | 100 | 100 |
| 252 | — | — | — | 78 | 2 | 100 | 89 |
| 253 | — | — | — | 99 | 3 | 100 | 100 |
| 254 | — | — | — | 99 | 0 | 100 | 99 |
| 255 | — | — | — | 100 | 1 | 100 | 100 |
| 256 | — | — | — | 97 | 0 | 100 | 99 |
| 257 | — | — | — | 100 | 29 | 100 | 100 |
| 258 | — | — | — | 100 | 0 | 100 | 100 |
| 259 | — | — | — | 99 | 0 | 92 | 99 |
| 260 | — | — | — | 99 | 1 | 98 | 100 |
| 261 | — | — | — | 100 | 92 | 100 | 100 |
| 262 | — | — | — | 0 | 41 | 99 | 98 |
| 263 | — | — | — | 99 | 0 | 87 | 99 |
| 264 | — | — | — | 0 | 0 | 0 | 99 |
| 265 | — | — | — | 99 | 1 | 100 | 100 |
| 266 | — | — | — | 100 | 0 | 100 | 100 |
| 267 | — | — | — | 100 | 3 | 100 | 100 |
| 268 | — | — | — | 82 | 0 | 100 | 95 |
| 269 | — | — | — | 99 | 73 | 100 | 100 |
| 270 | — | — | — | 99 | 75 | 100 | 100 |
| 271 | — | — | — | 42 | 2 | 100 | 92 |
| 272 | — | — | — | 69 | 0 | 100 | 99 |
| 273 | — | — | — | 11 | 2 | 98 | 69 |

What is claimed is:

1. A compound selected from Formula 1, N-oxides and salts thereof, 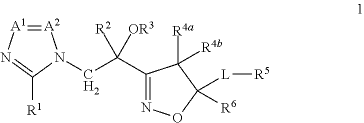
wherein
A¹ is CH or N;
A² is CH or N;
R¹ is hydrogen, halogen, SH, CN, SCN, C₁-C₆ alkylthio, C₁-C₆ haloalkylthio, C₂-C₆ alkenylthio, C₂-C₆ haloalkenylthio, C₂-C₆ alkynylthio or C₂-C₆ haloalkynylthio;
R² is C₁-C₆ alkyl, C₂-C₆ alkenyl or C₂-C₆ alkynyl, each optionally substituted with up to 5 substituents independently selected from R^{2a}; or C₃-C₈ cycloalkyl optionally substituted with up to 5 substituents independently selected from R^{2b}; or ZQ¹;
each R^{2a} is independently hydroxy, halogen, cyano, nitro, C₁-C₆ alkoxy, C₁-C₆ haloalkoxy, C₃-C₆ cycloalkyl or C₃-C₆ halocycloalkyl;
each R^{2b} is independently hydroxy, halogen, cyano, nitro, C₁-C₆ alkyl, C₁-C₆ haloalkyl, C₁-C₆ alkoxy or C₁-C₆ haloalkoxy;
Z is a direct bond, CH₂ or CH₂O wherein the carbon atom is attached to the remainder of Formula 1 and the oxygen atom is attached to Q¹;
Q¹ is a phenyl ring; or a 5- or 6-membered heteroaromatic ring containing ring members selected from carbon atoms and up to 4 heteroatoms independently selected from up to 2 O, up to 2 S and up to 4 N atoms, each ring optionally substituted with up to 3 substituents independently selected from R^{9a} on carbon atom ring members and R^{9b} on nitrogen atom ring members;
R³ is hydrogen, CHO, C₂-C₄ alkylcarbonyl, C₂-C₄ haloalkylcarbonyl, C₂-C₄ alkoxycarbonyl, C₂-C₄ haloalkoxycarbonyl, C₁-C₄ alkyl, C₁-C₄ haloalkyl, C₃-C₄ alkenyl, C₃-C₄ alkynyl or C₂-C₄ alkoxyalkyl;
R^{4a} is hydrogen, halogen, cyano, C₁-C₂ alkyl, C₁-C₂ haloalkyl or C₁-C₂ alkoxy;
R^{4b} is hydrogen, halogen, cyano, C₁-C₂ alkyl, C₁-C₂ haloalkyl or C₁-C₂ alkoxy;
L is a direct bond; or a 1-, 2-, 3- or 4-membered saturated, partially unsaturated or fully unsaturated chain containing chain members selected from carbon atoms and up to 2 heteroatoms independently selected from up to 1 O, up to 1 S, and up to 2 N, wherein the chain is optionally substituted with up to 4 substituents independently selected from R^{7a} on carbon atom chain members and R^{7b} on nitrogen atom chain members;
R⁵ is C₁-C₈ alkyl, C₁-C₈ haloalkyl, C₂-C₈ alkenyl, C₂-C₈ haloalkenyl, C₂-C₈ alkynyl, C₂-C₈ haloalkynyl, C₃-C₈ cycloalkyl, C₃-C₈ halocycloalkyl, C₄-C₁₀ alkylcycloalkyl, C₄-C₁₀ cycloalkylalkyl, C₄-C₁₀ halocycloalkylalkyl, C₅-C₁₀ alkylcycloalkylalkyl, C₂-C₈ alkoxyalkyl, C₂-C₈ haloalkoxyalkyl, C₄-C₁₀ cycloalkoxyalkyl, C₃-C₈ alkoxyalkoxyalkyl, C₂-C₈ alkylthioalkyl, C₂-C₈ haloalkylthioalkyl, C₂-C₈ alkylsulfinylalkyl, C₂-C₈ alkylsulfonylalkyl, C₂-C₈ alkylaminoalkyl, C₂-C₈ haloalkylaminoalkyl, C₃-C₈ dialkylaminoalkyl, C₄-C₁₀ cycloalkylaminoalkyl, C₃-C₈ alkoxycarbonylalkyl, C₃-C₈ haloalkoxycarbonylalkyl, C₁-C₈ alkoxy, C₁-C₈ haloalkoxy, C₂-C₈ alkenyloxy, C₂-C₈ haloalkenyloxy, C₃-C₈ alkynyloxy, C₃-C₈ haloalkynyloxy, C₃-C₈ cycloalkoxy, C₃-C₈ halocycloalkoxy, C₄-C₁₀ cycloalkylalkoxy, C₂-C₈ alkoxyalkoxy, C₂-C₈ alkylcarbonyloxy, C₂-C₈ haloalkylcarbonyloxy, C₁-C₈ alkylthio, C₁-C₈ haloalkylthio, C₃-C₈ cycloalkylthio, C₁-C₈ alkylamino, C₁-C₈ haloalkylamino, C₂-C₈ dialkylamino, C₂-C₈ halodialkylamino, C₃-C₈ cycloalkylamino, C₂-C₈ alkylcarbonylamino, C₂-C₈ haloalkylcarbonylamino, C₁-C₈ alkylsulfonylamino, C₁-C₈ haloalkylsulfonylamino or C₃-C₈ trialkylsilyl; or G;
G is phenyl or naphthalenyl, each optionally substituted with up to 5 substituents independently selected from R^{8a}; or a 5- or 6-membered heteroaromatic ring containing ring members selected from carbon atoms and up to 4 heteroatoms independently selected from up to 2 O, up to 2 S and up to 4 N atoms, each ring optionally substituted with up to 5 substituents independently selected from R^{8a}on carbon atom ring members and R^{8b} on nitrogen atom ring members;
R⁶ is hydrogen, halogen, cyano, C₁-C₂ alkyl or C₁-C₂ haloalkyl; or
R⁶ and R^{4a} are taken together with the linking atoms to which they are attached to form a 3- to 7-membered ring containing ring members, in addition to the linking atoms, selected from carbon atoms and up to 3 heteroatoms independently selected from up to 1 O, up to 1 S and up to 1 N atom, the ring optionally substituted with up to 2 substituents independently selected from halogen, C₁-C₂ alkyl, C₁-C₂ haloalkyl, C₁-C₂ alkoxy and C₁-C₂ haloalkoxy on carbon atom ring members and cyano, C₁-C₂ alkyl and C₁-C₂ alkoxy on nitrogen atom ring members;
each R^{7a} is independently cyano, halogen, hydroxy, C₁-C₃ alkyl, C₁-C₃ haloalkyl, C₁-C₃ alkoxy or C₁-C₃ haloalkoxy;
each R^{7b} is independently cyano, C₁-C₃ alkyl, C₁-C₃ haloalkyl, C₁-C₃ alkoxy or C₂-C₃ alkylcarbonyl;
each R^{8a} is independently halogen, cyano, hydroxy, amino, nitro, C₁-C₄ alkyl, C₂-C₄ alkenyl, C₂-C₄ alkynyl, C₃-C₆ cycloalkyl, C₄-C₈ alkylcycloalkyl, C₄-C₈ cycloalkylalkyl, C₁-C₄ haloalkyl, C₂-C₄ haloalkenyl, C₂-C₄ haloalkynyl, C₃-C₆ halocycloalkyl, C₂-C₄ alkoxyalkyl, C₂-C₄ hydroxyalkyl, C₁-C₄ alkoxy, C₁-C₄ haloalkoxy, C₂-C₄ alkylcarbonyloxy, C₁-C₄ alkylthio, C₁-C₄ haloalkylthio, C₂-C₄ alkylcarbonylthio, C₁-C₄ alkylsulfinyl, C₁-C₄ haloalkylsulfinyl, C₁-C₄ alkylsulfonyl, C₁-C₄ haloalkylsulfonyl, C₁-C₄ alkylamino, C₂-C₆ dialkylamino, C₃-C₆ cycloalkylamino, C₂-C₄ alkylcarbonyl, C₂-C₄ alkoxycarbonyl, C₂-C₄ alkylaminocarbonyl, C₃-C₆ dialkylaminocarbonyl, C₃-C₆ trialkylsilyl or Q²;
each R^{8b} is independently cyano, C₁-C₄ alkyl, C₁-C₄ haloalkyl, C₁-C₄ alkoxy, C₂-C₄ alkylcarbonyl, C₂-C₄ alkoxycarbonyl or C₃-C₆ cycloalkyl;
each Q² is independently a phenyl, benzyloxy, phenoxy, benzylthio, phenylthio or a 5- or 6-membered heteroaromatic ring each optionally substituted with up to 3 substituents independently selected from the group consisting of hydroxy, halogen, cyano, nitro, C₁-C₆ alkyl, C₁-C₆ haloalkyl, C₁-C₆ alkoxy and C₁-C₆ haloalkoxy;
each R^{9a} is independently hydroxy, halogen, cyano, nitro, C₁-C₆ alkyl, C₁-C₆ haloalkyl, C₁-C₆ alkoxy, C₁-C₆ haloalkoxy or phenoxy; and
each R^{9b} is independently cyano, C₁-C₆ alkyl, C₁-C₆ haloalkyl, C₁-C₆ alkoxy or C₂-C₆ alkylcarbonyl;
provided that
a) when A¹ is N then A² is N;
b) L is other than —O—, —S— or —NR^{7b}—;
c) when Z is a direct bond, then Q¹ is bonded to the remainder of Formula 1 via a carbon atom;
d) when L is a direct bond, then R⁵ is bonded to the remainder of Formula 1 via a carbon atom; and
e) when Z is CH₂, then Q¹ is other than a 6-membered heteroaromatic ring.

2. A compound of claim 1 wherein:
A¹ is CH and A² is CH or A¹ is CH and A² is N;
R¹ is hydrogen, SH, SCN, C₁-C₆ alkylthio or C₂-C₆ alkenylthio;
R² is C₁-C₆ alkyl optionally substituted with up to 5 substituents independently selected from R^{2a}; or C₃-C₈ cycloalkyl optionally substituted with up to 5 substituents independently selected from R^{2b}; or ZQ¹;
R³ is hydrogen, CHO, C₂-C₄ alkylcarbonyl, C₂-C₄ alkoxycarbonyl, C₁-C₄ alkyl, or C₂-C₄ alkoxyalkyl;
R^{4a} is hydrogen, halogen or C₁-C₂ alkyl;
R^{4b} is hydrogen, halogen or C₁-C₂ alkyl;
L is a direct bond; or a 1-, 2- or 3-membered saturated or partially unsaturated chain containing chain members selected from carbon atoms and up to 2 heteroatoms independently selected from up to 1 O, up to 1 S, and up to 2 N, wherein the chain is optionally substituted with up to 3 substituents independently selected from R^{7a} on carbon atom chain members and R^{7b} on nitrogen atom chain members;
R⁵ is C₁-C₈ alkyl, C₁-C₈ haloalkyl, C₃-C₈ cycloalkyl, C₃-C₈ halocycloalkyl, C₄-C₁₀ alkylcycloalkyl, C₂-C₈ alkoxyalkyl, C₂-C₈ haloalkoxyalkyl, C₁-C₈ alkoxy, C₁-C₈ haloalkoxy, C₃-C₈ cycloalkoxy, C₃-C₈ halocycloalkoxy, C₁-C₈ alkylthio, C₁-C₈ haloalkylthio, C₁-C₈ alkylamino, C₁-C₈ haloalkylamino, C₂-C₈ dialkylamino, C₂-C₈ halodialkylamino, or C₃-C₈ trialkylsilyl; or G; and
R⁶ is hydrogen, C₁-C₂ alkyl or C₁-C₂ haloalkyl; or R⁶ and R^{4a} are taken together with the linking atoms to which they are attached to form a 3- to 6-membered ring containing ring members, in addition to the linking atoms, selected from carbon atoms and up to 2 heteroatoms independently selected from up to 1 O, up to 1 S and up to 1 N atom, the ring optionally substituted with up to 3 substituents independently selected from halogen, cyano, C₁-C₂ alkyl, C₁-C₂ haloalkyl, on carbon atom ring members and C₁-C₂ alkyl on nitrogen atom ring members.

3. A compound of claim 2 wherein:
A¹ is CH and A² is N;
R¹ is hydrogen, SH, SCN, SCH₃ or SCH₂CH═CH₂;
R² is C₁-C₆ alkyl optionally substituted with up to 3 substituents independently selected from R^{2a}; or C₃-C₆ cycloalkyl optionally substituted with up to 3 substituents independently selected from R^{2b}; or ZQ¹;
each R^{2a} is independently halogen, cyano, C₁-C₆ alkoxy, C₁-C₆ haloalkoxy, C₃-C₆ cycloalkyl, phenyl or phenoxy;
each R^{2b} is independently halogen, cyano, C₁-C₆ alkyl, C₁-C₆ haloalkyl, C₁-C₆ alkoxy or C₁-C₆ haloalkoxy;
Z is a direct bond or CH₂;
Q¹ is selected from Q-1 through Q-65 depicted in Exhibit 1 wherein when R⁹ is attached to a carbon ring member, said R⁹ is selected from R^{9a}, and when R⁹ is attached to a nitrogen ring member, said R⁹ is selected from R^{9b}; and k is 0, 1, 2 or 3,
R³ is hydrogen or C₁-C₄ alkyl;
R^{4a} is hydrogen;
R^{4b} is hydrogen;
L is a direct bond; or a 1- or 2-membered saturated chain containing chain members selected from carbon atoms and up to 1 heteroatom selected from up to 1 O, up to 1 S, and up to 1 N, wherein the chain is optionally substituted with up to 2 substituents independently selected from R^{7a} on carbon atom chain members and R^{7b} on nitrogen atom chain members;
each R^{7a} is independently halogen, C₁-C₂ alkyl or C₁-C₂ haloalkyl;
each R^{7b} is independently C₁-C₂ alkyl, C₁-C₂ haloalkyl or C₂-C₃ alkylcarbonyl;
R⁵ is C₁-C₈ alkyl, C₁-C₈ haloalkyl, C₃-C₈ cycloalkyl, C₃-C₈ halocycloalkyl, C₁-C₈ alkoxy or C₁-C₈ haloalkoxy; or G;
G is selected from G-1 through G-65 depicted in Exhibit 2 wherein when R⁸ is attached to a carbon ring member, said R⁸ is selected from R^{8a}, and when R⁸ is attached to a nitrogen ring member, said R⁸ is selected from R^{8b}; and m is 0, 1, 2 or 3,
each R^{8a} is independently halogen, cyano, C₁-C₄ alkyl, C₂-C₄ alkenyl, C₂-C₄ alkynyl, C₁-C₄ haloalkyl, C₂-C₄ haloalkenyl, C₂-C₄ haloalkynyl, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalkylsulfinyl, C₁-C₄ alkylsulfonyl, C₁-C₄ haloalkylsulfonyl, C₁-C₄ alkylamino, C₂-C₆ dialkylamino, C₂-C₄ alkylcarbonyl, C₂-C₄ alkoxycarbonyl, C₃-C₆ trialkylsilyl; or Q²; and
R⁶ is hydrogen, C₁-C₂ alkyl or C₁-C₂ haloalkyl.

4. A compound of claim 3 wherein:
R¹ is hydrogen or SH;
Q¹ is selected from Q-1 through Q-3, Q-15, Q-25, Q-35, Q-50 and Q-54;
R³ is hydrogen or CH₃;
L is a direct bond; or —CH₂—, —CH₂O—, —CH₂S—, —CH₂NR^{7b}— or —CH₂CH₂— wherein the left bond is connected to the isoxazoline ring and the right bond is connected to R⁵;
R⁵ is G;
G is selected from G-1, G-2, G-3, G-12, G-13, G-14, G-25 and G-53;
each R^{8a} is independently halogen, cyano, C₁-C₄ alkyl, C₁-C₄ haloalkyl, C₁-C₄ alkoxy, C₁-C₄ haloalkoxy, C₁-C₄ alkylthio, C₁-C₄ haloalkylthio, C₃-C₆ trialkylsilyl; or Q²;
each Q² is independently a phenyl, benzyloxy or phenoxy;
each R^{8b} is independently C₁-C₄ alkyl; and
R⁶ is hydrogen or CH₃.

5. A compound of claim 4 wherein:
R¹ is hydrogen;
R² is C₄-C₆ alkyl; C₃-C₆ cycloalkyl; or ZQ¹ wherein Z is a direct bond or CH₂ and Q¹ is selected from Q-1, Q-15, Q-25, Q-35, Q-50 and Q-54;
R³ is hydrogen;
L is —CH₂—, —CH₂O—, —CH₂S— or —CH₂CH₂— wherein the left bond is connected to the isoxazoline ring and the right bond is connected to R⁵;
R⁵ is G-1;
each R^{8a} is independently halogen or CF₃;
R⁶ is hydrogen; and
R^{9a} is independently halogen, C₁-C₂ alkyl or C₁-C₂ haloalkyl.

6. A compound of claim 1 that is selected from the group consisting of:
α-[5-[(4-bromophenoxy)methyl]-4,5-dihydro-3-isoxazolyl]-α-(1,1-dimethylpropyl)-1H-1,2,4-triazole-1-ethanol,
α-[4,5-dihydro-5-[2-[4-(trifluoromethyl)phenyl]ethyl]-3-isoxazolyl]-α-(1,1-dimethylethyl)-1H-1,2,4-triazole-1-ethanol,
α-[5-[(4-chlorophenoxy)methyl]-4,5-dihydro-3-isoxazolyl]-α-(4-chlorophenyl)-1H-1,2,4-triazole-1-ethanol,
α-[4,5-dihydro-5-[[4-(trifluoromethyl)phenoxy]methyl]-3-isoxazolyl]-α-(1,1-dimethylethyl)-1H-1,2,4-triazole-1-ethanol,
α-(4-chlorophenyl)-α-[5-(4-chlorophenyl)-4,5-dihydro-3-isoxazolyl]-1H-1,2,4-triazole-1-ethanol,
α-[5-(4-chlorophenyl)-4,5-dihydro-3-isoxazolyl]-α-(1-methylcyclopropyl)-1H-1,2,4-triazole-1-ethanol,
α-(4-chlorophenyl)-α-[4,5-dihydro-5-(4-phenoxyphenyl)-3-isoxazolyl]-1H-1,2,4-triazole-1-ethanol,
α-(4-chlorophenyl)-α-[4,5-dihydro-5-[4-(trifluoromethyl)phenyl]-3-isoxazolyl]-1H-1,2,4-triazole-1-ethanol,
α-(4-chlorophenyl)-α-[5-[[(5-chloro-2-pyridinyl)oxy]methyl]-4,5-dihydro-3-isoxazolyl]-1H-1,2,4-triazole-1-ethanol,
α-[5-[(4-chlorophenoxy)methyl]-4,5-dihydro-3-isoxazolyl]-α-(1,1-dimethylethyl)-1H-1,2,4-triazole-1-ethanol and
α-[5-[(4-bromophenoxy)methyl]-4,5-dihydro-3-isoxazolyl]-α-(1,1-dimethylethyl)-1H-1,2,4-triazole-1-ethanol.

7. A fungicidal composition comprising (a) a compound of claim 1; and (b) at least one other fungicide.

8. A fungicidal composition comprising (a) a compound of claim 1; and (b) at least one additional component selected from the group consisting of surfactants, solid diluents and liquid diluents.

9. A method for controlling plant diseases caused by fungal plant pathogens comprising applying to the plant or portion thereof, or to the plant seed, a fungicidally effective amount of a compound of claim 1.
